SEQUENCE LISTING (418 sequences)

SEQ ID NO: 1 - DNA, 3924 bp, Acidaminococcus sp. BV3L6
  source 1..3924: mol_type = genomic DNA
atgacccaatttgaaggttttaccaatttataccaagtttcgaagacccttcgttttgaactgattccccaaggaaaaacactcaaacatatccaggagcaagggttcattgaggaggataaagctcgcaatgaccattacaaagagttaaaaccaatcattgaccgcatctataagacttatgctgatcaatgtctccaactggtacagcttgactgggagaatctatctgcagccatagactcctatcgtaaggaaaaaaccgaagaaacacgaaatgcgctgattgaggagcaagcaacatatagaaatgcgattcatgactactttataggtcggacggataatctgacagatgccataaataagcgccatgctgaaatctataaaggactttttaaagctgaacttttcaatggaaaagttttaaagcaattagggaccgtaaccacgacagaacatgaaaatgctctactccgttcgtttgacaaatttacgacctatttttccggcttttatgaaaaccgaaaaaatgtctttagcgctgaagatatcagcacggcaattccccatcgaatcgtccaggacaatttccctaaatttaaggaaaactgccatatttttacaagattgataaccgcagttccttctttgcgggagcattttgaaaatgtcaaaaaggccattggaatctttgttagtacgtctattgaagaagtcttttcctttcccttttataatcaacttctaacccaaacgcaaattgatctttataatcaacttctcggcggcatatctagggaagcaggcacagaaaaaatcaagggacttaatgaagttctcaatctggctatccaaaaaaatgatgaaacagcccatataatcgcgtccctgccgcatcgttttattcctctttttaaacaaattctttccgatcgaaatacgttatcctttattttggaagaattcaaaagcgatgaggaagtcatccaatccttctgcaaatataaaaccctcttgagaaacgaaaatgtactggagactgcagaagcccttttcaatgaattaaattccattgatttgactcatatctttatttcccataaaaagttagaaaccatctcttcagcgctttgtgaccattgggataccttgcgcaatgcactttacgaaagacggatttctgaactcactggcaaaataacaaaaagtgccaaagaaaaagttcaaaggtcattaaaacatgaggatataaatctccaagaaattatttctgctgcaggaaaagaactatcagaagcattcaaacaaaaaacaagtgaaattctttcccatgcccatgctgcacttgaccagcctcttcccacaacattaaaaaaacaggaagaaaaagaaatcctcaaatcacagctcgattcgcttttaggcctttatcatcttcttgattggtttgctgtcgatgaaagcaatgaagtcgacccagaattctcagcacggctgacaggcattaaactagaaatggaaccaagcctttcgttttataataaagcaagaaattatgcgacaaaaaagccctattcggtggaaaaatttaaattgaattttcaaatgccaacccttgcctctggttgggatgtcaataaagaaaaaaataatggagctattttattcgtaaaaaatggtctctattaccttggtatcatgcctaaacagaaggggcgctataaagccctgtcttttgagccgacagaaaaaacatcagaaggattcgataagatgtactatgactacttcccagatgccgcaaaaatgattcctaagtgttccactcagctaaaggctgtaaccgctcattttcaaactcataccacccccattcttctctcaaataatttcattgaacctcttgaaatcacaaaagaaatttatgacctgaacaatcctgaaaaggagcctaaaaagtttcaaacggcttatgcaaagaagacaggcgatcaaaaaggctatagagaagcgctttgcaaatggattgactttacgcgggattttctctctaaatatacgaaaacaacttcaatcgatttatcttcactccgcccttcttcgcaatataaagatttaggggaatattacgccgaactgaatccgcttctctatcatatctccttccaacgaattgctgaaaaggaaatcatggatgctgtagaaacgggaaaattgtatctgttccaaatctacaataaggattttgcgaagggccatcacgggaaaccaaatctccacaccctgtattggacaggtctcttcagtcctgaaaaccttgcgaaaaccagcatcaaacttaatggtcaagcagaattgttctatcgacctaaaagccgcatgaagcggatggcccatcgtcttggggaaaaaatgctgaacaaaaaactaaaggaccagaagacaccgattccagataccctctaccaagaactgtacgattatgtcaaccaccggctaagccatgatctttccgatgaagcaagggccctgcttccaaatgttatcaccaaagaagtctcccatgaaattataaaggatcggcggtttacttccgataaatttttcttccatgttcccattacactgaattatcaagcagccaatagtcccagtaaattcaaccagcgtgtcaatgcctaccttaaggagcatccggaaacgcccatcattggtatcgatcgtggagaacgcaatctaatctatattaccgtcattgacagtactgggaaaattttggagcagcgttccctgaataccatccagcaatttgactaccaaaaaaaattggacaacagggaaaaagagcgtgttgccgcccgtcaagcctggtccgtcgtcggaacgatcaaagaccttaaacaaggctacttgtcacaggtcatccatgaaattgtagacctgatgattcattaccaagctgttgtcgtccttgaaaacctcaacttcggatttaaatcaaaacggacaggcattgccgaaaaagcagtctaccaacaatttgaaaagatgctaatagataaactcaactgtttggttctcaaagattatcctgctgagaaagtgggaggcgtcttaaacccgtatcaacttacagatcagttcacgagctttgcaaaaatgggcacgcaaagcggcttccttttctatgtaccggccccttatacctcaaagattgatcccctgactggttttgtcgatccctttgtatggaagaccattaaaaatcatgaaagtcggaagcatttcctagaaggatttgatttcctgcattatgatgtcaaaacaggtgattttatcctccattttaaaatgaatcggaatctctctttccagagagggcttcctggcttcatgccagcttgggatattgttttcgaaaagaatgaaacccaatttgatgcaaaagggacgcccttcattgcaggaaaacgaattgttcctgtaatcgaaaatcatcgttttacgggtcgttacagagacctctatcccgctaatgaactcattgcccttctggaagaaaaaggcattgtctttagagacggaagtaatatattacccaaacttttagaaaatgatgattctcatgcaattgatacgatggtcgccttgattcgcagtgtactccaaatgagaaacagcaatgccgcaacgggggaagactacatcaactctcccgttagggatctgaacggggtgtgtttcgacagtcgattccaaaatccagaatggccaatggatgcggatgccaacggagcttatcatattgccttaaaagggcagcttcttctgaaccacctcaaagaaagcaaagatctgaaattacaaaacggcatcagcaaccaagattggctggcctacattcaggaactgagaaactga

SEQ ID NO: 2 - protein, 1307 aa, Acidaminococcus sp. BV3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - DNA, 3714 bp, Lachnospiraceae bacterium ND2006
  source 1..3714: mol_type = genomic DNA
atgagcaaactggaaaaatttacgaattgttatagcctgtccaagaccctgcgtttcaaagccatccccgttggcaaaacccaggagaatattgataataaacgtctgctggttgaggatgaaaaaagagcagaagactataagggagtcaaaaaactgctggatcggtactacctgagctttataaatgacgtgctgcatagcattaaactgaaaaatctgaataactatattagtctgttccgcaagaaaacccgaacagagaaagaaaataaagagctggaaaacctggagatcaatctgcgtaaagagatcgcaaaagcttttaaaggaaatgaaggttataaaagcctgttcaaaaaagacattattgaaaccatcctgccggaatttctggatgataaagacgagatagcgctcgtgaacagcttcaacgggttcacgaccgccttcacgggctttttcgataacagggaaaatatgttttcagaggaagccaaaagcacctcgatagcgttccgttgcattaatgaaaatttgacaagatatatcagcaacatggatattttcgagaaagttgatgcgatctttgacaaacatgaagtgcaggagattaaggaaaaaattctgaacagcgattatgatgttgaggattttttcgagggggaattttttaactttgtactgacacaggaaggtatagatgtgtataatgctattatcggcgggttcgttaccgaatccggcgagaaaattaagggtctgaatgagtacatcaatctgtataaccaaaagaccaaacagaaactgccaaaattcaaaccgctgtacaagcaagtcctgagcgatcgggaaagcttgagcttttacggtgaaggttataccagcgacgaggaggtactggaggtctttcgcaataccctgaacaagaacagcgaaattttcagctccattaaaaagctggagaaactgtttaagaattttgacgagtacagcagcgcaggtatttttgtgaagaacggacctgccataagcaccattagcaaggatatttttggagagtggaatgttatccgtgataaatggaacgcggaatatgatgacatacacctgaaaaagaaggctgtggtaactgagaaatatgaagacgatcgccgcaaaagctttaaaaaaatcggcagctttagcctggagcagctgcaggaatatgcggacgccgacctgagcgtggtcgagaaactgaaggaaattattatccaaaaagtggatgagatttacaaggtatatggtagcagcgaaaaactgtttgatgcggacttcgttctggaaaaaagcctgaaaaaaaatgatgctgttgttgcgatcatgaaagacctgctcgatagcgttaagagctttgaaaattacattaaagcattctttggcgagggcaaagaaacaaacagagacgaaagcttttatggcgacttcgtcctggcttatgacatcctgttgaaggtagatcatatatatgatgcaattcgtaattacgtaacccaaaagccgtacagcaaagataagttcaaactgtatttccagaacccgcagtttatgggtggctgggacaaagacaaggagacagactatcgcgccactattctgcgttacggcagcaagtactatctcgccatcatggacaaaaaatatgcaaagtgtctgcagaaaatcgataaagacgacgtgaacggaaattacgaaaagattaattataagctgctgccagggcccaacaagatgttaccgaaagtatttttttccaaaaaatggatggcatactataacccgagcgaggatatacagaagatttacaaaaatgggaccttcaaaaagggggatatgttcaatctgaatgactgccacaaactgatcgatttttttaaagatagcatcagccgttatcctaaatggtcaaacgcgtatgattttaatttctccgaaacggagaaatataaagacattgctggtttctatcgcgaagtcgaagaacagggttataaagttagctttgaatcggccagcaagaaagaggttgataaactggtggaggagggtaagctgtatatgtttcagatttataacaaagactttagcgacaaaagccacggtactcctaatctgcatacgatgtactttaaactgctgtttgatgagaataaccacggccaaatccgtctctccggtggagcagaactttttatgcggcgtgcgagcctaaaaaaggaagaactggtggtgcatcccgccaacagcccgattgctaacaaaaatccagataatcctaagaagaccaccacactgtcgtacgatgtctataaggataaacgtttctcggaagaccagtatgaattgcatataccgatagcaattaataaatgcccaaaaaacattttcaaaatcaacactgaagttcgtgtgctgctgaaacatgatgataatccgtatgtgatcggaattgaccgtggggagagaaatctgctgtatattgtagtcgttgatggcaagggcaacatcgttgagcagtatagcctgaatgaaataattaataattttaacggtatacgtattaaaaccgactatcatagcctgctggataaaaaggagaaagagcgttttgaggcacgccaaaattggacgagcatcgaaaacatcaaggaactgaaggcaggatatatcagccaagtagtccataaaatctgtgaactggtggagaagtacgacgctgtcattgccctggaagacctcaatagcggctttaaaaacagccgggtgaaggtggagaaacaggtataccaaaagtttgaaaagatgctcattgataagctgaactatatggttgataaaaagagcaacccgtgcgccactggcggtgcactgaaagggtaccaaattaccaataaatttgaaagctttaaaagcatgagcacgcagaatgggtttattttttatataccagcatggctgacgagcaagattgaccccagcactggttttgtcaatctgctgaaaaccaaatacacaagcattgcggatagcaaaaaatttatttcgagcttcgaccgtattatgtatgttccggaggaagatctgtttgaatttgccctggattataaaaacttcagccgcaccgatgcagattatatcaaaaaatggaagctgtacagttatggtaatcgtatacgtatcttccgtaatccgaagaaaaacaatgtgttcgattgggaagaggtctgtctgaccagcgcgtataaagaactgttcaacaagtacggaataaattatcagcaaggtgacattcgcgcactgctgtgtgaacagtcagataaagcattttatagcagctttatggcgctgatgagcctgatgctccagatgcgcaacagcataaccggtcgcacagatgttgactttctgatcagccctgtgaagaatagcgacggcatcttctacgattccaggaactatgaagcacaggaaaacgctattctgcctaaaaatgccgatgccaacggcgcctataatattgcacggaaggttctgtgggcgattggacagttcaagaaagcggaagatgagaagctggataaggtaaaaattgctattagcaataaggaatggctggagtacgcacagacatcggttaaacacgcggccgcttccctgcaggtaattaaataa

SEQ ID NO: 4 - protein, 1246 aa, Lachnospiraceae bacterium ND2006
MLKNVGIDRLDVEKGRKNMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 5 - DNA, 3924 bp, synthetic construct
  misc_feature 1..3924: note = Synthetic DNA
  source 1..3924: mol_type = other DNA
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagatgccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcttttttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaactga

SEQ ID NO: 6 - DNA, 3741 bp, synthetic construct
  misc_feature 1..3741: note = Synthetic DNA
  source 1..3741: mol_type = other DNA
atgctgaaaaacgtgggtattgatcgtctggatgttgaaaaaggtcgcaaaaatatgagcaaactggaaaagttcaccaactgttatagcctgagcaaaaccctgcgttttaaagcaattccggttggtaaaacccaagagaacattgataataaacgcctgctggtcgaagatgaaaaacgcgctgaagattataaaggcgtgaaaaaactgctggatcgctattatctgagcttcattaacgatgtgctgcacagcattaaactgaagaacctgaacaactatatcagcctgtttcgtaaaaaaacccgcaccgaaaaagaaaacaaagagctggaaaacctggaaatcaatctgcgtaaagaaatcgccaaagcgtttaaaggtaacgagggttataaaagcctgttcaagaaagacatcatcgaaaccattctgccggaatttctggatgataaagatgaaattgccctggtgaatagctttaatggctttaccaccgcatttaccggcttttttgataatcgcgaaaacatgttcagcgaagaagcaaaaagcaccagcattgcatttcgctgcattaatgaaaatctgacccgctacattagcaacatggatatctttgaaaaagtggacgcgatcttcgataaacacgaagtgcaagagatcaaagagaaaatcctgaacagcgattatgacgtcgaagatttttttgaaggcgagttctttaacttcgttctgacccaagaaggtatcgacgtttataacgcaattattggtggttttgttaccgaaagcggtgagaaaatcaaaggcctgaatgaatatatcaacctgtataaccagaaaaccaaacagaaactgccgaaattcaaaccgctgtataaacaggttctgagcgatcgtgaaagcctgagcttttatggtgaaggttataccagtgatgaagaggttctggaagtttttcgtaacaccctgaataaaaacagcgagatctttagcagcatcaaaaagcttgagaaactgttcaaaaactttgatgagtatagcagcgcaggcatctttgttaaaaatggtccggcaattagcaccatcagcaaagatatttttggcgaatggaatgtgatccgcgataaatggaatgccgaatatgatgatatccacctgaaaaaaaaggccgtggtgaccgagaaatatgaagatgatcgtcgtaaaagcttcaagaaaattggtagctttagcctggaacagctgcaagaatatgcagatgcagatctgagcgttgtggaaaaactgaaagaaatcatcattcagaaggtggacgagatctataaagtttatggtagcagcgaaaaactgttcgatgcagattttgttctggaaaaaagcctgaaaaagaatgatgccgttgtggccattatgaaagatctgctggatagcgttaagagcttcgagaattacatcaaagccttttttggtgagggcaaagaaaccaatcgtgatgaaagtttctatggcgattttgtgctggcctatgatattctgctgaaagtggaccatatttatgatgccattcgcaattatgttacccagaaaccgtatagcaaagacaagttcaaactgtactttcagaacccgcagtttatgggtggttgggataaagataaagaaaccgattatcgtgccaccatcctgcgttatggtagtaaatactatctggccatcatggacaaaaaatacgcaaaatgcctgcagaaaatcgacaaagatgatgtgaatggcaactatgaaaaaatcaactacaaactgctgcctggtccgaataaaatgctgccgaaagtgttctttagcaagaaatggatggcctattataacccgagcgaggatattcaaaagatctacaaaaatggcacctttaaaaagggcgacatgttcaatctgaacgattgccacaaactgatcgatttcttcaaagattcaatttcgcgttatccgaaatggtccaatgcctatgattttaactttagcgaaaccgaaaaatacaaagacattgccggtttttatcgcgaagtggaagaacagggctataaagtgagctttgaaagcgcaagcaaaaaagaggttgataagctggttgaagagggcaaactgtatatgttccagatttacaacaaagattttagcgacaaaagccatggcaccccgaatctgcataccatgtactttaaactgctgttcgacgaaaataaccatggtcagattcgtctgagcggtggtgccgaactgtttatgcgtcgtgcaagtctgaaaaaagaagaactggttgttcatccggcaaatagcccgattgcaaacaaaaatccggacaatccgaaaaaaaccacgacactgagctatgatgtgtataaagacaaacgttttagcgaggatcagtatgaactgcatatcccgattgccatcaataaatgcccgaaaaacatctttaagatcaacaccgaagttcgcgtgctgctgaaacatgatgataatccgtatgtgattggcattgatcgtggtgaacgtaacctgctgtatattgttgttgttgatggtaaaggcaacatcgtggaacagtatagtctgaacgaaattatcaacaactttaacggcatccgcatcaaaaccgactatcatagcctgctggacaagaaagaaaaagaacgttttgaagcacgtcagaactggaccagtattgaaaacatcaaagaactgaaagccggttatattagccaggtggttcataaaatctgtgagctggtagaaaaatacgatgcagttattgcactggaagatctgaatagcggtttcaaaaatagccgtgtgaaagtcgaaaaacaggtgtatcagaaattcgagaaaatgctgatcgacaaactgaactacatggtcgacaaaaaaagcaatccgtgtgcaaccggtggtgcactgaaaggttatcagattaccaacaaatttgaaagctttaaaagcatgagcacccagaacggctttatcttctatattccggcatggctgaccagcaaaattgatccgagcaccggttttgtgaacctgctgaaaacaaaatatacctccattgccgacagcaagaagtttattagcagctttgatcgcattatgtatgttccggaagaggacctgtttgaattcgcactggattacaaaaatttcagccgtaccgatgccgactacatcaaaaaatggaaactgtacagctatggtaaccgcattcgcatttttcgcaacccgaagaaaaacaatgtgttcgattgggaagaagtttgtctgaccagcgcatataaagaacttttcaacaaatacggcatcaactatcagcagggtgatattcgtgcactgctgtgtgaacagagcgataaagcgttttatagcagttttatggcactgatgagcctgatgctgcagatgcgtaatagcattaccggtcgcaccgatgtggattttctgattagtccggtgaaaaattccgatggcatcttttatgatagccgcaattacgaagcacaagaaaatgcaattctgccgaaaaacgcagatgcaaatggtgcatataacattgcacgtaaagttctgtgggcaattggccagtttaagaaagcagaagatgagaagctggacaaagtgaaaattgcgatcagcaataaagagtggctggaatacgcacagaccagcgttaaacattga

SEQ ID NO: 7 - protein, 1246 aa, synthetic construct; identical to SEQ ID NO: 4
  REGION 1..1246: note = Synthetic Amino Acid sequence

SEQ ID NO: 8 - DNA, 3924 bp, synthetic construct
  misc_feature 1..3924: note = Synthetic DNA
  source 1..3924: mol_type = other DNA
atgacccagttcgagggcttcaccaacctgtaccaggtgtccaagaccctgagattcgagctgatcccccagggcaagacactgaagcacatccaggaacagggcttcatcgaagaggacaaggcccggaacgaccactacaaagagctgaagcccatcatcgaccggatctacaagacctacgccgaccagtgcctgcagctggtgcagctggactgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagcggcacgccgagatctataagggcctgttcaaggccgagctgttcaacggcaaggtgctgaagcagctgggcaccgtgaccaccaccgagcacgaaaacgccctgctgcggagcttcgacaagttcaccacctacttcagcggcttctacgagaaccggaagaacgtgttcagcgccgaggacatcagcaccgccatcccccacagaatcgtgcaggacaacttccccaagttcaaagagaactgccacatcttcacccggctgatcaccgccgtgcccagcctgagagaacacttcgagaacgtgaagaaggccatcggcatcttcgtgtccaccagcatcgaggaagtgttcagcttcccattctacaaccagctgctgacccagacccagatcgacctgtataatcagctgctgggcggcatcagcagagaggccggcaccgagaagatcaagggcctgaacgaagtgctgaacctggccatccagaagaacgacgagacagcccacatcattgccagcctgccccaccggttcatccctctgttcaagcagatcctgagcgacagaaacaccctgagcttcatcctggaagagttcaagtccgatgaggaagtgatccagagcttctgcaagtataagaccctgctgaggaacgagaatgtgctggaaaccgccgaggccctgttcaatgagctgaacagcatcgacctgacccacatctttatcagccacaagaagctggaaacaatcagcagcgccctgtgcgaccactgggacacactgcggaatgccctgtacgagcggcggatctctgagctgaccggcaagatcaccaagagcgccaaagaaaaggtgcagcggagcctgaagcacgaggatatcaacctgcaggaaatcatcagcgccgctggcaaagaactgagcgaggcctttaagcagaaaaccagcgagatcctgtcccacgcccacgccgcactggatcagcctctgcctaccaccctgaagaagcaggaagagaaagagatcctgaagtcccagctggacagcctgctgggcctgtaccatctgctggattggttcgccgtggacgagagcaacgaggtggaccccgagttctccgccagactgacaggcatcaaactggaaatggaacccagcctgtccttctacaacaaggccagaaactacgccaccaagaaaccctacagcgtggaaaagtttaagctgaacttccagatgcccaccctggccagcggctgggacgtgaacaaagagaagaacaacggcgccatcctgttcgtgaagaacggactgtactacctgggcatcatgcctaagcagaagggcagatacaaggccctgtcctttgagcccaccgaaaagaccagcgagggctttgacaagatgtactacgattacttccccgacgccgccaagatgatccccaagtgcagcacccagctgaaggccgtgaccgcccactttcagacccacaccacccccatcctgctgagcaacaacttcatcgagcccctggaaatcaccaaagagatctacgacctgaacaaccccgagaaagagcccaagaagttccagaccgcctacgccaagaaaaccggcgaccagaagggctaccgcgaggctctgtgcaagtggatcgactttacccgggacttcctgagcaagtacaccaagaccacctccatcgatctgagcagcctgcggcccagctcccagtacaaggatctgggcgagtactacgccgagctgaaccctctgctgtaccacatcagcttccagcggatcgccgaaaaagaaatcatggacgccgtggaaaccggcaagctgtacctgttccagatctataacaaggacttcgccaagggccaccacggcaagcccaatctgcacaccctgtactggaccggcctgtttagccccgagaatctggccaagaccagcatcaagctgaacggccaggccgaactgttttaccggcccaagagccggatgaagcggatggcccatagactgggcgagaagatgctgaacaagaaactgaaggaccagaaaacccctatccccgacacactgtatcaggaactgtacgactacgtgaaccaccggctgagccacgacctgtccgacgaagctagagcactgctgcccaacgtgatcacaaaagaggtgtcccacgagatcatcaaggaccggcggtttacctccgataagttcttcttccacgtgcccatcaccctgaactaccaggccgccaacagccccagcaagttcaaccagagagtgaacgcctacctgaaagagcaccccgagacacccatcattggcatcgacagaggcgagcggaacctgatctacatcaccgtgatcgacagcacaggcaaaatcctggaacagagaagcctgaacaccatccagcagttcgactaccagaagaaactggacaaccgggaaaaagaacgggtggccgccagacaggcttggagcgtcgtgggcaccattaaggacctgaagcagggctacctgagccaagtgattcacgagatcgtggacctgatgatccactatcaggctgtggtggtgctggaaaacctgaacttcggcttcaagagcaagcggaccggaatcgccgagaaagccgtgtaccagcagtttgagaaaatgctgatcgacaagctgaattgcctggtgctgaaagactaccccgctgagaaagtgggaggcgtgctgaatccctaccagctgaccgaccagttcacctcctttgccaagatgggaacccagagcggcttcctgttctacgtgccagccccctacaccagcaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcccggcttcatgcctgcctgggatatcgtgttcgagaagaatgagacacagttcgacgccaagggaaccccctttatcgccggcaagaggatcgtgcctgtgatcgagaaccacagattcaccggcagataccgggacctgtaccccgccaacgagctgattgccctgctggaagagaagggcatcgtgttccgggacggcagcaacatcctgcccaagctgctggaaaatgacgacagccacgccatcgataccatggtggcactgatccgcagcgtgctgcagatgcggaacagcaatgccgccaccggcgaggactacatcaatagcccagtgcgggacctgaacggcgtgtgcttcgacagcagattccagaaccccgagtggcccatggatgccgacgccaatggcgcctaccacattgccctgaagggacagctgctgctgaaccatctgaaagagagcaaagacctgaaactgcagaacggcatctccaaccaggactggctggcctatatccaggaactgcggaactga

SEQ ID NO: 9 - DNA, 3738 bp, synthetic construct
  misc_feature 1..3738: note = Synthetic DNA
  source 1..3738: mol_type = other DNA
atgctgaagaacgtgggcatcgaccggctggacgtggaaaagggcagaaagaacatgagcaagctcgagaagttcaccaactgctacagcctgagcaagaccctgcggttcaaggccattcctgtgggcaagacccaagagaacatcgacaacaagcggctgctggtggaagatgagaagagagccgaggactacaagggcgtgaagaagctgctggaccggtactacctgagcttcatcaacgacgtgctgcacagcatcaagctcaagaacctgaacaactacatcagcctgttccggaagaaaacccggaccgagaaagagaacaaagagctggaaaacctcgagatcaacctgcggaaagagatcgccaaggccttcaagggcaacgagggctacaagagcctgttcaagaaggacatcatcgagacaatcctgcctgagttcctggacgacaaggacgagatcgccctggtcaacagcttcaacggcttcacaaccgccttcaccggctttttcgacaaccgcgagaatatgttcagcgaggaagccaagagcacctctatcgccttccggtgcatcaacgagaatctgacccggtacatcagcaacatggatatcttcgagaaggtggacgccatcttcgacaagcacgaggtgcaagagatcaaagaaaagatcctgaacagcgactacgacgtcgaggacttcttcgagggcgagttcttcaacttcgtgctgacacaagagggcatcgatgtgtacaacgccatcatcggcggcttcgtgacagagagcggcgagaagatcaagggcctgaacgagtacatcaacctctacaaccagaaaacgaagcagaagctgcccaagttcaagcccctgtacaaacaggtgctgagcgacagagagagcctgtccttttacggcgagggctataccagcgacgaagaggtgctggaagtgttcagaaacaccctgaacaagaacagcgagatcttcagctccatcaagaagctcgaaaagctgtttaagaacttcgacgagtacagcagcgccggcatcttcgtgaagaatggccctgccatcagcaccatctccaaggacatcttcggcgagtggaacgtgatccgggacaagtggaacgccgagtacgacgacatccacctgaagaaaaaggccgtggtcaccgagaagtacgaggacgacagaagaaagagcttcaagaagatcggcagcttcagcctggaacagctgcaagagtacgccgacgccgatctgagcgtggtggaaaagctgaaagagattatcatccagaaggtcgacgagatctacaaggtgtacggcagcagcgagaagctgttcgacgccgactttgtgctggaaaagagcctcaaaaagaacgacgccgtggtggccatcatgaaggacctgctggatagcgtgaagtccttcgagaactatattaaggccttctttggcgagggcaaagagacaaaccgggacgagagcttctacggcgatttcgtgctggcctacgacatcctgctgaaagtggaccacatctacgacgccatccggaactacgtgacccagaagccttacagcaaggacaagtttaagctgtacttccagaatccgcagttcatgggcggctgggacaaagacaaagaaaccgactaccgggccaccatcctgagatacggctccaagtactatctggccattatggacaagaaatacgccaagtgcctgcagaagatcgataaggacgacgtgaacggcaactacgagaagattaactacaagctgctgcccggacctaacaagatgctgcctaaggtgttctttagcaagaaatggatggcctactacaaccccagcgaggatatccagaaaatctacaagaacggcaccttcaagaaaggcgacatgttcaacctgaacgactgccacaagctgatcgatttcttcaaggacagcatcagcagataccccaagtggtccaacgcctacgacttcaatttcagcgagacagagaagtataaggatatcgccgggttctaccgcgaggtggaagaacagggctataaggtgtcctttgagagcgccagcaagaaagaggtggacaagctggtcgaagagggcaagctgtacatgttccagatctataacaaggacttctccgacaagagccacggcacccctaacctgcacaccatgtactttaagctgctgttcgatgagaacaaccacggccagatcagactgtctggcggagccgagctgtttatgagaagggccagcctgaaaaaagaggaactggtcgttcaccccgccaactctccaatcgccaacaagaaccccgacaatcccaagaaaaccaccacactgagctacgacgtgtacaaggataagcggttctccgaggaccagtacgagctgcacatccctatcgccatcaacaagtgccccaagaatatcttcaagatcaacaccgaagtgcgggtgctgctgaagcacgacgacaacccttacgtgatcggcatcgacagaggcgagcggaacctgctgtatatcgtggtggtggacggcaagggcaatatcgtggaacagtactccctgaatgagatcatcaacaacttcaatggcatccggatcaagacggactaccacagcctgctggacaaaaaagagaaagaacgcttcgaggcccggcagaactggaccagcatcgagaacatcaaagaactgaaggccggctacatctcccaggtggtgcacaagatctgcgagctggttgagaagtatgacgccgtgattgccctggaagatctgaatagcggctttaagaacagccgcgtgaaggtcgagaaacaggtgtaccagaaattcgagaagatgctgatcgacaagctgaactacatggtcgacaagaagtctaacccctgcgccacaggcggagccctgaagggatatcagatcaccaacaagttcgagtccttcaagagcatgagcacccagaatggcttcatcttctacatccccgcctggctgaccagcaagatcgatcctagcaccggattcgtgaacctgctcaagaccaagtacaccagcattgccgacagcaagaagttcatctccagcttcgaccggattatgtacgtgcccgaagaggacctgttcgaattcgccctggattacaagaacttcagccggaccgatgccgactatatcaagaagtggaagctgtatagctacggcaaccgcatccgcatcttcagaaacccgaagaaaaacaacgtgttcgactgggaagaagtgtgcctgaccagcgcctacaaagaactcttcaacaaatacggcatcaactaccagcagggcgacatcagagccctgctgtgcgagcagagcgacaaggccttttacagctccttcatggccctgatgtccctgatgctgcagatgcggaatagcatcaccggcaggaccgacgtggacttcctgatcagccctgtgaagaattccgacgggatcttctacgacagcagaaactacgaggctcaagagaacgccatcctgcctaagaacgccgatgccaacggcgcctataatatcgccagaaaggtgctgtgggccatcggccagtttaagaaggccgaggacgagaaactggacaaagtgaagatcgccatctctaacaaagagtggctggaatacgcccagaccagcgtgaaacac

SEQ ID NO: 10 - protein, 1246 aa, synthetic construct; identical to SEQ ID NO: 4
  REGION 1..1246: note = Synthetic Amino Acid Sequence

SEQ ID NO: 11 - DNA, 4086 bp, synthetic construct
  misc_feature 1..4086: note = Synthetic DNA
  source 1..4086: mol_type = other DNA
atgggtcgggatccaggtaaaccgattccgaatccgctgctgggtctggatagcaccgcaccgaaaaaaaaacgtaaagttggtattcatggtgttccggcagcaacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagatgccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcttttttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaaccctaaaaaaaaacgcaaagtgaagcttgcggccgcactcgagcaccaccaccaccaccactga

SEQ ID NO: 12 - protein, 1361 aa, synthetic construct
  REGION 1..1361: note = Synthetic Amino Acid Sequence
MGRDPGKPIPNPLLGLDSTAPKKKRKVGIHGVPAATQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNPKKKRKVKLAAALEHHHHHH

SEQ ID NO: 13 - DNA, 3876 bp, synthetic construct
  misc_feature 1..3876: note = Synthetic DNA
  source 1..3876: mol_type = other DNA
atgggtaaaccgattccgaatccgctgctgggtctggatagcaccgcaccgaaaaaaaaacgtaaagttggtattcatggtgttccggcagcactgaaaaacgtgggtattgatcgtctggatgttgaaaaaggtcgcaaaaatatgagcaaactggaaaagttcaccaactgttatagcctgagcaaaaccctgcgttttaaagcaattccggttggtaaaacccaagagaacattgataataaacgcctgctggtcgaagatgaaaaacgcgctgaagattataaaggcgtgaaaaaactgctggatcgctattatctgagcttcattaacgatgtgctgcacagcattaaactgaagaacctgaacaactatatcagcctgtttcgtaaaaaaacccgcaccgaaaaagaaaacaaagagctggaaaacctggaaatcaatctgcgtaaagaaatcgccaaagcgtttaaaggtaacgagggttataaaagcctgttcaagaaagacatcatcgaaaccattctgccggaatttctggatgataaagatgaaattgccctggtgaatagctttaatggctttaccaccgcatttaccggcttttttgataatcgcgaaaacatgttcagcgaagaagcaaaaagcaccagcattgcatttcgctgcattaatgaaaatctgacccgctacattagcaacatggatatctttgaaaaagtggacgcgatcttcgataaacacgaagtgcaagagatcaaagagaaaatcctgaacagcgattatgacgtcgaagatttttttgaaggcgagttctttaacttcgttctgacccaagaaggtatcgacgtttataacgcaattattggtggttttgttaccgaaagcggtgagaaaatcaaaggcctgaatgaatatatcaacctgtataaccagaaaaccaaacagaaactgccgaaattcaaaccgctgtataaacaggttctgagcgatcgtgaaagcctgagcttttatggtgaaggttataccagtgatgaagaggttctggaagtttttcgtaacaccctgaataaaaacagcgagatctttagcagcatcaaaaagcttgagaaactgttcaaaaactttgatgagtatagcagcgcaggcatctttgttaaaaatggtccggcaattagcaccatcagcaaagatatttttggcgaatggaatgtgatccgcgataaatggaatgccgaatatgatgatatccacctgaaaaaaaaggccgtggtgaccgagaaatatgaagatgatcgtcgtaaaagcttcaagaaaattggtagctttagcctggaacagctgcaagaatatgcagatgcagatctgagcgttgtggaaaaactgaaagaaatcatcattcagaaggtggacgagatctataaagtttatggtagcagcgaaaaactgttcgatgcagattttgttctggaaaaaagcctgaaaaagaatgatgccgttgtggccattatgaaagatctgctggatagcgttaagagcttcgagaattacatcaaagccttttttggtgagggcaaagaaaccaatcgtgatgaaagtttctatggcgattttgtgctggcctatgatattctgctgaaagtggaccatatttatgatgccattcgcaattatgttacccagaaaccgtatagcaaagacaagttcaaactgtactttcagaacccgcagtttatgggtggttgggataaagataaagaaaccgattatcgtgccaccatcctgcgttatggtagtaaatactatctggccatcatggacaaaaaatacgcaaaatgcctgcagaaaatcgacaaagatgatgtgaatggcaactatgaaaaaatcaactacaaactgctgcctggtccgaataaaatgctgccgaaagtgttctttagcaagaaatggatggcctattataacccgagcgaggatattcaaaagatctacaaaaatggcacctttaaaaagggcgacatgttcaatctgaacgattgccacaaactgatcgatttcttcaaagattcaatttcgcgttatccgaaatggtccaatgcctatgattttaactttagcgaaaccgaaaaatacaaagacattgccggtttttatcgcgaagtggaagaacagggctataaagtgagctttgaaagcgcaagcaaaaaagaggttgataagctggttgaagagggcaaactgtatatgttccagatttacaacaaagattttagcgacaaaagccatggcaccccgaatctgcataccatgtactttaaactgctgttcgacgaaaataaccatggtcagattcgtctgagcggtggtgccgaactgtttatgcgtcgtgcaagtctgaaaaaagaagaactggttgttcatccggcaaatagcccgattgcaaacaaaaatccggacaatccgaaaaaaaccacgacactgagctatgatgtgtataaagacaaacgttttagcgaggatcagtatgaactgcatatcccgattgccatcaataaatgcccgaaaaacatctttaagatcaacaccgaagttcgcgtgctgctgaaacatgatgataatccgtatgtgattggcattgatcgtggtgaacgtaacctgctgtatattgttgttgttgatggtaaaggcaacatcgtggaacagtatagtctgaacgaaattatcaacaactttaacggcatccgcatcaaaaccgactatcatagcctgctggacaagaaagaaaaagaacgttttgaagcacgtcagaactggaccagtattgaaaacatcaaagaactgaaagccggttatattagccaggtggttcataaaatctgtgagctggtagaaaaatacgatgcagttattgcactggaagatctgaatagcggtttcaaaaatagccgtgtgaaagtcgaaaaacaggtgtatcagaaattcgagaaaatgctgatcgacaaactgaactacatggtcgacaaaaaaagcaatccgtgtgcaaccggtggtgcactgaaaggttatcagattaccaacaaatttgaaagctttaaaagcatgagcacccagaacggctttatcttctatattccggcatggctgaccagcaaaattgatccgagcaccggttttgtgaacctgctgaaaacaaaatatacctccattgccgacagcaagaagtttattagcagctttgatcgcattatgtatgttccggaagaggacctgtttgaattcgcactggattacaaaaatttcagccgtaccgatgccgactacatcaaaaaatggaaactgtacagctatggtaaccgcattcgcatttttcgcaacccgaagaaaaacaatgtgttcgattgggaagaagtttgtctgaccagcgcatataaagaacttttcaacaaatacggcatcaactatcagcagggtgatattcgtgcactgctgtgtgaacagagcgataaagcgttttatagcagttttatggcactgatgagcctgatgctgcagatgcgtaatagcattaccggtcgcaccgatgtggattttctgattagtccggtgaaaaattccgatggcatcttttatgatagccgcaattacgaagcacaagaaaatgcaattctgccgaaaaacgcagatgcaaatggtgcatataacattgcacgtaaagttctgtgggcaattggccagtttaagaaagcagaagatgagaagctggacaaagtgaaaattgcgatcagcaataaagagtggctggaatacgcacagaccagcgttaaacatccgaaaaaaaaacgcaaagtgctcgagcaccaccaccaccaccactga

SEQ ID NO: 14 - protein, 1291 aa, synthetic construct
  REGION 1..1291: note = Synthetic Amino Acid Sequence
MGKPIPNPLLGLDSTAPKKKRKVGIHGVPAALKNVGIDRLDVEKGRKNMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHPKKKRKVLEHHHHHH

SEQ ID NO: 15 - DNA, 4071 bp, synthetic construct
  misc_feature 1..4071: note = Synthetic DNA
  source 1..4071: mol_type = other DNA
atgggcaagcccattcctaatcctctgctgggcctcgacagcacagcccctaagaaaaagcggaaagtgggcatccatggcgtgccagccgccacacagtttgagggcttcaccaacctgtaccaggtgtccaagacactgcgcttcgagctgatccctcagggcaagaccctgaagcacatccaagagcagggcttcatcgaagaggacaaggcccggaacgaccactacaaagagctgaagcccatcatcgaccggatctacaagacctacgccgaccagtgtctgcagctggtgcagctcgattgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagagacacgccgagatctataagggcctgttcaaggccgagctgttcaacggcaaggtgctgaagcagctgggcaccgtgacaaccaccgagcacgaaaatgccctgctgcggagcttcgacaagttcaccacctacttcagcggcttctacgagaaccggaagaacgtgttcagcgccgaggacatcagcaccgccattcctcacagaatcgtgcaggacaacttccccaagttcaaagagaactgccacatcttcacccggctgatcacagccgtgcctagcctgagagaacacttcgagaacgtgaagaaggccatcggcatcttcgtgtccaccagcatcgaggaagtgttcagcttcccattctacaaccagctgctgacccagacacagatcgacctgtataatcagctgctcggcggcatcagcagagaggccggaacagagaagatcaagggcctgaacgaagtgctgaacctggccatccagaagaacgacgagacagcccacatcattgccagcctgcctcaccggttcatccctctgttcaagcagatcctgagcgacagaaacaccctgagcttcatcctggaagagttcaagtccgatgaggaagtgatccagagcttctgcaagtataagaccctgctgaggaacgagaatgtgctggaaaccgccgaggctctgtttaacgagctgaacagcatcgatctgacccacatctttatcagccacaagaagctcgagacaatcagcagcgccctgtgcgaccactgggataccctgagaaacgccctgtacgagcggagaatcagcgagctgaccggcaagatcaccaagagcgccaaagaaaaggtgcagcggagcctgaaacacgaggatatcaacctgcaagagatcatcagcgccgctggcaaagaactgagcgaggcctttaagcagaaaaccagcgagatcctgtctcacgcccacgctgctcttgatcagcctctgcctaccacactgaagaagcaagaggaaaaagagatcctgaagtcccagctggacagcctgctgggactgtaccatctgctggattggttcgccgtggacgagagcaatgaggtggaccctgagttctccgccagactgacaggcatcaagctggaaatggaacccagcctgtccttctacaacaaggccagaaactacgccaccaagaagccctacagcgtcgagaagttcaagctcaacttccagatgcctacactggccagcggctgggacgtgaacaaagagaagaacaacggcgccatcctgttcgtgaagaacggactgtactacctgggcatcatgccaaagcagaagggcagatacaaggccctgtcctttgagcccaccgaaaagaccagcgagggcttcgataagatgtactacgattacttccccgacgccgccaagatgatccccaagtgtagcacacagctgaaggccgtgaccgctcactttcagacccacaccacacctatcctgctgagcaacaacttcatcgagcccctggaaatcaccaaagagatctacgacctgaacaaccccgagaaagagcccaagaagttccagaccgcctacgccaagaaaaccggcgaccagaagggctacagagaagccctgtgcaagtggatcgactttacccgggacttcctgagcaagtacaccaagaccacctccatcgacctgagcagcctgaggcctagcagccagtataaggacctgggcgagtactacgccgagctgaatccactgctgtaccacatcagcttccagcggatcgccgaaaaagaaatcatggacgccgtggaaaccggcaagctgtacctgttccagatatacaacaaagacttcgccaagggccaccacggcaagcctaatctgcacaccctgtactggaccggcctgtttagccctgagaatctggccaagacctctatcaagctgaacggccaggccgaactgttttacagacccaagagccggatgaagcggatggcccacagactgggagagaagatgctgaacaagaaactgaaggaccagaaaacgcccattccggacacactgtaccaagagctgtacgactacgtgaaccaccggctgagccacgatctgagcgacgaagctagagcactgctgcccaacgtgatcacaaaagaggtgtcccacgagatcattaaggaccggcggtttacctccgataagttcttcttccacgtgccgatcacactgaactaccaggccgccaactctcccagcaagttcaaccagagagtgaacgcctacctgaaagagcaccccgagacacccatcattggcatcgacagaggcgagcggaacctgatctacatcaccgtgatcgactccacaggcaagatcctggaacagcggtccctgaacaccatccagcagttcgactaccagaagaagctggacaaccgagagaaagaaagagtggccgccagacaggcttggagcgttgtgggcacaatcaaggatctgaagcagggctacctgagccaagtgattcacgagatcgtggacctgatgatccactatcaggctgtggtggtgctcgagaacctgaacttcggcttcaagagcaagcggaccggaatcgccgagaaagccgtgtaccagcagtttgagaaaatgctgatcgacaagctgaattgcctggtcctgaaggactaccccgctgagaaagttggcggagtgctgaatccctaccagctgaccgatcagttcaccagctttgccaagatgggaacccagagcggcttcctgttctacgtgccagctccttacacctccaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcctggcttcatgcctgcttgggatatcgtgttcgagaagaatgagactcagttcgacgccaaggggaccccttttatcgccggcaagagaattgtgcctgtgatcgagaaccacaggttcaccggcagataccgggatctgtaccccgccaatgagctgatcgccctgctggaagagaagggcatcgtgtttagagatggcagcaacatcctgcctaagctgctggaaaacgacgacagccacgccatcgataccatggtggcactgatcagatccgtgctgcagatgcggaacagcaatgccgctaccggcgaggactacatcaatagccccgtgcgggatctgaacggcgtgtgcttcgacagcagatttcagaaccccgagtggcctatggatgccgacgccaatggcgcctatcacattgccctgaaaggacagctgctgctgaaccatctgaaagagagcaaggacctgaaactgcagaacggcatctccaaccaggactggctggcctacattcaagagctgcggaatcccaaaaagaaacggaaagtgaagctggccgctgctctggaacaccaccaccatcaccat

SEQ ID NO: 16 - protein, 1357 aa, synthetic construct
  REGION 1..1357: note = Synthetic Amino Acid Sequence
MGKPIPNPLLGLDSTAPKKKRKVGIHGVPAATQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNPKKKRKVKLAAALEHHHHHH

SEQ ID NO: 17 - DNA, 3873 bp, synthetic construct
  misc_feature 1..3873: note = Synthetic DNA
  source 1..3873: mol_type = other DNA
atgggcaagcccattcctaatcctctgctgggcctcgacagcacagcccctaagaaaaagcggaaagtgggcatccatggcgtgccagccgctctgaagaatgtgggcatcgacagactggacgtggaaaagggcagaaagaacatgagcaagctcgagaagttcaccaactgctacagcctgagcaagaccctgcggttcaaggccattcctgtgggcaagacccaagagaacatcgacaacaagcggctgctggtggaagatgagaagagagccgaggactacaagggcgtgaagaagctgctggaccggtactacctgagcttcatcaacgacgtgctgcacagcatcaagctgaagaacctgaacaactacatcagcctgttccggaagaaaacccggaccgagaaagagaacaaagagctggaaaacctcgagatcaacctgcggaaagagatcgccaaggccttcaagggcaacgagggctacaagagcctgttcaagaaggacatcatcgagacaatcctgcctgagttcctggacgacaaggacgagatcgccctggtcaacagcttcaacggcttcacaaccgccttcaccggctttttcgacaaccgcgagaatatgttcagcgaggaagccaagagcacctctatcgccttccggtgcatcaacgagaatctgacccggtacatcagcaacatggatatcttcgagaaggtggacgccatcttcgacaagcacgaggtgcaagagatcaaagaaaagatcctgaacagcgactacgacgtcgaggacttcttcgagggcgagttcttcaacttcgtgctgacacaagagggcatcgatgtgtacaacgccatcatcggcggcttcgtgacagagagcggcgagaagatcaagggcctgaacgagtacatcaacctctacaaccagaaaacgaagcagaagctgcccaagttcaagcccctgtacaaacaggtgctgagcgacagagagagcctgtccttttacggcgagggctataccagcgacgaagaggtgctggaagtgttcagaaacaccctgaacaagaacagcgagatcttcagctccatcaagaagctcgaaaagctgtttaagaacttcgacgagtacagcagcgccggcatcttcgtgaagaatggccctgccatcagcaccatctccaaggacatcttcggcgagtggaacgtgatccgggacaagtggaacgccgagtacgacgacatccacctgaagaaaaaggccgtggtcaccgagaagtacgaggacgacagaagaaagagcttcaagaagatcggcagcttcagcctggaacagctgcaagagtacgccgacgccgatctgagcgtggtggaaaagctgaaagagattatcatccagaaggtcgacgagatctacaaggtgtacggcagcagcgagaagctgttcgacgccgactttgtgctggaaaagagcctcaaaaagaacgacgccgtggtggccatcatgaaggacctgctggatagcgtgaagtccttcgagaactatattaaggccttctttggcgagggcaaagagacaaaccgggacgagagcttctacggcgatttcgtgctggcctacgacatcctgctgaaagtggaccacatctacgacgccatccggaactacgtgacccagaagccttacagcaaggacaagtttaagctgtacttccagaatccgcagttcatgggcggctgggacaaagacaaagaaaccgactaccgggccaccatcctgagatacggctccaagtactatctggccattatggacaagaaatacgccaagtgcctgcagaagatcgataaggacgacgtgaacggcaactacgagaagattaactacaagctgctgcccggacctaacaagatgctgcctaaggtgttctttagcaagaaatggatggcctactacaaccccagcgaggatatccagaaaatctacaagaacggcaccttcaagaaaggcgacatgttcaacctgaacgactgccacaagctgatcgatttcttcaaggacagcatcagcagataccccaagtggtccaacgcctacgacttcaatttcagcgagacagagaagtataaggatatcgccgggttctaccgcgaggtggaagaacagggctataaggtgtcctttgagagcgccagcaagaaagaggtggacaagctggtcgaagagggcaagctgtacatgttccagatctataacaaggacttctccgacaagagccacggcacccctaacctgcacaccatgtactttaagctgctgttcgatgagaacaaccacggccagatcagactgtctggcggagccgagctgtttatgagaagggccagcctgaaaaaagaggaactggtcgttcaccccgccaactctccaatcgccaacaagaaccccgacaatcccaagaaaaccaccacactgagctacgacgtgtacaaggataagcggttctccgaggaccagtacgagctgcacatccctatcgccatcaacaagtgccccaagaatatcttcaagatcaacaccgaagtgcgggtgctgctgaagcacgacgacaacccttacgtgatcggcatcgatcggggcgagagaaacctgctgtatatcgtggtggtggacggcaagggcaatatcgtggaacagtactccctgaatgagatcatcaacaacttcaatggcatccggatcaagacggactaccacagcctgctggacaaaaaagagaaagaacgcttcgaggccaggcagaactggaccagcatcgagaacatcaaagaactgaaggccggctacatctcccaggtggtgcacaagatctgcgagctggttgagaagtatgacgccgtgattgccctggaagatctgaatagcggctttaagaacagccgcgtgaaggtcgagaaacaggtgtaccagaaattcgagaagatgctgatcgacaagctgaactacatggtcgacaagaagtctaacccctgcgccacaggcggagccctgaagggatatcagatcaccaacaagttcgagtccttcaagagcatgagcacccagaatggcttcatcttctacatccccgcctggctgaccagcaagatcgatcctagcaccggattcgtgaacctgctcaagaccaagtacaccagcattgccgacagcaagaagttcatctccagcttcgaccggattatgtacgtgcccgaagaggacctgttcgaattcgccctggattacaagaacttcagccggaccgatgccgactatatcaagaagtggaagctgtatagctacggcaaccgcatccgcatcttcagaaacccgaagaaaaacaacgtgttcgactgggaagaagtgtgcctgaccagcgcctacaaagaactcttcaacaaatacggcatcaactaccagcagggcgacatcagagccctgctgtgcgagcagagcgacaaggccttttacagctccttcatggccctgatgagcctgatgctgcagatgcggaatagcatcaccggcagaaccgacgtggacttcctgatcagccccgtgaaaaactccgacggcatcttttacgacagccggaattacgaggctcaagagaacgccatcctgcctaagaacgccgatgccaacggcgcctataatatcgccagaaaggtgctgtgggccatcggccagtttaagaaggccgaggacgagaaactggacaaagtgaagatcgccatctctaacaaagagtggctggaatacgcccagaccagcgtgaagcaccccaaaaagaaacggaaagtgctggaacaccaccaccatcaccac

SEQ ID NO: 18 - DNA, 4092 bp, synthetic construct
  misc_feature 1..4092: note = Synthetic DNA
  source 1..4092: mol_type = other DNA
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagatgccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcttttttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagttggtggtagcggtggttcaggtggtagtggcggtagtggtggctcagggggttctggtggctctggtggtagcctcgagcaccaccaccaccaccactga

SEQ ID NO: 19 - protein, 1363 aa, synthetic construct
  REGION 1..1363: note = Synthetic Amino Acid Sequence
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 20 - protein, 1281 aa, synthetic construct
  REGION 1..1281: note = Synthetic Amino Acid Sequence
MGDPLKNVGIDRLDVEKGRKNMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHGRSSDDEATADSQHAAPPKKKRKVLEHHHHHH

SEQ ID NO: 21 - DNA, 3843 bp, synthetic construct
  misc_feature 1..3843: note = Synthetic DNA
  source 1..3843: mol_type = other DNA
atgggcgaccctctgaagaacgtgggcatcgacagactggacgtggaaaagggcagaaagaacatgagcaagctcgagaagttcaccaactgctacagcctgagcaagaccctgcggttcaaggccattcctgtgggcaagacccaagagaacatcgacaacaagcggctgctggtggaagatgagaagagagccgaggactacaagggcgtgaagaagctgctggaccggtactacctgagcttcatcaacgacgtgctgcacagcatcaagctgaagaacctgaacaactacatcagcctgttccggaagaaaacccggaccgagaaagagaacaaagagctggaaaacctcgagatcaacctgcggaaagagatcgccaaggccttcaagggcaacgagggctacaagagcctgttcaagaaggacatcatcgagacaatcctgcctgagttcctggacgacaaggacgagatcgccctggtcaacagcttcaacggcttcacaaccgccttcaccggctttttcgacaaccgcgagaatatgttcagcgaggaagccaagagcacctctatcgccttccggtgcatcaacgagaatctgacccggtacatcagcaacatggatatcttcgagaaggtggacgccatcttcgacaagcacgaggtgcaagagatcaaagaaaagatcctgaacagcgactacgacgtcgaggacttcttcgagggcgagttcttcaacttcgtgctgacacaagagggcatcgatgtgtacaacgccatcatcggcggcttcgtgacagagagcggcgagaagatcaagggcctgaacgagtacatcaacctctacaaccagaaaacgaagcagaagctgcccaagttcaagcccctgtacaaacaggtgctgagcgacagagagagcctgtccttttacggcgagggctataccagcgacgaagaggtgctggaagtgttcagaaacaccctgaacaagaacagcgagatcttcagctccatcaagaagctcgaaaagctgtttaagaacttcgacgagtacagcagcgccggcatcttcgtgaagaatggccctgccatcagcaccatctccaaggacatcttcggcgagtggaacgtgatccgggacaagtggaacgccgagtacgacgacatccacctgaagaaaaaggccgtggtcaccgagaagtacgaggacgacagaagaaagagcttcaagaagatcggcagcttcagcctggaacagctgcaagagtacgccgacgccgatctgagcgtggtggaaaagctgaaagagattatcatccagaaggtcgacgagatctacaaggtgtacggcagcagcgagaagctgttcgacgccgactttgtgctggaaaagagcctcaaaaagaacgacgccgtggtggccatcatgaaggacctgctggatagcgtgaagtccttcgagaactatattaaggccttctttggcgagggcaaagagacaaaccgggacgagagcttctacggcgatttcgtgctggcctacgacatcctgctgaaagtggaccacatctacgacgccatccggaactacgtgacccagaagccttacagcaaggacaagtttaagctgtacttccagaatccgcagttcatgggcggctgggacaaagacaaagaaaccgactaccgggccaccatcctgagatacggctccaagtactatctggccattatggacaagaaatacgccaagtgcctgcagaagatcgataaggacgacgtgaacggcaactacgagaagattaactacaagctgctgcccggacctaacaagatgctgcctaaggtgttctttagcaagaaatggatggcctactacaaccccagcgaggatatccagaaaatctacaagaacggcaccttcaagaaaggcgacatgttcaacctgaacgactgccacaagctgatcgatttcttcaaggacagcatcagcagataccccaagtggtccaacgcctacgacttcaatttcagcgagacagagaagtataaggatatcgccgggttctaccgcgaggtggaagaacagggctataaggtgtcctttgagagcgccagcaagaaagaggtggacaagctggtcgaagagggcaagctgtacatgttccagatctataacaaggacttctccgacaagagccacggcacccctaacctgcacaccatgtactttaagctgctgttcgatgagaacaaccacggccagatcagactgtctggcggagccgagctgtttatgagaagggccagcctgaaaaaagaggaactggtcgttcaccccgccaactctccaatcgccaacaagaaccccgacaatcccaagaaaaccaccacactgagctacgacgtgtacaaggataagcggttctccgaggaccagtacgagctgcacatccctatcgccatcaacaagtgccccaagaatatcttcaagatcaacaccgaagtgcgggtgctgctgaagcacgacgacaacccttacgtgatcggcatcgatcggggcgagagaaacctgctgtatatcgtggtggtggacggcaagggcaatatcgtggaacagtactccctgaatgagatcatcaacaacttcaatggcatccggatcaagacggactaccacagcctgctggacaaaaaagagaaagaacgcttcgaggcccggcagaactggaccagcatcgagaacatcaaagaactgaaggccggctacatctcccaggtggtgcacaagatctgcgagctggttgagaagtatgacgccgtgattgccctggaagatctgaatagcggctttaagaacagccgcgtgaaggtcgagaaacaggtgtaccagaaattcgagaagatgctgatcgacaagctgaactacatggtcgacaagaagtctaacccctgcgccacaggcggagccctgaagggatatcagatcaccaacaagttcgagtccttcaagagcatgagcacccagaatggcttcatcttctacatccccgcctggctgaccagcaagatcgatcctagcaccggattcgtgaacctgctcaagaccaagtacaccagcattgccgacagcaagaagttcatctccagcttcgaccggattatgtacgtgcccgaagaggacctgttcgaattcgccctggattacaagaacttcagccggaccgatgccgactatatcaagaagtggaagctgtatagctacggcaaccgcatccgcatcttcagaaacccgaagaaaaacaacgtgttcgactgggaagaagtgtgcctgaccagcgcctacaaagaactcttcaacaaatacggcatcaactaccagcagggcgacatcagagccctgctgtgcgagcagagcgacaaggccttttacagctccttcatggccctgatgagcctgatgctgcagatgcggaatagcatcaccggcaggaccgacgtggacttcctgatcagccctgtgaagaattccgacgggatcttctacgacagcagaaactacgaggctcaagagaacgccatcctgcctaagaacgccgatgccaacggcgcctataatatcgccagaaaggtgctgtgggccatcggccagtttaagaaggccgaggacgagaaactggacaaagtgaagatcgccatctctaacaaagagtggctggaatacgcccagaccagcgtgaagcacggcagatctagtgacgatgaggccaccgccgatagccagcatgcagcccctccaaagaaaaagcggaaagtgctggaacaccaccaccatcaccac

SEQ ID NO: 22 - protein, 1363 aa, synthetic construct; identical to SEQ ID NO: 19
  REGION 1..1363: note = Synthetic Amino Acid Sequence

SEQ ID NO: 23 - DNA, 3846 bp, synthetic construct
  misc_feature 1..3846: note = Synthetic DNA
  source 1..3846: mol_type = other DNA
atgggggatccactgaaaaacgtgggtattgatcgtctggatgttgaaaaaggtcgcaaaaatatgagcaaactggaaaagttcaccaactgttatagcctgagcaaaaccctgcgttttaaagcaattccggttggtaaaacccaagagaacattgataataaacgcctgctggtcgaagatgaaaaacgcgctgaagattataaaggcgtgaaaaaactgctggatcgctattatctgagcttcattaacgatgtgctgcacagcattaaactgaagaacctgaacaactatatcagcctgtttcgtaaaaaaacccgcaccgaaaaagaaaacaaagagctggaaaacctggaaatcaatctgcgtaaagaaatcgccaaagcgtttaaaggtaacgagggttataaaagcctgttcaagaaagacatcatcgaaaccattctgccggaatttctggatgataaagatgaaattgccctggtgaatagctttaatggctttaccaccgcatttaccggcttttttgataatcgcgaaaacatgttcagcgaagaagcaaaaagcaccagcattgcatttcgctgcattaatgaaaatctgacccgctacattagcaacatggatatctttgaaaaagtggacgcgatcttcgataaacacgaagtgcaagagatcaaagagaaaatcctgaacagcgattatgacgtcgaagatttttttgaaggcgagttctttaacttcgttctgacccaagaaggtatcgacgtttataacgcaattattggtggttttgttaccgaaagcggtgagaaaatcaaaggcctgaatgaatatatcaacctgtataaccagaaaaccaaacagaaactgccgaaattcaaaccgctgtataaacaggttctgagcgatcgtgaaagcctgagcttttatggtgaaggttataccagtgatgaagaggttctggaagtttttcgtaacaccctgaataaaaacagcgagatctttagcagcatcaaaaagcttgagaaactgttcaaaaactttgatgagtatagcagcgcaggcatctttgttaaaaatggtccggcaattagcaccatcagcaaagatatttttggcgaatggaatgtgatccgcgataaatggaatgccgaatatgatgatatccacctgaaaaaaaaggccgtggtgaccgagaaatatgaagatgatcgtcgtaaaagcttcaagaaaattggtagctttagcctggaacagctgcaagaatatgcagatgcagatctgagcgttgtggaaaaactgaaagaaatcatcattcagaaggtggacgagatctataaagtttatggtagcagcgaaaaactgttcgatgcagattttgttctggaaaaaagcctgaaaaagaatgatgccgttgtggccattatgaaagatctgctggatagcgttaagagcttcgagaattacatcaaagccttttttggtgagggcaaagaaaccaatcgtgatgaaagtttctatggcgattttgtgctggcctatgatattctgctgaaagtggaccatatttatgatgccattcgcaattatgttacccagaaaccgtatagcaaagacaagttcaaactgtactttcagaacccgcagtttatgggtggttgggataaagataaagaaaccgattatcgtgccaccatcctgcgttatggtagtaaatactatctggccatcatggacaaaaaatacgcaaaatgcctgcagaaaatcgacaaagatgatgtgaatggcaactatgaaaaaatcaactacaaactgctgcctggtccgaataaaatgctgccgaaagtgttctttagcaagaaatggatggcctattataacccgagcgaggatattcaaaagatctacaaaaatggcacctttaaaaagggcgacatgttcaatctgaacgattgccacaaactgatcgatttcttcaaagattcaatttcgcgttatccgaaatggtccaatgcctatgattttaactttagcgaaaccgaaaaatacaaagacattgccggtttttatcgcgaagtggaagaacagggctataaagtgagctttgaaagcgcaagcaaaaaagaggttgataagctggttgaagagggcaaactgtatatgttccagatttacaacaaagattttagcgacaaaagccatggcaccccgaatctgcataccatgtactttaaactgctgttcgacgaaaataaccatggtcagattcgtctgagcggtggtgccgaactgtttatgcgtcgtgcaagtctgaaaaaagaagaactggttgttcatccggcaaatagcccgattgcaaacaaaaatccggacaatccgaaaaaaaccacgacactgagctatgatgtgtataaagacaaacgttttagcgaggatcagtatgaactgcatatcccgattgccatcaataaatgcccgaaaaacatctttaagatcaacaccgaagttcgcgtgctgctgaaacatgatgataatccgtatgtgattggcattgatcgtggtgaacgtaacctgctgtatattgttgttgttgatggtaaaggcaacatcgtggaacagtatagtctgaacgaaattatcaacaactttaacggcatccgcatcaaaaccgactatcatagcctgctggacaagaaagaaaaagaacgttttgaagcacgtcagaactggaccagtattgaaaacatcaaagaactgaaagccggttatattagccaggtggttcataaaatctgtgagctggtagaaaaatacgatgcagttattgcactggaagatctgaatagcggtttcaaaaatagccgtgtgaaagtcgaaaaacaggtgtatcagaaattcgagaaaatgctgatcgacaaactgaactacatggtcgacaaaaaaagcaatccgtgtgcaaccggtggtgcactgaaaggttatcagattaccaacaaatttgaaagctttaaaagcatgagcacccagaacggctttatcttctatattccggcatggctgaccagcaaaattgatccgagcaccggttttgtgaacctgctgaaaacaaaatatacctccattgccgacagcaagaagtttattagcagctttgatcgcattatgtatgttccggaagaggacctgtttgaattcgcactggattacaaaaatttcagccgtaccgatgccgactacatcaaaaaatggaaactgtacagctatggtaaccgcattcgcatttttcgcaacccgaagaaaaacaatgtgttcgattgggaagaagtttgtctgaccagcgcatataaagaacttttcaacaaatacggcatcaactatcagcagggtgatattcgtgcactgctgtgtgaacagagcgataaagcgttttatagcagttttatggcactgatgagcctgatgctgcagatgcgtaatagcattaccggtcgcaccgatgtggattttctgattagtccggtgaaaaattccgatggcatcttttatgatagccgcaattacgaagcacaagaaaatgcaattctgccgaaaaacgcagatgcaaatggtgcatataacattgcacgtaaagttctgtgggcaattggccagtttaagaaagcagaagatgagaagctggacaaagtgaaaattgcgatcagcaataaagagtggctggaatacgcacagaccagcgttaaacatggtcgtagcagtgatgatgaagcaaccgcagatagccagcatgcagcaccgccgaaaaaaaaacgcaaagtgctcgagcaccaccaccaccaccactga

SEQ ID NO: 24 - protein, 1278 aa, synthetic construct
  REGION 1..1278: note = Synthetic Amino Acid Sequence
MLKNVGIDRLDVEKGRKNMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHGRSSDDEATADSQHAAPPKKKRKVLEHHHHHH

SEQ ID NO: 25 - DNA, 21 bp, synthetic construct
  misc_feature 1..21: note = Synthetic DNA
  source 1..21: mol_type = other DNA
gtgtccaagaccctgagattc

SEQ ID NO: 26 - DNA, 20 bp, synthetic construct
  misc_feature 1..20: note = Synthetic DNA
  source 1..20: mol_type = other DNA
gggcttcagctctttgtagt

SEQ ID NO: 27 - DNA, 24 bp, synthetic construct
  misc_feature 1..24: note = Synthetic DNA
  source 1..24: mol_type = other DNA
  misc_feature 8..9: note = Napthyl-azo is present between guanine and adenine nucleotides
  misc_feature 24: note = Iowa Black dark quencher is attached to cytosine nucleotide
agggcaagacactgaagcacatcc

SEQ ID NO: 28 - DNA, 20 bp, synthetic construct
  misc_feature 1..20: note = Synthetic DNA
  source 1..20: mol_type = other DNA
cagaaactacgccaccaaga

SEQ ID NO: 29 - DNA, 22 bp, synthetic construct
  misc_feature 1..22: note = Synthetic DNA
  source 1..22: mol_type = other DNA
gccgttgttcttctctttgttc

SEQ ID NO: 30 - DNA, 25 bp, synthetic construct
  misc_feature 1..25: note = Synthetic DNA
  source 1..25: mol_type = other DNA
  misc_feature 9..10: note = Napthyl-azo is present between adenine and cytosine nucleotides
  misc_feature 25: note = Iowa Black dark quencher is attached to cytosine nucleotide
taagctgaacttccagatgcccacc

SEQ ID NO: 31 - DNA, 22 bp, synthetic construct
  misc_feature 1..22: note = Synthetic DNA
  source 1..22: mol_type = other DNA
gtggacctgatgatccactatc

SEQ ID NO: 32 - DNA, 18 bp, synthetic construct
  misc_feature 1..18: note = Synthetic DNA
  source 1..18: mol_type = other DNA
gctggtacacggctttct

SEQ ID NO: 33 - DNA, 24 bp, synthetic construct
  misc_feature 1..24: note = Synthetic DNA
  source 1..24: mol_type = other DNA
  misc_feature 9..10: note = Napthyl-azo is present between thymine nucleotides
  misc_feature 24: note = Iowa Black dark quencher is attached to adenine nucleotide
acctgaacttcggcttcaagagca

SEQ ID NO: 34 - DNA, 22 bp, synthetic construct
  misc_feature 1..22: note = Synthetic DNA
  source 1..22: mol_type = other DNA
tgctgaaccatctgaaagagag

SEQ ID NO: 35 - DNA, 22 bp, synthetic construct
  misc_feature 1..22: note = Synthetic DNA
  source 1..22: mol_type = other DNA
gttccgcagttcctggatatag

SEQ ID NO: 36 - DNA, 23 bp, synthetic construct
  misc_feature 1..23: note = Synthetic DNA
  source 1..23: mol_type = other DNA
  misc_feature 9..10: note = Napthyl-azo is present between thymine nucleotides
  misc_feature 23: note = Iowa Black dark quencher is attached to cytosine nucleotide
agtcctggttggagatgccgttc

SEQ ID NO: 37 - RNA, 44 nt, synthetic construct
  misc_feature 1..44: note = Synthetic RNA
  source 1..44: mol_type = other RNA
taatttctactcttgtagattaaacactgtttcatttcatccgt

SEQ ID NO: 38 - RNA, 44 nt, synthetic construct
  misc_feature 1..44: note = Synthetic RNA
  source 1..44: mol_type = other RNA
taatttctactcttgtagataccagcaagctgttaattacaaaa

SEQ ID NO: 39 - RNA, 44 nt, synthetic construct
  misc_feature 1..44: note = Synthetic RNA
  source 1..44: mol_type = other RNA
taatttctactcttgtagataccatctttaacctaaaagagttt

SEQ ID NO: 40 - RNA, 44 nt, synthetic construct
  misc_feature 1..44: note = Synthetic RNA
  source 1..44: mol_type = other RNA
taatttctactcttgtagatggttaaagatggttaaatgattga

SEQ ID NO: 41 - RNA, 44 nt, synthetic construct
  misc_feature 1..44: note = Synthetic RNA
  source 1..44: mol_type = other RNA
taatttctactcttgtagattgtgaaatggcttataattgctta

SEQ ID NO: 42 - RNA, 44 nt, synthetic construct
  misc_feature 1..44: note = Synthetic RNA
  source 1..44: mol_type = other RNA
taatttctactcttgtagataatgtaagtaattgcttctttttc

SEQ ID NO: 43 - RNA, 43 nt, synthetic construct
  misc_feature 1..43: note = Synthetic RNA
  source 1..43: mol_type = other RNA
aatttctactcttgtagattaaacactgtttcatttcatccgt

SEQ ID NO: 44 - RNA, 42 nt, synthetic construct
  misc_feature 1..42: note = Synthetic RNA
  source 1..42: mol_type = other RNA
atttctactcttgtagattaaacactgtttcatttcatccgt

SEQ ID NO: 45 - RNA, 41 nt, synthetic construct
  misc_feature 1..41: note = Synthetic RNA
  source 1..41: mol_type = other RNA
tttctactcttgtagattaaacactgtttcatttcatccgt

SEQ ID NO: 46 - RNA, 43 nt, synthetic construct
  misc_feature 1..43: note = Synthetic RNA
  source 1..43: mol_type = other RNA
aatttctactcttgtagataccagcaagctgttaattacaaaa

SEQ ID NO: 47 - RNA, 42 nt, synthetic construct
  misc_feature 1..42: note = Synthetic RNA
  source 1..42: mol_type = other RNA
atttctactcttgtagataccagcaagctgttaattacaaaa

SEQ ID NO: 48 - RNA, 41 nt, synthetic construct
  misc_feature 1..41: note = Synthetic RNA
  source 1..41: mol_type = other RNA
tttctactcttgtagataccagcaagctgttaattacaaaa

SEQ ID NO: 49 - RNA, 43 nt, synthetic construct
  misc_feature 1..43: note = Synthetic RNA
  source 1..43: mol_type = other RNA
aatttctactcttgtagataccatctttaacctaaaagagttt

SEQ ID NO: 50 - RNA, 42 nt, synthetic construct
  misc_feature 1..42: note = Synthetic RNA
  source 1..42: mol_type = other RNA
atttctactcttgtagataccatctttaacctaaaagagttt

SEQ ID NO: 51 - RNA, 41 nt, synthetic construct
  misc_feature 1..41: note = Synthetic RNA
  source 1..41: mol_type = other RNA
tttctactcttgtagataccatctttaacctaaaagagttt

SEQ ID NO: 52 - RNA, 43 nt, synthetic construct
  misc_feature 1..43: note = Synthetic RNA
  source 1..43: mol_type = other RNA
aatttctactcttgtagatggttaaagatggttaaatgattga

SEQ ID NO: 53 - RNA, 42 nt, synthetic construct
  misc_feature 1..42: note = Synthetic RNA
  source 1..42: mol_type = other RNA
atttctactcttgtagatggttaaagatggttaaatgattga

SEQ ID NO: 54 - RNA, 41 nt, synthetic construct
  misc_feature 1..41: note = Synthetic RNA
  source 1..41: mol_type = other RNA
tttctactcttgtagatggttaaagatggttaaatgattga

SEQ ID NO: 55 - RNA, 43 nt, synthetic construct
  misc_feature 1..43: note = Synthetic RNA
  source 1..43: mol_type = other RNA
aatttctactcttgtagattgtgaaatggcttataattgctta

SEQ ID NO: 56 - RNA, 42 nt, synthetic construct
  misc_feature 1..42: note = Synthetic RNA
  source 1..42: mol_type = other RNA
atttctactcttgtagattgtgaaatggcttataattgctta

SEQ ID NO: 57 - RNA, 41 nt, synthetic construct
  misc_feature 1..41: note = Synthetic RNA
  source 1..41: mol_type = other RNA
tttctactcttgtagattgtgaaatggcttataattgctta

SEQ ID NO: 58 - RNA, 43 nt, synthetic construct
  misc_feature 1..43: note = Synthetic RNA
  source 1..43: mol_type = other RNA
aatttctactcttgtagataatgtaagtaattgcttctttttc

SEQ ID NO: 59 - RNA, 42 nt, synthetic construct
  misc_feature 1..42: note = Synthetic RNA
  source 1..42: mol_type = other RNA
atttctactcttgtagataatgtaagtaattgcttctttttc

SEQ ID NO: 60 - RNA, 41 nt, synthetic construct
  misc_feature 1..41: note = Synthetic RNA
  source 1..41: mol_type = other RNA
tttctactcttgtagataatgtaagtaattgcttctttttc

SEQ ID NO: 61 - RNA, 41 nt, synthetic construct
  misc_feature 1..41: note = Synthetic RNA
  source 1..41: mol_type = other RNA
taatttctactcttgtagattaaacactgtttcatttcatc

SEQ ID NO: 62 - RNA, 40 nt, synthetic construct
  misc_feature 1..40: note = Synthetic RNA
  source 1..40: mol_type = other RNA
aatttctactcttgtagattaaacactgtttcatttcatc

SEQ ID NO: 63 - RNA, 39 nt, synthetic construct
  misc_feature 1..39: note = Synthetic RNA
  source 1..39: mol_type = other RNA
atttctactcttgtagattaaacactgtttcatttcatc

SEQ ID NO: 64 - RNA, 38 nt, synthetic construct
  misc_feature 1..38: note = Synthetic RNA
  source 1..38: mol_type = other RNA
tttctactcttgtagattaaacactgtttcatttcatc

SEQ ID NO: 65 - RNA, 41 nt, synthetic construct
  misc_feature 1..41: note = Synthetic RNA
  source 1..41: mol_type = other RNA
taatttctactcttgtagataccagcaagctgttaattaca

SEQ ID NO: 66 - RNA, 40 nt, synthetic construct
  misc_feature 1..40: note = Synthetic RNA
  source 1..40: mol_type = other RNA
aatttctactcttgtagataccagcaagctgttaattaca

SEQ ID NO: 67 - RNA, 39 nt, synthetic construct
  misc_feature 1..39: note = Synthetic RNA
  source 1..39: mol_type = other RNA
atttctactcttgtagataccagcaagctgttaattaca

SEQ ID NO: 68 - RNA, 38 nt, synthetic construct
  misc_feature 1..38: note = Synthetic RNA
  source 1..38: mol_type = other RNA
tttctactcttgtagataccagcaagctgttaattaca

SEQ ID NO: 69 - RNA, 41 nt, synthetic construct
  misc_feature 1..41: note = Synthetic RNA
  source 1..41: mol_type = other RNA
taatttctactcttgtagataccatctttaacctaaaagag

SEQ ID NO: 70 - RNA, 40 nt, synthetic construct
  misc_feature 1..40: note = Synthetic RNA
  source 1..40: mol_type = other RNA
aatttctactcttgtagataccatctttaacctaaaagag

SEQ ID NO: 71 - RNA, 39 nt, synthetic construct
  misc_feature 1..39: note = Synthetic RNA
  source 1..39: mol_type = other RNA
atttctactcttgtagataccatctttaacctaaaagag

SEQ ID NO: 72 - RNA, 38 nt, synthetic construct
  misc_feature 1..38: note = Synthetic RNA
  source 1..38: mol_type = other RNA
tttctactcttgtagataccatctttaacctaaaagag

SEQ ID NO: 73 - RNA, 41 nt, synthetic construct
  misc_feature 1..41: note = Synthetic RNA
  source 1..41: mol_type = other RNA
taatttctactcttgtagatggttaaagatggttaaatgat

SEQ ID NO: 74 - RNA, 40 nt, synthetic construct
  misc_feature 1..40: note = Synthetic RNA
  source 1..40: mol_type = other RNA
aatttctactcttgtagatggttaaagatggttaaatgat

SEQ ID NO: 75 - RNA, 39 nt, synthetic construct
  misc_feature 1..39: note = Synthetic RNA
  source 1..39: mol_type = other RNA
atttctactcttgtagatggttaaagatggttaaatgat

SEQ ID NO: 76 - RNA, 38 nt, synthetic construct
  misc_feature 1..38: note = Synthetic RNA
  source 1..38: mol_type = other RNA
tttctactcttgtagatggttaaagatggttaaatgat

SEQ ID NO: 77 - RNA, 41 nt, synthetic construct
  misc_feature 1..41: note = Synthetic RNA
  source 1..41: mol_type = other RNA
taatttctactcttgtagattgtgaaatggcttataattgc

SEQ ID NO: 78 - RNA, 40 nt, synthetic construct
  misc_feature 1..40: note = Synthetic RNA
  source 1..40: mol_type = other RNA
aatttctactcttgtagattgtgaaatggcttataattgc

SEQ ID NO: 79 - RNA, 39 nt, synthetic construct
  misc_feature 1..39: note = Synthetic RNA
  source 1..39: mol_type = other RNA
atttctactcttgtagattgtgaaatggcttataattgc

SEQ ID NO: 80 - RNA, 38 nt, synthetic construct
  misc_feature 1..38: note = Synthetic RNA
  source 1..38: mol_type = other RNA
tttctactcttgtagattgtgaaatggcttataattgc

SEQ ID NO: 81 - RNA, 41 nt, synthetic construct
  misc_feature 1..41: note = Synthetic RNA
  source 1..41: mol_type = other RNA
taatttctactcttgtagataatgtaagtaattgcttcttt

SEQ ID NO: 82 - RNA, 40 nt, synthetic construct
  misc_feature 1..40: note = Synthetic RNA
  source 1..40: mol_type = other RNA
aatttctactcttgtagataatgtaagtaattgcttcttt

SEQ ID NO: 83 - RNA, 39 nt, synthetic construct
  misc_feature 1..39: note = Synthetic RNA
  source 1..39: mol_type = other RNA
atttctactcttgtagataatgtaagtaattgcttcttt

SEQ ID NO: 84 - RNA, 38 nt, synthetic construct
  misc_feature 1..38: note = Synthetic RNA
  source 1..38: mol_type = other RNA
tttctactcttgtagataatgtaagtaattgcttcttt

SEQ ID NO: 85 - RNA, 39 nt, synthetic construct
  misc_feature 1..39: note = Synthetic RNA
  source 1..39: mol_type = other RNA
taatttctactcttgtagattaaacactgtttcatttca

SEQ ID NO: 86 - RNA, 38 nt, synthetic construct
  misc_feature 1..38: note = Synthetic RNA
  source 1..38: mol_type = other RNA
taatttctactcttgtagattaaacactgtttcatttc

SEQ ID NO: 87 - RNA, 37 nt, synthetic construct
  misc_feature 1..37: note = Synthetic RNA
  source 1..37: mol_type = other RNA
taatttctactcttgtagattaaacactgtttcattt

SEQ ID NO: 88 - RNA, 39 nt, synthetic construct
  misc_feature 1..39: note = Synthetic RNA
  source 1..39: mol_type = other RNA
taatttctactcttgtagataccagcaagctgttaatta

SEQ ID NO: 89 - RNA, 38 nt, synthetic construct
  misc_feature 1..38: note = Synthetic RNA
  source 1..38: mol_type = other RNA
taatttctactcttgtagataccagcaagctgttaatt

SEQ ID NO: 90 - RNA, 37 nt, synthetic construct
  misc_feature 1..37: note = Synthetic RNA
  source 1..37: mol_type = other RNA
taatttctactcttgtagataccagcaagctgttaat

SEQ ID NO: 91 - RNA, 39 nt, synthetic construct
  misc_feature 1..39: note = Synthetic RNA
  source 1..39: mol_type = other RNA
taatttctactcttgtagataccatctttaacctaaaag

SEQ ID NO: 92 - RNA, 38 nt, synthetic construct
  misc_feature 1..38: note = Synthetic RNA
  source 1..38: mol_type = other RNA
taatttctactcttgtagataccatctttaacctaaaa

SEQ ID NO: 93 - RNA, 37 nt, synthetic construct
  misc_feature 1..37: note = Synthetic RNA
  source 1..37: mol_type = other RNA
taatttctactcttgtagataccatctttaacctaaa

SEQ ID NO: 94 - RNA, 39 nt, synthetic construct
  misc_feature 1..39: note = Synthetic RNA
  source 1..39: mol_type = other RNA
taatttctactcttgtagatggttaaagatggttaaatg

SEQ ID NO: 95 - RNA, 38 nt, synthetic construct
  misc_feature 1..38: note = Synthetic RNA
  source 1..38: mol_type = other RNA
taatttctactcttgtagatggttaaagatggttaaat

SEQ ID NO: 96 - RNA, 37 nt, synthetic construct
  misc_feature 1..37: note = Synthetic RNA
  source 1..37: mol_type = other RNA
taatttctactcttgtagatggttaaagatggttaaa

SEQ ID NO: 97 - RNA, 39 nt, synthetic construct
  misc_feature 1..39: note = Synthetic RNA
  source 1..39: mol_type = other RNA
taatttctactcttgtagattgtgaaatggcttataatt

SEQ ID NO: 98 - RNA, 38 nt, synthetic construct
  misc_feature 1..38: note = Synthetic RNA
  source 1..38: mol_type = other RNA
taatttctactcttgtagattgtgaaatggcttataat

SEQ ID NO: 99 - RNA, 37 nt, synthetic construct
  misc_feature 1..37: note = Synthetic RNA
  source 1..37: mol_type = other RNA
taatttctactcttgtagattgtgaaatggcttataa

SEQ ID NO: 100 - RNA, 39 nt, synthetic construct
  misc_feature 1..39: note = Synthetic RNA
  source 1..39: mol_type = other RNA
taatttctactcttgtagataatgtaagtaattgcttct

SEQ ID NO: 101 - RNA, 38 nt, synthetic construct
  misc_feature 1..38: note = Synthetic RNA
  source 1..38: mol_type = other RNA
taatttctactcttgtagataatgtaagtaattgcttc

SEQ ID NO: 102 - RNA, 37 nt, synthetic construct
  misc_feature 1..37: note = Synthetic RNA
  source 1..37: mol_type = other RNA
taatttctactcttgtagataatgtaagtaattgctt

SEQ ID NO: 103 - RNA, 44 nt, synthetic construct; identical to SEQ ID NO: 41
  misc_feature 1..44: note = Synthetic RNA
  source 1..44: mol_type = other RNA

SEQ ID NO: 104 - RNA, 44 nt, synthetic construct; identical to SEQ ID NO: 41
  misc_feature 1..44: note = Synthetic RNA
  source 1..44: mol_type = other RNA
  modified_base 1: mod_base = OTHER; note = 2-Prime-O-methyl ribonucleotides

SEQ ID NO: 105 - RNA, 44 nt, synthetic construct; identical to SEQ ID NO: 41
  misc_feature 1..44: note = Synthetic RNA
  source 1..44: mol_type = other RNA
  modified_base 2: mod_base = OTHER; note = 2-Prime-O-methyl ribonucleotides

SEQ ID NO: 106 - RNA, 44 nt, synthetic construct; identical to SEQ ID NO: 41
  misc_feature 1..44: note = Synthetic RNA
  source 1..44: mol_type = other RNA
  modified_base 3: mod_base = OTHER; note = 2-Prime-O-methyl ribonucleotides

SEQ ID NO: 107 - RNA, 44 nt, synthetic construct; identical to SEQ ID NO: 41
  misc_feature 1..44: note = Synthetic RNA
  source 1..44: mol_type = other RNA
  modified_base 4: mod_base = OTHER; note = 2-Prime-O-methyl ribonucleotides

SEQ ID NO: 108 - RNA, 44 nt, synthetic construct; identical to SEQ ID NO: 41
  misc_feature 1..44: note = Synthetic RNA
  source 1..44: mol_type = other RNA
  modified_base 5: mod_base = OTHER; note = 2-Prime-O-methyl ribonucleotides

SEQ ID NO: 109 - RNA, 44 nt, synthetic construct; identical to SEQ ID NO: 41
  misc_feature 1..44: note = Synthetic RNA
  source 1..44: mol_type = other RNA
  modified_base 6: mod_base = OTHER; note = 2-Prime-O-methyl ribonucleotides

SEQ ID NO: 110 - RNA, 44 nt, synthetic construct; identical to SEQ ID NO: 41
  misc_feature 1..44: note = Synthetic RNA
  source 1..44: mol_type = other RNA
  modified_base 7: mod_base = OTHER; note = 2-Prime-O-methyl ribonucleotides

SEQ ID NO: 111 - RNA, 44 nt, synthetic construct; identical to SEQ ID NO: 41
  misc_feature 1..44: note = Synthetic RNA
  source 1..44: mol_type = other RNA
  modified_base 8: mod_base = OTHER; note = 2-Prime-O-methyl ribonucleotides

SEQ ID NO: 112 - RNA, 44 nt, synthetic construct; identical to SEQ ID NO: 41
  misc_feature 1..44: note = Synthetic RNA
  source 1..44: mol_type = other RNA
  modified_base 9: mod_base = OTHER; note = 2-Prime-O-methyl ribonucleotides

SEQ ID NO: 113 - RNA, 44 nt, synthetic construct; identical to SEQ ID NO: 41
  misc_feature 1..44: note = Synthetic RNA
  source 1..44: mol_type = other RNA
  modified_base 10: mod_base = OTHER; note = 2-Prime-O-methyl ribonucleotides

SEQ ID NO: 114 - RNA, 44 nt, synthetic construct; identical to SEQ ID NO: 41
  misc_feature 1..44: note = Synthetic RNA
  source 1..44: mol_type = other RNA
  modified_base 11: mod_base = OTHER; note = 2-Prime-O-methyl ribonucleotides

SEQ ID NO: 115 - RNA, 44 nt, synthetic construct; identical to SEQ ID NO: 41
  misc_feature 1..44: note = Synthetic RNA
  source 1..44: mol_type = other RNA
  modified_base 12: mod_base = OTHER; note = 2-Prime-O-methyl ribonucleotides

SEQ ID NO: 116 - RNA, 44 nt, synthetic construct; identical to SEQ ID NO: 41
  misc_feature 1..44: note = Synthetic RNA
  source 1..44: mol_type = other RNA
  modified_base 13: mod_base = OTHER; note = 2-Prime-O-methyl ribonucleotides

SEQ ID NO: 117 - RNA, 44 nt, synthetic construct; identical to SEQ ID NO: 41
  misc_feature 1..44: note = Synthetic RNA
  source 1..44: mol_type = other RNA
  modified_base 14: mod_base = OTHER; note = 2-Prime-O-methyl ribonucleotides

SEQ ID NO: 118 - RNA, 44 nt, synthetic construct; identical to SEQ ID NO: 41
  misc_feature 1..44: note = Synthetic RNA
  source 1..44: mol_type = other RNA
  modified_base 15: mod_base = OTHER; note = 2-Prime-O-methyl ribonucleotides

SEQ ID NO: 119 - RNA, 44 nt, synthetic construct; identical to SEQ ID NO: 41
  misc_feature 1..44: note = Synthetic RNA
  source 1..44: mol_type = other RNA
  modified_base 16: mod_base = OTHER; note = 2-Prime-O-methyl ribonucleotides

SEQ ID NO: 120 - RNA, 44 nt, synthetic construct; identical to SEQ ID NO: 41
  misc_feature 1..44: note = Synthetic RNA
  source 1..44: mol_type = other RNA
  modified_base 17: mod_base = OTHER; note = 2-Prime-O-methyl ribonucleotides

SEQ ID NO: 121 - RNA, 44 nt, synthetic construct; identical to SEQ ID NO: 41
  misc_feature 1..44: note = Synthetic RNA
  source 1..44: mol_type = other RNA
  modified_base 18: mod_base = OTHER; note = 2-Prime-O-methyl ribonucleotides

SEQ ID NO: 122 - RNA, 44 nt, synthetic construct; identical to SEQ ID NO: 41
  misc_feature 1..44: note = Synthetic RNA
  source 1..44: mol_type = other RNA
  modified_base 19: mod_base = OTHER; note = 2-Prime-O-methyl ribonucleotides

SEQ ID NO: 123 - RNA, 44 nt, synthetic construct; identical to SEQ ID NO: 41
  misc_feature 1..44: note = Synthetic RNA
  source 1..44: mol_type = other RNA
  modified_base 20: mod_base = OTHER; note = 2-Prime-O-methyl ribonucleotides

SEQ ID NO: 124 - RNA, 44 nt, synthetic construct; identical to SEQ ID NO: 41
  misc_feature 1..44: note = Synthetic RNA
  source 1..44: mol_type = other RNA
  modified_base 21: mod_base = OTHER; note = 2-Prime-O-methyl ribonucleotides

SEQ ID NO: 125 - RNA, 44 nt, synthetic construct; identical to SEQ ID NO: 41
  misc_feature 1..44: note = Synthetic RNA
  source 1..44: mol_type = other RNA
  modified_base 22: mod_base = OTHER; note = 2-Prime-O-methyl ribonucleotides

SEQ ID NO: 126 - RNA, 44 nt, synthetic construct; identical to SEQ ID NO: 41
  misc_feature 1..44: note = Synthetic RNA
  source 1..44: mol_type = other RNA
  modified_base 23: mod_base = OTHER; note = 2-Prime-O-methyl ribonucleotides

SEQ ID NO: 127 - RNA, 44 nt, synthetic construct; identical to SEQ ID NO: 41
  misc_feature 1..44: note = Synthetic RNA
  source 1..44: mol_type = other RNA
  modified_base 24: mod_base = OTHER; note = 2-Prime-O-methyl ribonucleotides

SEQ ID NO: 128 - RNA, 44 nt, synthetic construct; identical to SEQ ID NO: 41
  misc_feature 1..44: note = Synthetic RNA
  source 1..44: mol_type = other RNA
  modified_base 25: mod_base = OTHER; note = 2-Prime-O-methyl ribonucleotides

SEQ ID NO: 129 - RNA, 44 nt, synthetic construct; identical to SEQ ID NO: 41
  misc_feature 1..44: note = Synthetic RNA
  source 1..44: mol_type = other RNA
  modified_base 26: mod_base = OTHER; note = 2-Prime-O-methyl ribonucleotides

SEQ ID NO: 130 - RNA, 44 nt, synthetic construct; identical to SEQ ID NO: 41
  misc_feature 1..44: note = Synthetic RNA
  source 1..44: mol_type = other RNA
  modified_base 27: mod_base = OTHER; note = 2-Prime-O-methyl ribonucleotides

SEQ ID NO: 131 - RNA, 44 nt, synthetic construct; identical to SEQ ID NO: 41
  misc_feature 1..44: note = Synthetic RNA
  source 1..44: mol_type = other RNA
  modified_base 28: mod_base = OTHER; note = 2-Prime-O-methyl ribonucleotides

SEQ ID NO: 132 - RNA, 44 nt, synthetic construct; identical to SEQ ID NO: 41
  misc_feature 1..44: note = Synthetic RNA
  source 1..44: mol_type = other RNA
  modified_base 29: mod_base = OTHER; note = 2-Prime-O-methyl ribonucleotides

SEQ ID NO: 133 - RNA, 44 nt, synthetic construct; identical to SEQ ID NO: 41
  misc_feature 1..44: note = Synthetic RNA
  source 1..44: mol_type = other RNA
  modified_base 30: mod_base = OTHER; note = 2-Prime-O-methyl ribonucleotides

SEQ ID NO: 134 - RNA, 44 nt, synthetic construct; identical to SEQ ID NO: 41
  misc_feature 1..44: note = Synthetic RNA
  source 1..44: mol_type = other RNA
  modified_base 31: mod_base = OTHER; note = 2-Prime-O-methyl ribonucleotides

SEQ ID NO: 135 - RNA, 44 nt, synthetic construct; identical to SEQ ID NO: 41
  misc_feature 1..44: note = Synthetic RNA
  source 1..44: mol_type = other RNA
  modified_base 32: mod_base = OTHER; note = 2-Prime-O-methyl ribonucleotides

SEQ ID NO: 136 - RNA, 44 nt, synthetic construct; identical to SEQ ID NO: 41
  misc_feature 1..44: note = Synthetic RNA
  source 1..44: mol_type = other RNA
  modified_base 33: mod_base = OTHER; note = 2-Prime-O-methyl ribonucleotides

SEQ ID NO: 137 - RNA, 44 nt, synthetic construct; identical to SEQ ID NO: 41
  misc_feature 1..44: note = Synthetic RNA
  source 1..44: mol_type = other RNA
  modified_base 34: mod_base = OTHER; note = 2-Prime-O-methyl ribonucleotides

SEQ ID NO: 138 - RNA, 44 nt, synthetic construct; identical to SEQ ID NO: 41
  misc_feature 1..44: note = Synthetic RNA
  source 1..44: mol_type = other RNA
  modified_base 35: mod_base = OTHER; note = 2-Prime-O-methyl ribonucleotides

SEQ ID NO: 139 - RNA, 44 nt, synthetic construct; identical to SEQ ID NO: 41
  misc_feature 1..44: note = Synthetic RNA
  source 1..44: mol_type = other RNA
  modified_base 36: mod_base = OTHER; note = 2-Prime-O-methyl ribonucleotides

SEQ ID NO: 140 - RNA, 44 nt, synthetic construct; identical to SEQ ID NO: 41
  misc_feature 1..44: note = Synthetic RNA
  source 1..44: mol_type = other RNA
  modified_base 37: mod_base = OTHER; note = 2-Prime-O-methyl ribonucleotides

SEQ ID NO: 141 - RNA, 44 nt, synthetic construct; identical to SEQ ID NO: 41
  misc_feature 1..44: note = Synthetic RNA
  source 1..44: mol_type = other RNA
  modified_base 38: mod_base = OTHER; note = 2-Prime-O-methyl ribonucleotides

SEQ ID NO: 142 - RNA, 44 nt, synthetic construct; identical to SEQ ID NO: 41
  misc_feature 1..44: note = Synthetic RNA
  source 1..44: mol_type = other RNA
  modified_base 39: mod_base = OTHER; note = 2-Prime-O-methyl ribonucleotides

SEQ ID NO: 143 - RNA, 44 nt, synthetic construct; identical to SEQ ID NO: 41
  misc_feature 1..44: note = Synthetic RNA
  source 1..44: mol_type = other RNA
  modified_base 40: mod_base = OTHER; note = 2-Prime-O-methyl ribonucleotides

SEQ ID NO: 144 - RNA, 44 nt, synthetic construct; identical to SEQ ID NO: 41
  misc_feature 1..44: note = Synthetic RNA
  source 1..44: mol_type = other RNA
  modified_base 41: mod_base = OTHER; note = 2-Prime-O-methyl ribonucleotides

SEQ ID NO: 145 - RNA, 44 nt, synthetic construct; identical to SEQ ID NO: 41
  misc_feature 1..44: note = Synthetic RNA
  source 1..44: mol_type = other RNA
  modified_base 42: mod_base = OTHER; note = 2-Prime-O-methyl ribonucleotides

SEQ ID NO: 146 - RNA, 44 nt, synthetic construct; identical to SEQ ID NO: 41
  misc_feature 1..44: note = Synthetic RNA
  source 1..44: mol_type = other RNA
  modified_base 43: mod_base = OTHER; note = 2-Prime-O-methyl ribonucleotides

SEQ ID NO: 147 - RNA, 44 nt, synthetic construct; identical to SEQ ID NO: 41
  misc_feature 1..44: note = Synthetic RNA
  source 1..44: mol_type = other RNA
  modified_base 44: mod_base = OTHER; note = 2-Prime-O-methyl ribonucleotides

SEQ ID NO: 148 - RNA, 41 nt, synthetic construct; identical to SEQ ID NO: 77
  misc_feature 1..41: note = SYNTHETIC RNA
  source 1..41: mol_type = other RNA

SEQ ID NO: 149 - RNA, 41 nt, synthetic construct; identical to SEQ ID NO: 77
  misc_feature 1..41: note = SYNTHETIC RNA
  source 1..41: mol_type = other RNA
  modified_base 1: mod_base = OTHER; note = 2-Prime-O-methyl ribonucleotides

SEQ ID NO: 150 - RNA, 41 nt, synthetic construct; identical to SEQ ID NO: 77
  misc_feature 1..41: note = SYNTHETIC RNA
  source 1..41: mol_type = other RNA
  modified_base 2: mod_base = OTHER; note = 2-Prime-O-methyl ribonucleotides

SEQ ID NO: 151 - RNA, 41 nt, synthetic construct; identical to SEQ ID NO: 77
  misc_feature 1..41: note = SYNTHETIC RNA
  source 1..41: mol_type = other RNA
  modified_base 3: mod_base = OTHER; note = 2-Prime-O-methyl ribonucleotides

SEQ ID NO: 152 - RNA, 41 nt, synthetic construct; identical to SEQ ID NO: 77
  misc_feature 1..41: note = SYNTHETIC RNA
  source 1..41: mol_type = other RNA
  modified_base 4: mod_base = OTHER; note = 2-Prime-O-methyl ribonucleotides

SEQ ID NO: 153 - RNA, 41 nt, synthetic construct; identical to SEQ ID NO: 77
  misc_feature 1..41: note = SYNTHETIC RNA
  source 1..41: mol_type = other RNA
  modified_base 5: mod_base = OTHER; note = 2-Prime-O-methyl ribonucleotides

SEQ ID NO: 154 - RNA, 41 nt, synthetic construct; identical to SEQ ID NO: 77
  misc_feature 1..41: note = SYNTHETIC RNA
  source 1..41: mol_type = other RNA
  modified_base 6: mod_base = OTHER; note = 2-Prime-O-methyl ribonucleotides

SEQ ID NO: 155 - RNA, 41 nt, synthetic construct; identical to SEQ ID NO: 77
  misc_feature 1..41: note = SYNTHETIC RNA
  source 1..41: mol_type = other RNA
  modified_base 7: mod_base = OTHER; note = 2-Prime-O-methyl ribonucleotides

SEQ ID NO: 156 - RNA, 41 nt, synthetic construct; identical to SEQ ID NO: 77
  misc_feature 1..41: note = SYNTHETIC RNA
  source 1..41: mol_type = other RNA
  modified_base 8: mod_base = OTHER; note = 2-Prime-O-methyl ribonucleotides

SEQ ID NO: 157 - RNA, 41 nt, synthetic construct; identical to SEQ ID NO: 77
  misc_feature 1..41: note = SYNTHETIC RNA
  source 1..41: mol_type = other RNA
  modified_base 9: mod_base = OTHER; note = 2-Prime-O-methyl ribonucleotides

SEQ ID NO: 158 - RNA, 41 nt, synthetic construct; identical to SEQ ID NO: 77
  misc_feature 1..41: note = SYNTHETIC RNA
  source 1..41: mol_type = other RNA
  modified_base 10: mod_base = OTHER; note = 2-Prime-O-methyl ribonucleotides

SEQ ID NO: 159 - RNA, 41 nt, synthetic construct; identical to SEQ ID NO: 77
  misc_feature 1..41: note = SYNTHETIC RNA
  source 1..41: mol_type = other RNA
  modified_base 11: mod_base = OTHER; note = 2-Prime-O-methyl ribonucleotides

SEQ ID NO: 160 - RNA, 41 nt, synthetic construct; identical to SEQ ID NO: 77
  misc_feature 1..41: note = SYNTHETIC RNA
  source 1..41: mol_type = other RNA
  modified_base 12: mod_base = OTHER; note = 2-Prime-O-methyl ribonucleotides

SEQ ID NO: 161 - RNA, 41 nt, synthetic construct; identical to SEQ ID NO: 77
  misc_feature 1..41: note = SYNTHETIC RNA
  source 1..41: mol_type = other RNA
  modified_base 13: mod_base = OTHER; note = 2-Prime-O-methyl ribonucleotides

SEQ ID NO: 162 - RNA, 41 nt, synthetic construct; identical to SEQ ID NO: 77
  misc_feature 1..41: note = SYNTHETIC RNA
  source 1..41: mol_type = other RNA
  modified_base 14: mod_base = OTHER; note = 2-Prime-O-methyl ribonucleotides

SEQ ID NO: 163 - RNA, 41 nt, synthetic construct; identical to SEQ ID NO: 77
  misc_feature 1..41: note = SYNTHETIC RNA
  source 1..41: mol_type = other RNA
  modified_base 15: mod_base = OTHER; note = 2-Prime-O-methyl ribonucleotides

SEQ ID NO: 164 - RNA, 41 nt, synthetic construct; identical to SEQ ID NO: 77
  misc_feature 1..41: note = SYNTHETIC RNA
  source 1..41: mol_type = other RNA
  modified_base 16: mod_base = OTHER; note = 2-Prime-O-methyl ribonucleotides

SEQ ID NO: 165 - RNA, 41 nt, synthetic construct; identical to SEQ ID NO: 77
  misc_feature 1..41: note = SYNTHETIC RNA
  source 1..41: mol_type = other RNA
  modified_base 17: mod_base = OTHER; note = 2-Prime-O-methyl ribonucleotides

SEQ ID NO: 166 - RNA, 41 nt, synthetic construct; identical to SEQ ID NO: 77
  misc_feature 1..41: note = SYNTHETIC RNA
  source 1..41: mol_type = other RNA
  modified_base 18: mod_base = OTHER; note = 2-Prime-O-methyl ribonucleotides

SEQ ID NO: 167 - RNA, 41 nt, synthetic construct; identical to SEQ ID NO: 77
  misc_feature 1..41: note = SYNTHETIC RNA
  source 1..41: mol_type = other RNA
  modified_base 19: mod_base = OTHER; note = 2-Prime-O-methyl ribonucleotides

SEQ ID NO: 168 - RNA, 41 nt, synthetic construct; identical to SEQ ID NO: 77
  misc_feature 1..41: note = SYNTHETIC RNA
  source 1..41: mol_type = other RNA
  modified_base 20: mod_base = OTHER; note = 2-Prime-O-methyl ribonucleotides

SEQ ID NO: 169 - RNA, 41 nt, synthetic construct; identical to SEQ ID NO: 77
  misc_feature 1..41: note = SYNTHETIC RNA
  source 1..41: mol_type = other RNA
  modified_base 21: mod_base = OTHER; note = 2-Prime-O-methyl ribonucleotides

SEQ ID NO: 170 - RNA, 41 nt, synthetic construct; identical to SEQ ID NO: 77
  misc_feature 1..41: note = SYNTHETIC RNA
  source 1..41: mol_type = other RNA
  modified_base 22: mod_base = OTHER; note = 2-Prime-O-methyl ribonucleotides

SEQ ID NO: 171 - RNA, 41 nt, synthetic construct; identical to SEQ ID NO: 77
  misc_feature 1..41: note = SYNTHETIC RNA
  source 1..41: mol_type = other RNA
  modified_base 23: mod_base = OTHER; note = 2-Prime-O-methyl ribonucleotides

SEQ ID NO: 172 - RNA, 41 nt, synthetic construct; identical to SEQ ID NO: 77
  misc_feature 1..41: note = SYNTHETIC RNA
  source 1..41: mol_type = other RNA
  modified_base 24: mod_base = OTHER; note = 2-Prime-O-methyl ribonucleotides

SEQ ID NO: 173 - RNA, 41 nt, synthetic construct; identical to SEQ ID NO: 77
  misc_feature 1..41: note = SYNTHETIC RNA
  source 1..41: mol_type = other RNA
  modified_base 25: mod_base = OTHER; note = 2-Prime-O-methyl ribonucleotides

SEQ ID NO: 174 - RNA, 41 nt, synthetic construct; identical to SEQ ID NO: 77
  misc_feature 1..41: note = SYNTHETIC RNA
  source 1..41: mol_type = other RNA
  modified_base 26: mod_base = OTHER; note = 2-Prime-O-methyl ribonucleotides

SEQ ID NO: 175 - RNA, 41 nt, synthetic construct; identical to SEQ ID NO: 77
  misc_feature 1..41: note = SYNTHETIC RNA
  source 1..41: mol_type = other RNA
  modified_base 27: mod_base = OTHER; note = 2-Prime-O-methyl ribonucleotides

SEQ ID NO: 176 - RNA, 41 nt, synthetic construct; identical to SEQ ID NO: 77
  misc_feature 1..41: note = SYNTHETIC RNA
  source 1..41: mol_type = other RNA
  modified_base 28: mod_base = OTHER; note = 2-Prime-O-methyl ribonucleotides

SEQ ID NO: 177 - RNA, 41 nt, synthetic construct; identical to SEQ ID NO: 77
  misc_feature 1..41: note = SYNTHETIC RNA
  source 1..41: mol_type = other RNA
  modified_base 29: mod_base = OTHER; note = 2-Prime-O-methyl ribonucleotides

SEQ ID NO: 178 - RNA, 41 nt, synthetic construct; identical to SEQ ID NO: 77
  misc_feature 1..41: note = SYNTHETIC RNA
  source 1..41: mol_type = other RNA
  modified_base 30: mod_base = OTHER; note = 2-Prime-O-methyl ribonucleotides

SEQ ID NO: 179 - RNA, 41 nt, synthetic construct; identical to SEQ ID NO: 77
  misc_feature 1..41: note = SYNTHETIC RNA
  source 1..41: mol_type = other RNA
  modified_base 31: mod_base = OTHER; note = 2-Prime-O-methyl ribonucleotides

SEQ ID NO: 180 - RNA, 41 nt, synthetic construct; identical to SEQ ID NO: 77
  misc_feature 1..41: note = SYNTHETIC RNA
  source 1..41: mol_type = other RNA
  modified_base 32: mod_base = OTHER; note = 2-Prime-O-methyl ribonucleotides

SEQ ID NO: 181 - RNA, 41 nt, synthetic construct; identical to SEQ ID NO: 77
  misc_feature 1..41: note = SYNTHETIC RNA
  source 1..41: mol_type = other RNA
  modified_base 33: mod_base = OTHER; note = 2-Prime-O-methyl ribonucleotides

SEQ ID NO: 182 - RNA, 41 nt, synthetic construct; identical to SEQ ID NO: 77
  misc_feature 1..41: note = SYNTHETIC RNA
  source 1..41: mol_type = other RNA
  modified_base 34: mod_base = OTHER; note = 2-Prime-O-methyl ribonucleotides

SEQ ID NO: 183 - RNA, 41 nt, synthetic construct; identical to SEQ ID NO: 77
  misc_feature 1..41: note = SYNTHETIC RNA
  source 1..41: mol_type = other RNA
  modified_base 35: mod_base = OTHER; note = 2-Prime-O-methyl ribonucleotides

SEQ ID NO: 184 - RNA, 41 nt, synthetic construct; identical to SEQ ID NO: 77
  misc_feature 1..41: note = SYNTHETIC RNA
  source 1..41: mol_type = other RNA
  modified_base 36: mod_base = OTHER; note = 2-Prime-O-methyl ribonucleotides

SEQ ID NO: 185 - RNA, 41 nt, synthetic construct; identical to SEQ ID NO: 77
  misc_feature 1..41: note = SYNTHETIC RNA
  source 1..41: mol_type = other RNA
  modified_base 37: mod_base = OTHER; note = 2-Prime-O-methyl ribonucleotides

SEQ ID NO: 186 - RNA, 41 nt, synthetic construct; identical to SEQ ID NO: 77
  misc_feature 1..41: note = SYNTHETIC RNA
  source 1..41: mol_type = other RNA
  modified_base 38: mod_base = OTHER; note = 2-Prime-O-methyl ribonucleotides

SEQ ID NO: 187 - RNA, 41 nt, synthetic construct; identical to SEQ ID NO: 77
  misc_feature 1..41: note = SYNTHETIC RNA
  source 1..41: mol_type = other RNA
  modified_base 39: mod_base = OTHER; note = 2-Prime-O-methyl ribonucleotides

SEQ ID NO: 188 - RNA, 41 nt, synthetic construct; identical to SEQ ID NO: 77
  misc_feature 1..41: note = SYNTHETIC RNA
  source 1..41: mol_type = other RNA
  modified_base 40: mod_base = OTHER; note = 2-Prime-O-methyl ribonucleotides

SEQ ID NO: 189 - RNA, 41 nt, synthetic construct; identical to SEQ ID NO: 77
  misc_feature 1..41: note = SYNTHETIC RNA
  source 1..41: mol_type = other RNA
  modified_base 41: mod_base = OTHER; note = 2-Prime-O-methyl ribonucleotides

SEQ ID NO: 190 - RNA, 44 nt, synthetic construct
  misc_feature 1..44: note = SYNTHETIC RNA
  source 1..44: mol_type = other RNA
taatttctactcttgtagatggaaagagaattgttttctccttc

SEQ ID NO: 191 - RNA, 44 nt, synthetic construct; identical to SEQ ID NO: 190
  misc_feature 1..44: note = SYNTHETIC RNA
  source 1..44: mol_type = other RNA
  modified_base 1: mod_base = OTHER; note = 2-Prime-O-methyl ribonucleotides

SEQ ID NO: 192 - RNA, 44 nt, synthetic construct; identical to SEQ ID NO: 190
  misc_feature 1..44: note = SYNTHETIC RNA
  source 1..44: mol_type = other RNA
  modified_base 2: mod_base = OTHER; note = 2-Prime-O-methyl ribonucleotides

SEQ ID NO: 193 - RNA, 44 nt, synthetic construct; identical to SEQ ID NO: 190
  misc_feature 1..44: note = SYNTHETIC RNA
  source 1..44: mol_type = other RNA
  modified_base 3: mod_base = OTHER; note = 2-Prime-O-methyl ribonucleotides

SEQ ID NO: 194 - RNA, 44 nt, synthetic construct; identical to SEQ ID NO: 190
  misc_feature 1..44: note = SYNTHETIC RNA
  source 1..44: mol_type = other RNA
  modified_base 4: mod_base = OTHER; note = 2-Prime-O-methyl ribonucleotides

SEQ ID NO: 195 - RNA, 44 nt, synthetic construct; identical to SEQ ID NO: 190
  misc_feature 1..44: note = SYNTHETIC RNA
  source 1..44: mol_type = other RNA
  modified_base 5: mod_base = OTHER; note = 2-Prime-O-methyl ribonucleotides

SEQ ID NO: 196 - RNA, 44 nt, synthetic construct; identical to SEQ ID NO: 190
  misc_feature 1..44: note = SYNTHETIC RNA
  source 1..44: mol_type = other RNA
  modified_base 6: mod_base = OTHER; note = 2-Prime-O-methyl ribonucleotides

SEQ ID NO: 197 - RNA, 44 nt, synthetic construct; identical to SEQ ID NO: 190
  misc_feature 1..44: note = SYNTHETIC RNA
  source 1..44: mol_type = other RNA
  modified_base 7: mod_base = OTHER; note = 2-Prime-O-methyl ribonucleotides

SEQ ID NO: 198 - RNA, 44 nt, synthetic construct; identical to SEQ ID NO: 190
  misc_feature 1..44: note = SYNTHETIC RNA
  source 1..44: mol_type = other RNA
  modified_base 8: mod_base = OTHER; note = 2-Prime-O-methyl ribonucleotides

SEQ ID NO: 199 - RNA, 44 nt, synthetic construct; identical to SEQ ID NO: 190
  misc_feature 1..44: note = SYNTHETIC RNA
  source 1..44: mol_type = other RNA
  modified_base 9: mod_base = OTHER; note = 2-Prime-O-methyl ribonucleotides

SEQ ID NO: 200 - RNA, 44 nt, synthetic construct; identical to SEQ ID NO: 190
  misc_feature 1..44: note = SYNTHETIC RNA
  source 1..44: mol_type = other RNA
  modified_base 10: mod_base = OTHER; note = 2-Prime-O-methyl ribonucleotides

SEQ ID NO: 201 - RNA, 44 nt, synthetic construct; identical to SEQ ID NO: 190
  misc_feature 1..44: note = SYNTHETIC RNA
  source 1..44: mol_type = other RNA
  modified_base 11: mod_base = OTHER; note = 2-Prime-O-methyl ribonucleotides

SEQ ID NO: 202 - RNA, 44 nt, synthetic construct; identical to SEQ ID NO: 190
  misc_feature 1..44: note = SYNTHETIC RNA
  source 1..44: mol_type = other RNA
  modified_base 12: mod_base = OTHER; note = 2-Prime-O-methyl ribonucleotides

SEQ ID NO: 203 - RNA, 44 nt, synthetic construct; identical to SEQ ID NO: 190
  misc_feature 1..44: note = SYNTHETIC RNA
  source 1..44: mol_type = other RNA
  modified_base 13: mod_base = OTHER; note = 2-Prime-O-methyl ribonucleotides

SEQ ID NO: 204 - RNA, 44 nt, synthetic construct; identical to SEQ ID NO: 190
  misc_feature 1..44: note = SYNTHETIC RNA
  source 1..44: mol_type = other RNA
  modified_base 14: mod_base = OTHER; note = 2-Prime-O-methyl ribonucleotides

SEQ ID NO: 205 - RNA, 44 nt, synthetic construct; identical to SEQ ID NO: 190
  misc_feature 1..44: note = SYNTHETIC RNA
  source 1..44: mol_type = other RNA
  modified_base 15: mod_base = OTHER; note = 2-Prime-O-methyl ribonucleotides

SEQ ID NO: 206 - RNA, 44 nt, synthetic construct; identical to SEQ ID NO: 190
  misc_feature 1..44: note = SYNTHETIC RNA
  source 1..44: mol_type = other RNA
  modified_base 16: mod_base = OTHER; note = 2-Prime-O-methyl ribonucleotides

SEQ ID NO: 207 - RNA, 44 nt, synthetic construct; identical to SEQ ID NO: 190
  misc_feature 1..44: note = SYNTHETIC RNA
  source 1..44: mol_type = other RNA
  modified_base 17: mod_base = OTHER; note = 2-Prime-O-methyl ribonucleotides

SEQ ID NO: 208 - RNA, 44 nt, synthetic construct; identical to SEQ ID NO: 190
  misc_feature 1..44: note = SYNTHETIC RNA
  source 1..44: mol_type = other RNA
  modified_base 18: mod_base = OTHER; note = 2-Prime-O-methyl ribonucleotides

SEQ ID NO: 209 - RNA, 44 nt, synthetic construct; identical to SEQ ID NO: 190
  misc_feature 1..44: note = SYNTHETIC RNA
  source 1..44: mol_type = other RNA
  modified_base 19: mod_base = OTHER; note = 2-Prime-O-methyl ribonucleotides

SEQ ID NO: 210 - RNA, 44 nt, synthetic construct; identical to SEQ ID NO: 190
  misc_feature 1..44: note = SYNTHETIC RNA
  source 1..44: mol_type = other RNA
  modified_base 20: mod_base = OTHER; note = 2-Prime-O-methyl ribonucleotides

SEQ ID NO: 211 - RNA, 44 nt, synthetic construct; identical to SEQ ID NO: 190
  misc_feature 1..44: note = SYNTHETIC RNA
  source 1..44: mol_type = other RNA
  modified_base 21: mod_base = OTHER; note = 2-Prime-O-methyl ribonucleotides

SEQ ID NO: 212 - RNA, 44 nt, synthetic construct; identical to SEQ ID NO: 190
  misc_feature 1..44: note = SYNTHETIC RNA
  source 1..44: mol_type = other RNA
  modified_base 22: mod_base = OTHER; note = 2-Prime-O-methyl ribonucleotides

SEQ ID NO: 213 - RNA, 44 nt, synthetic construct; identical to SEQ ID NO: 190
  misc_feature 1..44: note = SYNTHETIC RNA
  source 1..44: mol_type = other RNA
  modified_base 23: mod_base = OTHER; note = 2-Prime-O-methyl ribonucleotides

SEQ ID NO: 214 - RNA, 44 nt, synthetic construct; identical to SEQ ID NO: 190
  misc_feature 1..44: note = SYNTHETIC RNA
  source 1..44: mol_type = other RNA
  modified_base 24: mod_base = OTHER; note = 2-Prime-O-methyl ribonucleotides

SEQ ID NO: 215 - RNA, 44 nt, synthetic construct; identical to SEQ ID NO: 190
  misc_feature 1..44: note = SYNTHETIC RNA
  source 1..44: mol_type = other RNA
  modified_base 25: mod_base = OTHER; note = 2-Prime-O-methyl ribonucleotides

SEQ ID NO: 216 - RNA, 44 nt, synthetic construct; identical to SEQ ID NO: 190
  misc_feature 1..44: note = SYNTHETIC RNA
  source 1..44: mol_type = other RNA
  modified_base 26: mod_base = OTHER; note = 2-Prime-O-methyl ribonucleotides

SEQ ID NO: 217 - RNA, 44 nt, synthetic construct; identical to SEQ ID NO: 190
  misc_feature 1..44: note = SYNTHETIC RNA
  source 1..44: mol_type = other RNA
  modified_base 27: mod_base = OTHER; note = 2-Prime-O-methyl ribonucleotides

SEQ ID NO: 218 - RNA, 44 nt, synthetic construct; identical to SEQ ID NO: 190
  misc_feature 1..44: note = SYNTHETIC RNA
  source 1..44: mol_type = other RNA
  modified_base 28: mod_base = OTHER; note = 2-Prime-O-methyl ribonucleotides

SEQ ID NO: 219 - RNA, 44 nt, synthetic construct; identical to SEQ ID NO: 190
  misc_feature 1..44: note = SYNTHETIC RNA
  source 1..44: mol_type = other RNA
  modified_base 29: mod_base = OTHER; note = 2-Prime-O-methyl ribonucleotides

SEQ ID NO: 220 - RNA, 44 nt, synthetic construct; identical to SEQ ID NO: 190
  misc_feature 1..44: note = SYNTHETIC RNA
  source 1..44: mol_type = other RNA
  modified_base 30: mod_base = OTHER; note = 2-Prime-O-methyl ribonucleotides

SEQ ID NO: 221 - RNA, 44 nt, synthetic construct; identical to SEQ ID NO: 190
  misc_feature 1..44: note = SYNTHETIC RNA
  source 1..44: mol_type = other RNA
  modified_base 31: mod_base = OTHER; note = 2-Prime-O-methyl ribonucleotides

SEQ ID NO: 222 - RNA, 44 nt, synthetic construct; identical to SEQ ID NO: 190
  misc_feature 1..44: note = SYNTHETIC RNA
  source 1..44: mol_type = other RNA
  modified_base 32: mod_base = OTHER; note = 2-Prime-O-methyl ribonucleotides

SEQ ID NO: 223 - RNA, 44 nt, synthetic construct; identical to SEQ ID NO: 190
  misc_feature 1..44: note = SYNTHETIC RNA
  source 1..44: mol_type = other RNA
  modified_base 33: mod_base = OTHER; note = 2-Prime-O-methyl ribonucleotides

SEQ ID NO: 224 - RNA, 44 nt, synthetic construct; identical to SEQ ID NO: 190
  misc_feature 1..44: note = SYNTHETIC RNA
  source 1..44: mol_type = other RNA
  modified_base 34: mod_base = OTHER; note = 2-Prime-O-methyl ribonucleotides

SEQ ID NO: 225 - RNA, 44 nt, synthetic construct; identical to SEQ ID NO: 190
  misc_feature 1..44: note = SYNTHETIC RNA
  source 1..44: mol_type = other RNA
  modified_base 35: mod_base = OTHER; note = 2-Prime-O-methyl ribonucleotides

SEQ ID NO: 226 - RNA, 44 nt, synthetic construct; identical to SEQ ID NO: 190
  misc_feature 1..44: note = SYNTHETIC RNA
  source 1..44: mol_type = other RNA
  modified_base 36: mod_base = OTHER; note = 2-Prime-O-methyl ribonucleotides

SEQ ID NO: 227 - RNA, 44 nt, synthetic construct; identical to SEQ ID NO: 190
  misc_feature 1..44: note = SYNTHETIC RNA
  source 1..44: mol_type = other RNA
  modified_base 37: mod_base = OTHER; note = 2-Prime-O-methyl ribonucleotides

SEQ ID NO: 228 - RNA, 44 nt, synthetic construct; identical to SEQ ID NO: 190
  misc_feature 1..44: note = SYNTHETIC RNA
  source 1..44: mol_type = other RNA
  modified_base 38: mod_base = OTHER; note = 2-Prime-O-methyl ribonucleotides

SEQ ID NO: 229 - RNA, 44 nt, synthetic construct; identical to SEQ ID NO: 190
  misc_feature 1..44: note = SYNTHETIC RNA
  source 1..44: mol_type = other RNA
  modified_base 39: mod_base = OTHER; note = 2-Prime-O-methyl ribonucleotides

SEQ ID NO: 230 - RNA, 44 nt, synthetic construct; identical to SEQ ID NO: 190
  misc_feature 1..44: note = SYNTHETIC RNA
  source 1..44: mol_type = other RNA
  modified_base 40: mod_base = OTHER; note = 2-Prime-O-methyl ribonucleotides

SEQ ID NO: 231 - RNA, 44 nt, synthetic construct; identical to SEQ ID NO: 190
  misc_feature 1..44: note = SYNTHETIC RNA
  source 1..44: mol_type = other RNA
  modified_base 41: mod_base = OTHER; note = 2-Prime-O-methyl ribonucleotides

SEQ ID NO: 232 - RNA, 44 nt, synthetic construct; identical to SEQ ID NO: 190
  misc_feature 1..44: note = SYNTHETIC RNA
  source 1..44: mol_type = other RNA
  modified_base 42: mod_base = OTHER; note = 2-Prime-O-methyl ribonucleotides

SEQ ID NO: 233 - RNA, 44 nt, synthetic construct; identical to SEQ ID NO: 190
  misc_feature 1..44: note = SYNTHETIC RNA
  source 1..44: mol_type = other RNA
  modified_base 43: mod_base = OTHER; note = 2-Prime-O-methyl ribonucleotides

SEQ ID NO: 234 - RNA, 44 nt, synthetic construct; identical to SEQ ID NO: 190
  misc_feature 1..44: note = SYNTHETIC RNA
  source 1..44: mol_type = other RNA
  modified_base 44: mod_base = OTHER; note = 2-Prime-O-methyl ribonucleotides

SEQ ID NO: 235 - RNA, 41 nt, synthetic construct
  misc_feature 1..41: note = SYNTHETIC RNA
  source 1..41: mol_type = other RNA
taatttctactcttgtagatggaaagagaattgttttctcc

SEQ ID NO: 236 - RNA, 41 nt, synthetic construct; identical to SEQ ID NO: 235
  misc_feature 1..41: note = SYNTHETIC RNA
  source 1..41: mol_type = other RNA
  modified_base 1: mod_base = OTHER; note = 2-Prime-O-methyl ribonucleotides

SEQ ID NO: 237 - RNA, 41 nt, synthetic construct; identical to SEQ ID NO: 235
  misc_feature 1..41: note = SYNTHETIC RNA
  source 1..41: mol_type = other RNA
  modified_base 2: mod_base = OTHER; note = 2-Prime-O-methyl ribonucleotides

SEQ ID NO: 238 - RNA, 41 nt, synthetic construct; identical to SEQ ID NO: 235
  misc_feature 1..41: note = SYNTHETIC RNA
  source 1..41: mol_type = other RNA
  modified_base 3: mod_base = OTHER; note = 2-Prime-O-methyl ribonucleotides

SEQ ID NO: 239 - RNA, 41 nt, synthetic construct; identical to SEQ ID NO: 235
  misc_feature 1..41: note = SYNTHETIC RNA
  source 1..41: mol_type = other RNA
  modified_base 4: mod_base = OTHER; note = 2-Prime-O-methyl ribonucleotides

SEQ ID NO: 240 - RNA, 41 nt, synthetic construct; identical to SEQ ID NO: 235
  misc_feature 1..41: note = SYNTHETIC RNA
  source 1..41: mol_type = other RNA
  modified_base 5: mod_base = OTHER; note = 2-Prime-O-methyl ribonucleotides

SEQ ID NO: 241 - RNA, 41 nt, synthetic construct; identical to SEQ ID NO: 235
  misc_feature 1..41: note = SYNTHETIC RNA
  source 1..41: mol_type = other RNA
  modified_base 6: mod_base = OTHER; note = 2-Prime-O-methyl ribonucleotides

SEQ ID NO: 242 - RNA, 41 nt, synthetic construct; identical to SEQ ID NO: 235
  misc_feature 1..41: note = SYNTHETIC RNA
  source 1..41: mol_type = other RNA
  modified_base 7: mod_base = OTHER; note = 2-Prime-O-methyl ribonucleotides

SEQ ID NO: 243 - RNA, 41 nt, synthetic construct; identical to SEQ ID NO: 235
  misc_feature 1..41: note = SYNTHETIC RNA
  source 1..41: mol_type = other RNA
  modified_base 8: mod_base = OTHER; note = 2-Prime-O-methyl ribonucleotides

SEQ ID NO: 244 - RNA, 41 nt, synthetic construct; identical to SEQ ID NO: 235
  misc_feature 1..41: note = SYNTHETIC RNA
  source 1..41: mol_type = other RNA
  modified_base 9: mod_base = OTHER; note = 2-Prime-O-methyl ribonucleotides

SEQ ID NO: 245 - RNA, 41 nt, synthetic construct; identical to SEQ ID NO: 235
  misc_feature 1..41: note = SYNTHETIC RNA
  source 1..41: mol_type = other RNA
  modified_base 10: mod_base = OTHER; note = 2-Prime-O-methyl ribonucleotides

SEQ ID NO: 246 - RNA, 41 nt, synthetic construct; identical to SEQ ID NO: 235
  misc_feature 1..41: note = SYNTHETIC RNA
  source 1..41: mol_type = other RNA
  modified_base 11: mod_base = OTHER; note = 2-Prime-O-methyl ribonucleotides

SEQ ID NO: 247 - RNA, 41 nt, synthetic construct; identical to SEQ ID NO: 235
  misc_feature 1..41: note = SYNTHETIC RNA
  source 1..41: mol_type = other RNA
  modified_base 12: mod_base = OTHER; note = 2-Prime-O-methyl ribonucleotides

SEQ ID NO: 248 - RNA, 41 nt, synthetic construct; identical to SEQ ID NO: 235
  misc_feature 1..41: note = SYNTHETIC RNA
  source 1..41: mol_type = other RNA
  modified_base 13: mod_base = OTHER; note = 2-Prime-O-methyl ribonucleotides

SEQ ID NO: 249 - RNA, 41 nt, synthetic construct; identical to SEQ ID NO: 235
  misc_feature 1..41: note = SYNTHETIC RNA
  source 1..41: mol_type = other RNA
  modified_base 14: mod_base = OTHER; note = 2-Prime-O-methyl ribonucleotides

SEQ ID NO: 250 - RNA, 41 nt, synthetic construct; identical to SEQ ID NO: 235
  misc_feature 1..41: note = SYNTHETIC RNA
  source 1..41: mol_type = other RNA
  modified_base 15: mod_base = OTHER; note = 2-Prime-O-methyl ribonucleotides

SEQ ID NO: 251 - RNA, 41 nt, synthetic construct; identical to SEQ ID NO: 235
  misc_feature 1..41: note = SYNTHETIC RNA
  source 1..41: mol_type = other RNA
  modified_base 16: mod_base = OTHER; note = 2-Prime-O-methyl ribonucleotides

SEQ ID NO: 252 - RNA, 41 nt, synthetic construct; identical to SEQ ID NO: 235
  misc_feature 1..41: note = SYNTHETIC RNA
  source 1..41: mol_type = other RNA
  modified_base 17: mod_base = OTHER; note = 2-Prime-O-methyl ribonucleotides

SEQ ID NO: 253 - RNA, 41 nt, synthetic construct; identical to SEQ ID NO: 235
  misc_feature 1..41: note = SYNTHETIC RNA
  source 1..41: mol_type = other RNA
  modified_base 18: mod_base = OTHER; note = 2-Prime-O-methyl ribonucleotides

SEQ ID NO: 254 - RNA, 41 nt, synthetic construct; identical to SEQ ID NO: 235
  misc_feature 1..41: note = SYNTHETIC RNA
  source 1..41: mol_type = other RNA
  modified_base 19: mod_base = OTHER; note = 2-Prime-O-methyl ribonucleotides

SEQ ID NO: 255 - RNA, 41 nt, synthetic construct; identical to SEQ ID NO: 235
  misc_feature 1..41: note = SYNTHETIC RNA
  source 1..41: mol_type = other RNA
  modified_base 20: mod_base = OTHER; note = 2-Prime-O-methyl ribonucleotides

SEQ ID NO: 256 - RNA, 41 nt, synthetic construct; identical to SEQ ID NO: 235
  misc_feature 1..41: note = SYNTHETIC RNA
  source 1..41: mol_type = other RNA
  modified_base 21: mod_base = OTHER; note = 2-Prime-O-methyl ribonucleotides

SEQ ID NO: 257 - RNA, 41 nt, synthetic construct; identical to SEQ ID NO: 235
  misc_feature 1..41: note = SYNTHETIC RNA
  source 1..41: mol_type = other RNA
  modified_base 22: mod_base = OTHER; note = 2-Prime-O-methyl ribonucleotides

SEQ ID NO: 258 - RNA, 41 nt, synthetic construct; identical to SEQ ID NO: 235
  misc_feature 1..41: note = SYNTHETIC RNA
  source 1..41: mol_type = other RNA
  modified_base 23: mod_base = OTHER; note = 2-Prime-O-methyl ribonucleotides

SEQ ID NO: 259 - RNA, 41 nt, synthetic construct; identical to SEQ ID NO: 235
  misc_feature 1..41: note = SYNTHETIC RNA
  source 1..41: mol_type = other RNA
  modified_base 24: mod_base = OTHER; note = 2-Prime-O-methyl ribonucleotides

SEQ ID NO: 260 - RNA, 41 nt, synthetic construct; identical to SEQ ID NO: 235
  misc_feature 1..41: note = SYNTHETIC RNA
  source 1..41: mol_type = other RNA
  modified_base 25: mod_base = OTHER; note = 2-Prime-O-methyl ribonucleotides

SEQ ID NO: 261 - RNA, 41 nt, synthetic construct; identical to SEQ ID NO: 235
  misc_feature 1..41: note = SYNTHETIC RNA
  source 1..41: mol_type = other RNA
  modified_base 26: mod_base = OTHER; note = 2-Prime-O-methyl ribonucleotides

SEQ ID NO: 262 - RNA, 41 nt, synthetic construct; identical to SEQ ID NO: 235
  misc_feature 1..41: note = SYNTHETIC RNA
  source 1..41: mol_type = other RNA
  modified_base 27: mod_base = OTHER; note = 2-Prime-O-methyl ribonucleotides

SEQ ID NO: 263 - RNA, 41 nt, synthetic construct; identical to SEQ ID NO: 235
  misc_feature 1..41: note = SYNTHETIC RNA
  source 1..41: mol_type = other RNA
  modified_base 28: mod_base = OTHER; note = 2-Prime-O-methyl ribonucleotides

SEQ ID NO: 264 - RNA, 41 nt, synthetic construct; identical to SEQ ID NO: 235
  misc_feature 1..41: note = SYNTHETIC RNA
  source 1..41: mol_type = other RNA
  modified_base 29: mod_base = OTHER; note = 2-Prime-O-methyl ribonucleotides

SEQ ID NO: 265 - RNA, 41 nt, synthetic construct; identical to SEQ ID NO: 235
  misc_feature 1..41: note = SYNTHETIC RNA
  source 1..41: mol_type = other RNA
  modified_base 30: mod_base = OTHER; note = 2-Prime-O-methyl ribonucleotides

SEQ ID NO: 266 - RNA, 41 nt, synthetic construct; identical to SEQ ID NO: 235
  misc_feature 1..41: note = SYNTHETIC RNA
  source 1..41: mol_type = other RNA
  modified_base 31: mod_base = OTHER; note = 2-Prime-O-methyl ribonucleotides

SEQ ID NO: 267 - RNA, 41 nt, synthetic construct; identical to SEQ ID NO: 235
  misc_feature 1..41: note = SYNTHETIC RNA
  source 1..41: mol_type = other RNA
  modified_base 32: mod_base = OTHER; note = 2-Prime-O-methyl ribonucleotides

SEQ ID NO: 268 - RNA, 41 nt, synthetic construct; identical to SEQ ID NO: 235
  misc_feature 1..41: note = SYNTHETIC RNA
  source 1..41: mol_type = other RNA
  modified_base 33: mod_base = OTHER; note = 2-Prime-O-methyl ribonucleotides

SEQ ID NO: 269 - RNA, 41 nt, synthetic construct; identical to SEQ ID NO: 235
  misc_feature 1..41: note = SYNTHETIC RNA
  source 1..41: mol_type = other RNA
  modified_base 34: mod_base = OTHER; note = 2-Prime-O-methyl ribonucleotides

SEQ ID NO: 270 - RNA, 41 nt, synthetic construct; identical to SEQ ID NO: 235
  misc_feature 1..41: note = SYNTHETIC RNA
  source 1..41: mol_type = other RNA
  modified_base 35: mod_base = OTHER; note = 2-Prime-O-methyl ribonucleotides

SEQ ID NO: 271 - RNA, 41 nt, synthetic construct; identical to SEQ ID NO: 235
  misc_feature 1..41: note = SYNTHETIC RNA
  source 1..41: mol_type = other RNA
  modified_base 36: mod_base = OTHER; note = 2-Prime-O-methyl ribonucleotides

SEQ ID NO: 272 - RNA, 41 nt, synthetic construct; identical to SEQ ID NO: 235
  misc_feature 1..41: note = SYNTHETIC RNA
  source 1..41: mol_type = other RNA
  modified_base 37: mod_base = OTHER; note = 2-Prime-O-methyl ribonucleotides

SEQ ID NO: 273 - RNA, 41 nt, synthetic construct; identical to SEQ ID NO: 235
  misc_feature 1..41: note = SYNTHETIC RNA
  source 1..41: mol_type = other RNA
  modified_base 38: mod_base = OTHER; note = 2-Prime-O-methyl ribonucleotides

SEQ ID NO: 274 - RNA, 41 nt, synthetic construct; identical to SEQ ID NO: 235
  misc_feature 1..41: note = SYNTHETIC RNA
  source 1..41: mol_type = other RNA
  modified_base 39: mod_base = OTHER; note = 2-Prime-O-methyl ribonucleotides

SEQ ID NO: 275 - RNA, 41 nt, synthetic construct; identical to SEQ ID NO: 235
  misc_feature 1..41: note = SYNTHETIC RNA
  source 1..41: mol_type = other RNA
  modified_base 40: mod_base = OTHER; note = 2-Prime-O-methyl ribonucleotides

SEQ ID NO: 276 - RNA, 41 nt, synthetic construct; identical to SEQ ID NO: 235
  misc_feature 1..41: note = SYNTHETIC RNA
  source 1..41: mol_type = other RNA
  modified_base 41: mod_base = OTHER; note = 2-Prime-O-methyl ribonucleotides

SEQ ID NO: 277 - RNA, 44 nt, synthetic construct; identical to SEQ ID NO: 41
  misc_feature 1..44: note = SYNTHETIC RNA
  source 1..44: mol_type = other RNA
  modified_base order(1,5..10,12..14,16..19): mod_base = OTHER; note = 2-Prime-O-methyl ribonucleotides

SEQ ID NO: 278 - RNA, 44 nt, synthetic construct; identical to SEQ ID NO: 41
  misc_feature 1..44: note = SYNTHETIC RNA
  source 1..44: mol_type = other RNA
  modified_base order(21..24,27..32,34..44): mod_base = OTHER; note = 2-Prime-O-methyl ribonucleotides

SEQ ID NO: 279 - RNA, 44 nt, synthetic construct; identical to SEQ ID NO: 41
  misc_feature 1..44: note = SYNTHETIC RNA
  source 1..44: mol_type = other RNA
  modified_base order(1,5..10,12..14,16..19,21..24,27..32,34..44): mod_base = OTHER; note = 2-Prime-O-methyl ribonucleotides

SEQ ID NO: 280 - RNA, 41 nt, synthetic construct; identical to SEQ ID NO: 77
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides

SEQ ID NO: 281 - RNA, 41 nt, synthetic construct; identical to SEQ ID NO: 77
  misc_feature 1..41: note = SYNTHETIC RNA
  source 1..41: mol_type = other RNA
  modified_base order(21..24,27..32,34..41): mod_base = OTHER; note = 2-Prime-O-methyl ribonucleotides

SEQ ID NO: 282 - RNA, 41 nt, synthetic construct; identical to SEQ ID NO: 77
  misc_feature 1..41: note = SYNTHETIC RNA
  source 1..41: mol_type = other RNA
  modified_base order(1,5..10,12..14,16..19,21..24,27..32,34..41): mod_base = OTHER; note = 2-Prime-O-methyl ribonucleotides

SEQ ID NO: 283 - RNA, 44 nt, synthetic construct; identical to SEQ ID NO: 190
  misc_feature 1..44: note = SYNTHETIC RNA
  source 1..44: mol_type = other RNA
  modified_base order(1,5..10,12..14,16..19): mod_base = OTHER; note = 2-Prime-O-methyl ribonucleotides

SEQ ID NO: 284 - RNA, 44 nt, synthetic construct; identical to SEQ ID NO: 190
  misc_feature 1..44: note = SYNTHETIC RNA
  source 1..44: mol_type = other RNA
  modified_base order(21..23,27..32,34..35,37..44): mod_base = OTHER; note = 2-Prime-O-methyl ribonucleotides

SEQ ID NO: 285 - RNA, 44 nt, synthetic construct; identical to SEQ ID NO: 190
  misc_feature 1..44: note = SYNTHETIC RNA
  source 1..44: mol_type = other RNA
  modified_base order(1,5..10,12..14,16..19,21..23,27..32,34..35,37..44): mod_base = OTHER; note = 2-Prime-O-methyl ribonucleotides

SEQ ID NO: 286 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides

SEQ ID NO: 287 - RNA, 41 nt, synthetic construct; identical to SEQ ID NO: 235
  misc_feature 1..41: note = SYNTHETIC RNA
  source 1..41: mol_type = other RNA
  modified_base order(21..23,27..32,34..35,37..41): mod_base = OTHER; note = 2-Prime-O-methyl ribonucleotides

SEQ ID NO: 288 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,21..23,27..32,34..35,37..41): mod_base = OTHER; note = 2-Prime-O-methyl ribonucleotides

SEQ ID NO: 289 - RNA, 41 nt, synthetic construct; identical to SEQ ID NO: 235
  misc_feature 1..41: note = SYNTHETIC RNA
  source 1..41: mol_type = other RNA
  modified_base 21..23: mod_base = OTHER; note = 2-Prime-O-methyl ribonucleotides

SEQ ID NO: 290 - RNA, 41 nt, synthetic construct; identical to SEQ ID NO: 235
  misc_feature 1..41: note = SYNTHETIC RNA
  source 1..41: mol_type = other RNA
  modified_base 27..32: mod_base = OTHER; note = 2-Prime-O-methyl ribonucleotides

SEQ ID NO: 291 - RNA, 41 nt, synthetic construct; identical to SEQ ID NO: 235
  misc_feature 1..41: note = SYNTHETIC RNA
  source 1..41: mol_type = other RNA
  modified_base 34..35: mod_base = OTHER; note = 2-Prime-O-methyl ribonucleotides

SEQ ID NO: 292 - RNA, 41 nt, synthetic construct; identical to SEQ ID NO: 235
  misc_feature 1..41: note = SYNTHETIC RNA
  source 1..41: mol_type = other RNA
  modified_base 37..41: mod_base = OTHER; note = 2-Prime-O-methyl ribonucleotides

SEQ ID NO: 293 - RNA, 41 nt, synthetic construct; identical to SEQ ID NO: 235
  misc_feature 1..41: note = SYNTHETIC RNA
  source 1..41: mol_type = other RNA
  modified_base order(21..22,28..31,38..41): mod_base = OTHER; note = 2-Prime-O-methyl ribonucleotides

SEQ ID NO: 294 - RNA, 41 nt, synthetic construct; identical to SEQ ID NO: 235
  misc_feature 1..41: note = SYNTHETIC RNA
  source 1..41: mol_type = other RNA
  misc_feature 1: note = C3 spacer

SEQ ID NO: 295 - RNA, 41 nt, synthetic construct; identical to SEQ ID NO: 235
  misc_feature 1..41: note = SYNTHETIC RNA
  source 1..41: mol_type = other RNA
  misc_feature 41: note = C3 spacer

SEQ ID NO: 296 - RNA, 41 nt, synthetic construct; identical to SEQ ID NO: 235
  misc_feature 1..41: note = SYNTHETIC RNA
  source 1..41: mol_type = other RNA
  misc_feature order(1,41): note = C3 spacer

SEQ ID NO: 297 - RNA, 41 nt, synthetic construct; identical to SEQ ID NO: 235
  misc_feature 1..41: note = SYNTHETIC RNA
  source 1..41: mol_type = other RNA
  modified_base 1..20: mod_base = OTHER; note = 2-Prime-O-methyl ribonucleotides

SEQ ID NO: 298 - RNA, 41 nt, synthetic construct; identical to SEQ ID NO: 235
  misc_feature 1..41: note = SYNTHETIC RNA
  source 1..41: mol_type = other RNA
  modified_base order(1..2,5..10,12..14,16..19): mod_base = OTHER; note = 2-Prime-O-methyl ribonucleotides

SEQ ID NO: 299 - RNA, 41 nt, synthetic construct; identical to SEQ ID NO: 235
  misc_feature 1..41: note = SYNTHETIC RNA
  source 1..41: mol_type = other RNA
  modified_base order(1,3,5..10,12..14,16..19): mod_base = OTHER; note = 2-Prime-O-methyl ribonucleotides

SEQ ID NO: 300 - RNA, 41 nt, synthetic construct; identical to SEQ ID NO: 235
  misc_feature 1..41: note = SYNTHETIC RNA
  source 1..41: mol_type = other RNA
  modified_base order(1,4..10,12..14,16..19): mod_base = OTHER; note = 2-Prime-O-methyl ribonucleotides

SEQ ID NO: 301 - RNA, 41 nt, synthetic construct; identical to SEQ ID NO: 235
  misc_feature 1..41: note = SYNTHETIC RNA
  source 1..41: mol_type = other RNA
  modified_base order(1,5..14,16..19): mod_base = OTHER; note = 2-Prime-O-methyl ribonucleotides

SEQ ID NO: 302 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..19): mod_base = OTHER; note = 2-Prime-O-methyl ribonucleotides

SEQ ID NO: 303 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..20): mod_base = OTHER; note = 2-Prime-O-methyl ribonucleotides

SEQ ID NO: 304 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides
  misc_feature order(1,41): note = C3 spacer

SEQ ID NO: 305 - RNA, 41 nt, synthetic construct; identical to SEQ ID NO: 235
  misc_feature 1..41: note = SYNTHETIC RNA
  source 1..41: mol_type = other RNA
  modified_base order(1..4,38..41): mod_base = OTHER; note = Phosphorothioate internucleotide linkages

SEQ ID NO: 306 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides
  modified_base order(1..4,38..41): mod_base = OTHER; note = Phosphorothioate internucleotide linkages

SEQ ID NO: 307 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides
  modified_base 1..20: mod_base = OTHER; note = Phosphorothioate internucleotide linkages

SEQ ID NO: 308 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides
  modified_base 20..41: mod_base = OTHER; note = Phosphorothioate internucleotide linkages

SEQ ID NO: 309 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,22..23): mod_base = OTHER; note = 2-Prime-O-methyl ribonucleotides

SEQ ID NO: 310 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,24..25): mod_base = OTHER; note = 2-Prime-O-methyl ribonucleotides

SEQ ID NO: 311 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,26..27): mod_base = OTHER; note = 2-Prime-O-methyl ribonucleotides

SEQ ID NO: 312 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,28..29): mod_base = OTHER; note = 2-Prime-O-methyl ribonucleotides

SEQ ID NO: 313 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,30..31): mod_base = OTHER; note = 2-Prime-O-methyl ribonucleotides

SEQ ID NO: 314 - RNA, 41 nt, synthetic construct; identical to SEQ ID NO: 235
  misc_feature 1..41: note = SYNTHETIC RNA
  source 1..41: mol_type = other RNA
  modified_base order(5..10,12..14,16..19,32..33): mod_base = OTHER; note = 2-Prime-O-methyl ribonucleotides

SEQ ID NO: 315 - RNA, 41 nt, synthetic construct; identical to SEQ ID NO: 235
  misc_feature 1..41: note = SYNTHETIC RNA
  source 1..41: mol_type = other RNA
  modified_base order(5..10,12..14,16..19,34..35): mod_base = OTHER; note = 2-Prime-O-methyl ribonucleotides

SEQ ID NO: 316 - RNA, 41 nt, synthetic construct; identical to SEQ ID NO: 235
  misc_feature 1..41: note = SYNTHETIC RNA
  source 1..41: mol_type = other RNA
  modified_base order(5..10,12..14,16..19,36..37): mod_base = OTHER; note = 2-Prime-O-methyl ribonucleotides

SEQ ID NO: 317 - RNA, 41 nt, synthetic construct; identical to SEQ ID NO: 235
  misc_feature 1..41: note = SYNTHETIC RNA
  source 1..41: mol_type = other RNA
  modified_base order(5..10,12..14,16..19,38..39): mod_base = OTHER; note = 2-Prime-O-methyl ribonucleotides

SEQ ID NO: 318 - RNA, 41 nt, synthetic construct; identical to SEQ ID NO: 235
  misc_feature 1..41: note = SYNTHETIC RNA
  source 1..41: mol_type = other RNA
  modified_base order(5..10,12..14,16..19,40..41): mod_base = OTHER; note = 2-Prime-O-methyl ribonucleotides

SEQ ID NO: 319 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,21..22): mod_base = OTHER; note = 2-Prime-O-methyl ribonucleotides

SEQ ID NO: 320 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,23..24): mod_base = OTHER; note = 2-Prime-O-methyl ribonucleotides

SEQ ID NO: 321 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,25..26): mod_base = OTHER; note = 2-Prime-O-methyl ribonucleotides

SEQ ID NO: 322 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,27..28): mod_base = OTHER; note = 2-Prime-O-methyl ribonucleotides

SEQ ID NO: 323 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,29..30): mod_base = OTHER; note = 2-Prime-O-methyl ribonucleotides

SEQ ID NO: 324 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,31..32): mod_base = OTHER; note = 2-Prime-O-methyl ribonucleotides

SEQ ID NO: 325 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,33..34): mod_base = OTHER; note = 2-Prime-O-methyl ribonucleotides

SEQ ID NO: 326 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,35..36): mod_base = OTHER; note = 2-Prime-O-methyl ribonucleotides

SEQ ID NO: 327 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,37..38): mod_base = OTHER; note = 2-Prime-O-methyl ribonucleotides

SEQ ID NO: 328 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19,39..40): mod_base = OTHER; note = 2-Prime-O-methyl ribonucleotides

SEQ ID NO: 329 - RNA, 41 nt, synthetic construct; identical to SEQ ID NO: 235
  source 1..41: mol_type = other RNA
  modified_base order(1,39..41): mod_base = OTHER; note = 2-Prime-O-methyl ribonucleotides
  modified_base order(1..4,38..41): mod_base = OTHER; note = Phosphorothioate internucleotide linkages

SEQ ID NO: 330 - RNA, 41 nt, synthetic construct; identical to SEQ ID NO: 235
  misc_feature 1..41: note = SYNTHETIC RNA
  source 1..41: mol_type = other RNA
  modified_base order(1,40..41): mod_base = OTHER; note = Locked nucleic acids
  modified_base 1: mod_base = OTHER; note = Thymidine

SEQ ID NO: 331 - RNA, 41 nt, synthetic construct; identical to SEQ ID NO: 235
  misc_feature 1..41: note = SYNTHETIC RNA
  source 1..41: mol_type = other RNA
  modified_base order(1,5..10,12..14,16..19): mod_base = OTHER; note = 2-Prime-fluoro ribonucleotides

SEQ ID NO: 332 - RNA, 41 nt, synthetic construct; identical to SEQ ID NO: 235
  misc_feature 1..41: note = SYNTHETIC RNA
  source 1..41: mol_type = other RNA
  modified_base 21..23: mod_base = OTHER; note = 2-Prime-fluoro ribonucleotides

SEQ ID NO: 333 - RNA, 41 nt, synthetic construct; identical to SEQ ID NO: 235
  misc_feature 1..41: note = SYNTHETIC RNA
  source 1..41: mol_type = other RNA
  modified_base 27..32: mod_base = OTHER; note = 2-Prime-fluoro ribonucleotides

SEQ ID NO: 334 - RNA, 41 nt, synthetic construct; identical to SEQ ID NO: 235
  misc_feature 1..41: note = SYNTHETIC RNA
  source 1..41: mol_type = other RNA
  modified_base 34..35: mod_base = OTHER; note = 2-Prime-fluoro ribonucleotides

SEQ ID NO: 335 - RNA, 41 nt, synthetic construct; identical to SEQ ID NO: 235
  misc_feature 1..41: note = SYNTHETIC RNA
  source 1..41: mol_type = other RNA
  modified_base 37..41: mod_base = OTHER; note = 2-Prime-fluoro ribonucleotides

SEQ ID NO: 336 - RNA, 41 nt, synthetic construct; identical to SEQ ID NO: 235
  misc_feature 1..41: note = SYNTHETIC RNA
  source 1..41: mol_type = other RNA
  misc_feature 1: note = Napthyl-azo is attached to uracil nucleotide
  misc_feature 41: note = Napthyl-azo is attached to cytosine nucleotide

SEQ ID NO: 337 - RNA, 41 nt, synthetic construct; identical to SEQ ID NO: 235
  misc_feature 1..41: note = SYNTHETIC RNA
  source 1..41: mol_type = other RNA
  modified_base 1: mod_base = OTHER; note = Napthyl-azo is attached to uracil nucleotide
  misc_feature 41: note = C3 spacer

SEQ ID NO: 338 - RNA, 41 nt, synthetic construct; identical to SEQ ID NO: 235
  misc_feature 1..41: note = SYNTHETIC RNA
  source 1..41: mol_type = other RNA
  misc_feature 41: note = Napthyl-azo is attached to cytosine nucleotide
  misc_feature 1: note = C3 spacer

SEQ ID NO: 339 - RNA, 41 nt, synthetic construct
  misc_feature 1..41: note = SYNTHETIC RNA
  source 1..41: mol_type = other RNA
taatttctactcttgtagatcttgggtgtgttaaaagtgac

SEQ ID NO: 340 - RNA, 41 nt, synthetic construct; identical to SEQ ID NO: 339
  misc_feature 1..41: note = SYNTHETIC RNA
  source 1..41: mol_type = other RNA
  misc_feature order(1,41): note = C3 spacer

SEQ ID NO: 341 - RNA, 41 nt, synthetic construct; identical to SEQ ID NO: 339
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides

SEQ ID NO: 342 - RNA, 41 nt, synthetic construct; identical to SEQ ID NO: 339
  misc_feature 1..41: note = SYNTHETIC RNA
  source 1..41: mol_type = other RNA
  modified_base order(1,39..41): mod_base = OTHER; note = 2-Prime-O-methyl ribonucleotides
  modified_base order(1..3,38..41): mod_base = OTHER; note = Phosphorothioate internucleotide linkages

SEQ ID NO: 343 - RNA, 41 nt, synthetic construct; identical to SEQ ID NO: 339
  misc_feature 1..41: note = SYNTHETIC RNA
  source 1..41: mol_type = other RNA
  modified_base order(1,5..10,12..14,16..19,39..41): mod_base = OTHER; note = 2-Prime-O-methyl ribonucleotides
  modified_base order(1..3,39..41): mod_base = OTHER; note = Phosphorothioate internucleotide linkages

SEQ ID NO: 344 - RNA, 41 nt, synthetic construct
  misc_feature 1..41: note = SYNTHETIC RNA
  source 1..41: mol_type = other RNA
taatttctactcttgtagatacataaaactcttttaggtta

SEQ ID NO: 345 - RNA, 41 nt, synthetic construct; identical to SEQ ID NO: 344
  misc_feature 1..41: note = SYNTHETIC RNA
  source 1..41: mol_type = other RNA
  modified_base 1..4: mod_base = OTHER; note = Phosphorothioate internucleotide linkages

SEQ ID NO: 346 - RNA, 41 nt, synthetic construct; identical to SEQ ID NO: 344
  misc_feature 1..41: note = SYNTHETIC RNA
  source 1..41: mol_type = other RNA
  modified_base 1..7: mod_base = OTHER; note = Phosphorothioate internucleotide linkages

SEQ ID NO: 347 - RNA, 41 nt, synthetic construct; identical to SEQ ID NO: 344
  misc_feature 1..41: note = SYNTHETIC RNA
  source 1..41: mol_type = other RNA
  modified_base 35..41: mod_base = OTHER; note = Phosphorothioate internucleotide linkages

SEQ ID NO: 348 - RNA, 41 nt, synthetic construct; identical to SEQ ID NO: 344
  misc_feature 1..41: note = SYNTHETIC RNA
  source 1..41: mol_type = other RNA
  modified_base order(1..4,38..41): mod_base = OTHER; note = Phosphorothioate internucleotide linkages

SEQ ID NO: 349 - RNA, 41 nt, synthetic construct; identical to SEQ ID NO: 344
  misc_feature 1..41: note = SYNTHETIC RNA
  source 1..41: mol_type = other RNA
  modified_base order(1..4,35..41): mod_base = OTHER; note = Phosphorothioate internucleotide linkages

SEQ ID NO: 350 - RNA, 41 nt, synthetic construct; identical to SEQ ID NO: 344
  misc_feature 1..41: note = SYNTHETIC RNA
  source 1..41: mol_type = other RNA
  modified_base order(1..7,35..41): mod_base = OTHER; note = Phosphorothioate internucleotide linkages

SEQ ID NO: 351 - RNA, 41 nt, synthetic construct; identical to SEQ ID NO: 344
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides
  modified_base 38..41: mod_base = OTHER; note = Phosphorothioate internucleotide linkages

SEQ ID NO: 352 - RNA, 41 nt, synthetic construct; identical to SEQ ID NO: 344
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides
  modified_base 35..41: mod_base = OTHER; note = Phosphorothioate internucleotide linkages

SEQ ID NO: 353 - RNA, 41 nt, synthetic construct; identical to SEQ ID NO: 344
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides
  modified_base order(1..4,38..41): mod_base = OTHER; note = Phosphorothioate internucleotide linkages

SEQ ID NO: 354 - RNA, 41 nt, synthetic construct; identical to SEQ ID NO: 344
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides
  modified_base order(1..4,37..41): mod_base = OTHER; note = Phosphorothioate internucleotide linkages

SEQ ID NO: 355 - RNA, 41 nt, synthetic construct; identical to SEQ ID NO: 344
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides
  modified_base order(1..4,36..41): mod_base = OTHER; note = Phosphorothioate internucleotide linkages

SEQ ID NO: 356 - RNA, 41 nt, synthetic construct; identical to SEQ ID NO: 344
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides
  modified_base order(1..4,35..41): mod_base = OTHER; note = Phosphorothioate internucleotide linkages

SEQ ID NO: 357 - RNA, 41 nt, synthetic construct; identical to SEQ ID NO: 344
  misc_feature 1..41: note = SYNTHETIC RNA
  source 1..41: mol_type = other RNA
  modified_base order(1,5..10,12..14,16..19): mod_base = OTHER; note = 2-Prime-O-methyl ribonucleotides
  modified_base order(1..7,35..41): mod_base = OTHER; note = Phosphorothioate internucleotide linkages

SEQ ID NO: 358 - RNA, 41 nt, synthetic construct
  misc_feature 1..41: note = SYNTHETIC RNA
  source 1..41: mol_type = other RNA
  misc_feature order(1,41): note = C3 spacer
taatttctactcttgtagatatagtctttccttgggtgtgt

SEQ ID NO: 359 - RNA, 42 nt, synthetic construct
  misc_feature 1..42: note = SYNTHETIC RNA
  source 1..42: mol_type = other RNA
  misc_feature order(1,42): note = C3 spacer
taatttctactaagtgtagatatagtctttccttgggtgtgt

SEQ ID NO: 360 - RNA, 44 nt, synthetic construct
  misc_feature 1..44: note = SYNTHETIC RNA
  source 1..44: mol_type = other RNA
  misc_feature order(1,44): note = C3 spacer
taatttctactaagtgtagatatagtctttccttgggtgtgtta

SEQ ID NO: 361 - RNA, 41 nt, synthetic construct; identical to SEQ ID NO: 339
  misc_feature 1..41: note = SYNTHETIC RNA
  source 1..41: mol_type = other RNA
  misc_feature order(1,41): note = C3 spacer

SEQ ID NO: 362 - RNA, 42 nt, synthetic construct
  misc_feature 1..42: note = SYNTHETIC RNA
  source 1..42: mol_type = other RNA
  misc_feature order(1,42): note = C3 spacer
taatttctactaagtgtagatcttgggtgtgttaaaagtgac

SEQ ID NO: 363 - RNA, 44 nt, synthetic construct
  misc_feature 1..44: note = SYNTHETIC RNA
  source 1..44: mol_type = other RNA
  misc_feature order(1,44): note = C3 spacer
taatttctactaagtgtagatcttgggtgtgttaaaagtgacca

SEQ ID NO: 364 - RNA, 41 nt, synthetic construct
  misc_feature 1..41: note = SYNTHETIC RNA
  source 1..41: mol_type = other RNA
  misc_feature order(1,41): note = C3 spacer
taatttctactcttgtagatacacacccaaggaaagactat

SEQ ID NO: 365 - RNA, 42 nt, synthetic construct
  misc_feature 1..42: note = SYNTHETIC RNA
  source 1..42: mol_type = other RNA
  misc_feature order(1,42): note = C3 spacer
taatttctactaagtgtagatacacacccaaggaaagactat

SEQ ID NO: 366 - RNA, 44 nt, synthetic construct
  misc_feature 1..44: note = SYNTHETIC RNA
  source 1..44: mol_type = other RNA
  misc_feature order(1,44): note = C3 spacer
taatttctactaagtgtagatacacacccaaggaaagactatga

SEQ ID NO: 367 - RNA, 41 nt, synthetic construct
  misc_feature 1..41: note = SYNTHETIC RNA
  source 1..41: mol_type = other RNA
  misc_feature order(1,41): note = C3 spacer
taatttctactcttgtagatatccgtgctgagtgtaccatg

SEQ ID NO: 368 - RNA, 42 nt, synthetic construct
  misc_feature 1..42: note = SYNTHETIC RNA
  source 1..42: mol_type = other RNA
  misc_feature order(1,42): note = C3 spacer
taatttctactaagtgtagatatccgtgctgagtgtaccatg

SEQ ID NO: 369 - RNA, 44 nt, synthetic construct
  misc_feature 1..44: note = SYNTHETIC RNA
  source 1..44: mol_type = other RNA
  misc_feature order(1,44): note = C3 spacer
taatttctactaagtgtagatatccgtgctgagtgtaccatgca

SEQ ID NO: 370 - RNA, 41 nt, synthetic construct; identical to SEQ ID NO: 61
  misc_feature 1..41: note = SYNTHETIC RNA
  source 1..41: mol_type = other RNA
  misc_feature order(1,41): note = C3 spacer

SEQ ID NO: 371 - RNA, 42 nt, synthetic construct
  misc_feature 1..42: note = SYNTHETIC RNA
  source 1..42: mol_type = other RNA
  misc_feature order(1,42): note = C3 spacer
taatttctactaagtgtagattaaacactgtttcatttcatc

SEQ ID NO: 372 - RNA, 44 nt, synthetic construct
  misc_feature 1..44: note = SYNTHETIC RNA
  source 1..44: mol_type = other RNA
  misc_feature order(1,44): note = C3 spacer
taatttctactaagtgtagattaaacactgtttcatttcatccg

SEQ ID NO: 373 - RNA, 41 nt, synthetic construct
  misc_feature 1..41: note = SYNTHETIC RNA
  source 1..41: mol_type = other RNA
  misc_feature order(1,41): note = C3 spacer
taatttctactcttgtagatgaaacgtcagtcttctctttt

SEQ ID NO: 374 - RNA, 42 nt, synthetic construct
  misc_feature 1..42: note = SYNTHETIC RNA
  source 1..42: mol_type = other RNA
  misc_feature order(1,42): note = C3 spacer
taatttctactaagtgtagatgaaacgtcagtcttctctttt

SEQ ID NO: 375 - RNA, 44 nt, synthetic construct
  misc_feature 1..44: note = SYNTHETIC RNA
  source 1..44: mol_type = other RNA
  misc_feature order(1,44): note = C3 spacer
taatttctactaagtgtagatgaaacgtcagtcttctcttttgt

SEQ ID NO: 376 - RNA, 41 nt, synthetic construct
  misc_feature 1..41: note = SYNTHETIC RNA
  source 1..41: mol_type = other RNA
  misc_feature order(1,41): note = C3 spacer
taatttctactcttgtagattaatgccctgtagtctctctg

SEQ ID NO: 377 - RNA, 42 nt, synthetic construct
  misc_feature 1..42: note = SYNTHETIC RNA
  source 1..42: mol_type = other RNA
  misc_feature order(1,42): note = C3 spacer
taatttctactaagtgtagattaatgccctgtagtctctctg

SEQ ID NO: 378 - RNA, 44 nt, synthetic construct
  misc_feature 1..44: note = SYNTHETIC RNA
  source 1..44: mol_type = other RNA
  misc_feature order(1,44): note = C3 spacer
taatttctactaagtgtagattaatgccctgtagtctctctgta

SEQ ID NO: 379 - RNA, 41 nt, synthetic construct
  misc_feature 1..41: note = SYNTHETIC RNA
  source 1..41: mol_type = other RNA
  misc_feature order(1,41): note = C3 spacer
taatttctactcttgtagattaattaacagcttgctggtga

SEQ ID NO: 380 - RNA, 42 nt, synthetic construct
  misc_feature 1..42: note = SYNTHETIC RNA
  source 1..42: mol_type = other RNA
  misc_feature order(1,42): note = C3 spacer
taatttctactaagtgtagattaattaacagcttgctggtga

SEQ ID NO: 381 - RNA, 44 nt, synthetic construct
  misc_feature 1..44: note = SYNTHETIC RNA
  source 1..44: mol_type = other RNA
  misc_feature order(1,44): note = C3 spacer
taatttctactaagtgtagattaattaacagcttgctggtgaaa

SEQ ID NO: 382 - RNA, 41 nt, synthetic construct; identical to SEQ ID NO: 73
  misc_feature 1..41: note = SYNTHETIC RNA
  source 1..41: mol_type = other RNA
  misc_feature order(1,41): note = C3 spacer

SEQ ID NO: 383 - RNA, 42 nt, synthetic construct
  misc_feature 1..42: note = SYNTHETIC RNA
  source 1..42: mol_type = other RNA
  misc_feature order(1,42): note = C3 spacer
taatttctactaagtgtagatggttaaagatggttaaatgat

SEQ ID NO: 384 - RNA, 44 nt, synthetic construct
  misc_feature 1..44: note = SYNTHETIC RNA
  source 1..44: mol_type = other RNA
  misc_feature order(1,44): note = C3 spacer
taatttctactaagtgtagatggttaaagatggttaaatgattg

SEQ ID NO: 385 - RNA, 41 nt, synthetic construct; identical to SEQ ID NO: 77
  misc_feature 1..41: note = SYNTHETIC RNA
  source 1..41: mol_type = other RNA
  misc_feature order(1,41): note = C3 spacer

SEQ ID NO: 386 - RNA, 42 nt, synthetic construct
  misc_feature 1..42: note = SYNTHETIC RNA
  source 1..42: mol_type = other RNA
  misc_feature order(1,42): note = C3 spacer
taatttctactaagtgtagattgtgaaatggcttataattgc

SEQ ID NO: 387 - RNA, 44 nt, synthetic construct
  misc_feature 1..44: note = SYNTHETIC RNA
  source 1..44: mol_type = other RNA
  misc_feature order(1,44): note = C3 spacer
taatttctactaagtgtagattgtgaaatggcttataattgctt

SEQ ID NO: 388 - RNA, 41 nt, synthetic construct
  misc_feature 1..41: note = SYNTHETIC RNA
  source 1..41: mol_type = other RNA
  misc_feature order(1,41): note = C3 spacer
taatttctactcttgtagatgttgttggatttgaaattcca

SEQ ID NO: 389 - RNA, 42 nt, synthetic construct
  misc_feature 1..42: note = SYNTHETIC RNA
  source 1..42: mol_type = other RNA
  misc_feature order(1,42): note = C3 spacer
taatttctactaagtgtagatgttgttggatttgaaattcca

SEQ ID NO: 390 - RNA, 44 nt, synthetic construct
  misc_feature 1..44: note = SYNTHETIC RNA
  source 1..44: mol_type = other RNA
  misc_feature order(1,44): note = C3 spacer
taatttctactaagtgtagatgttgttggatttgaaattccaga

SEQ ID NO: 391 - RNA, 41 nt, synthetic construct
  misc_feature 1..41: note = SYNTHETIC RNA
  source 1..41: mol_type = other RNA
  misc_feature order(1,41): note = C3 spacer
taatttctactcttgtagatttgtaggatatgcccttgact

SEQ ID NO: 392 - RNA, 42 nt, synthetic construct
  misc_feature 1..42: note = SYNTHETIC RNA
  source 1..42: mol_type = other RNA
  misc_feature order(1,42): note = C3 spacer
taatttctactaagtgtagatttgtaggatatgcccttgact

SEQ ID NO: 393 - DNA, 44 bp, synthetic construct
  misc_feature 1..44: note = SYNTHETIC DNA
  source 1..44: mol_type = other DNA
  misc_feature order(1,44): note = C3 spacer
taatttctactaagtgtagatttgtaggatatgcccttgactat

SEQ ID NO: 394 - DNA, 27 bp, synthetic construct
  misc_feature 1..27: note = SYNTHETIC DNA
  source 1..27: mol_type = other DNA
aagaatgttgtgataaaaggtgatgct

SEQ ID NO: 395 - DNA, 24 bp, synthetic construct
  misc_feature 1..24: note = SYNTHETIC DNA
  source 1..24: mol_type = other DNA
acacatccatgggacttctgcctc

SEQ ID NO: 396 - DNA, 3834 bp, synthetic construct
  source 1..3834: mol_type = other DNA
atgctgaagaacgtgggcatcgaccggctggacgtggaaaagggcagaaagaacatgagcaagctcgagaagttcaccaactgctacagcctgagcaagaccctgcggttcaaggccattcctgtgggcaagacccaagagaacatcgacaacaagcggctgctggtggaagatgagaagagagccgaggactacaagggcgtgaagaagctgctggaccggtactacctgagcttcatcaacgacgtgctgcacagcatcaagctcaagaacctgaacaactacatcagcctgttccggaagaaaacccggaccgagaaagagaacaaagagctggaaaacctcgagatcaacctgcggaaagagatcgccaaggccttcaagggcaacgagggctacaagagcctgttcaagaaggacatcatcgagacaatcctgcctgagttcctggacgacaaggacgagatcgccctggtcaacagcttcaacggcttcacaaccgccttcaccggctttttcgacaaccgcgagaatatgttcagcgaggaagccaagagcacctctatcgccttccggtgcatcaacgagaatctgacccggtacatcagcaacatggatatcttcgagaaggtggacgccatcttcgacaagcacgaggtgcaagagatcaaagaaaagatcctgaacagcgactacgacgtcgaggacttcttcgagggcgagttcttcaacttcgtgctgacacaagagggcatcgatgtgtacaacgccatcatcggcggcttcgtgacagagagcggcgagaagatcaagggcctgaacgagtacatcaacctctacaaccagaaaacgaagcagaagctgcccaagttcaagcccctgtacaaacaggtgctgagcgacagagagagcctgtccttttacggcgagggctataccagcgacgaagaggtgctggaagtgttcagaaacaccctgaacaagaacagcgagatcttcagctccatcaagaagctcgaaaagctgtttaagaacttcgacgagtacagcagcgccggcatcttcgtgaagaatggccctgccatcagcaccatctccaaggacatcttcggcgagtggaacgtgatccgggacaagtggaacgccgagtacgacgacatccacctgaagaaaaaggccgtggtcaccgagaagtacgaggacgacagaagaaagagcttcaagaagatcggcagcttcagcctggaacagctgcaagagtacgccgacgccgatctgagcgtggtggaaaagctgaaagagattatcatccagaaggtcgacgagatctacaaggtgtacggcagcagcgagaagctgttcgacgccgactttgtgctggaaaagagcctcaaaaagaacgacgccgtggtggccatcatgaaggacctgctggatagcgtgaagtccttcgagaactatattaaggccttctttggcgagggcaaagagacaaaccgggacgagagcttctacggcgatttcgtgctggcctacgacatcctgctgaaagtggaccacatctacgacgccatccggaactacgtgacccagaagccttacagcaaggacaagtttaagctgtacttccagaatccgcagttcatgggcggctgggacaaagacaaagaaaccgactaccgggccaccatcctgagatacggctccaagtactatctggccattatggacaagaaatacgccaagtgcctgcagaagatcgataaggacgacgtgaacggcaactacgagaagattaactacaagctgctgcccggacctaacaagatgctgcctaaggtgttctttagcaagaaatggatggcctactacaaccccagcgaggatatccagaaaatctacaagaacggcaccttcaagaaaggcgacatgttcaacctgaacgactgccacaagctgatcgatttcttcaaggacagcatcagcagataccccaagtggtccaacgcctacgacttcaatttcagcgagacagagaagtataaggatatcgccgggttctaccgcgaggtggaagaacagggctataaggtgtcctttgagagcgccagcaagaaagaggtggacaagctggtcgaagagggcaagctgtacatgttccagatctataacaaggacttctccgacaagagccacggcacccctaacctgcacaccatgtactttaagctgctgttcgatgagaacaaccacggccagatcagactgtctggcggagccgagctgtttatgagaagggccagcctgaaaaaagaggaactggtcgttcaccccgccaactctccaatcgccaacaagaaccccgacaatcccaagaaaaccaccacactgagctacgacgtgtacaaggataagcggttctccgaggaccagtacgagctgcacatccctatcgccatcaacaagtgccccaagaatatcttcaagatcaacaccgaagtgcgggtgctgctgaagcacgacgacaacccttacgtgatcggcatcgacagaggcgagcggaacctgctgtatatcgtggtggtggacggcaagggcaatatcgtggaacagtactccctgaatgagatcatcaacaacttcaatggcatccggatcaagacggactaccacagcctgctggacaaaaaagagaaagaacgcttcgaggcccggcagaactggaccagcatcgagaacatcaaagaactgaaggccggctacatctcccaggtggtgcacaagatctgcgagctggttgagaagtatgacgccgtgattgccctggaagatctgaatagcggctttaagaacagccgcgtgaaggtcgagaaacaggtgtaccagaaattcgagaagatgctgatcgacaagctgaactacatggtcgacaagaagtctaacccctgcgccacaggcggagccctgaagggatatcagatcaccaacaagttcgagtccttcaagagcatgagcacccagaatggcttcatcttctacatccccgcctggctgaccagcaagatcgatcctagcaccggattcgtgaacctgctcaagaccaagtacaccagcattgccgacagcaagaagttcatctccagcttcgaccggattatgtacgtgcccgaagaggacctgttcgaattcgccctggattacaagaacttcagccggaccgatgccgactatatcaagaagtggaagctgtatagctacggcaaccgcatccgcatcttcagaaacccgaagaaaaacaacgtgttcgactgggaagaagtgtgcctgaccagcgcctacaaagaactcttcaacaaatacggcatcaactaccagcagggcgacatcagagccctgctgtgcgagcagagcgacaaggccttttacagctccttcatggccctgatgtccctgatgctgcagatgcggaatagcatcaccggcaggaccgacgtggacttcctgatcagccctgtgaagaattccgacgggatcttctacgacagcagaaactacgaggctcaagagaacgccatcctgcctaagaacgccgatgccaacggcgcctataatatcgccagaaaggtgctgtgggccatcggccagtttaagaaggccgaggacgagaaactggacaaagtgaagatcgccatctctaacaaagagtggctggaatacgcccagaccagcgtgaagcacggcagatctagtgacgatgaggccaccgccgatagccagcatgcagcccctccaaagaaaaagcggaaagtgctggaacaccaccaccatcaccac

SEQ ID NO: 397 - protein, 15 aa, synthetic construct
GLNDIFEAQKIEWHE

SEQ ID NO: 398 - protein, 26 aa, synthetic construct
KRRWKKNFIAVSAANRFKKISSSGAL

SEQ ID NO: 399 - protein, 6 aa, synthetic construct
EEEEEE

SEQ ID NO: 400 - protein, 13 aa, synthetic construct
GAPVPYPDPLEPR

SEQ ID NO: 401 - protein, 8 aa, synthetic construct
DYKDDDDK

SEQ ID NO: 402 - protein, 9 aa, synthetic construct
YPYDVPDYA

SEQ ID NO: 403 - protein, 5 aa, synthetic construct
  VARIANT 5: note = H residue can be repeated up to five times
HHHHH

SEQ ID NO: 404 - protein, 6 aa, synthetic construct
HHHHHH

SEQ ID NO: 405 - protein, 10 aa, synthetic construct
EQKLISEEDL

SEQ ID NO: 406 - protein, 18 aa, synthetic construct
TKENPRSNQEESYDDNES

SEQ ID NO: 407 - protein, 15 aa, synthetic construct
KETAAAKFERQHMDS

SEQ ID NO: 408 - protein, 38 aa, synthetic construct
MDEKTTGWRGGHVVEGLAGELEQLRARLEHHPQGQREP

SEQ ID NO: 409 - protein, 13 aa, synthetic construct
SLAELLNAGLGGS

SEQ ID NO: 410 - protein, 8 aa, synthetic construct
TQDPSRVG

SEQ ID NO: 411 - protein, 8 aa, synthetic construct
WSHPQFEK

SEQ ID NO: 412 - protein, 6 aa, synthetic construct
CCPGCC

SEQ ID NO: 413 - protein, 14 aa, synthetic construct
GKPIPNPLLGLDST

SEQ ID NO: 414 - protein, 11 aa, synthetic construct
YTDIEMNRLGK

SEQ ID NO: 415 - protein, 8 aa, synthetic construct
DLYDDDDK

SEQ ID NO: 416 - protein, 16 aa, synthetic construct
TDKDMTITFTNKKDAE

SEQ ID NO: 417 - protein, 13 aa, synthetic construct
AHIVMVDAYKPTK

SEQ ID NO: 418 - protein, 12 aa, synthetic construct
KLGDIEFIKVNK

(19) United States
(12) Patent Application Publication
(10) Pub. No.: US 2026/0098249 A1
(43) Pub. Date: Apr. 9, 2026

(54) CRISPR/CPF1 Systems and Methods

(71) Applicant: Integrated DNA Technologies, Inc., Coralville, IA (US)

(72) Inventors: Mark Aaron BEHLKE, Coralville, IA (US); Michael Allen COLLINGWOOD, Solon, IA (US); Rolf TURK, Iowa City, IA (US); Christopher Anthony VAKULSKAS, North Liberty, IA (US)

(73) Assignee: Integrated DNA Technologies, Inc., Coralville, IA (US)

(21) Appl. No.: 19/412,076

(22) Filed: Dec. 8, 2025

Related U.S. Application Data
(63) Continuation of application No. 17/469,578, filed on Sep. 8, 2021.
Application No. 17/469,578 is a continuation of application No. 15/821,736, filed on Nov. 22, 2017, now Pat. No. 11,136,567.
(60) Provisional application No. 62/425,307, filed on Nov. 22, 2016.
(60) Provisional application No. 62/482,896, filed on Apr. 7, 2017.

(51) Int. Cl. C12N 9/22; C12N 15/10; C12N 15/11; C12N 15/113; C12N 15/63; C12N 15/85; C12N 15/90

(52) U.S. Cl. CPC C12N 9/22; C12N 15/102; C12N 15/111; C12N 15/113; C12N 15/63; C12N 15/8509; C12N 15/90; C12N 2310/20; C12Y 301/30

(57) ABSTRACT

This invention pertains to recombinant AsCpf1 and LbCpf1 nucleic acids and polypeptides for use in CRISPR/Cpf1 endonuclease systems and mammalian cell lines encoding recombinant AsCpf1 or LbCpf1 polypeptides. The invention includes recombinant ribonucleoprotein complexes and CRSPR/Cpf1 endonuclease systems having a suitable AsCpf1 crRNA is selected from a length-truncated AsCpf1 crRNA, a chemically-modified AsCpf1 crRNA, or an AsCpf1 crRNA comprising both length truncations and chemical modifications. Methods of performing gene editing using these systems and reagents are also provided.

### CROSS-REFERENCE TO RELATED APPLICATIONS

This application is a continuation application of U.S. patent application Ser. No. 17/469,578 filed Sep. 8, 2021 and entitled “CRISPR/CPF1 SYSTEMS AND METHODS”, which is a continuation application of U.S. patent application Ser. No. 15/821,736, filed Nov. 22, 2017 and entitled “CRISPR/CPF1 SYSTEMS AND METHODS,” which claims benefit of priority under 35 U.S.C. 119 to U.S. Provisional Patent Application Ser. No. 62/425,307, filed Nov. 22, 2016 and entitled “CPF1 CRISPR SYSTEMS AND METHODS,” and U.S. Provisional Patent Application Ser. No. 62/482,896, filed Apr. 7, 2017 and entitled “HEK293 CELL LINE WITH STABLE EXPRESSION OF ACIDAMINOCOCCUS SP. BV3L6 CPF1,” the contents of which are herein incorporated by reference in their entirety.

### SEQUENCE LISTING

The instant application contains a Sequence Listing that has been submitted in xml format via Patent Center and is hereby incorporated by reference in its entirety. The xml copy, created on Dec. 8, 2025, is named 6391-0030US03, and is 760,069 bytes in size.

### FIELD OF THE INVENTION

This invention pertains to Cpf1-based CRISPR genes, polypeptides encoded by the same, mammalian cell lines that stably express Cpf1, crRNAs and the use of these materials in compositions of CRISPR-Cpf1 systems and methods.

### BACKGROUND OF THE INVENTION

The use of clustered regularly interspaced short palindromic repeats (CRISPR) and associated Cas proteins (CRISPR-Cas system) for site-specific DNA cleavage has shown great potential for a number of biological applications. CRISPR is used for genome editing; the genome-scale-specific targeting of transcriptional repressors (CRISPRi) and activators (CRISPRa) to endogenous genes; and other applications of RNA-directed DNA targeting with Cas enzymes.

CRISPR-Cas systems are native to bacteria and Archaea and provide adaptive immunity against viruses and plasmids. Three classes of CRISPR-Cas systems could potentially be adapted for research and therapeutic reagents. Type-II CRISPR systems have a desirable characteristic in utilizing a single CRISPR associated (Cas) nuclease (specifically Cas9) in a complex with the appropriate guide RNAs (gRNAs). In bacteria or Archaea, Cas9 guide RNAs comprise 2 separate RNA species. A target-specific CRISPR-activating RNA (crRNA) directs the Cas9/gRNA complex to bind and target a specific DNA sequence. The crRNA has 2 functional domains, a 5′-domain that is target specific and a 3′-domain that directs binding of the crRNA to the transactivating crRNA (tracrRNA). The tracrRNA is a longer, universal RNA that binds the crRNA and mediates binding of the gRNA complex to Cas9. Binding of the tracrRNA induces an alteration of Cas9 structure, shifting from an inactive to an active conformation. The gRNA function can also be provided as an artificial single guide RNA (sgRNA), where the crRNA and tracrRNA are fused into a single species (see Jinek, M., et al., Science 337 p816-21, 2012). The sgRNA format permits transcription of a functional gRNA from a single transcription unit that can be provided by a double-stranded DNA (dsDNA) cassette containing a transcription promoter and the sgRNA sequence. In mammalian systems, these RNAs have been introduced by transfection of DNA cassettes containing RNA Pol III promoters (such as U6 or H1) driving RNA transcription, viral vectors, and single-stranded RNA following in vitro transcription (see Xu, T., et al., Appl Environ Microbiol, 2014. 80(5): p. 1544-52). In bacterial systems, these RNAs are expressed as part of a primitive immune system, or can be artificially expressed from a plasmid that is introduced by transformation (see Fonfara, I., et al., Nature, 2016. 532(7600): p. 517-21).

In the CRISPR-Cas system, using the system present in Streptococcus pyogenes as an example (S.py. or Spy), native crRNAs are about 42 bases long and contain a 5′-region of about 20 bases in length that is complementary to a target sequence (also referred to as a protospacer sequence or protospacer domain of the crRNA) and a 3′ region typically of about 22 bases in length that is complementary to a region of the tracrRNA sequence and mediates binding of the crRNA to the tracrRNA. A crRNA:tracrRNA complex comprises a functional gRNA capable of directing Cas9 cleavage of a complementary target DNA. The native tracrRNAs are about 85-90 bases long and have a 5′-region containing the region complementary to the crRNA. The remaining 3′ region of the tracrRNA includes secondary structure motifs (herein referred to as the “tracrRNA 3′-tail”) that mediate binding of the crRNA:tracrRNA complex to Cas9.

Jinek et al. extensively investigated the physical domains of the crRNA and tracrRNA that are required for proper functioning of the CRISPR-Cas system (Science, 2012. 337(6096): p. 816-21). They devised a truncated crRNA:tracrRNA fragment that could still function in CRISPR-Cas wherein the crRNA was the wild type 42 nucleotides and the tracrRNA was truncated to 75 nucleotides. They also developed an embodiment wherein the crRNA and tracrRNA are attached with a linker loop, forming a single guide RNA (sgRNA), which varies between 99-123 nucleotides in different embodiments.

At least three groups have elucidated the crystal structure of Streptococcus pyogenes Cas9 (SpyCas9). In Jinek, M., et al., the structure did not show the nuclease in complex with either a guide RNA or target DNA. They carried out molecular modeling experiments to reveal predictive interactions between the protein in complex with RNA and DNA (Science, 2014. 343, p. 1215, DOI: 10.1126/science/1247997).

In Nishimasu, H., et al., the crystal structure of Spy Cas9 is shown in complex with sgRNA and its target DNA at 2.5 angstrom resolution (Cell, 2014. 156(5): p. 935-49, incorporated herein in its entirety). The crystal structure identified two lobes to the Cas9 enzyme: a recognition lobe (REC) and a nuclease lobe (NUC). The sgRNA:target DNA heteroduplex (negatively charged) sits in the positively charged groove between the two lobes. The REC lobe, which shows no structural similarity with known proteins and therefore likely a Cas9-specific functional domain, interacts with the portions of the crRNA and tracrRNA that are complementary to each other.

Another group, Briner et al. (Mol Cell, 2014. 56(2): p. 333-9, incorporated herein in its entirety), identified and characterized the six conserved modules within native crRNA:tracrRNA duplexes and sgRNA. Anders et al. (Nature, 2014, 513(7519) p. 569-73) elucidated the structural basis for DNA sequence recognition of protospacer associate motif (PAM) sequences by Cas9 in association with an sgRNA guide.

The CRISPR-Cas endonuclease system is utilized in genomic engineering as follows: the gRNA complex (either a crRNA:tracrRNA complex or an sgRNA) binds to Cas9, inducing a conformational change that activates Cas9 and opens the DNA binding cleft, the protospacer domain of the crRNA (or sgRNA) aligns with the complementary target DNA and Cas9 binds the PAM sequence, initiating unwinding of the target DNA followed by annealing of the protospacer domain to the target, after which cleavage of the target DNA occurs. The Cas9 contains two domains, homologous to endonucleases HNH and RuvC respectively, wherein the HNH domain cleaves the DNA strand complementary to the crRNA and the RuvC-like domain cleaves the non-complementary strand. This results in a double-stranded break in the genomic DNA. When repaired by non-homologous end joining (NHEJ) the break is typically repaired in an imprecise fashion, resulting in the DNA sequence being shifted by 1 or more bases, leading to disruption of the natural DNA sequence and, in many cases, leading to a frameshift mutation if the event occurs in a coding exon of a protein-encoding gene. The break may also be repaired by homology directed recombination (HDR), which permits insertion of new genetic material based upon exogenous DNA introduced into the cell with the Cas9/gRNA complex, which is introduced into the cut site created by Cas9 cleavage.

While SpyCas9 is the protein being most widely used, it does hold some barriers to its effectiveness. SpyCas9 recognizes targeted sequences in the genome that are immediately followed by a GG dinucleotide sequence, and this system is therefore limited to

GC-rich regions of the genome. AT-rich species or genomic regions are therefore often not targetable with the SpyCas9 system. Furthermore, the fact that the Cas9 system includes a gRNA having both a crRNA and a tracrRNA moiety that comprise over 100 bases means that more RNA must be optimized and synthesized for sequence-specific targeting. As such, a shorter simpler gRNA would be desirable.

A second class 2 CRISPR system, assigned to type V, has been identified. This type V CRISPR-associated system contains Cpf1, which is a ˜1300 amino acid protein—slightly smaller than Cas9 from S. pyogenes. The PAM recognition sequence of Cpf1 from Acidaminococcus sp. BV3L6 or Lachnospiraceae bacterium ND2006 is TTTN, in contrast to the NGG PAM recognition domain of S. pyogenes Cas9 (FIG. 1). Having the ability to target AT-rich areas of the genome will be greatly beneficial to manipulate and study gene targets in regions that are lacking GG dinucleotide motifs. The Cpf1 system is also remarkably simple in that it does not utilize a separate tracrRNA, and only requires a single short crRNA of 40-45 base length that both specifies target DNA sequence and directs binding of the RNA to the Cpf1 nuclease.

In contrast to Cas9 which produces blunt-ended cleavage products, Cpf1 facilitates double stranded breaks with 4-5 nucleotide overhangs. The advantage of this is that it may ensure proper orientation as well as providing microhomology during non-homologous end joining (NHEJ). This could also be advantageous in non-dividing cell types that tend to be resistant to homology-directed repair (HDR). Furthermore, when Cpf1 cleaves, it does so further away from PAM than Cas9, which is also further away from the target site. As a result, the protospacer, and especially the seed sequence of the protospacer, are less likely to be edited, thereby leaving open the potential for a second round of cleavage if the desired repair event doesn't happen the first time.

The Cpf1 protein forms a complex with a single stranded RNA oligonucleotide to mediate targeted DNA cleavage. The single strand guide RNA oligonucleotide consists of a constant region of 20 nt and a target region of 21-24 nt for an overall length of 41-44 nt. There are many known orthologs of Cpf1 from a variety of different bacterial and Archaea sources that differ with respect to activity and target preference and may be candidates for use in genome editing applications. For the purposes of this invention, we primarily studied, as representative examples, the Cpf1 nucleases from A.s. (Acidaminococcus sp. BV3L6) Cpf1 and L.b. (Lachnospiraceae bacterium ND2006), both of which have already been shown to be active in mammalian cells as a tool for genome editing. Of note, the PAM recognition sequence is TTTN. The structure of the Cpf1 crRNA and relationship of RNA binding to the PAM site in genomic DNA is shown in FIG. 1.

Since the discovery of Cpf1 as another CRISPR pathway with potential utility for genome editing in mammalian cells, several publications have confirmed that the system works in mammals, can be used for embryo engineering, and the crystal structure and mechanism of PAM site recognition have been described. This system has also shown utility for screening purposes in genetically-tractable bacterial species such as E. coli. The system therefore has proven utility and developing optimized reagents to perform genome editing using Cpf1 would be beneficial.

Previous work done on the SpyCas9 crRNA and tracrRNA demonstrated that significant shortening of the naturally occurring crRNA and tracrRNA species could be done for RNAs made by chemical synthesis and that such shortened RNAs were 1) higher quality, 2) less costly to manufacture, and 3) showed improved performance in mammalian genome editing compared with the wild-type (WT) RNAs. See Collingwood, M. A., Jacobi, A. M., Rettig, G. R., Schubert, M. S., and Behlke, M. A., “CRISPR-BASED COMPOSITIONS AND METHOD OF USE,” U.S. patent application Ser. No. 14/975,709, filed Dec. 18, 2015, published now as U.S. Patent Application Publication No. US2016/0177304A1 on Jun. 23, 2016 and issued as U.S. Pat. No. 9,840,702 on Dec. 12, 2017. SEP

Prior work demonstrated that reducing the length of the FnCpf1 crRNA from 22 to 18 base length with deletions from the 3′-end supported cleavage of target DNA but that lengths of 17 or shorter showed reduced activity. Deletions or mutations that disrupted base-pairing in the universal loop domain disrupted activity. See Zetsche, B., Gootenberg, J. S., Abudayyeh, O. O., Slaymaker, I. M., Makarova, K. S., Essletzbichler, P., Volz, S. E., Joung, J., van der Oost, J., Regev, A., Koonin, E. V., and Zhang, F. (2015) Cpf1 is a single RNA-guided endonuclease of a class 2 CRISPR-Cas system. Cell 163:1-13. The FnCpf1 nuclease, however, does not work in mammalian cells to perform genome editing. It is unknown if the same length rules apply to the AsCpf1 crRNA as were observed for the FnCpf1 crRNA. We establish herein the shortest version of AsCpf1 crRNAs having full activity in mammalian genome editing applications. We also establish chemical modification patterns that maintain or improve functioning of synthetic Cpf1 crRNAs when used in mammalian or prokaryotic cells.

### BRIEF SUMMARY OF THE INVENTION

This invention pertains to Cpf1-based CRISPR genes, polypeptides encoded by the same, mammalian cell lines that stably express Cpf1, and chemically synthesized Cpf1 crRNAs and their use in compositions of CRISPR-Cpf1 systems and methods. Examples are shown employing the Cpf1 systems from Acidaminococcus sp. BV3L6 and Lachnospiraceae bacterium ND2006, however this is not intended to limit scope, which extends to Cpf1 homologs or orthologs isolated from other species.

In a first aspect, an isolated nucleic acid is provided. The isolated nucleic acid encodes an As Cpf1 polypeptide codon optimized for expression in H. sapiens as seen in SEQ ID NO:8, SEQ ID NO:15 and SEQ ID NO:22 which includes the use of nuclear localization signals as well as an epitope tag. The isolated nucleic acid also encodes as As Cpf1 polypeptide codon optimized for expression in E. coli which comprises SEQ ID NO:5 and may be fused or linked to a nuclear localization signal, multiple nuclear localization signals, or sequences encoding an epitope tag enabling detection by antibodies or other methods, and/or an affinity tag that enables simple purification of recombinants proteins expressed from the nucleic acid, such as a His-Tag as seen in SEQ ID NO: 12 and SEQ ID NO: 19.

In a second aspect, an isolated polypeptide encoding a wild-type As Cpf1 protein is provided. In a first respect, the isolated polypeptide comprises SEQ ID NO:2. The protein may be fused or linked to a nuclear localization signal, multiple nuclear localization signals, or sequences encoding an epitope tag enabling detection by antibodies or other methods, and/or an affinity tag that enables simple purification of recombinants proteins expressed from the nucleic acid, such as a His-Tag as seen in SEQ ID NO: 12, SEQ ID NO: 16 and SEQ ID NO: 19.

In a third aspect, an isolated nucleic acid is provided. The isolated nucleic acid encodes an Lb Cpf1 polypeptide codon optimized for expression in H. sapiens as seen in SEQ ID NO:9 and SEQ ID NO: 17, which includes the use of nuclear localization signals as well as an epitope tag. The isolated nucleic acid also encodes as Lb Cpf1 polypeptide codon optimized for expression in E. coli which comprises SEQ ID NO:6 and may be fused or linked to a nuclear localization signal, multiple nuclear localization signals, or sequences encoding an epitope tag enabling detection by antibodies or other methods, and/or an affinity tag that enables simple purification of recombinants proteins expressed from the nucleic acid, such as a His-Tag as seen in SEQ ID NO:13.

In a fourth aspect, an isolated polypeptide encoding a wild-type Lb Cpf1 protein is provided. In a first respect, the isolated polypeptide comprises SEQ ID NO:7 and SEQ ID NO: 10. The protein may be fused or linked to a nuclear localization signal, multiple nuclear localization signals, or sequences encoding an epitope tag enabling detection by antibodies or other methods, and/or an affinity tag that enables simple purification of recombinants proteins expressed from the nucleic acid, such as a His-Tag as seen in SEQ ID NO: 14.

In a fifth aspect, an isolated expression vector encoding SEQ ID NO:11, SEQ ID NO: 13, SEQ ID NO:15 and SEQ ID NO:17 is provided. The isolated expression vectors include a transcriptional initiator element, such as a promoter and enhancer, operably-linked to SEQ ID NO:11, SEQ ID NO: 13, SEQ ID NO:15 or SEQ ID NO: 17 to permit expression of the polypeptide encoded by SEQ ID NO: 12, SEQ ID NO: 14 or SEQ ID NO: 16.

In a sixth aspect, a host cell including an isolated expression vector encoding SEQ ID NO:11, SEQ ID NO: 13, SEQ ID NO: 15 and SEQ ID NO: 17 is provided. The isolated expression vector encoding SEQ ID NO:11, SEQ ID NO:13, SEQ ID NO: 15 or SEQ ID NO:17 is operably linked to a suitable promoter and other genetic elements (as necessary) to permit expression of a polypeptide comprising SEQ ID NO: 12, SEQ ID NO: 14 or SEQ ID NO:16.

In a seventh aspect, an isolated CRISPR/Cpf1 endonuclease system is provided. The system includes an AsCpf1 polypeptide and a suitable AsCpf1 crRNA.

In an eighth aspect, an isolated CRISPR/Cpf1 endonuclease system is provided. The system includes a human cell line expressing a AsCpf1 polypeptide and a suitable AsCpf1 crRNA.

In a ninth aspect, an isolated AsCpf1 crRNA is provided. The isolated AsCpf1 crRNA is active in a Clustered Regularly Interspaced Short Palindromic Repeats (CRISPR)/CRISPR-associated protein endonuclease system. Different variants of the crRNA are provided including species optimized for performance in mammalian cells and species optimized for performance in bacteria.

In a tenth aspect, a method of performing gene editing is provided. The method includes the step of contacting a candidate editing target site locus with an active CRISPR/Cpf1 endonuclease system having a wild-type AsCpf1 polypeptide and a suitable AsCpf1 crRNA.

### BRIEF DESCRIPTION OF THE DRAWINGS

FIG. 1 is a graphical representation of Cpf1 PAM recognition sites and alignment of guide crRNA to target DNA. Genomic DNA sequence of the human HPRT1 gene is shown at site ‘38595’. The “TTTN” PAM site that identifies As Cpf1 sites is highlighted and the sequence of the guide-binding site is underlined. DNA is shown in uppercase and RNA is shown in lowercase. In the Cpf1 crRNA, the protospacer target-specific domain is underlined and comprises the 3′-domain. The universal hairpin RNA sequence that mediates binding to Cpf1 protein comprises the 5′-domain.

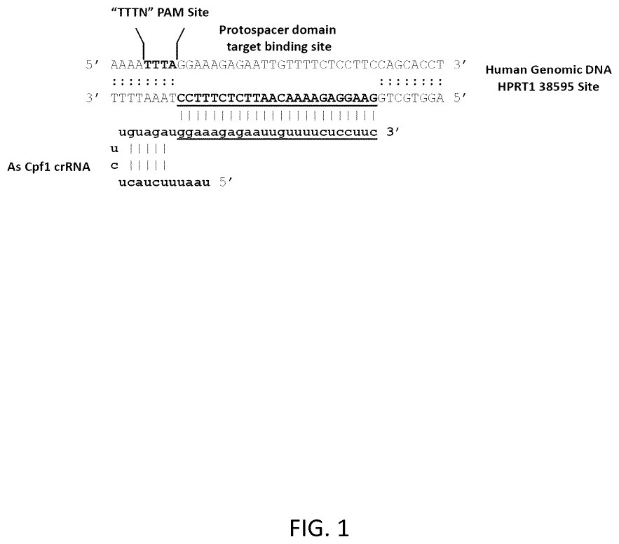

FIG. 2 depicts the map of a plasmid vector designed to express recombinant, synthetic, codon-optimized AsCpf1.

FIG. 3 depicts a schematic showing the final plasmid construct used to generate AsCpf1 stable cell lines.

FIG. 4 depicts an exemplary Western blot showing expression of V5-tagged proteins. Cell extract from a monoclonal HEK cell line that stably expresses Cas9 with a V5 tag was run in Lane 2. Cell extract from the new polyclonal HEK cell culture that expresses a V5-tagged AsCpf1 was run in Lane 3. Beta-actin is indicated and represents a mass loading control. Lane 1 was run with mass standard markers.

FIG. 5 depicts exemplary expression profiles of AsCpf1 mRNA normalized to internal control HPRT1 mRNA in 10 clonal transgenic cell lines. RT-qPCR assay locations vary in position along the AsCpf1 mRNA. Negative control non-transgenic HEK1 cells are shown on the far right.

FIG. 6 depicts exemplary Western blot showing relative expression levels of AsCpf1 protein in 10 monoclonal transgenic cell lines based on detection of the V5 epitope. Beta-actin loading control is seen below the AsCpf1 bands.

FIG. 7 depicts a modification tolerance map of AsCpf1 crRNAs at 2 sequence target sites, HPRT1-38351 (panel (i)) and HPRT1-38595 (panel (ii)), wherein the sequence of the universal 5′-loop domain is shown (5′-3′ orientation) for both the 24-nt protospacer domains (panels (i.a) and (ii.a)) and the 21-nt protospacer domains (panels (i.b) and (ii.b)). The sequence of the variable 3′-target specific protospacer domain is indicated as “N” bases, as this sequence varies for every target. Positions that did not suffer loss of activity when modified as a 2′OMe RNA residue in the single base walk are indicated in upper case whereas positions that showed loss of activity with modification are indicated in lower case. Above the lower case residues an arrow is shown that indicates the relative magnitude of the loss of activity, wherein a large arrow represents a large loss of activity, a mid-sized arrow represents a medium loss of activity, and a small arrow represents a minor loss of activity when the respective RNA residues are changed to 2′OMe RNA.

FIG. 8 depicts exemplary modified variants AsCpf1 crRNAs that are active in genome editing applications in mammalian cells at multiple target sites and therefore are not site-specific. The sequence of the universal 5′-loop domain is shown (5′-3′ orientation) and indicated with underline. The sequence of the variable 3′-target specific protospacer domain is indicated as “N” bases, as this sequence varies for every target. 2′OMe RNA modifications are indicated in uppercase and RNA residues are indicated in lowercase. “X” indicates a terminal non-base modifier, such as a C3 spacer (propanediol) or ZEN (napthyl-azo) group. “*” indicates a phosphorothioate (PS) internucleotide linkage.

FIG. 9 depicts exemplary results that compare the target editing activity of LbCpf1 with that of AsCpf1 and SpyCas9 for 12 regions of the HPRT gene with low GC content via T7EI mismatch endonuclease assay. In this study, all enzymes and crRNA were delivered as RNP complexes (5 μM), into HEK293 cells by nucleofection using the Amaxa system from Lonza, and DNA was extracted after 48 hr. Percent editing was determined by T7E1 mismatch endonuclease assay. Error bars represent standard errors of the means. Of note, the crRNA's for LbCpf1 were tested at the native 23mer nucleotide length as well as the previously optimized AsCpf1 length of 21 bases.

### DETAILED DESCRIPTION OF THE INVENTION

The methods and compositions of the invention described herein provide wild-type AsCpf1 nucleic acids and polypeptides for use in a CRISPR/Cpf1 system. The present invention describes an HEK293 cell line that has stable, low levels of expression of AsCpf1 in HEK293 and can be used as a platform for investigation and optimization of the nucleic acid components of the system. AsCpf1 provides a useful complement to SpyCas9 by expanding the range of PAM sequences that can be targeted from GC-rich areas (Cas9) to AT-rich areas of the genome (Cpf1), thereby expanding the range of sequences that can be modified using CRISPR genome engineering methods. In addition to having a T-rich PAM site, another advantage of the AsCpf1 system compared with Cas9 is the use of a single, short RNA molecule. However, unlike Cas9 that shows activity at most sites in the human genome, AsCpf1 shows little to no activity at half of TTTN PAM sites. Thus, exploiting the full potential of the AsCpf1 CRISPR system will be enhanced by the availability of suitable predictive software that enriches for high activity sites based on sequence context. The use of a stable constitutive Cpf1-expressing cell line makes the development of an algorithm easier to develop with reduced effort and cost as compared to using alternative methods, such as electroporation of ribonucleoprotein protein (RNP) complexes. HEK293 cells are an immortalized cell line that are easily cultured, passaged and cryogenically preserved. We established clonal cell lines that constitutively express SpyCas9 and AsCpf1 as suitable test vehicles for algorithm development or rapid testing/optimization of the chemical structure of guide RNAs. The present invention describes length and chemical modification of length-optimized variants of the AsCpf1 and LbCpf1 crRNAs that improve function in genome editing.

### AsCpf1-Encoded Genes, Polypeptides, Expression Vectors and Host Cells

The term “wild-type AsCpf1 protein” (“WT-AsCpf1” or “WT-AsCpf1 protein”) encompasses a protein having the identical amino acid sequence of the naturally-occurring Acidaminococcus sp. BV3L6 Cpf1 (e.g., SEQ ID NO:2) and that has biochemical and biological activity when combined with a suitable crRNA to form an active CRISPR/Cpf1 endonuclease system.

The term “wild-type LbCpf1 protein” (“WT-LbCpf1” or “WT-LbCpf1 protein”) encompasses a protein having the identical amino acid sequence of the naturally-occurring Lachnospiraceae bacterium ND2006 Cpf1 (e.g., SEQ ID NO:4) and that has biochemical and biological activity when combined with a suitable crRNA to form an active CRISPR/Cpf1 endonuclease system.

The term “wild-type CRISPR/Cpf1 endonuclease system” refers to a CRISPR/Cpf1 endonuclease system that includes wild-type AsCpf1 protein and a suitable AsCpf1 crRNA as a guide RNA.

The term “polypeptide” refers to any linear or branched peptide comprising more than one amino acid. Polypeptide includes protein or fragment thereof or fusion thereof, provided such protein, fragment or fusion retains a useful biochemical or biological activity.

Fusion proteins typically include extra amino acid information that is not native to the protein to which the extra amino acid information is covalently attached. Such extra amino acid information may include tags that enable purification or identification of the fusion protein. Such extra amino acid information may include peptides that enable the fusion proteins to be transported into cells and/or transported to specific locations within cells. Examples of tags for these purposes include the following: AviTag, which is a peptide allowing biotinylation by the enzyme BirA so the protein can be isolated by streptavidin (GLNDIFEAQKIEWHE (SEQ ID NO: 397)); Calmodulin-tag, which is a peptide bound by the protein calmodulin (KRRWKKNFIAVSAANRFKKISSSGAL (SEQ ID NO: 398)); polyglutamate tag, which is a peptide binding efficiently to anion-exchange resin such as Mono-Q (EEEEEE (SEQ ID NO: 399)); E-tag, which is a peptide recognized by an antibody (GAPVPYPDPLEPR (SEQ ID NO: 400)); FLAG-tag, which is a peptide recognized by an antibody (DYKDDDDK (SEQ ID NO: 401)); HA-tag, which is a peptide from hemagglutinin recognized by an antibody (YPYDVPDYA (SEQ ID NO: 402)); His-tag, which is typically 5-10 histidines (SEQ ID NO: 403) and can direct binding to a nickel or cobalt chelate (HHHHHH (SEQ ID NO: 404)); Myc-tag, which is a peptide derived from c-myc recognized by an antibody (EQKLISEEDL (SEQ ID NO: 405)); NE-tag, which is a novel 18-amino-acid synthetic peptide (TKENPRSNQEESYDDNES (SEQ ID NO: 406)) recognized by a monoclonal IgGI antibody, which is useful in a wide spectrum of applications including Western blotting, ELISA, flow cytometry, immunocytochemistry, immunoprecipitation, and affinity purification of recombinant proteins; S-tag, which is a peptide derived from Ribonuclease A (KETAAAKFERQHMDS (SEQ ID NO: 407)); SBP-tag, which is a peptide which binds to streptavidin; (MDEKTTGWRGGHVVEGLAGELEQLRARLEHHPQGQREP (SEQ ID NO: 408)); Softag 1, which is intended for mammalian expression (SLAELLNAGLGGS (SEQ ID NO: 409)); Softag 3, which is intended for prokaryotic expression (TQDPSRVG (SEQ ID NO: 410)); Strep-tag, which is a peptide which binds to streptavidin or the modified streptavidin called streptactin (Strep-tag II: WSHPQFEK (SEQ ID NO: 411)); TC tag, which is a tetracysteine tag that is recognized by FLASH and ReAsH biarsenical compounds (CCPGCC (SEQ ID NO: 412)) V5 tag, which is a peptide recognized by an antibody (GKPIPNPLLGLDST (SEQ ID NO: 413)); VSV-tag, a peptide recognized by an antibody (YTDIEMNRLGK (SEQ ID NO: 414)); Xpress tag (DLYDDDDK (SEQ ID NO: 415)); Isopeptag, which is a peptide which binds covalently to pilin-C protein (TDKDMTITFTNKKDAE (SEQ ID NO: 416)); SpyTag, which is a peptide which binds covalently to SpyCatcher protein (AHIVMVDAYKPTK (SEQ ID NO: 417)); SnoopTag, a peptide which binds covalently to SnoopCatcher protein (KLGDIEFIKVNK (SEQ ID NO: 418)); BCCP (Biotin Carboxyl Carrier Protein), which is a protein domain biotinylated by BirA to enable recognition by streptavidin; Glutathione-S-transferase-tag, which is a protein that binds to immobilized glutathione; Green fluorescent protein-tag, which is a protein which is spontaneously fluorescent and can be bound by antibodies; HaloTag, which is a mutated bacterial haloalkane dehalogenase that covalently attaches to a reactive haloalkane substrate to allow attachment to a wide variety of substrates; Maltose binding protein-tag, a protein which binds to amylose agarose; Nus-tag; Thioredoxin-tag; and Fc-tag, derived from immunoglobulin Fc domain, which allows dimerization and solubilization and can be used for purification on Protein-A Sepharose.

Nuclear localization signals (NLS), such as those obtained from SV40, allow for proteins to be transported to the nucleus immediately upon entering the cell. Given that the native AsCpf1 protein is bacterial in origin and therefore does not naturally comprise a NLS motif, addition of one or more NLS motifs to the recombinant AsCpf1 protein is expected to show improved genome editing activity when used in eukaryotic cells where the target genomic DNA substrate resides in the nucleus. Functional testing in HEK293 cells revealed that using a bipartite NLS (nucleoplasmin) increased editing in comparison to the current commercial design (3 SV40 NLS) and the use of single or dual OpT NLS that showed promise in the Cpf1 protein. Additional combinations of NLS elements including the bipartite are envisioned. Of note, the nucleoplasmin functions best in mammalian cells while the SV40 NLS appears to function in almost any nucleated cell. The bipartite SV40 NLS is functional in both Cas9 and Cpf1. Having two different NLS domains may expand effectiveness across a broad spectrum of species.

One skilled in the art would appreciate these various fusion tag technologies, as well as how to make and use fusion proteins that include them.

The term “isolated nucleic acid” include DNA, RNA, cDNA, and vectors encoding the same, where the DNA, RNA, cDNA and vectors are free of other biological materials from which they may be derived or associated, such as cellular components. Typically, an isolated nucleic acid will be purified from other biological materials from which they may be derived or associated, such as cellular components.

The term “isolated wild-type AsCpf1 nucleic acid” is an isolated nucleic acid that encodes a wild-type AsCpf1 protein. Examples of an isolated wild-type AsCpf1 nucleic acid include SEQ ID NO:1.

The term “isolated wild-type LbCpf1 nucleic acid” is an isolated nucleic acid that encodes a wild-type LbCpf1 protein. Examples of an isolated wild-type LbCpf1 nucleic acid include SEQ ID NO:3.

In a first aspect, an isolated nucleic acid is provided. The isolated nucleic acid encodes an As Cpf1 polypeptide codon optimized for expression in H. sapiens. In a first respect, the isolated nucleic acid comprises SEQ ID NO:8, SEQ ID NO: 15 and SEQ ID NO: 22 which includes the use of nuclear localization signals as well as an epitope tag. The isolated nucleic acid also encodes as As Cpf1 polypeptide codon optimized for expression in E. coli which comprises SEQ ID NO:5 and may be fused or linked to a nuclear localization signal, multiple nuclear localization signals, or sequences encoding an epitope tag enabling detection by antibodies or other methods, and/or an affinity tag that enables simple purification of recombinants proteins expressed from the nucleic acid, such as a His-Tag as seen in SEQ ID NO:12 and SEQ ID NO:19.

In a second aspect, an isolated polypeptide encoding a wild-type As Cpf1 protein is provided. In a first respect, the isolated polypeptide comprises SEQ ID NO:2, SEQ ID NO: 12, SEQ ID NO: 16 or SEQ ID NO: 19.

In a third aspect, an isolated expression vector encoding SEQ ID NO: 15 is provided. The isolated expression vector includes transcriptional initiator elements, such as a promoter and enhancer, operably-linked to SEQ ID NO:15 to permit expression of the polypeptide encoded by SEQ ID NO: 16. The isolated expression vector may additionally include transcriptional termination elements, posttranscriptional processing elements (for example, splicing donor and acceptor sequences and/or polyadenylation signaling sequences), mRNA stability elements and mRNA translational enhancer elements. Such genetic elements are understood and used by those having ordinary skill in the art.

In a fourth aspect, a host cell comprising an isolated expression vector encoding SEQ ID NO:15 is provided. The isolated expression vector encoding SEQ ID NO:15 is operably linked to a suitable promoter and other genetic elements (as necessary) to permit expression of a polypeptide comprising SEQ ID NO: 16. In a first respect, the host cell includes a human cell. In a second respect, the human cell comprises an immortalized cell line. In a third respect, the immortalized cell line is a HEK293 cell line. As a further elaboration of this third respect, the immortalized cell line comprises an isolated AsCpf1 crRNA capable of forming a ribonucleoprotein complex with the polypeptide comprising SEQ ID NO:2 to form a wild-type CRISPR/Cpf1 endonuclease.

Length- and Chemical Structure-Optimized AsCpf1 crRNAs

The term “length-modified,” as that term modifies RNA, refers to a shortened or truncated form of a reference RNA lacking nucleotide sequences or an elongated form of a reference RNA including additional nucleotide sequences.

The term “chemically-modified,” as that term modifies RNA, refers to a form of a reference RNA containing a chemically-modified nucleotide or a non-nucleotide chemical group covalently linked to the RNA. Chemically-modified RNA, as described herein, generally refers to synthetic RNA prepared using oligonucleotide synthesis procedures wherein modified nucleotides are incorporated during synthesis of an RNA oligonucleotide. However, chemically-modified RNA also includes synthetic RNA oligonucleotides modified with suitable modifying agents post-synthesis.

A competent CRISPR/Cpf1 endonuclease system includes a ribonucleoprotein (RNP) complex formed with isolated AsCpf1 protein and a guide RNA consisting of an isolated AsCpf1 crRNA. In some embodiments, an isolated length-modified and/or chemically-modified form of AsCpf1 crRNA is combined with purified AsCpf1 protein, an isolated mRNA encoding AsCpf1 protein or a gene encoding AsCpf1 protein in an expression vector. In certain assays, an isolated length-modified and/or chemically-modified form of AsCpf1 crRNA can be introduced into cell lines that stably express AsCpf1 protein from an endogenous expression cassette encoding the AsCpf1 gene.

It is desirable for synthesis of synthetic RNAs that sequences are shortened of unnecessary bases but not so shortened that loss of function results. The 5′-constant regions that mediates binding of the crRNA to the Cpf1 nuclease shows loss of activity if truncated below 20 residues. The 3′-variable domain that comprises the protospacer guide region which confers target sequence specificity to the crRNA naturally occurs as long as 25 bases. This domain can be shortened to around 20-21 bases with no loss of functional activity. The optimized length of the Cpf1 crRNA is therefore 40-41 bases, comprising a 20 base 5′-constant domain and a 20-21 base 3′-variable domain.

The present invention provides suitable guide RNAs for triggering DNA nuclease activity of the AsCpf1 nuclease. These optimized reagents, both in terms of length-modified and/or chemically-modified forms of crRNA's, provide for improved genome editing in any application with AsCpf1. The applications of CRISPR-based tools include, but are not limited to: plant gene editing, yeast gene editing, rapid generation of knockout/knockin animal lines, generating an animal model of disease state, correcting a disease state, inserting reporter genes, and whole genome functional screening. The “tool-kit” could be further expanded by including nickase versions and a dead mutant of AsCpf1 as a fusion protein with transcriptional activators CRISPRa) and repressors (CRISPRi).

RNA-guided DNA cleavage by AsCpf1 is primarily useful for its ability to target AT-rich gene regions (as compared with the GC-rich targeting by SpyCas9). The newly-discovered AsCpf1 crRNA truncation and modification variants will be suitable to promote AsCpf1-mediated staggered cutting and beneficial in gene silencing, homology directed repair or exon excision. The present invention defines the shortest AsCpf1 guide RNA that has full potency to direct gene editing by the CRISPR/Cpf1 endonuclease. This is useful for manufacturing to synthesize the shortest compound that fully functions, leading to higher quality, lower cost, while maximizing functionality.

Unlike S.py. Cas9 which requires a complex of 2 RNAs to recognize and cleave a target DNA sequence (comprising a hybridized crRNA:tracrRNA pair) or a long synthetic single-guide sgRNA, the Cpf1 nuclease only requires a short, single crRNA species to direct target recognition. This RNA comprises 2 domains, a 5′-domain of 20 RNA residues that is universal and mediates binding of the RNA species to the Cpf1 protein and a 3′domain of 21-24 RNA residues which is target specific and mediates binding of the RNP complex to a precise DNA sequence. A functional nuclease complex comprises a single crRNA (41-44 bases in length) and isolated Cpf1 protein, which combine in a 1:1 molar ratio to form an active complex. The guide crRNA species can be expressed in mammalian cells from expression plasmids or viral vectors. The crRNA can also be made as an in vitro transcript (IVT) and isolated as a pure enzymatic RNA species. More preferably, the crRNAs can be manufactured as a synthetic chemical RNA oligonucleotide. Chemical manufacturing enables use of modified residues, which have many advantages as will be outlined below.

Synthetic nucleic acids are attacked by cellular nucleases and rapidly degrade in mammalian cells or in serum. Chemical modification can confer relative nuclease resistance to the synthetic nucleic acids and prolong their half-lives, thereby dramatically improving functional performance and potency. As a further complication, synthetic nucleic acids are often recognized by the antiviral surveillance machinery in mammalian cells that are part of the innate immune system and lead to interferon response pathway activation, which can lead to cell death. Chemical modification can reduce or eliminate unwanted immune responses to synthetic RNAs. It is therefore useful to establish methods to chemically modify synthetic RNA oligonucleotides intended for use in live cells. Nucleic acid species that have specific interactions with protein factors, however, cannot be blindly modified as chemical modification will change tertiary structure of the nucleic acid and can block critical contact points between the nucleic acid and amino-acid residues. For example, the 2′-O-methyl RNA modification (2′OMe) will block the 2′-oxygen of RNA from interaction with amino-acid residues that in turn can disrupt functional interaction between a modified RNA and a protein. Likewise, a phosphorothioate modification can disrupt protein binding along the phosphate backbone of a nucleic acid through substitution of a non-bridging oxygen at the phosphate.

The 2′OMe modification is particularly useful in this setting as it has previously been shown to increase nuclease stability of antisense oligonucleotides (ASOs) and siRNAs and at the same kind can also reduce the risk that a chemically-synthesized RNA will trigger an innate immune response when introduced into mammalian cells. Specific modification patterns have been established that permit incorporation of this modified residue into an ASO or siRNA and retain function. Likewise, we have recently developed chemical modification patterns that improved the stability of the crRNA and tracrRNA that serve as guide RNA in the SpyCas9 system. Use of 2′OMe-modified residues in a CRISPR guide RNA improves RNA stability to nucleases and boosts the overall efficiency of editing in nuclease-rich environments while at the same time reduces cell death and toxicity associated with immunogenic triggers (such as is seen with long, unmodified RNAs).

The present invention relates to defining chemical modification patterns for the AsCpf1 crRNA that retain function in forming an active RNP complex capable of use in genome editing in mammalian cells. Modification ‘walks’ were performed where a single 2′OMe residue was place sequentially at every position with the Cpf1 crRNA. Sites that reduced or killed function of the RNP complex in genome editing were identified. Chemical modification patterns were defined that were compatible with high efficiency genome editing. The utility of 2′-fluoro (2′F) and locked nucleic acid (LNA) modifications at ‘modification competent’ position in the crRNA were also demonstrated. The use of phosphorothioate internucleotide linkages to modify select sites to reduce nuclease susceptibility was shown, as well as successful use of non-base modifiers as end blocks to reduce exonuclease attack on the synthetic RNAs. Taken together, these studies provide a ‘map’ of sites in the Cpf1 crRNA amenable to chemical modification along with a suite of modification chemistries demonstrated to function in the intended application in mammalian cells.

Specific examples of modification patterns are shown in the examples below. The 20-base 5′-constant domain could be heavily modified and retain function. In particular, using a 20-base 5′-constant region and counting from the 5′-end, RNA residues at position 1, 5, 6, 7, 8, 9, 10, 12, 13, 14, 16, 17, 18, and 19 can all be substituted with 2′OMe RNA residues with no loss of activity. Such substitutions can be made single, multiply, or all 14 residues modified, such that 14/20 residues have been changed in this domain from RNA to 2′OMe RNA. Maximum modification patterns that are tolerated in the 21-base 3′-variable domain vary with sequence of the domain. Within this domain, residues 21, 22, 23, 28, 29, 30, 32, 34, 35, 39, 40, and 41 (counting from the first base of the 5′-constant region) can be substituted with 2′OMe residues with no loss of activity.

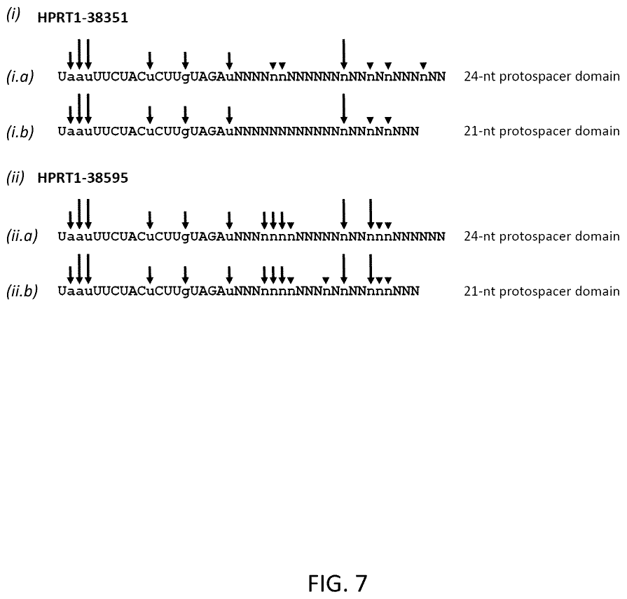

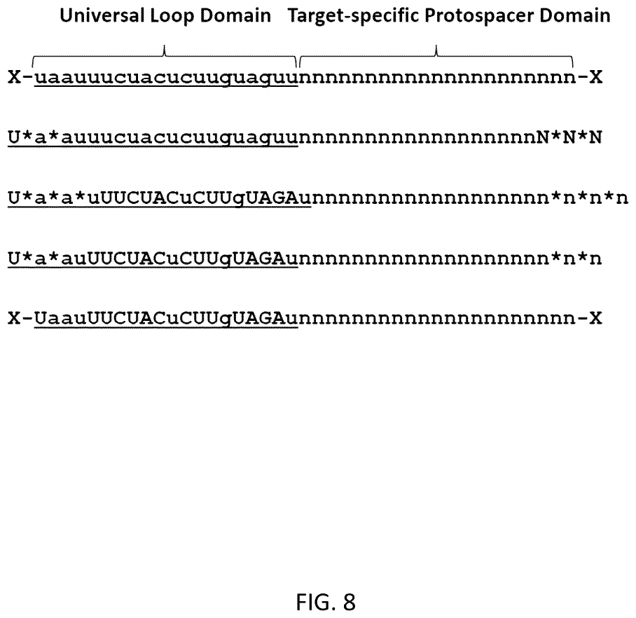

Only select positions within the 21-24-base 3′-target specific domain can be modified without compromising activity. Based on the crystal structure of Cpf1, there are many protein contact points within the constant region as well as the target region. For constant region modification, there is no obvious correlation that emerges when comparing the Cpf1 crystal structure contact points with the identified functional positions that can be modified-meaning that a good modification pattern cannot be predicted from the crystal structure. Likewise, empirical testing was needed to determine target region modification patterns. Based on the early 2′OMe modification testing, selected areas within the Cpf1 crRNA were modified using 2′OMe as an attempt to narrow down an area that will tolerate modification. The position of single residues within the Cpf1 crRNA that are sensitive to 2′OMe modification are shown in FIG. 7. Higher-level modification patterns that are potent triggers of Cpf1-mediated genome editing are shown in FIG. 8. 2′F modifications can be positioned at any residue that is tolerant to 2′OMe modification. Further, the 3′-variable domain is more tolerate of large blocks of 2′F modification than large blocks of 2′OMe modification. Hence a highly modified version of the Cpf1 crRNA comprises 2′OMe modification in the 3′-domain and 2′F modification in the 5′-domain. For medium or light modification patterns, either 2′OMe or 2′F (or both) modifications can be used in both domains. Also, LNA residues can be incorporated into the crRNA without compromising function, as defined in the examples below.

As an alternative to extensive use of 2′OMe or other modified sugar approaches, blocking exonuclease attack with non-base modifiers at the 3′-end and 5′-end are compatible with crRNA function and improve function in cells. Small C3 spacer (propanediol) or large ZEN groups work equally well for this approach. Further, phosphorothioate internucleotide linkages can be placed at select sites, such as between the terminal 2-3 bases on each end of the crRNA, but complete PS modification of the crRNA or complete modification of either the loop domain or the protospacer domain show reduced activity.

Guide RNAs are required in RNA-directed dsDNA cleavage by AsCpf1, which initiate the subsequent repair events that are involved in most CRISPR applications in mammalian cells. The use of modified synthetic AsCpf1 crRNAs as guides for AsCpf1 genome editing is provided. The utility of 2′OMe-modified AsCpf1 crRNAs, 2′F-modified AsCpf1 crRNAs, LNA modified AsCpf1 crRNAs, and end-blocked AsCpf1 crRNAs for CRISPR/Cpf1 applications in mammalian cells is demonstrated. Those with skill in the art will recognize and appreciate additional chemical modifications are possible based upon this disclosure. It is expected that many of these base modifying groups will likewise function according to the patterns taught in the present invention. Heretofore, all crRNAs used with Cpf1 for genome editing were unmodified RNA. In the present invention, functional modification patterns that improve properties of the AsCpf1 crRNA and lower risk of toxicity are provided.

AsCpf1 crRNAs can be made in cells from RNA transcription vectors, as in vitro transcripts (IVTs), or by chemical synthesis. Synthetic RNA oligonucleotides offer a distinct advantage because they alone allow for precise insertion of modified bases at specific sites in the molecule. The present invention provides a map of positions amenable to chemical modification that can be used to improve AsCpf1 crRNA performance in cells. For some applications, “minimal modification” approaches will be sufficient. In higher nuclease environments or for use in cells with particularly high innate immune reactivity, “high modification” approaches may work better. The present invention provides methods for low, medium, or high modification needs.

The applications of AsCpf1-based tools are many and varied. They include, but are not limited to: bacterial gene editing, plant gene editing, yeast gene editing, mammalian gene editing, editing of cells in the organs of live animals, editing of embryos, rapid generation of knockout/knock-in animal lines, generating an animal model of disease state, correcting a disease state, inserting a reporter gene, and whole genome functional screening.

In a fifth aspect, an isolated CRISPR/Cpf1 endonuclease system is provided. The system includes an AsCpf1 polypeptide and a suitable AsCpf1 crRNA. In a first respect, the AsCpf1 polypeptide comprises SEQ ID NO:2. In a second respect, the suitable AsCpf1 crRNA is selected from a length-truncated AsCpf1 crRNA or a chemically-modified AsCpf1 crRNA, or an AsCpf1 crRNA containing both length truncations and chemical modifications.

In a sixth aspect, an isolated CRISPR/Cpf1 endonuclease system is provided. The system includes a human cell line expressing an AsCpf1 polypeptide and a suitable AsCpf1 crRNA. In a first respect, the AsCpf1 polypeptide comprises at least one member selected from the group consisting of SEQ ID NO:2, SEQ ID NO: 12, SEQ ID NO: 16 and SEQ ID NO: 19. In a second respect, the suitable AsCpf1 crRNA is selected from a length-truncated AsCpf1 crRNA or a chemically-modified AsCpf1 crRNA, or an AsCpf1 crRNA containing both length truncations and chemical modifications.

In a seventh aspect, an isolated AsCpf1 crRNA is provided. The isolated AsCpf1 crRNA is active in a Clustered Regularly Interspaced Short Palindromic Repeats (CRISPR)/CRISPR-associated protein endonuclease system. In a first respect, the isolated AsCpf1 crRNA is selected from length-truncated AsCpf1 crRNA, a chemically-modified AsCpf1 crRNA, or an AsCpf1 crRNA containing both length truncations and chemical modifications.

In an eighth aspect, a method of performing gene editing is provided. The method includes the step of contacting a candidate editing target site locus with an active CRISPR/Cpf1 endonuclease system having a wild-type AsCpf1 polypeptide and a suitable AsCpf1 crRNA. In a first respect, the wild-type AsCpf1 polypeptide comprises at least one member selected from the group consisting of SEQ ID NO:2, SEQ ID NO:12, SEQ ID NO: 16 and SEQ ID NO: 19. In a second respect, the suitable AsCpf1 crRNA is selected from a length-truncated AsCpf1 crRNA, a chemically-modified AsCpf1 crRNA, or an AsCpf1 crRNA containing both length truncations and chemical modifications.

In another aspect, an isolated nucleic acid encoding an Lb Cpf1 polypeptide codon optimized for expression in H. sapiens is provided. In a first respect the isolated nucleic acid comprises SEQ ID NO:17 or SEQ ID NO:396.

In another aspect, an isolated polypeptide encoding a wild-type Lp Cpf1 protein is provided. In a first respect, the isolated polypeptide comprises SEQ ID NO:14 or SEQ ID NO: 24.

In another aspect, an isolated expression vector encoding SEQ ID NO:17 or SEQ ID NO: 396 is provided.

In another aspect, a host cell including an isolated expression vector encoding SEQ ID NO:17 or SEQ ID NO:396 is provided. The isolated expression vector encoding SEQ ID NO:17 or SEQ ID NO:396 is operably linked to a suitable promoter to permit expression of a polypeptide comprising SEQ ID NO: 14 or SEQ ID NO:24, respectively. In a first respect, the host cell comprises a human cell. In a second respect, the human cell comprises an immortalized cell line. In a third respect, the immortalized cell line is a HEK293 cell line. In a further elaboration of this respect, the host cell includes an isolated Lb Cpf1 crRNA capable of forming a ribonucleoprotein complex with the polypeptide selected from the group consisting of SEQ ID NO:4, SEQ ID NO:14, SEQ ID NO:20 and SEQ ID NO:24 to form a wild-type CRISPR/Cpf1 endonuclease.

In another aspect, an isolated CRISPR/Cpf1 endonuclease system having an Lb Cpf1 polypeptide and a suitable Cpf1 crRNA is provided. In a first respect, the CRISPR/Cpf1 endonuclease system includes a Lb Cpf1 polypeptide in the form of SEQ ID NO: 14. In a second respect, the isolated CRISPR/Cpf1 endonuclease system includes a suitable Cpf1 crRNA selected from a length-truncated Cpf1 crRNA or a chemically-modified Cpf1 crRNA, or a Cpf1 crRNA comprising both length truncations and chemical modifications.

In another aspect, an isolated CRISPR/Cpf1 endonuclease system having a human cell line expressing an Lb Cpf1 polypeptide and a suitable Cpf1 crRNA is provided. In a first respect, the Lb Cpf1 polypeptide is SEQ ID NO:14 or SEQ ID NO:24. In a second respect, the suitable Cpf1 crRNA is selected from a length-truncated Cpf1 crRNA or a chemically-modified Cpf1 crRNA, or a Cpf1 crRNA comprising both length truncations and chemical modifications.

In another respect, a method of performing gene editing is provided. The method includes the steps of contacting a candidate editing target site locus with an active CRISPR/Cpf1 endonuclease system having a wild-type Lb Cpf1 polypeptide and a suitable Cpf1 crRNA. In a first respect, the method includes a wild-type Lb Cpf1 polypeptide selected from the group consisting of SEQ ID NO:4, SEQ ID NO:14, SEQ ID NO:20 and SEQ ID NO:24. In a second respect, the suitable Cpf1 crRNA is selected from a length-truncated Cpf1 crRNA, a chemically-modified Cpf1 crRNA, or a Cpf1 crRNA comprising both length truncations and chemical modifications.

In another respect, a CRISPR endonuclease system having a recombinant Cpf1 fusion protein and a suitable crRNA is provided. In a first respect, the recombinant Cpf1 fusion protein is an isolated, purified protein. In a second respect, the recombinant Cpf1 fusion protein includes an N-terminal NLS, a C-terminal NLS and a plurality of affinity tags located at either the N-terminal or C-terminal ends. In one preferred embodiment, the recombinant Cpf1 fusion protein includes an N-terminal NLS, a C-terminal NLS and 3 N-terminal FLAG tags and a C-terminal 6×His (SEQ ID NO: 404) tag. In a third respect, the recombinant Cpf1 fusion protein and a suitable crRNA is provided in a 1:1 stoichiometric ratio (that is, in equimolar amounts).

### Example 1

### DNA and Amino Acid Sequences of Wild Type as Cpf1 Polypeptide, as Encoded in Isolated Nucleic Acid Vectors

The list below shows wild type (WT) As Cpf1 nucleases expressed as a polypeptide fusion protein described in the present invention. It will be appreciated by one with skill in the art that many different DNA sequences can encode/express the same amino acid (AA) sequence since in many cases more than one codon can encode for the same amino acid. The DNA sequences shown below only serve as example and other DNA sequences that encode the same protein (e.g., same amino acid sequence) are contemplated. It is further appreciated that additional features, elements or tags may be added to said sequences, such as NLS domains and the like. Examples are shown for WT AsCpf1 showing amino acid and DNA sequences for those proteins as Cpf1 alone and Cpf1 fused to both C-terminal and N-terminal SV40 NLS domains and a HIS-tag. Amino acid sequences that represent NLS sequences, domain linkers, or purification tags are indicated in bold font.

AsCpf1 Native Nucleotide Sequence
SEQ ID NO: 1
ATGACCCAATTTGAAGGTTTTACCAATTTATACCAAGTTTCGAAGACCCTTCGTTTTGAACTGATTC
CCCAAGGAAAAACACTCAAACATATCCAGGAGCAAGGGTTCATTGAGGAGGATAAAGCTCGCAATGA
CCATTACAAAGAGTTAAAACCAATCATTGACCGCATCTATAAGACTTATGCTGATCAATGTCTCCAA
CTGGTACAGCTTGACTGGGAGAATCTATCTGCAGCCATAGACTCCTATCGTAAGGAAAAAACCGAAG
AAACACGAAATGCGCTGATTGAGGAGCAAGCAACATATAGAAATGCGATTCATGACTACTTTATAGG
TCGGACGGATAATCTGACAGATGCCATAAATAAGCGCCATGCTGAAATCTATAAAGGACTTTTTAAA
GCTGAACTTTTCAATGGAAAAGTTTTAAAGCAATTAGGGACCGTAACCACGACAGAACATGAAAATG
CTCTACTCCGTTCGTTTGACAAATTTACGACCTATTTTTCCGGCTTTTATGAAAACCGAAAAAATGT
CTTTAGCGCTGAAGATATCAGCACGGCAATTCCCCATCGAATCGTCCAGGACAATTTCCCTAAATTT
AAGGAAAACTGCCATATTTTTACAAGATTGATAACCGCAGTTCCTTCTTTGCGGGAGCATTTTGAAA
ATGTCAAAAAGGCCATTGGAATCTTTGTTAGTACGTCTATTGAAGAAGTCTTTTCCTTTCCCTTTTA
TAATCAACTTCTAACCCAAACGCAAATTGATCTTTATAATCAACTTCTCGGCGGCATATCTAGGGAA
GCAGGCACAGAAAAAATCAAGGGACTTAATGAAGTTCTCAATCTGGCTATCCAAAAAAATGATGAAA
CAGCCCATATAATCGCGTCCCTGCCGCATCGTTTTATTCCTCTTTTTAAACAAATTCTTTCCGATCG
AAATACGTTATCCTTTATTTTGGAAGAATTCAAAAGCGATGAGGAAGTCATCCAATCCTTCTGCAAA
TATAAAACCCTCTTGAGAAACGAAAATGTACTGGAGACTGCAGAAGCCCTTTTCAATGAATTAAATT
CCATTGATTTGACTCATATCTTTATTTCCCATAAAAAGTTAGAAACCATCTCTTCAGCGCTTTGTGA
CCATTGGGATACCTTGCGCAATGCACTTTACGAAAGACGGATTTCTGAACTCACTGGCAAAATAACA
AAAAGTGCCAAAGAAAAAGTTCAAAGGTCATTAAAACATGAGGATATAAATCTCCAAGAAATTATTT
CTGCTGCAGGAAAAGAACTATCAGAAGCATTCAAACAAAAAACAAGTGAAATTCTTTCCCATGCCCA
TGCTGCACTTGACCAGCCTCTTCCCACAACATTAAAAAAACAGGAAGAAAAAGAAATCCTCAAATCA
CAGCTCGATTCGCTTTTAGGCCTTTATCATCTTCTTGATTGGTTTGCTGTCGATGAAAGCAATGAAG
TCGACCCAGAATTCTCAGCACGGCTGACAGGCATTAAACTAGAAATGGAACCAAGCCTTTCGTTTTA
TAATAAAGCAAGAAATTATGCGACAAAAAAGCCCTATTCGGTGGAAAAATTTAAATTGAATTTTCAA
ATGCCAACCCTTGCCTCTGGTTGGGATGTCAATAAAGAAAAAAATAATGGAGCTATTTTATTCGTAA
AAAATGGTCTCTATTACCTTGGTATCATGCCTAAACAGAAGGGGCGCTATAAAGCCCTGTCTTTTGA
GCCGACAGAAAAAACATCAGAAGGATTCGATAAGATGTACTATGACTACTTCCCAGATGCCGCAAAA
ATGATTCCTAAGTGTTCCACTCAGCTAAAGGCTGTAACCGCTCATTTTCAAACTCATACCACCCCCA
TTCTTCTCTCAAATAATTTCATTGAACCTCTTGAAATCACAAAAGAAATTTATGACCTGAACAATCC
TGAAAAGGAGCCTAAAAAGTTTCAAACGGCTTATGCAAAGAAGACAGGCGATCAAAAAGGCTATAGA
GAAGCGCTTTGCAAATGGATTGACTTTACGCGGGATTTTCTCTCTAAATATACGAAAACAACTTCAA
TCGATTTATCTTCACTCCGCCCTTCTTCGCAATATAAAGATTTAGGGGAATATTACGCCGAACTGAA
TCCGCTTCTCTATCATATCTCCTTCCAACGAATTGCTGAAAAGGAAATCATGGATGCTGTAGAAACG
GGAAAATTGTATCTGTTCCAAATCTACAATAAGGATTTTGCGAAGGGCCATCACGGGAAACCAAATC
TCCACACCCTGTATTGGACAGGTCTCTTCAGTCCTGAAAACCTTGCGAAAACCAGCATCAAACTTAA
TGGTCAAGCAGAATTGTTCTATCGACCTAAAAGCCGCATGAAGCGGATGGCCCATCGTCTTGGGGAA
AAAATGCTGAACAAAAAACTAAAGGACCAGAAGACACCGATTCCAGATACCCTCTACCAAGAACTGT
ACGATTATGTCAACCACCGGCTAAGCCATGATCTTTCCGATGAAGCAAGGGCCCTGCTTCCAAATGT
TATCACCAAAGAAGTCTCCCATGAAATTATAAAGGATCGGCGGTTTACTTCCGATAAATTTTTCTTC
CATGTTCCCATTACACTGAATTATCAAGCAGCCAATAGTCCCAGTAAATTCAACCAGCGTGTCAATG
CCTACCTTAAGGAGCATCCGGAAACGCCCATCATTGGTATCGATCGTGGAGAACGCAATCTAATCTA
TATTACCGTCATTGACAGTACTGGGAAAATTTTGGAGCAGCGTTCCCTGAATACCATCCAGCAATTT
GACTACCAAAAAAAATTGGACAACAGGGAAAAAGAGCGTGTTGCCGCCCGTCAAGCCTGGTCCGTCG
TCGGAACGATCAAAGACCTTAAACAAGGCTACTTGTCACAGGTCATCCATGAAATTGTAGACCTGAT
GATTCATTACCAAGCTGTTGTCGTCCTTGAAAACCTCAACTTCGGATTTAAATCAAAACGGACAGGC
ATTGCCGAAAAAGCAGTCTACCAACAATTTGAAAAGATGCTAATAGATAAACTCAACTGTTTGGTTC
TCAAAGATTATCCTGCTGAGAAAGTGGGAGGCGTCTTAAACCCGTATCAACTTACAGATCAGTTCAC
GAGCTTTGCAAAAATGGGCACGCAAAGCGGCTTCCTTTTCTATGTACCGGCCCCTTATACCTCAAAG
ATTGATCCCCTGACTGGTTTTGTCGATCCCTTTGTATGGAAGACCATTAAAAATCATGAAAGTCGGA
AGCATTTCCTAGAAGGATTTGATTTCCTGCATTATGATGTCAAAACAGGTGATTTTATCCTCCATTT
TAAAATGAATCGGAATCTCTCTTTCCAGAGAGGGCTTCCTGGCTTCATGCCAGCTTGGGATATTGTT
TTCGAAAAGAATGAAACCCAATTTGATGCAAAAGGGACGCCCTTCATTGCAGGAAAACGAATTGTTC
CTGTAATCGAAAATCATCGTTTTACGGGTCGTTACAGAGACCTCTATCCCGCTAATGAACTCATTGC
CCTTCTGGAAGAAAAAGGCATTGTCTTTAGAGACGGAAGTAATATATTACCCAAACTTTTAGAAAAT
GATGATTCTCATGCAATTGATACGATGGTCGCCTTGATTCGCAGTGTACTCCAAATGAGAAACAGCA
ATGCCGCAACGGGGGAAGACTACATCAACTCTCCCGTTAGGGATCTGAACGGGGTGTGTTTCGACAG
TCGATTCCAAAATCCAGAATGGCCAATGGATGCGGATGCCAACGGAGCTTATCATATTGCCTTAAAA
GGGCAGCTTCTTCTGAACCACCTCAAAGAAAGCAAAGATCTGAAATTACAAAACGGCATCAGCAACC
AAGATTGGCTGGCCTACATTCAGGAACTGAGAAACTGA
AsCpf1 Native Protein Sequence
SEQ ID NO: 2
MTQFEGFTNLYQVSKTLRFELIPQGKTLKHIQEQGFIEEDKARNDHYKELKPIIDRIYKTYADQCLQ
LVQLDWENLSAAIDSYRKEKTEETRNALIEEQATYRNAIHDYFIGRTDNLTDAINKRHAEIYKGLFK
AELFNGKVLKQLGTVTTTEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLTQTQIDLYNQLLGGISRE
AGTEKIKGLNEVLNLAIQKNDETAHIIASLPHRFIPLFKQILSDRNTLSFILEEFKSDEEVIQSFCK
YKTLLRNENVLETAEALFNELNSIDLTHIFISHKKLETISSALCDHWDTLRNALYERRISELTGKIT
KSAKEKVQRSLKHEDINLQEIISAAGKELSEAFKQKTSEILSHAHAALDQPLPTTLKKQEEKEILKS
QLDSLLGLYHLLDWFAVDESNEVDPEFSARLTGIKLEMEPSLSFYNKARNYATKKPYSVEKFKLNFQ
MPTLASGWDVNKEKNNGAILFVKNGLYYLGIMPKQKGRYKALSFEPTEKTSEGFDKMYYDYFPDAAK
MIPKCSTQLKAVTAHFQTHTTPILLSNNFIEPLEITKEIYDLNNPEKEPKKFQTAYAKKTGDQKGYR
EALCKWIDFTRDFLSKYTKTTSIDLSSLRPSSQYKDLGEYYAELNPLLYHISFQRIAEKEIMDAVET
GKLYLFQTYNKDFAKGHHGKPNLHTLYWTGLFSPENLAKTSIKLNGQAELFYRPKSRMKRMAHRLGE
KMLNKKLKDQKTPIPDTLYQELYDYVNHRLSHDLSDEARALLPNVITKEVSHEIIKDRRFTSDKFFF
HVPITLNYQAANSPSKFNQRVNAYLKEHPETPIIGIDRGERNLIYITVIDSTGKILEQRSLNTIQQF
DYQKKLDNREKERVAARQAWSVVGTIKDLKQGYLSQVIHEIVDLMIHYQAVVVLENLNFGFKSKRTG
IAEKAVYQQFEKMLIDKLNCLVLKDYPAEKVGGVLNPYQLTDQFTSFAKMGTQSGFLFYVPAPYTSK
IDPLTGFVDPFVWKTIKNHESRKHFLEGFDFLHYDVKTGDFILHFKMNRNLSFQRGLPGFMPAWDIV
FEKNETQFDAKGTPFIAGKRIVPVIENHRFTGRYRDLYPANELIALLEEKGIVERDGSNILPKLLEN
DDSHAIDTMVALIRSVLQMRNSNAATGEDYINSPVRDLNGVCFDSRFQNPEWPMDADANGAYHIALK
GQLLLNHLKESKDLKLQNGISNQDWLAYIQELRN
E. coli optimized AsCpf1 DNA
SEQ ID NO: 5
ATGACCCAGTTTGAAGGTTTCACCAATCTGTATCAGGTTAGCAAAACCCTGCGTTTTGAACTGATTC
CGCAGGGTAAAACCCTGAAACATATTCAAGAACAGGGCTTCATCGAAGAGGATAAAGCACGTAACGA
TCACTACAAAGAACTGAAACCGATTATCGACCGCATCTATAAAACCTATGCAGATCAGTGTCTGCAG
CTGGTTCAGCTGGATTGGGAAAATCTGAGCGCAGCAATTGATAGTTATCGCAAAGAAAAAACCGAAG
AAACCCGTAATGCACTGATTGAAGAACAGGCAACCTATCGTAATGCCATCCATGATTATTTCATTGG
TCGTACCGATAATCTGACCGATGCAATTAACAAACGTCACGCCGAAATCTATAAAGGCCTGTTTAAA
GCCGAACTGTTTAATGGCAAAGTTCTGAAACAGCTGGGCACCGTTACCACCACCGAACATGAAAATG
CACTGCTGCGTAGCTTTGATAAATTCACCACCTATTTCAGCGGCTTTTATGAGAATCGCAAAAACGT
GTTTAGCGCAGAAGATATTAGCACCGCAATTCCGCATCGTATTGTGCAGGATAATTTCCCGAAATTC
AAAGAGAACTGCCACATTTTTACCCGTCTGATTACCGCAGTTCCGAGCCTGCGTGAACATTTTGAAA
ACGTTAAAAAAGCCATCGGCATCTTTGTTAGCACCAGCATTGAAGAAGTTTTTAGCTTCCCGTTTTA
CAATCAGCTGCTGACCCAGACCCAGATTGATCTGTATAACCAACTGCTGGGTGGTATTAGCCGTGAA
GCAGGCACCGAAAAAATCAAAGGTCTGAATGAAGTGCTGAATCTGGCCATTCAGAAAAATGATGAAA
CCGCACATATTATTGCAAGCCTGCCGCATCGTTTTATTCCGCTGTTCAAACAAATTCTGAGCGATCG
TAATACCCTGAGCTTTATTCTGGAAGAATTCAAATCCGATGAAGAGGTGATTCAGAGCTTTTGCAAA
TACAAAACGCTGCTGCGCAATGAAAATGTTCTGGAAACTGCCGAAGCACTGTTTAACGAACTGAATA
GCATTGATCTGACCCACATCTTTATCAGCCACAAAAAACTGGAAACCATTTCAAGCGCACTGTGTGA
TCATTGGGATACCCTGCGTAATGCCCTGTATGAACGTCGTATTAGCGAACTGACCGGTAAAATTACC
AAAAGCGCGAAAGAAAAAGTTCAGCGCAGTCTGAAACATGAGGATATTAATCTGCAAGAGATTATTA
GCGCAGCCGGTAAAGAACTGTCAGAAGCATTTAAACAGAAAACCAGCGAAATTCTGTCACATGCACA
TGCAGCACTGGATCAGCCGCTGCCGACCACCCTGAAAAAACAAGAAGAAAAAGAAATCCTGAAAAGC
CAGCTGGATAGCCTGCTGGGTCTGTATCATCTGCTGGACTGGTTTGCAGTTGATGAAAGCAATGAAG
TTGATCCGGAATTTAGCGCACGTCTGACCGGCATTAAACTGGAAATGGAACCGAGCCTGAGCTTTTA
TAACAAAGCCCGTAATTATGCCACCAAAAAACCGTATAGCGTCGAAAAATTCAAACTGAACTTTCAG
ATGCCGACCCTGGCAAGCGGTTGGGATGTTAATAAAGAAAAAAACAACGGTGCCATCCTGTTCGTGA
AAAATGGCCTGTATTATCTGGGTATTATGCCGAAACAGAAAGGTCGTTATAAAGCGCTGAGCTTTGA
ACCGACGGAAAAAACCAGTGAAGGTTTTGATAAAATGTACTACGACTATTTTCCGGATGCAGCCAAA
ATGATTCCGAAATGTAGCACCCAGCTGAAAGCAGTTACCGCACATTTTCAGACCCATACCACCCCGA
TTCTGCTGAGCAATAACTTTATTGAACCGCTGGAAATCACCAAAGAGATCTACGATCTGAATAACCC
GGAAAAAGAGCCGAAAAAATTCCAGACCGCATATGCAAAAAAAACCGGTGATCAGAAAGGTTATCGT
GAAGCGCTGTGTAAATGGATTGATTTCACCCGTGATTTTCTGAGCAAATACACCAAAACCACCAGTA
TCGATCTGAGCAGCCTGCGTCCGAGCAGCCAGTATAAAGATCTGGGCGAATATTATGCAGAACTGAA
TCCGCTGCTGTATCATATTAGCTTTCAGCGTATTGCCGAGAAAGAAATCATGGACGCAGTTGAAACC
GGTAAACTGTACCTGTTCCAGATCTACAATAAAGATTTTGCCAAAGGCCATCATGGCAAACCGAATC
TGCATACCCTGTATTGGACCGGTCTGTTTAGCCCTGAAAATCTGGCAAAAACCTCGATTAAACTGAA
TGGTCAGGCGGAACTGTTTTATCGTCCGAAAAGCCGTATGAAACGTATGGCACATCGTCTGGGTGAA
AAAATGCTGAACAAAAAACTGAAAGACCAGAAAACCCCGATCCCGGATACACTGTATCAAGAACTGT
ATGATTATGTGAACCATCGTCTGAGCCATGATCTGAGTGATGAAGCACGTGCCCTGCTGCCGAATGT
TATTACCAAAGAAGTTAGCCACGAGATCATTAAAGATCGTCGTTTTACCAGCGACAAATTCTTTTTT
CATGTGCCGATTACCCTGAATTATCAGGCAGCAAATAGCCCGAGCAAATTTAACCAGCGTGTTAATG
CATATCTGAAAGAACATCCAGAAACGCCGATTATTGGTATTGATCGTGGTGAACGTAACCTGATTTA
TATCACCGTTATTGATAGCACCGGCAAAATCCTGGAACAGCGTAGCCTGAATACCATTCAGCAGTTT
GATTACCAGAAAAAACTGGATAATCGCGAGAAAGAACGTGTTGCAGCACGTCAGGCATGGTCAGTTG
TTGGTACAATTAAAGACCTGAAACAGGGTTATCTGAGCCAGGTTATTCATGAAATTGTGGATCTGAT
GATTCACTATCAGGCCGTTGTTGTGCTGGAAAACCTGAATTTTGGCTTTAAAAGCAAACGTACCGGC
ATTGCAGAAAAAGCAGTTTATCAGCAGTTCGAGAAAATGCTGATTGACAAACTGAATTGCCTGGTGC
TGAAAGATTATCCGGCTGAAAAAGTTGGTGGTGTTCTGAATCCGTATCAGCTGACCGATCAGTTTAC
CAGCTTTGCAAAAATGGGCACCCAGAGCGGATTTCTGTTTTATGTTCCGGCACCGTATACGAGCAAA
ATTGATCCGCTGACCGGTTTTGTTGATCCGTTTGTTTGGAAAACCATCAAAAACCATGAAAGCCGCA
AACATTTTCTGGAAGGTTTCGATTTTCTGCATTACGACGTTAAAACGGGTGATTTCATCCTGCACTT
TAAAATGAATCGCAATCTGAGTTTTCAGCGTGGCCTGCCTGGTTTTATGCCTGCATGGGATATTGTG
TTTGAGAAAAACGAAACACAGTTCGATGCAAAAGGCACCCCGTTTATTGCAGGTAAACGTATTGTTC
CGGTGATTGAAAATCATCGTTTCACCGGTCGTTATCGCGATCTGTATCCGGCAAATGAACTGATCGC
ACTGCTGGAAGAGAAAGGTATTGTTTTTCGTGATGGCTCAAACATTCTGCCGAAACTGCTGGAAAAT
GATGATAGCCATGCAATTGATACCATGGTTGCACTGATTCGTAGCGTTCTGCAGATGCGTAATAGCA
ATGCAGCAACCGGTGAAGATTACATTAATAGTCCGGTTCGTGATCTGAATGGTGTTTGTTTTGATAG
CCGTTTTCAGAATCCGGAATGGCCGATGGATGCAGATGCAAATGGTGCATATCATATTGCACTGAAA
GGACAGCTGCTGCTGAACCACCTGAAAGAAAGCAAAGATCTGAAACTGCAAAACGGCATTAGCAATC
AGGATTGGCTGGCATATATCCAAGAACTGCGTAACTGA
AsCpf1 Human Codon Optimized Nucleotide Sequence
SEQ ID NO: 8
ATGACCCAGTTCGAGGGCTTCACCAACCTGTACCAGGTGTCCAAGACCCTGAGATTCGAGCTGATCC
CCCAGGGCAAGACACTGAAGCACATCCAGGAACAGGGCTTCATCGAAGAGGACAAGGCCCGGAACGA
CCACTACAAAGAGCTGAAGCCCATCATCGACCGGATCTACAAGACCTACGCCGACCAGTGCCTGCAG
CTGGTGCAGCTGGACTGGGAGAATCTGAGCGCCGCCATCGACAGCTACCGGAAAGAGAAAACCGAGG
AAACCCGGAACGCCCTGATCGAGGAACAGGCCACCTACAGAAACGCCATCCACGACTACTTCATCGG
CCGGACCGACAACCTGACCGACGCCATCAACAAGCGGCACGCCGAGATCTATAAGGGCCTGTTCAAG
GCCGAGCTGTTCAACGGCAAGGTGCTGAAGCAGCTGGGCACCGTGACCACCACCGAGCACGAAAACG
CCCTGCTGCGGAGCTTCGACAAGTTCACCACCTACTTCAGCGGCTTCTACGAGAACCGGAAGAACGT
GTTCAGCGCCGAGGACATCAGCACCGCCATCCCCCACAGAATCGTGCAGGACAACTTCCCCAAGTTC
AAAGAGAACTGCCACATCTTCACCCGGCTGATCACCGCCGTGCCCAGCCTGAGAGAACACTTCGAGA
ACGTGAAGAAGGCCATCGGCATCTTCGTGTCCACCAGCATCGAGGAAGTGTTCAGCTTCCCATTCTA
CAACCAGCTGCTGACCCAGACCCAGATCGACCTGTATAATCAGCTGCTGGGCGGCATCAGCAGAGAG
GCCGGCACCGAGAAGATCAAGGGCCTGAACGAAGTGCTGAACCTGGCCATCCAGAAGAACGACGAGA
CAGCCCACATCATTGCCAGCCTGCCCCACCGGTTCATCCCTCTGTTCAAGCAGATCCTGAGCGACAG
AAACACCCTGAGCTTCATCCTGGAAGAGTTCAAGTCCGATGAGGAAGTGATCCAGAGCTTCTGCAAG
TATAAGACCCTGCTGAGGAACGAGAATGTGCTGGAAACCGCCGAGGCCCTGTTCAATGAGCTGAACA
GCATCGACCTGACCCACATCTTTATCAGCCACAAGAAGCTGGAAACAATCAGCAGCGCCCTGTGCGA
CCACTGGGACACACTGCGGAATGCCCTGTACGAGCGGCGGATCTCTGAGCTGACCGGCAAGATCACC
AAGAGCGCCAAAGAAAAGGTGCAGCGGAGCCTGAAGCACGAGGATATCAACCTGCAGGAAATCATCA
GCGCCGCTGGCAAAGAACTGAGCGAGGCCTTTAAGCAGAAAACCAGCGAGATCCTGTCCCACGCCCA
CGCCGCACTGGATCAGCCTCTGCCTACCACCCTGAAGAAGCAGGAAGAGAAAGAGATCCTGAAGTCC
CAGCTGGACAGCCTGCTGGGCCTGTACCATCTGCTGGATTGGTTCGCCGTGGACGAGAGCAACGAGG
TGGACCCCGAGTTCTCCGCCAGACTGACAGGCATCAAACTGGAAATGGAACCCAGCCTGTCCTTCTA
CAACAAGGCCAGAAACTACGCCACCAAGAAACCCTACAGCGTGGAAAAGTTTAAGCTGAACTTCCAG
ATGCCCACCCTGGCCAGCGGCTGGGACGTGAACAAAGAGAAGAACAACGGCGCCATCCTGTTCGTGA
AGAACGGACTGTACTACCTGGGCATCATGCCTAAGCAGAAGGGCAGATACAAGGCCCTGTCCTTTGA
GCCCACCGAAAAGACCAGCGAGGGCTTTGACAAGATGTACTACGATTACTTCCCCGACGCCGCCAAG
ATGATCCCCAAGTGCAGCACCCAGCTGAAGGCCGTGACCGCCCACTTTCAGACCCACACCACCCCCA
TCCTGCTGAGCAACAACTTCATCGAGCCCCTGGAAATCACCAAAGAGATCTACGACCTGAACAACCC
CGAGAAAGAGCCCAAGAAGTTCCAGACCGCCTACGCCAAGAAAACCGGCGACCAGAAGGGCTACCGC
GAGGCTCTGTGCAAGTGGATCGACTTTACCCGGGACTTCCTGAGCAAGTACACCAAGACCACCTCCA
TCGATCTGAGCAGCCTGCGGCCCAGCTCCCAGTACAAGGATCTGGGCGAGTACTACGCCGAGCTGAA
CCCTCTGCTGTACCACATCAGCTTCCAGCGGATCGCCGAAAAAGAAATCATGGACGCCGTGGAAACC
GGCAAGCTGTACCTGTTCCAGATCTATAACAAGGACTTCGCCAAGGGCCACCACGGCAAGCCCAATC
TGCACACCCTGTACTGGACCGGCCTGTTTAGCCCCGAGAATCTGGCCAAGACCAGCATCAAGCTGAA
CGGCCAGGCCGAACTGTTTTACCGGCCCAAGAGCCGGATGAAGCGGATGGCCCATAGACTGGGCGAG
AAGATGCTGAACAAGAAACTGAAGGACCAGAAAACCCCTATCCCCGACACACTGTATCAGGAACTGT
ACGACTACGTGAACCACCGGCTGAGCCACGACCTGTCCGACGAAGCTAGAGCACTGCTGCCCAACGT
GATCACAAAAGAGGTGTCCCACGAGATCATCAAGGACCGGCGGTTTACCTCCGATAAGTTCTTCTTC
CACGTGCCCATCACCCTGAACTACCAGGCCGCCAACAGCCCCAGCAAGTTCAACCAGAGAGTGAACG
CCTACCTGAAAGAGCACCCCGAGACACCCATCATTGGCATCGACAGAGGCGAGCGGAACCTGATCTA
CATCACCGTGATCGACAGCACAGGCAAAATCCTGGAACAGAGAAGCCTGAACACCATCCAGCAGTTC
GACTACCAGAAGAAACTGGACAACCGGGAAAAAGAACGGGTGGCCGCCAGACAGGCTTGGAGCGTCG
TGGGCACCATTAAGGACCTGAAGCAGGGCTACCTGAGCCAAGTGATTCACGAGATCGTGGACCTGAT
GATCCACTATCAGGCTGTGGTGGTGCTGGAAAACCTGAACTTCGGCTTCAAGAGCAAGCGGACCGGA
ATCGCCGAGAAAGCCGTGTACCAGCAGTTTGAGAAAATGCTGATCGACAAGCTGAATTGCCTGGTGC
TGAAAGACTACCCCGCTGAGAAAGTGGGAGGCGTGCTGAATCCCTACCAGCTGACCGACCAGTTCAC
CTCCTTTGCCAAGATGGGAACCCAGAGCGGCTTCCTGTTCTACGTGCCAGCCCCCTACACCAGCAAG
ATCGACCCTCTGACCGGCTTCGTGGACCCCTTCGTGTGGAAAACCATCAAGAACCACGAGTCCCGGA
AGCACTTCCTGGAAGGCTTTGACTTCCTGCACTACGACGTGAAAACAGGCGATTTCATCCTGCACTT
CAAGATGAATCGGAATCTGTCCTTCCAGAGGGGCCTGCCCGGCTTCATGCCTGCCTGGGATATCGTG
TTCGAGAAGAATGAGACACAGTTCGACGCCAAGGGAACCCCCTTTATCGCCGGCAAGAGGATCGTGC
CTGTGATCGAGAACCACAGATTCACCGGCAGATACCGGGACCTGTACCCCGCCAACGAGCTGATTGC
CCTGCTGGAAGAGAAGGGCATCGTGTTCCGGGACGGCAGCAACATCCTGCCCAAGCTGCTGGAAAAT
GACGACAGCCACGCCATCGATACCATGGTGGCACTGATCCGCAGCGTGCTGCAGATGCGGAACAGCA
ATGCCGCCACCGGCGAGGACTACATCAATAGCCCAGTGCGGGACCTGAACGGCGTGTGCTTCGACAG
CAGATTCCAGAACCCCGAGTGGCCCATGGATGCCGACGCCAATGGCGCCTACCACATTGCCCTGAAG
GGACAGCTGCTGCTGAACCATCTGAAAGAGAGCAAAGACCTGAAACTGCAGAACGGCATCTCCAACC
AGGACTGGCTGGCCTATATCCAGGAACTGCGGAACTGA
E. coli optimized As Cpf1 with flanking NLS's, V5 tag
and 6x His - DNA
SEQ ID NO: 11
ATGGGTCGGGATCCAGGTAAACCGATTCCGAATCCGCTGCTGGGTCTGGATAGCACCGCACCGAAAA
AAAAACGTAAAGTTGGTATTCATGGTGTTCCGGCAGCAACCCAGTTTGAAGGTTTCACCAATCTGTA
TCAGGTTAGCAAAACCCTGCGTTTTGAACTGATTCCGCAGGGTAAAACCCTGAAACATATTCAAGAA
CAGGGCTTCATCGAAGAGGATAAAGCACGTAACGATCACTACAAAGAACTGAAACCGATTATCGACC
GCATCTATAAAACCTATGCAGATCAGTGTCTGCAGCTGGTTCAGCTGGATTGGGAAAATCTGAGCGC
AGCAATTGATAGTTATCGCAAAGAAAAAACCGAAGAAACCCGTAATGCACTGATTGAAGAACAGGCA
ACCTATCGTAATGCCATCCATGATTATTTCATTGGTCGTACCGATAATCTGACCGATGCAATTAACA
AACGTCACGCCGAAATCTATAAAGGCCTGTTTAAAGCCGAACTGTTTAATGGCAAAGTTCTGAAACA
GCTGGGCACCGTTACCACCACCGAACATGAAAATGCACTGCTGCGTAGCTTTGATAAATTCACCACC
TATTTCAGCGGCTTTTATGAGAATCGCAAAAACGTGTTTAGCGCAGAAGATATTAGCACCGCAATTC
CGCATCGTATTGTGCAGGATAATTTCCCGAAATTCAAAGAGAACTGCCACATTTTTACCCGTCTGAT
TACCGCAGTTCCGAGCCTGCGTGAACATTTTGAAAACGTTAAAAAAGCCATCGGCATCTTTGTTAGC
ACCAGCATTGAAGAAGTTTTTAGCTTCCCGTTTTACAATCAGCTGCTGACCCAGACCCAGATTGATC
TGTATAACCAACTGCTGGGTGGTATTAGCCGTGAAGCAGGCACCGAAAAAATCAAAGGTCTGAATGA
AGTGCTGAATCTGGCCATTCAGAAAAATGATGAAACCGCACATATTATTGCAAGCCTGCCGCATCGT
TTTATTCCGCTGTTCAAACAAATTCTGAGCGATCGTAATACCCTGAGCTTTATTCTGGAAGAATTCA
AATCCGATGAAGAGGTGATTCAGAGCTTTTGCAAATACAAAACGCTGCTGCGCAATGAAAATGTTCT
GGAAACTGCCGAAGCACTGTTTAACGAACTGAATAGCATTGATCTGACCCACATCTTTATCAGCCAC
AAAAAACTGGAAACCATTTCAAGCGCACTGTGTGATCATTGGGATACCCTGCGTAATGCCCTGTATG
AACGTCGTATTAGCGAACTGACCGGTAAAATTACCAAAAGCGCGAAAGAAAAAGTTCAGCGCAGTCT
GAAACATGAGGATATTAATCTGCAAGAGATTATTAGCGCAGCCGGTAAAGAACTGTCAGAAGCATTT
AAACAGAAAACCAGCGAAATTCTGTCACATGCACATGCAGCACTGGATCAGCCGCTGCCGACCACCC
TGAAAAAACAAGAAGAAAAAGAAATCCTGAAAAGCCAGCTGGATAGCCTGCTGGGTCTGTATCATCT
GCTGGACTGGTTTGCAGTTGATGAAAGCAATGAAGTTGATCCGGAATTTAGCGCACGTCTGACCGGC
ATTAAACTGGAAATGGAACCGAGCCTGAGCTTTTATAACAAAGCCCGTAATTATGCCACCAAAAAAC
CGTATAGCGTCGAAAAATTCAAACTGAACTTTCAGATGCCGACCCTGGCAAGCGGTTGGGATGTTAA
TAAAGAAAAAAACAACGGTGCCATCCTGTTCGTGAAAAATGGCCTGTATTATCTGGGTATTATGCCG
AAACAGAAAGGTCGTTATAAAGCGCTGAGCTTTGAACCGACGGAAAAAACCAGTGAAGGTTTTGATA
AAATGTACTACGACTATTTTCCGGATGCAGCCAAAATGATTCCGAAATGTAGCACCCAGCTGAAAGC
AGTTACCGCACATTTTCAGACCCATACCACCCCGATTCTGCTGAGCAATAACTTTATTGAACCGCTG
GAAATCACCAAAGAGATCTACGATCTGAATAACCCGGAAAAAGAGCCGAAAAAATTCCAGACCGCAT
ATGCAAAAAAAACCGGTGATCAGAAAGGTTATCGTGAAGCGCTGTGTAAATGGATTGATTTCACCCG
TGATTTTCTGAGCAAATACACCAAAACCACCAGTATCGATCTGAGCAGCCTGCGTCCGAGCAGCCAG
TATAAAGATCTGGGCGAATATTATGCAGAACTGAATCCGCTGCTGTATCATATTAGCTTTCAGCGTA
TTGCCGAGAAAGAAATCATGGACGCAGTTGAAACCGGTAAACTGTACCTGTTCCAGATCTACAATAA
AGATTTTGCCAAAGGCCATCATGGCAAACCGAATCTGCATACCCTGTATTGGACCGGTCTGTTTAGC
CCTGAAAATCTGGCAAAAACCTCGATTAAACTGAATGGTCAGGCGGAACTGTTTTATCGTCCGAAAA
GCCGTATGAAACGTATGGCACATCGTCTGGGTGAAAAAATGCTGAACAAAAAACTGAAAGACCAGAA
AACCCCGATCCCGGATACACTGTATCAAGAACTGTATGATTATGTGAACCATCGTCTGAGCCATGAT
CTGAGTGATGAAGCACGTGCCCTGCTGCCGAATGTTATTACCAAAGAAGTTAGCCACGAGATCATTA
AAGATCGTCGTTTTACCAGCGACAAATTCTTTTTTCATGTGCCGATTACCCTGAATTATCAGGCAGC
AAATAGCCCGAGCAAATTTAACCAGCGTGTTAATGCATATCTGAAAGAACATCCAGAAACGCCGATT
ATTGGTATTGATCGTGGTGAACGTAACCTGATTTATATCACCGTTATTGATAGCACCGGCAAAATCC
TGGAACAGCGTAGCCTGAATACCATTCAGCAGTTTGATTACCAGAAAAAACTGGATAATCGCGAGAA
AGAACGTGTTGCAGCACGTCAGGCATGGTCAGTTGTTGGTACAATTAAAGACCTGAAACAGGGTTAT
CTGAGCCAGGTTATTCATGAAATTGTGGATCTGATGATTCACTATCAGGCCGTTGTTGTGCTGGAAA
ACCTGAATTTTGGCTTTAAAAGCAAACGTACCGGCATTGCAGAAAAAGCAGTTTATCAGCAGTTCGA
GAAAATGCTGATTGACAAACTGAATTGCCTGGTGCTGAAAGATTATCCGGCTGAAAAAGTTGGTGGT
GTTCTGAATCCGTATCAGCTGACCGATCAGTTTACCAGCTTTGCAAAAATGGGCACCCAGAGCGGAT
TTCTGTTTTATGTTCCGGCACCGTATACGAGCAAAATTGATCCGCTGACCGGTTTTGTTGATCCGTT
TGTTTGGAAAACCATCAAAAACCATGAAAGCCGCAAACATTTTCTGGAAGGTTTCGATTTTCTGCAT
TACGACGTTAAAACGGGTGATTTCATCCTGCACTTTAAAATGAATCGCAATCTGAGTTTTCAGCGTG
GCCTGCCTGGTTTTATGCCTGCATGGGATATTGTGTTTGAGAAAAACGAAACACAGTTCGATGCAAA
AGGCACCCCGTTTATTGCAGGTAAACGTATTGTTCCGGTGATTGAAAATCATCGTTTCACCGGTCGT
TATCGCGATCTGTATCCGGCAAATGAACTGATCGCACTGCTGGAAGAGAAAGGTATTGTTTTTCGTG
ATGGCTCAAACATTCTGCCGAAACTGCTGGAAAATGATGATAGCCATGCAATTGATACCATGGTTGC
ACTGATTCGTAGCGTTCTGCAGATGCGTAATAGCAATGCAGCAACCGGTGAAGATTACATTAATAGT
CCGGTTCGTGATCTGAATGGTGTTTGTTTTGATAGCCGTTTTCAGAATCCGGAATGGCCGATGGATG
CAGATGCAAATGGTGCATATCATATTGCACTGAAAGGACAGCTGCTGCTGAACCACCTGAAAGAAAG
CAAAGATCTGAAACTGCAAAACGGCATTAGCAATCAGGATTGGCTGGCATATATCCAAGAACTGCGT
AACCCTAAAAAAAAACGCAAAGTGAAGCTTGCGGCCGCACTCGAGCACCACCACCACCACCACTGA
E. coli optimized As Cpf1 with 5′- and 3′-flanking
NLS's, 5′-V5 tag and 3′-6x His
SEQ ID NO: 12
MGRDPGKPIPNPLLGLDSTAPKKKRKVGIHGVPAATQFEGFTNLYQVSKTLRFELIPQGKTLKHIQE
QGFIEEDKARNDHYKELKPIIDRIYKTYADQCLQLVQLDWENLSAAIDSYRKEKTEETRNALIEEQA
TYRNAIHDYFIGRTDNLTDAINKRHAEIYKGLFKAELFNGKVLKQLGTVTTTEHENALLRSFDKFTT
YFSGFYENRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEALFNELNSIDLTHIFISH
KKLETISSALCDHWDTLRNALYERRISELTGKITKSAKEKVQRSLKHEDINLQEIISAAGKELSEAF
KQKTSEILSHAHAALDQPLPTTLKKQEEKEILKSQLDSLLGLYHLLDWFAVDESNEVDPEFSARLTG
IKLEMEPSLSFYNKARNYATKKPYSVEKFKLNFQMPTLASGWDVNKEKNNGAILFVKNGLYYLGIMP
KQKGRYKALSFEPTEKTSEGFDKMYYDYFPDAAKMIPKCSTQLKAVTAHFQTHTTPILLSNNFIEPL
EITKEIYDLNNPEKEPKKFQTAYAKKTGDQKGYREALCKWIDFTRDFLSKYTKTTSIDLSSLRPSSQ
YKDLGEYYAELNPLLYHISFQRIAEKEIMDAVETGKLYLFQTYNKDFAKGHHGKPNLHTLYWTGLFS
PENLAKTSIKLNGQAELFYRPKSRMKRMAHRLGEKMLNKKLKDQKTPIPDTLYQELYDYVNHRLSHD
LSDEARALLPNVITKEVSHEIIKDRRFTSDKFFFHVPITLNYQAANSPSKFNQRVNAYLKEHPETPI
IGIDRGERNLIYITVIDSTGKILEQRSLNTIQQFDYQKKLDNREKERVAARQAWSVVGTIKDLKQGY
LSQVIHEIVDLMIHYQAVVVLENLNFGFKSKRTGIAEKAVYQQFEKMLIDKLNCLVLKDYPAEKVGG
VLNPYQLTDQFTSFAKMGTQSGFLFYVPAPYTSKIDPLTGFVDPFVWKTIKNHESRKHFLEGFDFLH
YDVKTGDFILHFKMNRNLSFQRGLPGFMPAWDIVFEKNETQFDAKGTPFIAGKRIVPVIENHRFTGR
YRDLYPANELIALLEEKGIVERDGSNILPKLLENDDSHAIDTMVALIRSVLQMRNSNAATGEDYINS
PVRDLNGVCFDSRFQNPEWPMDADANGAYHIALKGQLLLNHLKESKDLKLQNGISNQDWLAYIQELR
NPKKKRKVKLAAALEHHHHHH
Hs optimized As Cpf1 with flanking NLS's, V5 tag and
6x His - DNA
SEQ ID NO: 15
ATGGGCAAGCCCATTCCTAATCCTCTGCTGGGCCTCGACAGCACAGCCCCTAAGAAAAAGCGGAAAG
TGGGCATCCATGGCGTGCCAGCCGCCACACAGTTTGAGGGCTTCACCAACCTGTACCAGGTGTCCAA
GACACTGCGCTTCGAGCTGATCCCTCAGGGCAAGACCCTGAAGCACATCCAAGAGCAGGGCTTCATC
GAAGAGGACAAGGCCCGGAACGACCACTACAAAGAGCTGAAGCCCATCATCGACCGGATCTACAAGA
CCTACGCCGACCAGTGTCTGCAGCTGGTGCAGCTCGATTGGGAGAATCTGAGCGCCGCCATCGACAG
CTACCGGAAAGAGAAAACCGAGGAAACCCGGAACGCCCTGATCGAGGAACAGGCCACCTACAGAAAC
GCCATCCACGACTACTTCATCGGCCGGACCGACAACCTGACCGACGCCATCAACAAGAGACACGCCG
AGATCTATAAGGGCCTGTTCAAGGCCGAGCTGTTCAACGGCAAGGTGCTGAAGCAGCTGGGCACCGT
GACAACCACCGAGCACGAAAATGCCCTGCTGCGGAGCTTCGACAAGTTCACCACCTACTTCAGCGGC
TTCTACGAGAACCGGAAGAACGTGTTCAGCGCCGAGGACATCAGCACCGCCATTCCTCACAGAATCG
TGCAGGACAACTTCCCCAAGTTCAAAGAGAACTGCCACATCTTCACCCGGCTGATCACAGCCGTGCC
TAGCCTGAGAGAACACTTCGAGAACGTGAAGAAGGCCATCGGCATCTTCGTGTCCACCAGCATCGAG
GAAGTGTTCAGCTTCCCATTCTACAACCAGCTGCTGACCCAGACACAGATCGACCTGTATAATCAGC
TGCTCGGCGGCATCAGCAGAGAGGCCGGAACAGAGAAGATCAAGGGCCTGAACGAAGTGCTGAACCT
GGCCATCCAGAAGAACGACGAGACAGCCCACATCATTGCCAGCCTGCCTCACCGGTTCATCCCTCTG
TTCAAGCAGATCCTGAGCGACAGAAACACCCTGAGCTTCATCCTGGAAGAGTTCAAGTCCGATGAGG
AAGTGATCCAGAGCTTCTGCAAGTATAAGACCCTGCTGAGGAACGAGAATGTGCTGGAAACCGCCGA
GGCTCTGTTTAACGAGCTGAACAGCATCGATCTGACCCACATCTTTATCAGCCACAAGAAGCTCGAG
ACAATCAGCAGCGCCCTGTGCGACCACTGGGATACCCTGAGAAACGCCCTGTACGAGCGGAGAATCA
GCGAGCTGACCGGCAAGATCACCAAGAGCGCCAAAGAAAAGGTGCAGCGGAGCCTGAAACACGAGGA
TATCAACCTGCAAGAGATCATCAGCGCCGCTGGCAAAGAACTGAGCGAGGCCTTTAAGCAGAAAACC
AGCGAGATCCTGTCTCACGCCCACGCTGCTCTTGATCAGCCTCTGCCTACCACACTGAAGAAGCAAG
AGGAAAAAGAGATCCTGAAGTCCCAGCTGGACAGCCTGCTGGGACTGTACCATCTGCTGGATTGGTT
CGCCGTGGACGAGAGCAATGAGGTGGACCCTGAGTTCTCCGCCAGACTGACAGGCATCAAGCTGGAA
ATGGAACCCAGCCTGTCCTTCTACAACAAGGCCAGAAACTACGCCACCAAGAAGCCCTACAGCGTCG
AGAAGTTCAAGCTCAACTTCCAGATGCCTACACTGGCCAGCGGCTGGGACGTGAACAAAGAGAAGAA
CAACGGCGCCATCCTGTTCGTGAAGAACGGACTGTACTACCTGGGCATCATGCCAAAGCAGAAGGGC
AGATACAAGGCCCTGTCCTTTGAGCCCACCGAAAAGACCAGCGAGGGCTTCGATAAGATGTACTACG
ATTACTTCCCCGACGCCGCCAAGATGATCCCCAAGTGTAGCACACAGCTGAAGGCCGTGACCGCTCA
CTTTCAGACCCACACCACACCTATCCTGCTGAGCAACAACTTCATCGAGCCCCTGGAAATCACCAAA
GAGATCTACGACCTGAACAACCCCGAGAAAGAGCCCAAGAAGTTCCAGACCGCCTACGCCAAGAAAA
CCGGCGACCAGAAGGGCTACAGAGAAGCCCTGTGCAAGTGGATCGACTTTACCCGGGACTTCCTGAG
CAAGTACACCAAGACCACCTCCATCGACCTGAGCAGCCTGAGGCCTAGCAGCCAGTATAAGGACCTG
GGCGAGTACTACGCCGAGCTGAATCCACTGCTGTACCACATCAGCTTCCAGCGGATCGCCGAAAAAG
AAATCATGGACGCCGTGGAAACCGGCAAGCTGTACCTGTTCCAGATATACAACAAAGACTTCGCCAA
GGGCCACCACGGCAAGCCTAATCTGCACACCCTGTACTGGACCGGCCTGTTTAGCCCTGAGAATCTG
GCCAAGACCTCTATCAAGCTGAACGGCCAGGCCGAACTGTTTTACAGACCCAAGAGCCGGATGAAGC
GGATGGCCCACAGACTGGGAGAGAAGATGCTGAACAAGAAACTGAAGGACCAGAAAACGCCCATTCC
GGACACACTGTACCAAGAGCTGTACGACTACGTGAACCACCGGCTGAGCCACGATCTGAGCGACGAA
GCTAGAGCACTGCTGCCCAACGTGATCACAAAAGAGGTGTCCCACGAGATCATTAAGGACCGGCGGT
TTACCTCCGATAAGTTCTTCTTCCACGTGCCGATCACACTGAACTACCAGGCCGCCAACTCTCCCAG
CAAGTTCAACCAGAGAGTGAACGCCTACCTGAAAGAGCACCCCGAGACACCCATCATTGGCATCGAC
AGAGGCGAGCGGAACCTGATCTACATCACCGTGATCGACTCCACAGGCAAGATCCTGGAACAGCGGT
CCCTGAACACCATCCAGCAGTTCGACTACCAGAAGAAGCTGGACAACCGAGAGAAAGAAAGAGTGGC
CGCCAGACAGGCTTGGAGCGTTGTGGGCACAATCAAGGATCTGAAGCAGGGCTACCTGAGCCAAGTG
ATTCACGAGATCGTGGACCTGATGATCCACTATCAGGCTGTGGTGGTGCTCGAGAACCTGAACTTCG
GCTTCAAGAGCAAGCGGACCGGAATCGCCGAGAAAGCCGTGTACCAGCAGTTTGAGAAAATGCTGAT
CGACAAGCTGAATTGCCTGGTCCTGAAGGACTACCCCGCTGAGAAAGTTGGCGGAGTGCTGAATCCC
TACCAGCTGACCGATCAGTTCACCAGCTTTGCCAAGATGGGAACCCAGAGCGGCTTCCTGTTCTACG
TGCCAGCTCCTTACACCTCCAAGATCGACCCTCTGACCGGCTTCGTGGACCCCTTCGTGTGGAAAAC
CATCAAGAACCACGAGTCCCGGAAGCACTTCCTGGAAGGCTTTGACTTCCTGCACTACGACGTGAAA
ACAGGCGATTTCATCCTGCACTTCAAGATGAATCGGAATCTGTCCTTCCAGAGGGGCCTGCCTGGCT
TCATGCCTGCTTGGGATATCGTGTTCGAGAAGAATGAGACTCAGTTCGACGCCAAGGGGACCCCTTT
TATCGCCGGCAAGAGAATTGTGCCTGTGATCGAGAACCACAGGTTCACCGGCAGATACCGGGATCTG
TACCCCGCCAATGAGCTGATCGCCCTGCTGGAAGAGAAGGGCATCGTGTTTAGAGATGGCAGCAACA
TCCTGCCTAAGCTGCTGGAAAACGACGACAGCCACGCCATCGATACCATGGTGGCACTGATCAGATC
CGTGCTGCAGATGCGGAACAGCAATGCCGCTACCGGCGAGGACTACATCAATAGCCCCGTGCGGGAT
CTGAACGGCGTGTGCTTCGACAGCAGATTTCAGAACCCCGAGTGGCCTATGGATGCCGACGCCAATG
GCGCCTATCACATTGCCCTGAAAGGACAGCTGCTGCTGAACCATCTGAAAGAGAGCAAGGACCTGAA
ACTGCAGAACGGCATCTCCAACCAGGACTGGCTGGCCTACATTCAAGAGCTGCGGAATCCCAAAAAG
AAACGGAAAGTGAAGCTGGCCGCTGCTCTGGAACACCACCACCATCACCAT
Hs optimized As Cpf1 with 5′- and 3′-flanking NLS's,
5′-V5 tag and 3′-6x His - AA
SEQ ID NO: 16
MGKPIPNPLLGLDSTAPKKKRKVGIHGVPAATQFEGFTNLYQVSKTLRFELIPQGKTLKHIQEQGFI
EEDKARNDHYKELKPIIDRIYKTYADQCLQLVQLDWENLSAAIDSYRKEKTEETRNALIEEQATYRN
AIHDYFIGRTDNLTDAINKRHAEIYKGLFKAELFNGKVLKQLGTVTTTEHENALLRSFDKFTTYFSG
FYENRKNVFSAEDISTAIPHRIVQDNFPKFKENCHIFTRLITAVPSLREHFENVKKAIGIFVSTSIE
EVFSFPFYNQLLTQTQIDLYNQLLGGISREAGTEKIKGLNEVLNLAIQKNDETAHIIASLPHRFIPL
FKQILSDRNTLSFILEEFKSDEEVIQSFCKYKTLLRNENVLETAEALFNELNSIDLTHIFISHKKLE
TISSALCDHWDTLRNALYERRISELTGKITKSAKEKVQRSLKHEDINLQEIISAAGKELSEAFKQKT
SEILSHAHAALDQPLPTTLKKQEEKEILKSQLDSLLGLYHLLDWFAVDESNEVDPEFSARLTGIKLE
MEPSLSFYNKARNYATKKPYSVEKFKLNFQMPTLASGWDVNKEKNNGAILFVKNGLYYLGIMPKQKG
RYKALSFEPTEKTSEGFDKMYYDYFPDAAKMIPKCSTQLKAVTAHFQTHTTPILLSNNFIEPLEITK
EIYDLNNPEKEPKKFQTAYAKKTGDQKGYREALCKWIDFTRDFLSKYTKTTSIDLSSLRPSSQYKDL
GEYYAELNPLLYHISFQRIAEKEIMDAVETGKLYLFQTYNKDFAKGHHGKPNLHTLYWTGLFSPENL
AKTSIKLNGQAELFYRPKSRMKRMAHRLGEKMLNKKLKDQKTPIPDTLYQELYDYVNHRLSHDLSDE
ARALLPNVITKEVSHEIIKDRRFTSDKFFFHVPITLNYQAANSPSKFNQRVNAYLKEHPETPIIGID
RGERNLIYITVIDSTGKILEQRSLNTIQQFDYQKKLDNREKERVAARQAWSVVGTIKDLKQGYLSQV
IHEIVDLMIHYQAVVVLENLNFGFKSKRTGIAEKAVYQQFEKMLIDKLNCLVLKDYPAEKVGGVLNP
YQLTDQFTSFAKMGTQSGFLFYVPAPYTSKIDPLTGFVDPFVWKTIKNHESRKHFLEGFDFLHYDVK
TGDFILHFKMNRNLSFQRGLPGFMPAWDIVFEKNETQFDAKGTPFIAGKRIVPVIENHRFTGRYRDL
YPANELIALLEEKGIVERDGSNILPKLLENDDSHAIDTMVALIRSVLQMRNSNAATGEDYINSPVRD
LNGVCFDSRFQNPEWPMDADANGAYHIALKGQLLLNHLKESKDLKLQNGISNQDWLAYIQELRNPKK
KRKVKLAAALEHHHHHH
E. coli optimized As Cpf1 with OpT NLS and 6x His - DNA
SEQ ID NO: 18
ATGACCCAGTTTGAAGGTTTCACCAATCTGTATCAGGTTAGCAAAACCCTGCGTTTTGAACTGATTC
CGCAGGGTAAAACCCTGAAACATATTCAAGAACAGGGCTTCATCGAAGAGGATAAAGCACGTAACGA
TCACTACAAAGAACTGAAACCGATTATCGACCGCATCTATAAAACCTATGCAGATCAGTGTCTGCAG
CTGGTTCAGCTGGATTGGGAAAATCTGAGCGCAGCAATTGATAGTTATCGCAAAGAAAAAACCGAAG
AAACCCGTAATGCACTGATTGAAGAACAGGCAACCTATCGTAATGCCATCCATGATTATTTCATTGG
TCGTACCGATAATCTGACCGATGCAATTAACAAACGTCACGCCGAAATCTATAAAGGCCTGTTTAAA
GCCGAACTGTTTAATGGCAAAGTTCTGAAACAGCTGGGCACCGTTACCACCACCGAACATGAAAATG
CACTGCTGCGTAGCTTTGATAAATTCACCACCTATTTCAGCGGCTTTTATGAGAATCGCAAAAACGT
GTTTAGCGCAGAAGATATTAGCACCGCAATTCCGCATCGTATTGTGCAGGATAATTTCCCGAAATTC
AAAGAGAACTGCCACATTTTTACCCGTCTGATTACCGCAGTTCCGAGCCTGCGTGAACATTTTGAAA
ACGTTAAAAAAGCCATCGGCATCTTTGTTAGCACCAGCATTGAAGAAGTTTTTAGCTTCCCGTTTTA
CAATCAGCTGCTGACCCAGACCCAGATTGATCTGTATAACCAACTGCTGGGTGGTATTAGCCGTGAA
GCAGGCACCGAAAAAATCAAAGGTCTGAATGAAGTGCTGAATCTGGCCATTCAGAAAAATGATGAAA
CCGCACATATTATTGCAAGCCTGCCGCATCGTTTTATTCCGCTGTTCAAACAAATTCTGAGCGATCG
TAATACCCTGAGCTTTATTCTGGAAGAATTCAAATCCGATGAAGAGGTGATTCAGAGCTTTTGCAAA
TACAAAACGCTGCTGCGCAATGAAAATGTTCTGGAAACTGCCGAAGCACTGTTTAACGAACTGAATA
GCATTGATCTGACCCACATCTTTATCAGCCACAAAAAACTGGAAACCATTTCAAGCGCACTGTGTGA
TCATTGGGATACCCTGCGTAATGCCCTGTATGAACGTCGTATTAGCGAACTGACCGGTAAAATTACC
AAAAGCGCGAAAGAAAAAGTTCAGCGCAGTCTGAAACATGAGGATATTAATCTGCAAGAGATTATTA
GCGCAGCCGGTAAAGAACTGTCAGAAGCATTTAAACAGAAAACCAGCGAAATTCTGTCACATGCACA
TGCAGCACTGGATCAGCCGCTGCCGACCACCCTGAAAAAACAAGAAGAAAAAGAAATCCTGAAAAGC
CAGCTGGATAGCCTGCTGGGTCTGTATCATCTGCTGGACTGGTTTGCAGTTGATGAAAGCAATGAAG
TTGATCCGGAATTTAGCGCACGTCTGACCGGCATTAAACTGGAAATGGAACCGAGCCTGAGCTTTTA
TAACAAAGCCCGTAATTATGCCACCAAAAAACCGTATAGCGTCGAAAAATTCAAACTGAACTTTCAG
ATGCCGACCCTGGCAAGCGGTTGGGATGTTAATAAAGAAAAAAACAACGGTGCCATCCTGTTCGTGA
AAAATGGCCTGTATTATCTGGGTATTATGCCGAAACAGAAAGGTCGTTATAAAGCGCTGAGCTTTGA
ACCGACGGAAAAAACCAGTGAAGGTTTTGATAAAATGTACTACGACTATTTTCCGGATGCAGCCAAA
ATGATTCCGAAATGTAGCACCCAGCTGAAAGCAGTTACCGCACATTTTCAGACCCATACCACCCCGA
TTCTGCTGAGCAATAACTTTATTGAACCGCTGGAAATCACCAAAGAGATCTACGATCTGAATAACCC
GGAAAAAGAGCCGAAAAAATTCCAGACCGCATATGCAAAAAAAACCGGTGATCAGAAAGGTTATCGT
GAAGCGCTGTGTAAATGGATTGATTTCACCCGTGATTTTCTGAGCAAATACACCAAAACCACCAGTA
TCGATCTGAGCAGCCTGCGTCCGAGCAGCCAGTATAAAGATCTGGGCGAATATTATGCAGAACTGAA
TCCGCTGCTGTATCATATTAGCTTTCAGCGTATTGCCGAGAAAGAAATCATGGACGCAGTTGAAACC
GGTAAACTGTACCTGTTCCAGATCTACAATAAAGATTTTGCCAAAGGCCATCATGGCAAACCGAATC
TGCATACCCTGTATTGGACCGGTCTGTTTAGCCCTGAAAATCTGGCAAAAACCTCGATTAAACTGAA
TGGTCAGGCGGAACTGTTTTATCGTCCGAAAAGCCGTATGAAACGTATGGCACATCGTCTGGGTGAA
AAAATGCTGAACAAAAAACTGAAAGACCAGAAAACCCCGATCCCGGATACACTGTATCAAGAACTGT
ATGATTATGTGAACCATCGTCTGAGCCATGATCTGAGTGATGAAGCACGTGCCCTGCTGCCGAATGT
TATTACCAAAGAAGTTAGCCACGAGATCATTAAAGATCGTCGTTTTACCAGCGACAAATTCTTTTTT
CATGTGCCGATTACCCTGAATTATCAGGCAGCAAATAGCCCGAGCAAATTTAACCAGCGTGTTAATG
CATATCTGAAAGAACATCCAGAAACGCCGATTATTGGTATTGATCGTGGTGAACGTAACCTGATTTA
TATCACCGTTATTGATAGCACCGGCAAAATCCTGGAACAGCGTAGCCTGAATACCATTCAGCAGTTT
GATTACCAGAAAAAACTGGATAATCGCGAGAAAGAACGTGTTGCAGCACGTCAGGCATGGTCAGTTG
TTGGTACAATTAAAGACCTGAAACAGGGTTATCTGAGCCAGGTTATTCATGAAATTGTGGATCTGAT
GATTCACTATCAGGCCGTTGTTGTGCTGGAAAACCTGAATTTTGGCTTTAAAAGCAAACGTACCGGC
ATTGCAGAAAAAGCAGTTTATCAGCAGTTCGAGAAAATGCTGATTGACAAACTGAATTGCCTGGTGC
TGAAAGATTATCCGGCTGAAAAAGTTGGTGGTGTTCTGAATCCGTATCAGCTGACCGATCAGTTTAC
CAGCTTTGCAAAAATGGGCACCCAGAGCGGATTTCTGTTTTATGTTCCGGCACCGTATACGAGCAAA
ATTGATCCGCTGACCGGTTTTGTTGATCCGTTTGTTTGGAAAACCATCAAAAACCATGAAAGCCGCA
AACATTTTCTGGAAGGTTTCGATTTTCTGCATTACGACGTTAAAACGGGTGATTTCATCCTGCACTT
TAAAATGAATCGCAATCTGAGTTTTCAGCGTGGCCTGCCTGGTTTTATGCCTGCATGGGATATTGTG
TTTGAGAAAAACGAAACACAGTTCGATGCAAAAGGCACCCCGTTTATTGCAGGTAAACGTATTGTTC
CGGTGATTGAAAATCATCGTTTCACCGGTCGTTATCGCGATCTGTATCCGGCAAATGAACTGATCGC
ACTGCTGGAAGAGAAAGGTATTGTTTTTCGTGATGGCTCAAACATTCTGCCGAAACTGCTGGAAAAT
GATGATAGCCATGCAATTGATACCATGGTTGCACTGATTCGTAGCGTTCTGCAGATGCGTAATAGCA
ATGCAGCAACCGGTGAAGATTACATTAATAGTCCGGTTCGTGATCTGAATGGTGTTTGTTTTGATAG
CCGTTTTCAGAATCCGGAATGGCCGATGGATGCAGATGCAAATGGTGCATATCATATTGCACTGAAA
GGACAGCTGCTGCTGAACCACCTGAAAGAAAGCAAAGATCTGAAACTGCAAAACGGCATTAGCAATC
AGGATTGGCTGGCATATATCCAAGAACTGCGTAACGGTCGTAGCAGTGATGATGAAGCAACCGCAGA
TAGCCAGCATGCAGCACCGCCTAAAAAGAAACGTAAAGTTGGTGGTAGCGGTGGTTCAGGTGGTAGT
GGCGGTAGTGGTGGCTCAGGGGGTTCTGGTGGCTCTGGTGGTAGCCTCGAGCACCACCACCACCACC
ACTGA
Amino acid sequence for AsCpf1 fusion with OpT NLS
and 6x His used for gene editing in both E. coli and human cells
SEQ ID NO: 19
MTQFEGFTNLYQVSKTLRFELIPQGKTLKHIQEQGFIEEDKARNDHYKELKPIIDRIYKTYADQCLQ
LVQLDWENLSAAIDSYRKEKTEETRNALIEEQATYRNAIHDYFIGRTDNLTDAINKRHAEIYKGLFK
AELFNGKVLKQLGTVTTTEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLTQTQIDLYNQLLGGISRE
AGTEKIKGLNEVLNLAIQKNDETAHIIASLPHRFIPLFKQILSDRNTLSFILEEFKSDEEVIQSFCK
YKTLLRNENVLETAEALFNELNSIDLTHIFISHKKLETISSALCDHWDTLRNALYERRISELTGKIT
KSAKEKVQRSLKHEDINLQEIISAAGKELSEAFKQKTSEILSHAHAALDQPLPTTLKKQEEKEILKS
QLDSLLGLYHLLDWFAVDESNEVDPEFSARLTGIKLEMEPSLSFYNKARNYATKKPYSVEKFKLNFQ
MPTLASGWDVNKEKNNGAILFVKNGLYYLGIMPKQKGRYKALSFEPTEKTSEGFDKMYYDYFPDAAK
MIPKCSTQLKAVTAHFQTHTTPILLSNNFIEPLEITKEIYDLNNPEKEPKKFQTAYAKKTGDQKGYR
EALCKWIDFTRDFLSKYTKTTSIDLSSLRPSSQYKDLGEYYAELNPLLYHISFQRIAEKEIMDAVET
GKLYLFQTYNKDFAKGHHGKPNLHTLYWTGLFSPENLAKTSIKLNGQAELFYRPKSRMKRMAHRLGE
KMLNKKLKDQKTPIPDTLYQELYDYVNHRLSHDLSDEARALLPNVITKEVSHEIIKDRRFTSDKFFF
HVPITLNYQAANSPSKFNQRVNAYLKEHPETPIIGIDRGERNLIYITVIDSTGKILEQRSLNTIQQF
DYQKKLDNREKERVAARQAWSVVGTIKDLKQGYLSQVIHEIVDLMIHYQAVVVLENLNFGFKSKRTG
IAEKAVYQQFEKMLIDKLNCLVLKDYPAEKVGGVLNPYQLTDQFTSFAKMGTQSGFLFYVPAPYTSK
IDPLTGFVDPFVWKTIKNHESRKHFLEGFDFLHYDVKTGDFILHFKMNRNLSFQRGLPGFMPAWDIV
FEKNETQFDAKGTPFIAGKRIVPVIENHRFTGRYRDLYPANELIALLEEKGIVERDGSNILPKLLEN
DDSHAIDTMVALIRSVLQMRNSNAATGEDYINSPVRDLNGVCFDSRFQNPEWPMDADANGAYHIALK
GQLLLNHLKESKDLKLQNGISNQDWLAYIQELRNGRSSDDEATADSQHAAPPKKKRKVGGSGGSGGS
GGSGGSGGSGGSGGSLEHHHHHH
Hs optimized As Cpf1 with OpT NLS and 6x His - DNA
SEQ ID NO: 21
ATGGGCGACCCTCTGAAGAACGTGGGCATCGACAGACTGGACGTGGAAAAGGGCAGAAAGAACATGA
GCAAGCTCGAGAAGTTCACCAACTGCTACAGCCTGAGCAAGACCCTGCGGTTCAAGGCCATTCCTGT
GGGCAAGACCCAAGAGAACATCGACAACAAGCGGCTGCTGGTGGAAGATGAGAAGAGAGCCGAGGAC
TACAAGGGCGTGAAGAAGCTGCTGGACCGGTACTACCTGAGCTTCATCAACGACGTGCTGCACAGCA
TCAAGCTGAAGAACCTGAACAACTACATCAGCCTGTTCCGGAAGAAAACCCGGACCGAGAAAGAGAA
CAAAGAGCTGGAAAACCTCGAGATCAACCTGCGGAAAGAGATCGCCAAGGCCTTCAAGGGCAACGAG
GGCTACAAGAGCCTGTTCAAGAAGGACATCATCGAGACAATCCTGCCTGAGTTCCTGGACGACAAGG
ACGAGATCGCCCTGGTCAACAGCTTCAACGGCTTCACAACCGCCTTCACCGGCTTTTTCGACAACCG
CGAGAATATGTTCAGCGAGGAAGCCAAGAGCACCTCTATCGCCTTCCGGTGCATCAACGAGAATCTG
ACCCGGTACATCAGCAACATGGATATCTTCGAGAAGGTGGACGCCATCTTCGACAAGCACGAGGTGC
AAGAGATCAAAGAAAAGATCCTGAACAGCGACTACGACGTCGAGGACTTCTTCGAGGGCGAGTTCTT
CAACTTCGTGCTGACACAAGAGGGCATCGATGTGTACAACGCCATCATCGGCGGCTTCGTGACAGAG
AGCGGCGAGAAGATCAAGGGCCTGAACGAGTACATCAACCTCTACAACCAGAAAACGAAGCAGAAGC
TGCCCAAGTTCAAGCCCCTGTACAAACAGGTGCTGAGCGACAGAGAGAGCCTGTCCTTTTACGGCGA
GGGCTATACCAGCGACGAAGAGGTGCTGGAAGTGTTCAGAAACACCCTGAACAAGAACAGCGAGATC
TTCAGCTCCATCAAGAAGCTCGAAAAGCTGTTTAAGAACTTCGACGAGTACAGCAGCGCCGGCATCT
TCGTGAAGAATGGCCCTGCCATCAGCACCATCTCCAAGGACATCTTCGGCGAGTGGAACGTGATCCG
GGACAAGTGGAACGCCGAGTACGACGACATCCACCTGAAGAAAAAGGCCGTGGTCACCGAGAAGTAC
GAGGACGACAGAAGAAAGAGCTTCAAGAAGATCGGCAGCTTCAGCCTGGAACAGCTGCAAGAGTACG
CCGACGCCGATCTGAGCGTGGTGGAAAAGCTGAAAGAGATTATCATCCAGAAGGTCGACGAGATCTA
CAAGGTGTACGGCAGCAGCGAGAAGCTGTTCGACGCCGACTTTGTGCTGGAAAAGAGCCTCAAAAAG
AACGACGCCGTGGTGGCCATCATGAAGGACCTGCTGGATAGCGTGAAGTCCTTCGAGAACTATATTA
AGGCCTTCTTTGGCGAGGGCAAAGAGACAAACCGGGACGAGAGCTTCTACGGCGATTTCGTGCTGGC
CTACGACATCCTGCTGAAAGTGGACCACATCTACGACGCCATCCGGAACTACGTGACCCAGAAGCCT
TACAGCAAGGACAAGTTTAAGCTGTACTTCCAGAATCCGCAGTTCATGGGCGGCTGGGACAAAGACA
AAGAAACCGACTACCGGGCCACCATCCTGAGATACGGCTCCAAGTACTATCTGGCCATTATGGACAA
GAAATACGCCAAGTGCCTGCAGAAGATCGATAAGGACGACGTGAACGGCAACTACGAGAAGATTAAC
TACAAGCTGCTGCCCGGACCTAACAAGATGCTGCCTAAGGTGTTCTTTAGCAAGAAATGGATGGCCT
ACTACAACCCCAGCGAGGATATCCAGAAAATCTACAAGAACGGCACCTTCAAGAAAGGCGACATGTT
CAACCTGAACGACTGCCACAAGCTGATCGATTTCTTCAAGGACAGCATCAGCAGATACCCCAAGTGG
TCCAACGCCTACGACTTCAATTTCAGCGAGACAGAGAAGTATAAGGATATCGCCGGGTTCTACCGCG
AGGTGGAAGAACAGGGCTATAAGGTGTCCTTTGAGAGCGCCAGCAAGAAAGAGGTGGACAAGCTGGT
CGAAGAGGGCAAGCTGTACATGTTCCAGATCTATAACAAGGACTTCTCCGACAAGAGCCACGGCACC
CCTAACCTGCACACCATGTACTTTAAGCTGCTGTTCGATGAGAACAACCACGGCCAGATCAGACTGT
CTGGCGGAGCCGAGCTGTTTATGAGAAGGGCCAGCCTGAAAAAAGAGGAACTGGTCGTTCACCCCGC
CAACTCTCCAATCGCCAACAAGAACCCCGACAATCCCAAGAAAACCACCACACTGAGCTACGACGTG
TACAAGGATAAGCGGTTCTCCGAGGACCAGTACGAGCTGCACATCCCTATCGCCATCAACAAGTGCC
CCAAGAATATCTTCAAGATCAACACCGAAGTGCGGGTGCTGCTGAAGCACGACGACAACCCTTACGT
GATCGGCATCGATCGGGGCGAGAGAAACCTGCTGTATATCGTGGTGGTGGACGGCAAGGGCAATATC
GTGGAACAGTACTCCCTGAATGAGATCATCAACAACTTCAATGGCATCCGGATCAAGACGGACTACC
ACAGCCTGCTGGACAAAAAAGAGAAAGAACGCTTCGAGGCCCGGCAGAACTGGACCAGCATCGAGAA
CATCAAAGAACTGAAGGCCGGCTACATCTCCCAGGTGGTGCACAAGATCTGCGAGCTGGTTGAGAAG
TATGACGCCGTGATTGCCCTGGAAGATCTGAATAGCGGCTTTAAGAACAGCCGCGTGAAGGTCGAGA
AACAGGTGTACCAGAAATTCGAGAAGATGCTGATCGACAAGCTGAACTACATGGTCGACAAGAAGTC
TAACCCCTGCGCCACAGGCGGAGCCCTGAAGGGATATCAGATCACCAACAAGTTCGAGTCCTTCAAG
AGCATGAGCACCCAGAATGGCTTCATCTTCTACATCCCCGCCTGGCTGACCAGCAAGATCGATCCTA
GCACCGGATTCGTGAACCTGCTCAAGACCAAGTACACCAGCATTGCCGACAGCAAGAAGTTCATCTC
CAGCTTCGACCGGATTATGTACGTGCCCGAAGAGGACCTGTTCGAATTCGCCCTGGATTACAAGAAC
TTCAGCCGGACCGATGCCGACTATATCAAGAAGTGGAAGCTGTATAGCTACGGCAACCGCATCCGCA
TCTTCAGAAACCCGAAGAAAAACAACGTGTTCGACTGGGAAGAAGTGTGCCTGACCAGCGCCTACAA
AGAACTCTTCAACAAATACGGCATCAACTACCAGCAGGGCGACATCAGAGCCCTGCTGTGCGAGCAG
AGCGACAAGGCCTTTTACAGCTCCTTCATGGCCCTGATGAGCCTGATGCTGCAGATGCGGAATAGCA
TCACCGGCAGGACCGACGTGGACTTCCTGATCAGCCCTGTGAAGAATTCCGACGGGATCTTCTACGA
CAGCAGAAACTACGAGGCTCAAGAGAACGCCATCCTGCCTAAGAACGCCGATGCCAACGGCGCCTAT
AATATCGCCAGAAAGGTGCTGTGGGCCATCGGCCAGTTTAAGAAGGCCGAGGACGAGAAACTGGACA
AAGTGAAGATCGCCATCTCTAACAAAGAGTGGCTGGAATACGCCCAGACCAGCGTGAAGCACGGCAG
ATCTAGTGACGATGAGGCCACCGCCGATAGCCAGCATGCAGCCCCTCCAAAGAAAAAGCGGAAAGTG
CTGGAACACCACCACCATCACCAC
Hs optimized As Cpf1 with OpT NLS and 6x His - AA
SEQ ID NO: 22
MTQFEGFTNLYQVSKTLRFELIPQGKTLKHIQEQGFIEEDKARNDHYKELKPIIDRIYKTYADQCLQ
LVQLDWENLSAAIDSYRKEKTEETRNALIEEQATYRNAIHDYFIGRTDNLTDAINKRHAEIYKGLFK
AELFNGKVLKQLGTVTTTEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLTQTQIDLYNQLLGGISRE
AGTEKIKGLNEVLNLAIQKNDETAHIIASLPHRFIPLFKQILSDRNTLSFILEEFKSDEEVIQSFCK
YKTLLRNENVLETAEALFNELNSIDLTHIFISHKKLETISSALCDHWDTLRNALYERRISELTGKIT
KSAKEKVQRSLKHEDINLQEIISAAGKELSEAFKQKTSEILSHAHAALDQPLPTTLKKQEEKEILKS
QLDSLLGLYHLLDWFAVDESNEVDPEFSARLTGIKLEMEPSLSFYNKARNYATKKPYSVEKFKLNFQ
MPTLASGWDVNKEKNNGAILFVKNGLYYLGIMPKQKGRYKALSFEPTEKTSEGFDKMYYDYFPDAAK
MIPKCSTQLKAVTAHFQTHTTPILLSNNFIEPLEITKEIYDLNNPEKEPKKFQTAYAKKTGDQKGYR
EALCKWIDFTRDFLSKYTKTTSIDLSSLRPSSQYKDLGEYYAELNPLLYHISFQRIAEKEIMDAVET
GKLYLFQTYNKDFAKGHHGKPNLHTLYWTGLFSPENLAKTSIKLNGQAELFYRPKSRMKRMAHRLGE
KMLNKKLKDQKTPIPDTLYQELYDYVNHRLSHDLSDEARALLPNVITKEVSHEIIKDRRFTSDKFFF
HVPITLNYQAANSPSKFNQRVNAYLKEHPETPIIGIDRGERNLIYITVIDSTGKILEQRSLNTIQQF
DYQKKLDNREKERVAARQAWSVVGTIKDLKQGYLSQVIHEIVDLMIHYQAVVVLENLNFGFKSKRTG
IAEKAVYQQFEKMLIDKLNCLVLKDYPAEKVGGVLNPYQLTDQFTSFAKMGTQSGFLFYVPAPYTSK
IDPLTGFVDPFVWKTIKNHESRKHFLEGFDFLHYDVKTGDFILHFKMNRNLSFQRGLPGFMPAWDIV
FEKNETQFDAKGTPFIAGKRIVPVIENHRFTGRYRDLYPANELIALLEEKGIVERDGSNILPKLLEN
DDSHAIDTMVALIRSVLQMRNSNAATGEDYINSPVRDLNGVCFDSRFQNPEWPMDADANGAYHIALK
GQLLLNHLKESKDLKLQNGISNQDWLAYIQELRNGRSSDDEATADSQHAAPPKKKRKVGGSGGSGGS
GGSGGSGGSGGSGGSLEHHHHHH

### Example 2

### Preparation of Isolated Vectors Expressing Nucleic Acid Encoding Human Codon-Optimized AsCpf1 Polypeptide Fusion Protein and Human Cell Lines Stably Expressing the as Cpf1 Polypeptide Fusion Protein.

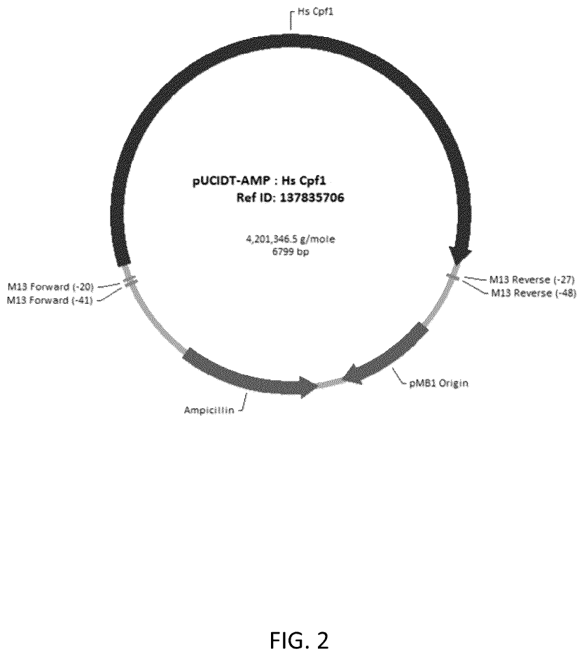

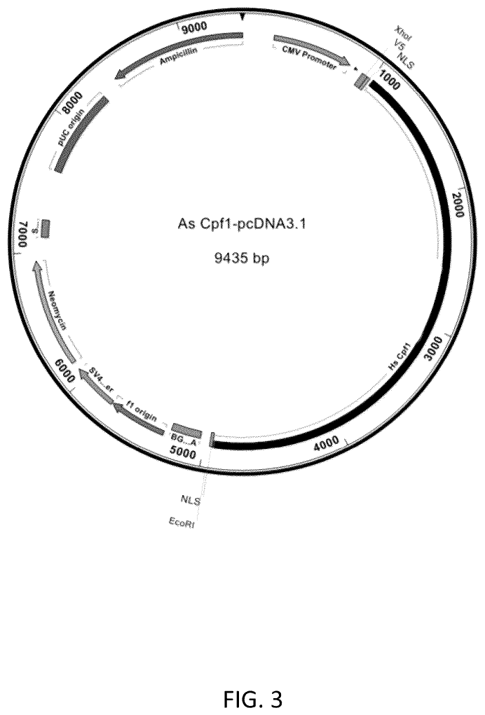

The reference amino acid for AsCpf1 has been published. See Zetsche, B., Gootenberg, J. S., Abudayyeh, O. O., Slaymaker, I. M., Makarova, K. S., Essletzbichler, P., Volz, S. E., Joung, J., van der Oost, J., Regev, A., Koonin, E. V., and Zhang, F. (2015) Cpf1 is a single RNA-guided endonuclease of a class 2 CRISPR-Cas system. Cell 163:1-13. A plasmid encoding human codon optimized AsCpf1, flanking nuclear localization signals (NLS) and 5′-V5 epitope tag, was generated by the Synthetic Biology department at Integrated DNA Technologies. Flanking the expression cassette was a 5′ XhoI and 3′ EcoRI restriction enzyme sites (FIG. 2). The Cpf1 plasmid was digested with XhoI and EcoRI (NEB), gel purified using a column based purification system (Qiagen) and ligated using T4 DNA Ligase (NEB) into a predigested mammalian expression vector, pcDNA3.1-, from Life Technologies (FIG. 3). The resulting ligated construct was transformed into DH5a chemically competent E. coli cells. The resulting colonies were grown in LB media at 37° C. overnight and subjected to DNA isolation using a Promega miniprep plasmid DNA kit. Flanking primers (T7 forward and BGH reverse) as well as 10 internal Cpf1 specific primers were used for sequence verification of correct insertion using automated Sanger sequencing with BigDye Terminator reagents (ABI). The nucleic acid sequence of the Cpf1 clone employed herein is shown in SEQ ID NO:15. The amino acid sequence of the expressed recombinant protein is shown in SEQ ID NO:16.

The AsCpf1-pcDNA3.1 vector was linearized with PvuI (NEB), which is located within the ampicillin resistance gene, and transfected into HEK293 cells. Transfection employed 500,000 HEK293 cells plated in 100 mm dishes 24 hours prior to transfection. Using the transfection reagent TransIT-X2 (Mirus), the linearized vector containing AsCpf1 and a neomycin-resistance gene was complexed and transfected into adherent cells. The transfection media was removed after 24 hrs and the cells were cultured in complete media for 48 hours. Using methods previously optimized for generation of stable transgenic HEK293 cells containing a stably integrated pcDNA3.1(−) vector neomycin resistance, we cultured transfected cells in the presence of the antibiotic Geneticin (G418; Gibco), which is a neomycin analog, in the complete media to select for cells that had been transfected with AsCpf1-pcDNA3.1(−) and would thus be resistant to this antibiotic. Initial G418 dosing was at 800 μg/ml with periodic media changes until the surviving cells began to recover and grow over a 10-day period. The parent HEK293 cell line was confirmed to be sensitive to the minimum dose of G418. The resulting polyclonal AsCpf1-pcDNA3.1(−) cell line, which showed G418 resistance, was split using limited dilutions. The cells were trypsinized, resuspended in complete media, counted to determine concentration and diluted in 96-well plates to a concentration of theoretically less than one cell per well.

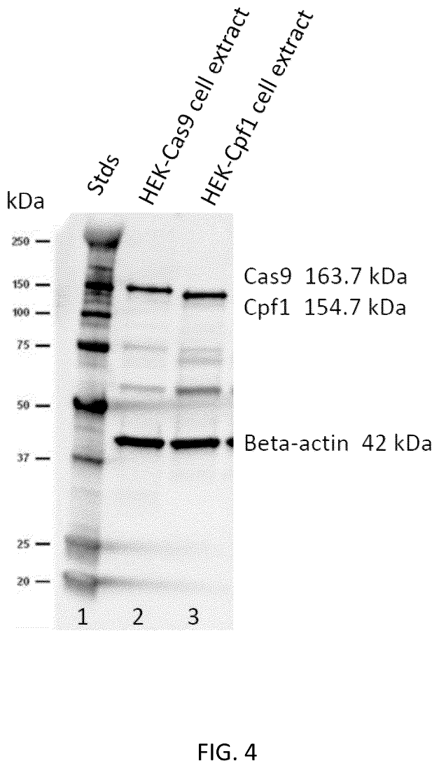

At this time, aliquots of the cells were taken and lysed with a protein lysis buffer (RIPA) to determine, via western blot, if AsCpf1 was expressed. Cellular protein was quantitated using the Bio-Rad Protein Assay (Bio-Rad) and 15 ug total protein was loaded onto an SDS-PAGE Stainfree 4-20% gradient gel (Bio-Rad). As a positive control, protein from a previous cell line, SpyCas9-pcDNA3.1(−), was run in parallel for size and expression comparisons. The gel was run for 45 minutes at 180 volts and transferred to a PVDF membrane with the Bio-Rad TransBlot for 7 minutes. The blot was then blocked in SuperBlock T20 Blocking Buffer (Thermo), followed by a 1:1000 dilution of V5 primary antibody (Abcam) and 1:5000 β-actin primary antibody (Abcam) for 1 hour at room temperature. Next, the blot was washed 3 times for 15 minutes each in tris-buffered saline with Tween-20 (TBST). Goat anti-mouse HRP secondary antibody was used at a 1:3000 dilution along with the ladder specific StrepTactin secondary antibody and incubated at room temperature for 1 hour at room temperature. The blot was then washed 3 times for 15 minutes in TBST. Luminescence detection was done using the Pierce West-Femto ECL (Thermo) substrate and results are shown in FIG. 4, which confirm expression of a recombinant protein of the expected size.

Cells were continuously grown under selection in G418-containing media, and individual cells (monoclonal colonies) were allowed to expand. Viable colonies were characterized for the presence of AsCpf1 by RT-qPCR, Western blotting and functional testing of crRNA guided dsDNA cleavage. Four RT-qPCR assays were designed to detect different locations within the large AsCpf1 mRNA. Sequences are shown in Table 1 below.

TABLE 1
RT-qPCR assays in AsCpf1

| Assay# | Location | Primers and Probe | SEQ ID NO |
|---|---|---|---|
| 1 | 34-153 | F34 GTGTCCAAGACCCTGAGATTC | 25 |
| | | R153 GGGCTTCAGCTCTTTGTAGT | 26 |
| | | P68 FAM-AGGGCAAG(ZEN)ACACTGAAGCACATCC-IBFQ | 27 |
| 2 | 1548-1656 | F1548 CAGAAACTACGCCACCAAGA | 28 |
| | | R1656 GCCGTTGTTCTTCTCTTTGTTC | 29 |
| | | P1590 HEX-TAAGCTGAA(ZEN)CTTCCAGATGCCCACC-IBFQ | 30 |
| 3 | 2935-3037 | F2935 GTGGACCTGATGATCCACTATC | 31 |
| | | R3037 GCTGGTACACGGCTTTCT | 32 |
| | | P2978 FAM-ACCTGAACT(ZEN)TCGGCTTCAAGAGCA-IBFQ | 33 |
| 4 | 3827-3918 | F3827 TGCTGAACCATCTGAAAGAGAG | 34 |
| | | R3918 GTTCCGCAGTTCCTGGATATAG | 35 |
| | | P3889 HEX-AGTCCTGGT(ZEN)TGGAGATGCCGTTC-IBFQ | 36 |

DNA bases are shown 5′-3′ orientation. Location is specified within the AsCpf1 gene construct employed herein. FAM-6 carboxyfluorescein, HEX = hexachlorofluorescein, IBFQ = Iowa Black dark quencher, and ZEN = internal ZEN dark quencher.

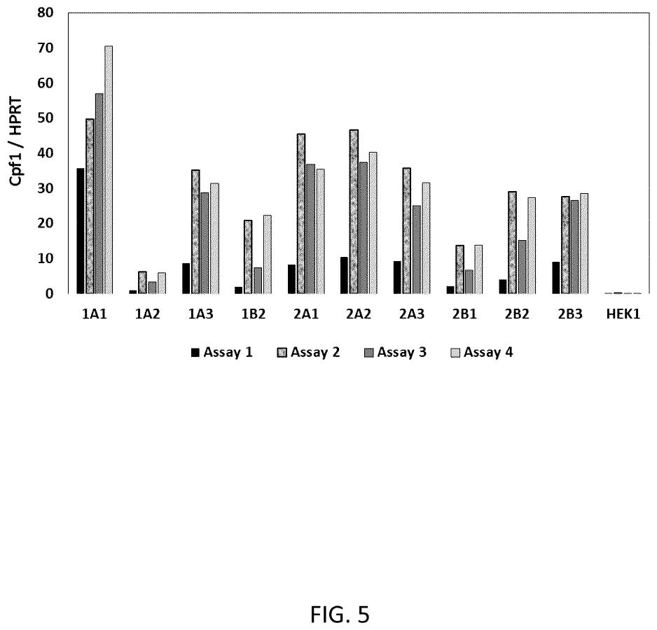

Monoclonal cell lines resistant to G418 were plated in 6-well plates and cultured for 24 hrs. Cells were lysed with GITC-containing buffer and RNA was isolated using the Wizard 96-well RNA isolation binding plates (Promega) on a Corbett liquid handling robot. Liquid handling robotics (Perkin Elmer) were used to synthesize complementary DNA (cDNA) using SuperScriptII (Invitrogen) and set-up qPCR assays using Immolase (Bioline) along with 500 nmol primers and 250 nmol probes (IDT). qPCR plates were run on the AB7900-HT and analyzed using the associated software (Applied Biosystems). FIG. 5 shows the relative level of AsCpf1 mRNA expression normalized to HPRT1 expression for a series of clonal lines. Not surprisingly, different clones showed different levels of AsCpf1 mRNA expression.

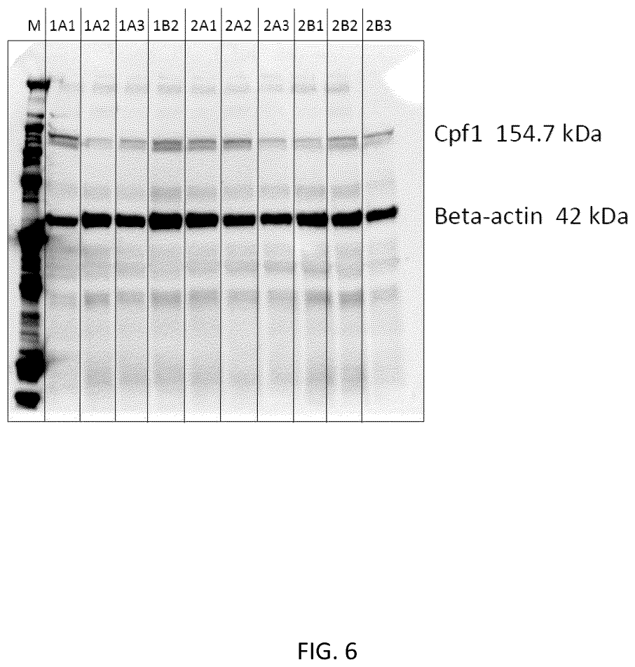

Total protein was isolated from the same AsCpf1-expressing monoclonal cells lines in cultures grown in parallel. Cells were lysed in RIPA buffer in the presence of a proteinase inhibitor. Protein concentration in each lysate was determined by BCA assay (Pierce). Fifteen micrograms of total protein from each sample was loaded onto an SDS-PAGE stainfree 4-20% gradient gel (Bio-Rad) and run at 180V for 45 minutes in 1×Tris/Glycine running buffer alongside the broad-range molecular weight marker (Bio-Rad). Protein was transferred to a PDVF membrane using Bio-Rad TransBlot transfer unit for 7 minutes. The blot was blocked in SuperBlock T20 Blocking Buffer (Thermo), followed by incubation with a 1:1000 dilution of V5 primary antibody (Abcam) and 1:5000 β-actin primary antibody (Abcam) for 1 hour at room temperature. The blot was washed 3 times for 15 minutes each in tris-buffered saline with Tween-20 (TBST). Goat anti-mouse HRP secondary antibody was used at a 1:3000 dilution along with the ladder specific StrepTactin secondary antibody and incubated at room temperature for 1 hour at room temperature. The blot was then washed 3 times for 15 minutes in TBST. Luminescence detection was done using the Pierce West-Femto ECL (Thermo) substrate. FIG. 6 shows detection of V5-tagged AsCpf1 recombinant protein expression levels in 10 monoclonal cell lines. There is good concordance between observed protein levels seen in FIG. 6 and the corresponding mRNA levels from the same cell lines shown in FIG. 5.

Three monoclonal AsCpf1 stable cell lines (1A1, 2A2 and 2B1) were expanded and tested for the ability to support AsCpf1-directed genome editing. Based on AsCpf1 mRNA and protein levels previously determined, 1A1 is a “high” expressing line, 2A2 is a “medium” expressing line, and 2B1 is a “low” expressing line. The cell lines were transfected with 6 different crRNAs targeting different sites within an exon of the human HRPT1 gene, shown below in Table 2. The crRNAs comprise a universal 20 base Cpf1-binding domain at the 5′-end and a 24 base target-specific protospacer domain at the 3′-end.

TABLE 2
AsCpf1 crRNAs targeting human HPRT1

| | | SEQ ID |
|---|---|---|
| Site | Sequence | NO: |
| 38171_AS | uaauuucuacucuuguagauuaaacacuguuucauuucauccgu | 37 |
| 38254_AS | uaauuucuacucuuguagauaccagcaagcuguuaauuacaaaa | 38 |
| 38325_S | uaauuucuacucuuguagauaccaucuuuaaccuaaaagaguuu | 39 |
| 38337_AS | uaauuucuacucuuguagaugguuaaagaugguuaaaugauuga | 40 |
| 38351_S | uaauuucuacucuuguagauugugaaauggcuuauaauugcuua | 41 |
| 38538_S | uaauuucuacucuuguagauaauguaaguaauugcuucuuuuuc | 42 |

RNA bases are shown 5′-3′ orientation, RNA bases are shown in lower case. Locations are specified within the human HPRT1 gene with orientation relative to the sense coding strand indicated (S = sense, AS = antisense).

In a reverse transfection format, anti-HPRT1 crRNAs were individually mixed with Lipofectamine RNAiMAX (Life Technologies) and transfected into each of the 3 HEK-Cpf1 cell lines. Transfections were done with 40,000 cells per well in 96 well plate format. RNAs were introduced at a final concentration of 30 nM in 0.75 μl of the lipid reagent. Cells were incubated at 37° C. for 48 hours. Genomic DNA was isolated using QuickExtract solution (Epicentre). Genomic DNA was amplified with KAPA HiFi DNA Polymerase (Roche) and primers targeting the HPRT region of interest (HPRT-low forward primer: AAGAATGTTGTGATAAAAGGTGATGCT (SEQ ID NO:394); HPRT-low reverse primer: ACACATCCATGGGACTTCTGCCTC (SEQ ID NO:395). PCR products were melted and re-annealed in NEB buffer 2 (New England Biolabs) to allow for heteroduplex formation followed by digestion with 2 units of T7 endonuclease 1 (T7EI; New England Biolabs) for 1 hour at 37° C. The digested products were visualized on a Fragment Analyzer (Advanced Analytical Technologies). Percent cleavage of targeted DNA was calculated as the average molar concentration of the cut products/(average molar concentration of the cut products+molar concentration of the uncut band)×100. The cleavage efficiencies seen in the 3 cell lines are shown in Table 3 below.

TABLE 3
Gene targeting efficiency of 6 HPRT1 crRNAs
in 3 HEK-Cpf1 cell lines

| | % Cleavage in T7EI assays | | |
|---|---|---|---|
| Site | 1A1 | 2A2 | 2B1 |
| 38171_AS | 19 | 19.1 | 8.3 |
| 38254_AS | 41 | 42.4 | 30.3 |
| 38325_S | 27.8 | 26.5 | 14.8 |
| 38337_AS | 65.3 | 73.7 | 71.6 |
| 38351_S | 73.3 | 78.6 | 73.4 |
| 38538_S | 44.6 | 47.9 | 32.8 |

Locations of the crRNAs are specified within the human HPRT1 gene with orientation relative to the sense coding strand indicated (S = sense, AS = antisense). % Cleavage demonstrates alteration in the sequence of the cell line after Cpf1-mediated genome editing at the HPRT1 locus relative to wild-type.

As expected, the different crRNAs targeting different sites in HPRT1 showed different levels of gene editing activity. In cell line 1A1 this ranged from 18% to 73%. The “high” and “medium” Cpf1-expressing clones 1A1 and 2A2 showed nearly identical gene editing activity, indicating that both clones expressed Cpf1 at sufficient levels to reach maximal gene editing activity at each site. Clone 2B1, the “low” expressing clone, showed reduced editing activity. Clones 1A1 and 2A2 are therefore both suitable for Cpf1 crRNA optimization and site screening.

### Example 3

crRNA Length Optimization: Testing Truncation of the 5′-20-Base Universal Loop Domain.

A set of 6 sites in the human HPRT1 gene were chosen to study length optimization of AsCpf1 crRNAs. A series of crRNAs were synthesized all having a 3′-24 base target-specific protospacer domain and having 5′-loop domains of 20, 19, 18, and 17 bases, representing a set of serial 1-base deletions from the 5′-end. A second set of crRNAs were synthesized at the same sites all having a 3′-21 base target-specific protospacer domain, likewise with 5′-loop domains of 20, 19, 18, and 17 bases.

An HEK cell line that stably expresses the AsCpf1 endonuclease was employed in these studies (Example 2). In a reverse transfection format, anti-HPRT1 crRNAs were individually mixed with Lipofectamine RNAiMAX (Life Technologies) and transfected into the HEK-Cpf1 cell line. Transfections were done with 40,000 cells per well in 96 well plate format. RNAs were introduced at a final concentration of 30 nM in 0.75 μl of the lipid reagent. Cells were incubated at 37° C. for 48 hours. Genomic DNA was isolated using QuickExtract solution (Epicentre). Genomic DNA was amplified with KAPA HiFi DNA Polymerase (Roche) and primers targeting the HPRT region of interest (HPRT-low forward primer: AAGAATGTTGTGATAAAAGGTGATGCT (SEQ ID NO:394); HPRT-low reverse primer: ACACATCCATGGGACTTCTGCCTC (SEQ ID NO:395). PCR products were melted and re-annealed in NEB buffer 2 (New England Biolabs) to allow for heteroduplex formation followed by digestion with 2 units of T7 endonuclease 1 (T7EI; New England Biolabs) for 1 hour at 37° C. The digested products were visualized on a Fragment Analyzer (Advanced Analytical Technologies). Percent cleavage of targeted DNA was calculated as the average molar concentration of the cut products/(average molar concentration of the cut products+molar concentration of the uncut band)×100. Results are shown in Table 4 below and demonstrate that 5′-universal loop domains of 20 and 19 base lengths work well but a significant loss of activity is seen when 18 or 17 base loops domains are employed. The observations are nearly identical whether a 24 base or 21 base protospacer domain is employed.

TABLE 4
Effect of truncation in the 5′-loop domain with 24 or 21 base
3′-protospacer domains

| | | | SEQ |
|---|---|---|---|
| | | % Cleavage | ID |
| Seq Name | Sequence 5′-3′ | T7E1 Assay | NO: |
| 38171_AS 20-24 | uaauuucuacucuuguagauuaaacacuguuucauuucauccgu | 12% | 37 |
| 38171-AS 19-24 | aauuucuacucuuguagauuaaacacuguuucauuucauccgu | 15% | 43 |
| 38171-AS 18-24 | auuucuacucuuguagauuaaacacuguuucauuucauccgu | 4% | 44 |
| 38171-AS 17-24 | uuucuacucuuguagauuaaacacuguuucauuucauccgu | 1% | 45 |
| 38254_AS 20-24 | uaauuucuacucuuguagauaccagcaagcuguuaauuacaaaa | 15% | 38 |
| 38254-AS 19-24 | aauuucuacucuuguagauaccagcaagcuguuaauuacaaaa | 36% | 46 |
| 38254-AS 18-24 | auuucuacucuuguagauaccagcaagcuguuaauuacaaaa | 23% | 47 |
| 38254-AS 17-24 | uuucuacucuuguagauaccagcaagcuguuaauuacaaaa | 0% | 48 |
| 38325_S 20-24 | uaauuucuacucuuguagauaccaucuuuaaccuaaaagaguuu | 9% | 39 |
| 38325-S 19-24 | aauuucuacucuuguagauaccaucuuuaaccuaaaagaguuu | 37% | 49 |
| 38325-S 18-24 | auuucuacucuuguagauaccaucuuuaaccuaaaagaguuu | 27% | 50 |
| 38325-S 17-24 | uuucuacucuuguagauaccaucuuuaaccuaaaagaguuu | 0% | 51 |
| 38337_AS 20-24 | uaauuucuacucuuguagaugguuaaagaugguuaaaugauuga | 63% | 40 |
| 38337-AS 19-24 | aauuucuacucuuguagaugguuaaagaugguuaaaugauuga | 65% | 52 |
| 38337-AS 18-24 | auuucuacucuuguagaugguuaaagaugguuaaaugauuga | 46% | 53 |
| 38337-AS 17-24 | uuucuacucuuguagaugguuaaagaugguuaaaugauuga | 4% | 54 |
| 38351_S 20-24 | uaauuucuacucuuguagauugugaaauggcuuauaauugcuua | 57% | 41 |
| 38351-S 19-24 | aauuucuacucuuguagauugugaaauggcuuauaauugcuua | 76% | 55 |
| 38351-S 18-24 | auuucuacucuuguagauugugaaauggcuuauaauugcuua | 6% | 56 |
| 38351-S 17-24 | uuucuacucuuguagauugugaaauggcuuauaauugcuua | 0% | 57 |
| 38538_S 20-24 | uaauuucuacucuuguagauaauguaaguaauugcuucuuuuuc | 16% | 42 |
| 38538-S 19-24 | aauuucuacucuuguagauaauguaaguaauugcuucuuuuuc | 34% | 58 |
| 38538-S 18-24 | auuucuacucuuguagauaauguaaguaauugcuucuuuuuc | 2% | 59 |
| 38538-S 17-24 | uuucuacucuuguagauaauguaaguaauugcuucuuuuuc | 1% | 60 |
| 38171-AS 20-21 | uaauuucuacucuuguagauuaaacacuguuucauuucauc | 32% | 61 |
| 38171-AS 19-21 | aauuucuacucuuguagauuaaacacuguuucauuucauc | 44% | 62 |
| 38171-AS 18-21 | auuucuacucuuguagauuaaacacuguuucauuucauc | 16% | 63 |
| 38171-AS 17-21 | uuucuacucuuguagauuaaacacuguuucauuucauc | 1% | 64 |
| 38254-AS 20-21 | uaauuucuacucuuguagauaccagcaagcuguuaauuaca | 45% | 65 |
| 38254-AS 19-21 | aauuucuacucuuguagauaccagcaagcuguuaauuaca | 28% | 66 |
| 38254-AS 18-21 | auuucuacucuuguagauaccagcaagcuguuaauuaca | 50% | 67 |
| 38254-AS 17-21 | uuucuacucuuguagauaccagcaagcuguuaauuaca | 0% | 68 |
| 38325-S 20-21 | uaauuucuacucuuguagauaccaucuuuaaccuaaaagag | 50% | 69 |
| 38325-S 19-21 | aauuucuacucuuguagauaccaucuuuaaccuaaaagag | 49% | 70 |
| 38325-S 18-21 | auuucuacucuuguagauaccaucuuuaaccuaaaagag | 36% | 71 |
| 38325-S 17-21 | uuucuacucuuguagauaccaucuuuaaccuaaaagag | 0% | 72 |
| 38337-AS 20-21 | uaauuucuacucuuguagaugguuaaagaugguuaaaugau | 72% | 73 |
| 38337-AS 19-21 | aauuucuacucuuguagaugguuaaagaugguuaaaugau | 73% | 74 |
| 38337-AS 18-21 | auuucuacucuuguagaugguuaaagaugguuaaaugau | 62% | 75 |
| 38337-AS 17-21 | uuucuacucuuguagaugguuaaagaugguuaaaugau | 12% | 76 |
| 38351-S 20-21 | uaauuucuacucuuguagauugugaaauggcuuauaauugc | 81% | 77 |
| 38351-S 19-21 | aauuucuacucuuguagauugugaaauggcuuauaauugc | 81% | 78 |
| 38351-S 18-21 | auuucuacucuuguagauugugaaauggcuuauaauugc | 20% | 79 |
| 38351-S 17-21 | uuucuacucuuguagauugugaaauggcuuauaauugc | 0% | 80 |
| 38538-S 20-21 | uaauuucuacucuuguagauaauguaaguaauugcuucuuu | 65% | 81 |
| 38538-S 19-21 | aauuucuacucuuguagauaauguaaguaauugcuucuuu | 41% | 82 |
| 38538-S 18-21 | auuucuacucuuguagauaauguaaguaauugcuucuuu | 11% | 83 |
| 38538-S 17-21 | uuucuacucuuguagauaauguaaguaauugcuucuuu | 1% | 84 |

RNA bases are shown in lower case. Locations are specified within the human HPRT1 gene with orientation relative to the sense coding strand indicated (S = sense, AS = antisense). Sequence names include length of the 5′-universal loop domain (17-20 bases) and the 3′-target specific protospacer domain (24 or 21 bases).

### Example 4

crRNA Length Optimization: Testing Truncation of the 3′-24-Base Target Specific Protospacer Domain.

The same set of 6 sites in the human HPRT1 gene was used to study the effects of truncation in the 3′-protospacer (target specific) domain. A series of AsCpf1 crRNAs were synthesized all having the same 5′-20 base universal loop domain. These were paired with 3′-target specific protospacer domains of 21, 19, 18, or 17 bases, having serial deletions from the 3′-end.

An HEK cell line that stably expresses the AsCpf1 endonuclease was employed in these studies (Example 2). In a reverse transfection format, anti-HPRT1 AsCpf1 crRNAs were individually mixed with Lipofectamine RNAiMAX (Life Technologies) and transfected into the HEK-Cpf1 cell line. Transfections were done with 40,000 cells per well in 96 well plate format. RNAs were introduced at a final concentration of 30 nM in 0.75 μl of the lipid reagent. Cells were incubated at 37° C. for 48 hours. Genomic DNA was isolated using QuickExtract solution (Epicentre). Genomic DNA was amplified with KAPA HiFi DNA Polymerase (Roche) and primers targeting the HPRT region of interest (HPRT-low forward primer: AAGAATGTTGTGATAAAAGGTGATGCT (SEQ ID NO:394); HPRT-low reverse primer: ACACATCCATGGGACTTCTGCCTC (SEQ ID NO:395). PCR products were melted and re-annealed in NEB buffer 2 (New England Biolabs) to allow for heteroduplex formation followed by digestion with 2 units of T7 endonuclease 1 (T7EI; New England Biolabs) for 1 hour at 37° C. The digested products were visualized on a Fragment Analyzer (Advanced Analytical Technologies). Percent cleavage of targeted DNA was calculated as the average molar concentration of the cut products/(average molar concentration of the cut products+molar concentration of the uncut band)×100. Results are shown in Table 5 below and demonstrate that a 3′-protospacer (target specific) domain of 21 base lengths work well but loss of activity is observed in a sequence/site dependent fashion as this domain is shortened. Some highly active sites (such as 38351) maintain appreciate activity even when truncated to 17 bases, however to maintain the highest likelihood of functionality at all sites a protospacer of 21 bases is recommended. Therefore, a prudent minimal length AsCpf1 crRNA is 41 bases, comprising a 20-base 5′-universal loop domain and a 21-base 3′-protospacer target-specific domain.

TABLE 5
Effect of truncation in the 3′-protospacer domain with a 20 base
5′-loop domain

| | | % Cleavage | SEQ ID |
|---|---|---|---|
| Seq Name | Sequence 5′-3′ | T7E1 Assay | NO: |
| 38171-AS 20-21 | uaauuucuacucuuguagauuaaacacuguuucauuucauc | 59% | 61 |
| 38171-AS 20-19 | uaauuucuacucuuguagauuaaacacuguuucauuuca | 13% | 85 |
| 38171-AS 20-18 | uaauuucuacucuuguagauuaaacacuguuucauuuc | 2% | 86 |
| 38171-AS 20-17 | uaauuucuacucuuguagauuaaacacuguuucauuu | 3% | 87 |
| 38254-AS 20-21 | uaauuucuacucuuguagauaccagcaagcuguuaauuaca | 61% | 65 |
| 38254-AS 20-19 | uaauuucuacucuuguagauaccagcaagcuguuaauua | 5% | 88 |
| 38254-AS 20-18 | uaauuucuacucuuguagauaccagcaagcuguuaauu | 0% | 89 |
| 38254-AS 20-17 | uaauuucuacucuuguagauaccagcaagcuguuaau | 0% | 90 |
| 38325-S 20-21 | uaauuucuacucuuguagauaccaucuuuaaccuaaaagag | 70% | 69 |
| 38325-S 20-19 | uaauuucuacucuuguagauaccaucuuuaaccuaaaag | 34% | 91 |
| 38325-S 20-18 | uaauuucuacucuuguagauaccaucuuuaaccuaaaa | 0% | 92 |
| 38325-S 20-17 | uaauuucuacucuuguagauaccaucuuuaaccuaaa | 0% | 93 |
| 38337-AS 20-21 | uaauuucuacucuuguagaugguuaaagaugguuaaaugau | 80% | 73 |
| 38337-AS 20-19 | uaauuucuacucuuguagaugguuaaagaugguuaaaug | 78% | 94 |
| 38337-AS 20-18 | uaauuucuacucuuguagaugguuaaagaugguuaaau | 3% | 95 |
| 38337-AS 20-17 | uaauuucuacucuuguagaugguuaaagaugguuaaa | 0% | 96 |
| 38351-S 20-21 | uaauuucuacucuuguagauugugaaauggcuuauaauugc | 85% | 77 |
| 38351-S 20-19 | uaauuucuacucuuguagauugugaaauggcuuauaauu | 87% | 97 |
| 38351-S 20-18 | uaauuucuacucuuguagauugugaaauggcuuauaau | 85% | 98 |
| 38351-S 20-17 | uaauuucuacucuuguagauugugaaauggcuuauaa | 67% | 99 |
| 38538-S 20-21 | uaauuucuacucuuguagauaauguaaguaauugcuucuuu | 75% | 81 |
| 38538-S 20-19 | uaauuucuacucuuguagauaauguaaguaauugcuucu | 55% | 100 |
| 38538-S 20-18 | uaauuucuacucuuguagauaauguaaguaauugcuuc | 11% | 101 |
| 38538-S 20-17 | uaauuucuacucuuguagauaauguaaguaauugcuu | 0% | 102 |

RNA bases are shown in lower case. Locations are specified within the human HPRT1 gene with orientation relative to the sense-coding strand indicated (S = sense, AS = antisense). Sequence names include length of the 5′-universal loop domain (20 bases) and the 3′-protospacer target-specific domain (21, 19, 18, or 17 bases).

### Example 5

a Single-Base 2′OMe Modification Walk Through Two AsCpf1 crRNAs.

Two sites in the human HPRT1 gene were chosen (38351 and 38595) to study the effects of replacement of a single RNA residue with a 2′OMe-RNA residue at every possible position within AsCpf1 crRNAs. Given the possibility of sequence-specific tolerance to modification, it was necessary to perform this screening at two sites. A series of crRNAs were synthesized having a single 2′OMe residue at every possible position in single-base steps. The crRNAs were either 44 base or 41 base lengths. All had a 5′-end 20 base universal loop domain followed by a 3′-end 21 or 24 base protospacer target-specific domain.

An HEK cell line that stably expresses the AsCpf1 endonuclease was employed in these studies (HEK-Cpf1) (Example 2). In a reverse transfection format, anti-HPRT1 crRNAs were individually mixed with Lipofectamine RNAiMAX (Life Technologies) and transfected into the HEK-Cpf1 cell line. Transfections were done with 40,000 cells per well in 96 well plate format. RNAs were introduced at a final concentration of 30 nM in 0.75 μl of the lipid reagent. Cells were incubated at 37° C. for 48 hours. Genomic DNA was isolated using QuickExtract solution (Epicentre). Genomic DNA was amplified with KAPA HiFi DNA Polymerase (Roche) and primers targeting the HPRT region of interest (HPRT-low forward primer: AAGAATGTTGTGATAAAAGGTGATGCT (SEQ ID NO:394); HPRT-low reverse primer: ACACATCCATGGGACTTCTGCCTC (SEQ ID NO:395). PCR products were melted and re-annealed in NEB buffer 2 (New England Biolabs) to allow for heteroduplex formation followed by digestion with 2 units of T7 endonuclease 1 (T7EI; New England Biolabs) for 1 hour at 37° C. The digested products were visualized on a Fragment Analyzer (Advanced Analytical Technologies). Percent cleavage of targeted DNA was calculated as the average molar concentration of the cut products/(average molar concentration of the cut products+molar concentration of the uncut band)×100. Results for HPRT1 site 38351 are shown in Table 6 below and for HRPT1 site 38595 in Table 7 below. The results demonstrate the locations of sites that reduce activity or totally kill activity of Cpf1 to cleave dsDNA when the 2′OMe modified replaced an RNA residue. The results are nearly identical whether a 24 base or 21 base protospacer domain is employed.

Sites where substitution of a 2′OMe RNA residue for an RNA residue showed loss of activity in the genome editing assay were mapped to location within the 5′-universal loop domain or the 3′-target specific protospacer domain. Results are summarized in FIG. 7. Modification of residues A2, A3, U4, U11, G15, and U20 within the universal loop domain leads to loss of activity; the same sites were identified for all 4 crRNA classes studied (Site 38351 44mer, Site 38351 41mer, Site 38595 44mer, and Site 38595 41mer). In contrast, the precise pattern of modification effects varied for sites within the protospacer domain, which is expected as it is common for modification tolerance to vary with sequence context and the protospacer domain has a different sequence for every target site. For the sequences studied, positions 5, 6, 13, 16, and 18 showed loss of activity with modification for all 4 crRNA classes and therefore are identified positions to avoid the 2′OMe RNA chemical modification.

TABLE 6
Single-base 2′OMe modification walk through HPRT1 Site 38351
AsCpf1 crRNAs

| | | % | |
|---|---|---|---|
| | | Cleavage | SEQ |
| | | T7E1 | ID |
| Seq Name | Sequence 5′-3′ | Assay | NO: |
| 38351-44 | uaauuucuacucuuguagauugugaaauggcuuauaauugcuua | 77% | 103 |
| unmod | | | |
| 38351-44- | uaauuucuacucuuguagauugugaaauggcuuauaauugcuua | 83% | 104 |
| L1 | | | |
| 38351-44- | uaauuucuacucuuguagauugugaaauggcuuauaauugcuua | 32% | 105 |
| L2 | | | |
| 38351-44- | uaauuucuacucuuguagauugugaaauggcuuauaauugcuua | 4% | 106 |
| L3 | | | |
| 38351-44- | uaauuucuacucuuguagauugugaaauggcuuauaauugcuua | 2% | 107 |
| L4 | | | |
| 38351-44- | uaauuucuacucuuguagauugugaaauggcuuauaauugcuua | 88% | 108 |
| L5 | | | |
| 38351-44- | uaauuucuacucuuguagauugugaaauggcuuauaauugcuua | 87% | 109 |
| L6 | | | |
| 38351-44- | uaauuucuacucuuguagauugugaaauggcuuauaauugcuua | 85% | 110 |
| L7 | | | |
| 38351-44- | uaauuucuacucuuguagauugugaaauggcuuauaauugcuua | 76% | 111 |
| L8 | | | |
| 38351-44- | uaauuucuacucuuguagauugugaaauggcuuauaauugcuua | 89% | 112 |
| L9 | | | |
| 38351-44- | uaauuucuacucuuguagauugugaaauggcuuauaauugcuua | 85% | 113 |
| L10 | | | |
| 38351-44- | uaauuucuacucuuguagauugugaaauggcuuauaauugcuua | 34% | 114 |
| L11 | | | |
| 38351-44- | uaauuucuacucuuguagauugugaaauggcuuauaauugcuua | 86% | 115 |
| L12 | | | |
| 38351-44- | uaauuucuacucuuguagauugugaaauggcuuauaauugcuua | 85% | 116 |
| L13 | | | |
| 38351-44- | uaauuucuacucuuguagauugugaaauggcuuauaauugcuua | 86% | 117 |
| L14 | | | |
| 38351-44- | uaauuucuacucuuguagauugugaaauggcuuauaauugcuua | 58% | 118 |
| L15 | | | |
| 38351-44- | uaauuucuacucuuguagauugugaaauggcuuauaauugcuua | 89% | 119 |
| L16 | | | |
| 38351-44- | uaauuucuacucuuguagauugugaaauggcuuauaauugcuua | 88% | 120 |
| L17 | | | |
| 38351-44- | uaauuucuacucuuguagauugugaaauggcuuauaauugcuua | 82% | 121 |
| L18 | | | |
| 38351-44- | uaauuucuacucuuguagauugugaaauggcuuauaauugcuua | 87% | 122 |
| L19 | | | |
| 38351-44- | uaauuucuacucuuguagauugugaaauggcuuauaauugcuua | 52% | 123 |
| L20 | | | |
| 38351-44- | uaauuucuacucuuguagauugugaaauggcuuauaauugcuua | 87% | 124 |
| T1 | | | |
| 38351-44- | uaauuucuacucuuguagauugugaaauggcuuauaauugcuua | 79% | 125 |
| T2 | | | |
| 38351-44- | uaauuucuacucuuguagauugugaaauggcuuauaauugcuua | 86% | 126 |
| T3 | | | |
| 38351-44- | uaauuucuacucuuguagauugugaaauggcuuauaauugcuua | 81% | 127 |
| T4 | | | |
| 38351-44- | uaauuucuacucuuguagauugugaaauggcuuauaauugcuua | 69% | 128 |
| T5 | | | |
| 38351-44- | uaauuucuacucuuguagauugugaaauggcuuauaauugcuua | 57% | 129 |
| T6 | | | |
| 38351-44- | uaauuucuacucuuguagauugugaaauggcuuauaauugcuua | 84% | 130 |
| T7 | | | |
| 38351-44- | uaauuucuacucuuguagauugugaaauggcuuauaauugcuua | 90% | 131 |
| T8 | | | |
| 38351-44- | uaauuucuacucuuguagauugugaaauggcuuauaauugcuua | 86% | 132 |
| T9 | | | |
| 38351-44- | uaauuucuacucuuguagauugugaaauggcuuauaauugcuua | 89% | 133 |
| T10 | | | |
| 38351-44- | uaauuucuacucuuguagauugugaaauggcuuauaauugcuua | 86% | 134 |
| T11 | | | |
| 38351-44- | uaauuucuacucuuguagauugugaaauggcuuauaauugcuua | 90% | 135 |
| T12 | | | |
| 38351-44- | uaauuucuacucuuguagauugugaaauggcuuauaauugcuua | 15% | 136 |
| T13 | | | |
| 38351-44- | uaauuucuacucuuguagauugugaaauggcuuauaauugcuua | 71% | 137 |
| T14 | | | |
| 38351-44- | uaauuucuacucuuguagauugugaaauggcuuauaauugcuua | 72% | 138 |
| T15 | | | |
| 38351-44- | uaauuucuacucuuguagauugugaaauggcuuauaauugcuua | 68% | 139 |
| T16 | | | |
| 38351-44- | uaauuucuacucuuguagauugugaaauggcuuauaauugcuua | 72% | 140 |
| T17 | | | |
| 38351-44- | uaauuucuacucuuguagauugugaaauggcuuauaauugcuua | 64% | 141 |
| T18 | | | |
| 38351-44- | uaauuucuacucuuguagauugugaaauggcuuauaauugcuua | 75% | 142 |
| T19 | | | |
| 38351-44- | uaauuucuacucuuguagauugugaaauggcuuauaauugcuua | 71% | 143 |
| T20 | | | |
| 38351-44- | uaauuucuacucuuguagauugugaaauggcuuauaauugcuua | 72% | 144 |
| T21 | | | |
| 38351-44- | uaauuucuacucuuguagauugugaaauggcuuauaauugcuua | 69% | 145 |
| T22 | | | |
| 38351-44- | uaauuucuacucuuguagauugugaaauggcuuauaauugcuua | 72% | 146 |
| T23 | | | |
| 38351-44- | uaauuucuacucuuguagauugugaaauggcuuauaauugcuua | 70% | 147 |
| T24 | | | |
| 38351-41 | uaauuucuacucuuguagauugugaaauggcuuauaauugc | 77% | 148 |
| unmod | | | |
| 38351-41- | uaauuucuacucuuguagauugugaaauggcuuauaauugc | 87% | 149 |
| L1 | | | |
| 38351-41- | uaauuucuacucuuguagauugugaaauggcuuauaauugc | 63% | 150 |
| L2 | | | |
| 38351-41- | uaauuucuacucuuguagauugugaaauggcuuauaauugc | 15% | 151 |
| L3 | | | |
| 38351-41- | uaauuucuacucuuguagauugugaaauggcuuauaauugc | 6% | 152 |
| L4 | | | |
| 38351-41- | uaauuucuacucuuguagauugugaaauggcuuauaauugc | 88% | 153 |
| L5 | | | |
| 38351-41- | uaauuucuacucuuguagauugugaaauggcuuauaauugc | 88% | 154 |
| L6 | | | |
| 38351-41- | uaauuucuacucuuguagauugugaaauggcuuauaauugc | 81% | 155 |
| L7 | | | |
| 38351-41- | uaauuucuacucuuguagauugugaaauggcuuauaauugc | 78% | 156 |
| L8 | | | |
| 38351-41- | uaauuucuacucuuguagauugugaaauggcuuauaauugc | 90% | 157 |
| L9 | | | |
| 38351-41- | uaauuucuacucuuguagauugugaaauggcuuauaauugc | 88% | 158 |
| L10 | | | |
| 38351-41- | uaauuucuacucuuguagauugugaaauggcuuauaauugc | 59% | 159 |
| L11 | | | |
| 38351-41- | uaauuucuacucuuguagauugugaaauggcuuauaauugc | 88% | 160 |
| L12 | | | |
| 38351-41- | uaauuucuacucuuguagauugugaaauggcuuauaauugc | 89% | 161 |
| L13 | | | |
| 38351-41- | uaauuucuacucuuguagauugugaaauggcuuauaauugc | 88% | 162 |
| L14 | | | |
| 38351-41- | uaauuucuacucuuguagauugugaaauggcuuauaauugc | 41% | 163 |
| L15 | | | |
| 38351-41- | uaauuucuacucuuguagauugugaaauggcuuauaauugc | 90% | 164 |
| L16 | | | |
| 38351-41- | uaauuucuacucuuguagauugugaaauggcuuauaauugc | 89% | 165 |
| L17 | | | |
| 38351-41- | uaauuucuacucuuguagauugugaaauggcuuauaauugc | 89% | 166 |
| L18 | | | |
| 38351-41- | uaauuucuacucuuguagauugugaaauggcuuauaauugc | 88% | 167 |
| L19 | | | |
| 38351-41- | uaauuucuacucuuguagauugugaaauggcuuauaauugc | 77% | 168 |
| L20 | | | |
| 38351-41- | uaauuucuacucuuguagauugugaaauggcuuauaauugc | 89% | 169 |
| T1 | | | |
| 38351-41- | uaauuucuacucuuguagauugugaaauggcuuauaauugc | 84% | 170 |
| T2 | | | |
| 38351-41- | uaauuucuacucuuguagauugugaaauggcuuauaauugc | 87% | 171 |
| T3 | | | |
| 38351-41- | uaauuucuacucuuguagauugugaaauggcuuauaauugc | 86% | 172 |
| T4 | | | |
| 38351-41- | uaauuucuacucuuguagauugugaaauggcuuauaauugc | 80% | 173 |
| T5 | | | |
| 38351-41- | uaauuucuacucuuguagauugugaaauggcuuauaauugc | 79% | 174 |
| T6 | | | |
| 38351-41- | uaauuucuacucuuguagauugugaaauggcuuauaauugc | 86% | 175 |
| T7 | | | |
| 38351-41- | uaauuucuacucuuguagauugugaaauggcuuauaauugc | 89% | 176 |
| T8 | | | |
| 38351-41- | uaauuucuacucuuguagauugugaaauggcuuauaauugc | 89% | 177 |
| T9 | | | |
| 38351-41- | uaauuucuacucuuguagauugugaaauggcuuauaauugc | 89% | 178 |
| T10 | | | |
| 38351-41- | uaauuucuacucuuguagauugugaaauggcuuauaauugc | 89% | 179 |
| T11 | | | |
| 38351-41- | uaauuucuacucuuguagauugugaaauggcuuauaauugc | 88% | 180 |
| T12 | | | |
| 38351-41- | uaauuucuacucuuguagauugugaaauggcuuauaauugc | 23% | 181 |
| T13 | | | |
| 38351-41- | uaauuucuacucuuguagauugugaaauggcuuauaauugc | 75% | 182 |
| T14 | | | |
| 38351-41- | uaauuucuacucuuguagauugugaaauggcuuauaauugc | 77% | 183 |
| T15 | | | |
| 38351-41- | uaauuucuacucuuguagauugugaaauggcuuauaauugc | 72% | 184 |
| T16 | | | |
| 38351-41- | uaauuucuacucuuguagauugugaaauggcuuauaauugc | 76% | 185 |
| T17 | | | |
| 38351-41- | uaauuucuacucuuguagauugugaaauggcuuauaauugc | 71% | 186 |
| T18 | | | |
| 38351-41- | uaauuucuacucuuguagauugugaaauggcuuauaauugc | 77% | 187 |
| T19 | | | |
| 38351-41- | uaauuucuacucuuguagauugugaaauggcuuauaauugc | 75% | 188 |
| T20 | | | |
| 38351-41- | uaauuucuacucuuguagauugugaaauggcuuauaauugc | 77% | 189 |
| T21 | | | |

Oligonucleotide sequences are shown 5′-3′. Lowercase = RNA; Underlined lowercase = 2′-O-methyl RNA. The relative functional activity of each species is indicated by the % cleavage in a T7EI heteroduplex assay. The sequence name indicates if the crRNA is a 44mer with a 24 base target domain or a 41mer with a 21 base target domain. The position of the 2′OMe residue with either the loop domain (L) or target domain (T) is indicated.

TABLE 7
Single-base 2′OMe modification walk through HPRT1 Site 38595
AsCpf1 crRNAs

| | | % | SEQ |
|---|---|---|---|
| | | Cleavage | ID |
| Seq Name | Sequence 5′-3′ | T7E1 Assay | NO: |
| 38595-44 | uaauuucuacucuuguagauggaaagagaauuguuuucuccuuc | 49% | 190 |
| unmod | | | |
| 38595-44- | uaauuucuacucuuguagauggaaagagaauuguuuucuccuuc | 48% | 191 |
| L1 | | | |
| 38595-44- | uaauuucuacucuuguagauggaaagagaauuguuuucuccuuc | 34% | 192 |
| L2 | | | |
| 38595-44- | uaauuucuacucuuguagauggaaagagaauuguuuucuccuuc | 6% | 193 |
| L3 | | | |
| 38595-44- | uaauuucuacucuuguagauggaaagagaauuguuuucuccuuc | 3% | 194 |
| L4 | | | |
| 38595-44- | uaauuucuacucuuguagauggaaagagaauuguuuucuccuuc | 59% | 195 |
| L5 | | | |
| 38595-44- | uaauuucuacucuuguagauggaaagagaauuguuuucuccuuc | 54% | 196 |
| L6 | | | |
| 38595-44- | uaauuucuacucuuguagauggaaagagaauuguuuucuccuuc | 56% | 197 |
| L7 | | | |
| 38595-44- | uaauuucuacucuuguagauggaaagagaauuguuuucuccuuc | 52% | 198 |
| L8 | | | |
| 38595-44- | uaauuucuacucuuguagauggaaagagaauuguuuucuccuuc | 60% | 199 |
| L9 | | | |
| 38595-44- | uaauuucuacucuuguagauggaaagagaauuguuuucuccuuc | 56% | 200 |
| L10 | | | |
| 38595-44- | uaauuucuacucuuguagauggaaagagaauuguuuucuccuuc | 23% | 201 |
| L11 | | | |
| 38595-44- | uaauuucuacucuuguagauggaaagagaauuguuuucuccuuc | 51% | 202 |
| L12 | | | |
| 38595-44- | uaauuucuacucuuguagauggaaagagaauuguuuucuccuuc | 58% | 203 |
| L13 | | | |
| 38595-44- | uaauuucuacucuuguagauggaaagagaauuguuuucuccuuc | 52% | 204 |
| L14 | | | |
| 38595-44- | uaauuucuacucuuguagauggaaagagaauuguuuucuccuuc | 33% | 205 |
| L15 | | | |
| 38595-44- | uaauuucuacucuuguagauggaaagagaauuguuuucuccuuc | 55% | 206 |
| L16 | | | |
| 38595-44- | uaauuucuacucuuguagauggaaagagaauuguuuucuccuuc | 58% | 207 |
| L17 | | | |
| 38595-44- | uaauuucuacucuuguagauggaaagagaauuguuuucuccuuc | 61% | 208 |
| L18 | | | |
| 38595-44- | uaauuucuacucuuguagauggaaagagaauuguuuucuccuuc | 54% | 209 |
| L19 | | | |
| 38595-44- | uaauuucuacucuuguagauggaaagagaauuguuuucuccuuc | 29% | 210 |
| L20 | | | |
| 38595-44- | uaauuucuacucuuguagauggaaagagaauuguuuucuccuuc | 55% | 211 |
| T1 | | | |
| 38595-44- | uaauuucuacucuuguagauggaaagagaauuguuuucuccuuc | 53% | 212 |
| T2 | | | |
| 38595-44- | uaauuucuacucuuguagauggaaagagaauuguuuucuccuuc | 49% | 213 |
| T3 | | | |
| 38595-44- | uaauuucuacucuuguagauggaaagagaauuguuuucuccuuc | 20% | 214 |
| T4 | | | |
| 38595-44- | uaauuucuacucuuguagauggaaagagaauuguuuucuccuuc | 17% | 215 |
| T5 | | | |
| 38595-44- | uaauuucuacucuuguagauggaaagagaauuguuuucuccuuc | 23% | 216 |
| T6 | | | |
| 38595-44- | uaauuucuacucuuguagauggaaagagaauuguuuucuccuuc | 47% | 217 |
| T7 | | | |
| 38595-44- | uaauuucuacucuuguagauggaaagagaauuguuuucuccuuc | 52% | 218 |
| T8 | | | |
| 38595-44- | uaauuucuacucuuguagauggaaagagaauuguuuucuccuuc | 51% | 219 |
| T9 | | | |
| 38595-44- | uaauuucuacucuuguagauggaaagagaauuguuuucuccuuc | 55% | 220 |
| T10 | | | |
| 38595-44- | uaauuucuacucuuguagauggaaagagaauuguuuucuccuuc | 53% | 221 |
| T11 | | | |
| 38595-44- | uaauuucuacucuuguagauggaaagagaauuguuuucuccuuc | 58% | 222 |
| T12 | | | |
| 38595-44- | uaauuucuacucuuguagauggaaagagaauuguuuucuccuuc | 20% | 223 |
| T13 | | | |
| 38595-44- | uaauuucuacucuuguagauggaaagagaauuguuuucuccuuc | 62% | 224 |
| T14 | | | |
| 38595-44- | uaauuucuacucuuguagauggaaagagaauuguuuucuccuuc | 60% | 225 |
| T15 | | | |
| 38595-44- | uaauuucuacucuuguagauggaaagagaauuguuuucuccuuc | 15% | 226 |
| T16 | | | |
| 38595-44- | uaauuucuacucuuguagauggaaagagaauuguuuucuccuuc | 49% | 227 |
| T17 | | | |
| 38595-44- | uaauuucuacucuuguagauggaaagagaauuguuuucuccuuc | 46% | 228 |
| T18 | | | |
| 38595-44- | uaauuucuacucuuguagauggaaagagaauuguuuucuccuuc | 64% | 229 |
| T19 | | | |
| 38595-44- | uaauuucuacucuuguagauggaaagagaauuguuuucuccuuc | 57% | 230 |
| T20 | | | |
| 38595-44- | uaauuucuacucuuguagauggaaagagaauuguuuucuccuuc | 55% | 231 |
| T21 | | | |
| 38595-44- | uaauuucuacucuuguagauggaaagagaauuguuuucuccuuc | 54% | 232 |
| T22 | | | |
| 38595-44- | uaauuucuacucuuguagauggaaagagaauuguuuucuccuuc | 56% | 233 |
| T23 | | | |
| 38595-44- | uaauuucuacucuuguagauggaaagagaauuguuuucuccuuc | 54% | 234 |
| T24 | | | |
| 38595-41 | uaauuucuacucuuguagauggaaagagaauuguuuucucc | 59% | 235 |
| unmod | | | |
| 38595-41- | uaauuucuacucuuguagauggaaagagaauuguuuucucc | 60% | 236 |
| L1 | | | |
| 38595-41- | uaauuucuacucuuguagauggaaagagaauuguuuucucc | 49% | 237 |
| L2 | | | |
| 38595-41- | uaauuucuacucuuguagauggaaagagaauuguuuucucc | 10% | 238 |
| L3 | | | |
| 38595-41- | uaauuucuacucuuguagauggaaagagaauuguuuucucc | 5% | 239 |
| L4 | | | |
| 38595-41- | uaauuucuacucuuguagauggaaagagaauuguuuucucc | 63% | 240 |
| L5 | | | |
| 38595-41- | uaauuucuacucuuguagauggaaagagaauuguuuucucc | 55% | 241 |
| L6 | | | |
| 38595-41- | uaauuucuacucuuguagauggaaagagaauuguuuucucc | 56% | 242 |
| L7 | | | |
| 38595-41- | uaauuucuacucuuguagauggaaagagaauuguuuucucc | 55% | 243 |
| L8 | | | |
| 38595-41- | uaauuucuacucuuguagauggaaagagaauuguuuucucc | 63% | 244 |
| L9 | | | |
| 38595-41- | uaauuucuacucuuguagauggaaagagaauuguuuucucc | 64% | 245 |
| L10 | | | |
| 38595-41- | uaauuucuacucuuguagauggaaagagaauuguuuucucc | 35% | 246 |
| L11 | | | |
| 38595-41- | uaauuucuacucuuguagauggaaagagaauuguuuucucc | 55% | 247 |
| L12 | | | |
| 38595-41- | uaauuucuacucuuguagauggaaagagaauuguuuucucc | 56% | 248 |
| L13 | | | |
| 38595-41- | uaauuucuacucuuguagauggaaagagaauuguuuucucc | 58% | 249 |
| L14 | | | |
| 38595-41- | uaauuucuacucuuguagauggaaagagaauuguuuucucc | 47% | 250 |
| L15 | | | |
| 38595-41- | uaauuucuacucuuguagauggaaagagaauuguuuucucc | 55% | 251 |
| L16 | | | |
| 38595-41- | uaauuucuacucuuguagauggaaagagaauuguuuucucc | 64% | 252 |
| L17 | | | |
| 38595-41- | uaauuucuacucuuguagauggaaagagaauuguuuucucc | 69% | 253 |
| L18 | | | |
| 38595-41- | uaauuucuacucuuguagauggaaagagaauuguuuucucc | 63% | 254 |
| L19 | | | |
| 38595-41- | uaauuucuacucuuguagauggaaagagaauuguuuucucc | 45% | 255 |
| L20 | | | |
| 38595-41- | uaauuucuacucuuguagauggaaagagaauuguuuucucc | 60% | 256 |
| T1 | | | |
| 38595-41- | uaauuucuacucuuguagauggaaagagaauuguuuucucc | 59% | 257 |
| T2 | | | |
| 38595-41- | uaauuucuacucuuguagauggaaagagaauuguuuucucc | 53% | 258 |
| T3 | | | |
| 38595-41- | uaauuucuacucuuguagauggaaagagaauuguuuucucc | 21% | 259 |
| T4 | | | |
| 38595-41- | uaauuucuacucuuguagauggaaagagaauuguuuucucc | 20% | 260 |
| T5 | | | |
| 38595-41- | uaauuucuacucuuguagauggaaagagaauuguuuucucc | 25% | 261 |
| T6 | | | |
| 38595-41- | uaauuucuacucuuguagauggaaagagaauuguuuucucc | 50% | 262 |
| T7 | | | |
| 38595-41- | uaauuucuacucuuguagauggaaagagaauuguuuucucc | 64% | 263 |
| T8 | | | |
| 38595-41- | uaauuucuacucuuguagauggaaagagaauuguuuucucc | 54% | 264 |
| T9 | | | |
| 38595-41- | uaauuucuacucuuguagauggaaagagaauuguuuucucc | 57% | 265 |
| T10 | | | |
| 38595-41- | uaauuucuacucuuguagauggaaagagaauuguuuucucc | 45% | 266 |
| T11 | | | |
| 38595-41- | uaauuucuacucuuguagauggaaagagaauuguuuucucc | 52% | 267 |
| T12 | | | |
| 38595-41- | uaauuucuacucuuguagauggaaagagaauuguuuucucc | 14% | 268 |
| T13 | | | |
| 38595-41- | uaauuucuacucuuguagauggaaagagaauuguuuucucc | 66% | 269 |
| T14 | | | |
| 38595-41- | uaauuucuacucuuguagauggaaagagaauuguuuucucc | 63% | 270 |
| T15 | | | |
| 38595-41- | uaauuucuacucuuguagauggaaagagaauuguuuucucc | 16% | 271 |
| T16 | | | |
| 38595-41- | uaauuucuacucuuguagauggaaagagaauuguuuucucc | 47% | 272 |
| T17 | | | |
| 38595-41- | uaauuucuacucuuguagauggaaagagaauuguuuucucc | 52% | 273 |
| T18 | | | |
| 38595-41- | uaauuucuacucuuguagauggaaagagaauuguuuucucc | 64% | 274 |
| T19 | | | |
| 38595-41- | uaauuucuacucuuguagauggaaagagaauuguuuucucc | 64% | 275 |
| T20 | | | |
| 38595-41- | uaauuucuacucuuguagauggaaagagaauuguuuucucc | 66% | 276 |
| T21 | | | |

Oligonucleotide sequences are shown 5′-3′. Lowercase = RNA; Underlined lowercase = 2′-O-methyl RNA. The relative functional activity of each species is indicated by the % cleavage in a T7EI heteroduplex assay. The sequence name indicates if the crRNA is a 44mer with a 24 base target domain or a 41mer with a 21 base target domain. The position of the 2′OMe residue with either the loop domain (L) or target domain (T) is indicated.

### Example 6

Modification of Blocks of Sequence in AsCpf1 crRNAs.

Three sites in the human HPRT1 gene were chosen (38351, 38595, and 38104) to study the effects of replacement of a blocks of RNA residues with 2′OMe-RNA, 2′F RNA, or LNA residues within the AsCpf1 crRNA. Modification of internucleotide linkages with phosphorothioate bonds (PS) as well as non-nucleotide end-modifiers were also tested. The crRNAs were either 44 base or 41 base lengths. All had a 5′-end 20 base universal loop domain followed by a 3′-end 21 or 24 base protospacer target-specific domain.

An HEK cell line that stably expresses the AsCpf1 endonuclease was employed in these studies (HEK-Cpf1) (Example 2). In a reverse transfection format, anti-HPRT1 crRNAs were individually mixed with Lipofectamine RNAiMAX (Life Technologies) and transfected into the HEK-Cpf1 cell line. Transfections were done with 40,000 cells per well in 96 well plate format. RNAs were introduced at a final concentration of 30 nM in 0.75 μl of the lipid reagent. Cells were incubated at 37° C. for 48 hours. Genomic DNA was isolated using QuickExtract solution (Epicentre). Genomic DNA was amplified with KAPA HiFi DNA Polymerase (Roche) and primers targeting the HPRT region of interest (HPRT-low forward primer: AAGAATGTTGTGATAAAAGGTGATGCT (SEQ ID NO:394); HPRT-low reverse primer: ACACATCCATGGGACTTCTGCCTC (SEQ ID NO:395). PCR products were melted and re-annealed in NEB buffer 2 (New England Biolabs) to allow for heteroduplex formation followed by digestion with 2 units of T7 endonuclease 1 (T7EI; New England Biolabs) for 1 hour at 37° C. The digested products were visualized on a Fragment Analyzer (Advanced Analytical Technologies). Percent cleavage of targeted DNA was calculated as the average molar concentration of the cut products/(average molar concentration of the cut products+molar concentration of the uncut band)×100. Results are shown in Table 8 below.

Large blocks of the universal 5-loop domain can be modified and retain activity (14/20 bases). However, the target-specific 3′-protospacer domain shows significant loss of activity when 2-3 consecutive 2′OMe residues replace RNA residues, even when those positions did not show any loss of activity in the single base walk (Example 5). Modification patterns in the protospacer domain are often expected to be impacted by sequence context, such that one modification pattern works well for one sequence but not for another sequence. The modification map shown in FIG. 7 displays modification patterns that range from minimal to high levels of modification that showed high performance at several sites and likely can be used regardless of sequence context.

2′F residues could be placed at any position that was tolerant of 2′OMe modification. LNA residues can also be placed within the AsCpf1 crRNA, and use of end-modifiers are shown below in Table 8. The phosphorothioate (PS) internucleotide linkage confers nuclease resistance and can be placed at the ends of the crRNA to block exonuclease attack or in the central regions to block endonuclease attack. Modification of large blocks of the crRNA (such as entire modification of the loop domain or the protospacer domain) with PS linkages are not compatible with crRNA function and significant loss of activity is seen when this modification pattern is employed. Limited use, such as 2-3 internucleotide linkages at each end, can be effectively employed, and such patterns are useful to block exonuclease attack. Non-base modifiers (such as a C3 spacer propanediol group or a ZEN modifier napthyl-azo group) can be placed at one or both ends of the crRNA without loss of activity and also block exonuclease attack.

TABLE 8
Functional impact of extensive modification of AsCpf1 crRNAs

| | | % | |
|---|---|---|---|
| | | Cleavage | |
| | | T7E1 | SEQ |
| Seq Name | Sequence 5′-3′ | Assay | ID NO: |
| 38351-44-L | uaauuucuacucuuguagauugugaaauggcuuauaauugcuua | 51% | 277 |
| 38351-44-T | uaauuucuacucuuguagauugugaaauggcuuauaauugcuua | 1% | 278 |
| 38351-44-LT | uaauuucuacucuuguagauugugaaauggcuuauaauugcuua | 1% | 279 |
| 38351-41-L | uaauuucuacucuuguagauugugaaauggcuuauaauugc | 53% | 280 |
| 38351-41-T | uaauuucuacucuuguagauugugaaauggcuuauaauugc | 1% | 281 |
| 38351-41-LT | uaauuucuacucuuguagauugugaaauggcuuauaauugc | 1% | 282 |
| 38595-44-L | uaauuucuacucuuguagauggaaagagaauuguuuucuccuuc | 51% | 283 |
| 38595-44-T | uaauuucuacucuuguagauggaaagagaauuguuuucuccuuc | 1% | 284 |
| 38595-44-LT | uaauuucuacucuuguagauggaaagagaauuguuuucuccuuc | 1% | 285 |
| 38595-41-L | uaauuucuacucuuguagauggaaagagaauuguuuucucc | 51% | 286 |
| 38595-41-T | uaauuucuacucuuguagauggaaagagaauuguuuucucc | 1% | 287 |
| 38595-41-LT | uaauuucuacucuuguagauggaaagagaauuguuuucucc | 1% | 288 |
| 38595-41 | uaauuucuacucuuguagauggaaagagaauuguuuucucc | 35% | 235 |
| unmod | | | |
| 38595-41- | uaauuucuacucuuguagauggaaagagaauuguuuucucc | 24% | 289 |
| T1-3 | | | |
| 38595-41- | uaauuucuacucuuguagauggaaagagaauuguuuucucc | 2% | 290 |
| T7-12 | | | |
| 38595-41- | uaauuucuacucuuguagauggaaagagaauuguuuucucc | 37% | 291 |
| T1445 | | | |
| 38595-41- | uaauuucuacucuuguagauggaaagagaauuguuuucucc | 22% | 292 |
| T17-21 | | | |
| 38595-41- | uaauuucuacucuuguagauggaaagagaauuguuuucucc | 1% | 293 |
| T6-9, 18-21 | | | |
| 38595-41- | C3-uaauuucuacucuuguagauggaaagagaauuguuuucucc | 35% | 294 |
| 5′C3 | | | |
| 38595-41- | uaauuucuacucuuguagauggaaagagaauuguuuucucc-C3 | 41% | 295 |
| 3′C3 | | | |
| 38595-41- | C3-uaauuucuacucuuguagauggaaagagaauuguuuucucc-C3 | 41% | 296 |
| 2xC3 | | | |
| 38595-41- | uaauuucuacucuuguagauggaaagagaauuguuuucucc | 1% | 297 |
| L1-20 | | | |
| 38595-41- | uaauuucuacucuuguagauggaaagagaauuguuuucucc | 2% | 298 |
| L + 2 | | | |
| 38595-41- | uaauuucuacucuuguagauggaaagagaauuguuuucucc | 1% | 299 |
| L + 3 | | | |
| 38595-41- | uaauuucuacucuuguagauggaaagagaauuguuuucucc | 1% | 300 |
| L + 4 | | | |
| 38595-41- | uaauuucuacucuuguagauggaaagagaauuguuuucucc | 5% | 301 |
| L + 11 | | | |
| 38595-41- | uaauuucuacucuuguagauggaaagagaauuguuuucucc | 38% | 302 |
| L + 15 | | | |
| 38595-41- | uaauuucuacucuuguagauggaaagagaauuguuuucucc | 2% | 303 |
| L + 20 | | | |
| 38595-41-61 | C3-uaauuucuacucuuguagauggaaagagaauuguuuucucc-C3 | 67% | 304 |
| 38595-41-62 | u*a*a*uuucuacucuuguagauggaaagagaauuguuuuc*u*c*c | 58% | 305 |
| 38595-41-63 | u*a*a*uuucuacucuuguagauggaaagagaauuguuuuc*u*c*c | 63% | 306 |
| 38595-41-64 | u*a*a*u*u*u*c*u*a*c*u*c*u*u*g*u*a*g*a*uggaaagagaa | 10% | 307 |
| | uuguuuucucc | | |
| 38595-41-65 | uaauuucuacucuuguagau*g*g*a*a*a*g*a*g*a*a*u*u*g*u* | 2% | 308 |
| | u*u*u*c*u*c*c | | |
| 38595-41-66 | uaauuucuacucuuguagauggaaagagaauuguuuucucc | 57% | 309 |
| 38595-41-67 | uaauuucuacucuuguagauggaaagagaauuguuuucucc | 51% | 310 |
| 38595-41-68 | uaauuucuacucuuguagauggaaagagaauuguuuucucc | 20% | 311 |
| 38595-41-69 | uaauuucuacucuuguagauggaaagagaauuguuuucucc | 19% | 312 |
| 38595-41-70 | uaauuucuacucuuguagauggaaagagaauuguuuucucc | 27% | 313 |
| 38595-41-71 | uaauuucuacucuuguagauggaaagagaauuguuuucucc | 37% | 314 |
| 38595-41-72 | uaauuucuacucuuguagauggaaagagaauuguuuucucc | 65% | 315 |
| 38595-41-73 | uaauuucuacucuuguagauggaaagagaauuguuuucucc | 67% | 316 |
| 38595-41-74 | uaauuucuacucuuguagauggaaagagaauuguuuucucc | 65% | 317 |
| 38595-41-75 | uaauuucuacucuuguagauggaaagagaauuguuuucucc | 57% | 318 |
| 38595-41-76 | uaauuucuacucuuguagauggaaagagaauuguuuucucc | 65% | 319 |
| 38595-41-77 | uaauuucuacucuuguagauggaaagagaauuguuuucucc | 16% | 320 |
| 38595-41-78 | uaauuucuacucuuguagauggaaagagaauuguuuucucc | 49% | 321 |
| 38595-41-79 | uaauuucuacucuuguagauggaaagagaauuguuuucucc | 70% | 322 |
| 38595-41-80 | uaauuucuacucuuguagauggaaagagaauuguuuucucc | 1% | 323 |
| 38595-41-81 | uaauuucuacucuuguagauggaaagagaauuguuuucucc | 13% | 324 |
| 38595-41-82 | uaauuucuacucuuguagauggaaagagaauuguuuucucc | 51% | 325 |
| 38595-41-83 | uaauuucuacucuuguagauggaaagagaauuguuuucucc | 64% | 326 |
| 38595-41-84 | uaauuucuacucuuguagauggaaagagaauuguuuucucc | 69% | 327 |
| 38595-41-85 | uaauuucuacucuuguagauggaaagagaauuguuuucucc | 69% | 328 |
| 38595-41-86 | u*a*a*uuucuacucuuguagauggaaagagaauuguuuuc*u*c*c | 61% | 329 |
| 38595-41-87 | +taauuucuacucuuguagauggaaagagaauuguuuucu+c+c | 60% | 330 |
| 38595-41-88 | uaauuucuacucuuguagauggaaagagaauuguuuucucc | 63% | 331 |
| 38595-41-89 | uaauuucuacucuuguagauggaaagagaauuguuuucucc | 34% | 332 |
| 38595-41-90 | uaauuucuacucuuguagauggaaagagaauuguuuucucc | 65% | 333 |
| 38595-41-91 | uaauuucuacucuuguagauggaaagagaauuguuuucucc | 66% | 334 |
| 38595-41-92 | uaauuucuacucuuguagauggaaagagaauuguuuucucc | 60% | 335 |
| 38595-41-93 | ZEN-uaauuucuacucuuguagauggaaagagaauuguuuucucc-ZEN | 61% | 336 |
| 38595-41-94 | ZEN-uaauuucuacucuuguagauggaaagagaauuguuuucucc-C3 | 59% | 337 |
| 38595-41-95 | C3-uaauuucuacucuuguagauggaaagagaauuguuuucucc-ZEN | 58% | 338 |
| 38104-41-96 | uaauuucuacucuuguagaucuuggguguguuaaaagugac | 63% | 339 |
| 38104-41-97 | C3-uaauuucuacucuuguagaucuuggguguguuaaaagugac-C3 | 63% | 340 |
| 38104-41-98 | uaauuucuacucuuguagaucuuggguguguuaaaagugac | 63% | 341 |
| 38104-41-99 | u*a*auuucuacucuuguagaucuuggguguguuaaaagu*g*a*c | 67% | 342 |

Oligonucleotide sequences are shown 5′-3′. Lowercase = RNA; Underlined lowercase = 2′-O-methyl RNA; Italics lowercase = 2′-fluoro RNA; +a, +c, +t, +g = LNA; C3 = C3 spacer (propanediol modifier); * = phosphorothioate internucleotide linkage; ZEN-napthyl-azo modifier. The relative functional activity of each species is indicated by the % cleavage in a T7EI heteroduplex assay. The sequence name indicates if the crRNA ia a 44 mer with a 24 base target domain or a 41 mer with a 21 base target domain and the HPRT target site is indicated (38104, 38351, or 38595).

### Example 7

Use of Modified crRNAs with AsCpf1 Protein Delivered as an RNP Complex.

A site in the human HPRT1 gene (38104) was chosen to study the ability to use chemically modified crRNAs with AsCpf1 protein to perform genome editing in HEK-293 cells using electroporation to deliver the ribonucleoprotein (RNP) complex into the cells.

Purified recombinant AsCpf1 protein was employed in this example, isolated from E. coli using standard techniques. The amino-acid sequence of the recombinant protein is shown in SEQ ID NO: 12.

The AsCpf1 crRNAs were heated to 95° C. for 5 minutes then allowed to cool to room temperature. The crRNAs were mixed with AsCpf1 protein at a molar ratio of 1.2:1 RNA:protein in phosphate buffered saline (PBS) (202 pmoles RNA with 168 pmoles protein in 6 μL volume, for a single transfection). The RNP complex was allowed to form at room temperature for 15 minutes. HEK293 cells were resuspended following trypsinization and washed in medium and washed a second time in PBS before use. Cells were resuspended in at a final concentration of 3.5×10⁵ cells in 20 μL of Nucleofection solution. 20 μL of cell suspension was placed in the V-bottom 96-well plate and 5 μL of the Cpf1 RNP complex was added to each well (5 μM final concentration) and 3 μL of Cpf1 Electroporation Enhancer Solution was added to each well (Integrated DNA Technologies). 25 μL of the final mixture was transferred to each well of a 96 well Nucleocuvette electroporation module. Cells were electroporated using Amaxa 96 well shuttle protocol, program 96-DS-150. Following electroporation, 75 μL of medium was added to each well and 25 μL of the final cell mixture was transferred to 175 μL of pre-warmed medium in 96 well incubation plates (final volume 200 μL). Cells were incubated at 37° C. for 48 hours. Genomic DNA was isolated using QuickExtract solution (Epicentre). Genomic DNA was amplified with KAPA HiFi DNA Polymerase (Roche) and primers targeting the HPRT region of interest (HPRT-low forward primer: AAGAATGTTGTGATAAAAGGTGATGCT (SEQ ID NO: 394); HPRT-low reverse primer: ACACATCCATGGGACTTCTGCCTC (SEQ ID NO: 395). PCR products were melted and re-annealed in NEB buffer 2 (New England Biolabs) to allow for heteroduplex formation followed by digestion with 2 units of T7 endonuclease 1 (T7EI; New England Biolabs) for 1 hour at 37° C. The digested products were visualized on a Fragment Analyzer (Advanced Analytical Technologies). Percent cleavage of targeted DNA was calculated as the average molar concentration of the cut products/(average molar concentration of the cut products+molar concentration of the uncut band)×100. Results are shown in Table 9 below. AsCpf1 crRNAs bearing low or high levels of modification, as shown below, are compatible with delivery via electroporation as an RNP complex to mediate genome editing in mammalian cells.

TABLE 9
Editing in mammalian cells using chemically-modified crRNAs
with recombinant AsCpf1 as RNP complexes

| | | % | |
|---|---|---|---|
| | | Cleavage | |
| | | T7E1 | SEQ |
| Seq Name | Sequence 5′-3′ | Assay | ID NO: |
| 38104-41-96 | uaauuucuacucuuguagaucuuggguguguuaaaagugac | 57% | 339 |
| 38104-41-97 | C3-uaauuucuacucuuguagaucuuggguguguuaaaagugac-C3 | 53% | 340 |
| 38104-41-98 | uaauuucuacucuuguagaucuuggguguguuaaaagugac | 42% | 341 |
| 38104-41-99 | u*a*auuucuacucuuguagaucuuggguguguuaaaagu*g*a*c | 43% | 342 |
| 38104-41-101 | u*a*auuucuacucuuguagaucuuggguguguuaaaagug*a*c | 43% | 343 |

Oligonucleotide sequences are shown 5′-3′. Lowercase = RNA; Underlined = 2′-O-methyl RNA; C3 = C3 spacer (propanediol modifier); * = phosphorothioate internucleotide linkage. The relative functional activity of each species is indicated by the % cleavage in a T7E1 heteroduplex assay. The sequence name indicates that the crRNAs are all 41 mers with a 21 base target domain.

### Example 8

Use of Modified crRNAs with an AsCpf1 Expression Plasmid in E. coli.

A site in the human HPRT1 gene (38346) was cloned onto an E. coli plasmid and was used to study the ability to use chemically modified crRNAs to perform site-specific cleavage in E. coli cells. AsCpf1 was expressed from a plasmid. Electroporation was used to deliver both the AsCpf1 expression plasmid and chemically-synthesized crRNAs.

The AsCpf1 protein was expressed from a plasmid in this example, using a phage T7 promoter and standard E. coli translation elements. The amino-acid sequence of the expression construct is shown in SEQ ID NO:16).

The AsCpf1 crRNAs were heated to 95° C. for 5 minutes then allowed to cool to room temperature. The crRNAs and AsCpf1 plasmid were mixed in TE (60 femtomoles AsCpf1 plasmid with 400 pmoles RNA in 5 μL volume, for a single transformation), and added directly to 20 μL of competent E. coli cells). A bacterial strain where survival is linked to successful cleavage by Cpf1 was made competent by growing cells to mid-log phase, washing 3 times in ice cold 10% glycerol, and final suspension in 1:100^{th} volume 10% glycerol. Electroporations were performed by adding the 25 μL transformation mixture to a pre-chilled 0.1 cm electroporation cuvette and pulsing 1.8 kV exponential decay. Following electroporation, 980 μL of SOB medium was added to the electroporation cuvette with mixing and the resulting cell suspension was transferred to a sterile 15 ml culture tube. Cells were incubated with shaking (250 rpm) at 37° C. for 1.5 hours, at which time IPTG was added (1 mM) followed by further shaking incubation at 37° C. for 1 hour. Following incubation cells were plated on selective media to assess survival.

This example demonstrates that chemically-modified synthetic crRNAs can be used with Cpf1 for gene editing in bacteria. However, high efficiency is only seen using RNAs that have been more extensively modified with exonuclease-blocking PS internucleotide linkages. The modification patterns that work best in bacterial cells perform poorly in mammalian cells (Table 10).

TABLE 10
Chemically-modified crRNAs compatible with Cpf1 function in bacteria

| | | % | % | SEQ |
|---|---|---|---|---|
| | | Cleavage | Cleavage | ID |
| Seq Name | Sequence 5′-3′ | Human | Bacteria | NO: |
| 38346-41-1 | uaauuucuacucuuguagauacauaaaacucuuuuagguua | 21% | 0% | 344 |
| 38346-41-2 | u*a*a*uuucuacucuuguagauacauaaaacucuuuuagguua | 17% | 0% | 345 |
| 38346-41-3 | u*a*a*u*u*u*cuacucuuguagauacauaaaacucuuuuagguua | 10% | 2% | 346 |
| 38346-41-4 | uaauuucuacucuuguagauacauaaaacucuuuu*a*g*g*u*u*a | 14% | 18% | 347 |
| 38346-41-5 | u*a*a*uuucuacucuuguagauacauaaaacucuuuuagg*u*u*a | 8% | 5% | 348 |
| 38346-41-6 | u*a*a*uuucuacucuuguagauacauaaaacucuuuu*a*g*g*u*u*a | 5% | 40% | 349 |
| 38346-41-7 | u*a*a*u*u*u*cuacucuuguagauacauaaaacucuuuu*a*g*g*u*u*a | 2% | 88% | 350 |
| 38346-41-8 | uaauuucuacucuuguagauacauaaaacucuuuuagg*u*u*a | 14% | 7% | 351 |
| 38346-41-9 | uaauuucuacucuuguagauacauaaaacucuuuu*a*g*g*u*u*a | 8% | 35% | 352 |
| 38346-41-10 | u*a*a*uuucuacucuuguagauacauaaaacucuuuuagg*u*u*a | 12% | 27% | 353 |
| 38346-41-11 | u*a*a*uuucuacucuuguagauacauaaaacucuuuuag*g*u*u*a | 8% | 85% | 354 |
| 38346-41-12 | u*a*a*uuucuacucuuguagauacauaaaacucuuuua*g*g*u*u*a | 5% | 92% | 355 |
| 38346-41-13 | u*a*a*uuucuacucuuguagauacauaaaacucuuuu*a*g*g*u*u*a | 4% | 100% | 356 |
| 38346-41-14 | u*a*a*u*u*u*cuacucuuguagauacauaaaacucuuuu*a*g*g*u*u*a | 1% | 90% | 357 |

Oligonucleotide sequences are shown 5′-3′. Lowercase = RNA; Underlined lowercase = 2′-O-methyl RNA; C3 = C3 spacer (propanediol modifier); * = phosphorothioate internucleotide linkage. The relative functional activity in human cells is indicated by the % cleavage in a T7E1 heteroduplex
assay, and in bacteria is indicated by % survival in a Cpf1 reporter strain. The sequence name indicates that the crRNAs are all 41 mers with a 21 base target domain.

### Example 9

### DNA and Amino Acid Sequences of Wild Type Lb Cpf1 Polypeptide, as Encoded in Isolated Nucleic Acid Vectors

The list below shows wild type (WT) Lb Cpf1 nucleases expressed as polypeptide fusion proteins as described in the present invention. It will be appreciated by one with skill in the art that many different DNA sequences can encode/express the same amino acid (AA) sequence since in many cases more than one codon can encode for the same amino acid. The DNA sequences shown below only serve as examples, and other DNA sequences that encode the same protein (e.g., same amino acid sequence) are contemplated. It is further appreciated that additional features, elements or tags may be added to said sequences, such as NLS domains and the like.

Examples are shown for WT LbCpf1 showing amino acid and DNA sequences for those proteins as LbCpf1 alone and LbCpf1 fused to an N-terminal V5-tag, an N-terminal SV40 NLS domain, a C-terminal SV40 NLS domain, and a C-terminal 6×His-tag (SEQ ID NO: 404).

LbCpf1 Native DNA Sequence
SEQ ID NO: 3
ATGAGCAAACTGGAAAAATTTACGAATTGTTATAGCCTGTCCAAGACCCTGCGTTTCAAAGCCATCC
CCGTTGGCAAAACCCAGGAGAATATTGATAATAAACGTCTGCTGGTTGAGGATGAAAAAAGAGCAGA
AGACTATAAGGGAGTCAAAAAACTGCTGGATCGGTACTACCTGAGCTTTATAAATGACGTGCTGCAT
AGCATTAAACTGAAAAATCTGAATAACTATATTAGTCTGTTCCGCAAGAAAACCCGAACAGAGAAAG
AAAATAAAGAGCTGGAAAACCTGGAGATCAATCTGCGTAAAGAGATCGCAAAAGCTTTTAAAGGAAA
TGAAGGTTATAAAAGCCTGTTCAAAAAAGACATTATTGAAACCATCCTGCCGGAATTTCTGGATGAT
AAAGACGAGATAGCGCTCGTGAACAGCTTCAACGGGTTCACGACCGCCTTCACGGGCTTTTTCGATA
ACAGGGAAAATATGTTTTCAGAGGAAGCCAAAAGCACCTCGATAGCGTTCCGTTGCATTAATGAAAA
TTTGACAAGATATATCAGCAACATGGATATTTTCGAGAAAGTTGATGCGATCTTTGACAAACATGAA
GTGCAGGAGATTAAGGAAAAAATTCTGAACAGCGATTATGATGTTGAGGATTTTTTCGAGGGGGAAT
TTTTTAACTTTGTACTGACACAGGAAGGTATAGATGTGTATAATGCTATTATCGGCGGGTTCGTTAC
CGAATCCGGCGAGAAAATTAAGGGTCTGAATGAGTACATCAATCTGTATAACCAAAAGACCAAACAG
AAACTGCCAAAATTCAAACCGCTGTACAAGCAAGTCCTGAGCGATCGGGAAAGCTTGAGCTTTTACG
GTGAAGGTTATACCAGCGACGAGGAGGTACTGGAGGTCTTTCGCAATACCCTGAACAAGAACAGCGA
AATTTTCAGCTCCATTAAAAAGCTGGAGAAACTGTTTAAGAATTTTGACGAGTACAGCAGCGCAGGT
ATTTTTGTGAAGAACGGACCTGCCATAAGCACCATTAGCAAGGATATTTTTGGAGAGTGGAATGTTA
TCCGTGATAAATGGAACGCGGAATATGATGACATACACCTGAAAAAGAAGGCTGTGGTAACTGAGAA
ATATGAAGACGATCGCCGCAAAAGCTTTAAAAAAATCGGCAGCTTTAGCCTGGAGCAGCTGCAGGAA
TATGCGGACGCCGACCTGAGCGTGGTCGAGAAACTGAAGGAAATTATTATCCAAAAAGTGGATGAGA
TTTACAAGGTATATGGTAGCAGCGAAAAACTGTTTGATGCGGACTTCGTTCTGGAAAAAAGCCTGAA
AAAAAATGATGCTGTTGTTGCGATCATGAAAGACCTGCTCGATAGCGTTAAGAGCTTTGAAAATTAC
ATTAAAGCATTCTTTGGCGAGGGCAAAGAAACAAACAGAGACGAAAGCTTTTATGGCGACTTCGTCC
TGGCTTATGACATCCTGTTGAAGGTAGATCATATATATGATGCAATTCGTAATTACGTAACCCAAAA
GCCGTACAGCAAAGATAAGTTCAAACTGTATTTCCAGAACCCGCAGTTTATGGGTGGCTGGGACAAA
GACAAGGAGACAGACTATCGCGCCACTATTCTGCGTTACGGCAGCAAGTACTATCTCGCCATCATGG
ACAAAAAATATGCAAAGTGTCTGCAGAAAATCGATAAAGACGACGTGAACGGAAATTACGAAAAGAT
TAATTATAAGCTGCTGCCAGGGCCCAACAAGATGTTACCGAAAGTATTTTTTTCCAAAAAATGGATG
GCATACTATAACCCGAGCGAGGATATACAGAAGATTTACAAAAATGGGACCTTCAAAAAGGGGGATA
TGTTCAATCTGAATGACTGCCACAAACTGATCGATTTTTTTAAAGATAGCATCAGCCGTTATCCTAA
ATGGTCAAACGCGTATGATTTTAATTTCTCCGAAACGGAGAAATATAAAGACATTGCTGGTTTCTAT
CGCGAAGTCGAAGAACAGGGTTATAAAGTTAGCTTTGAATCGGCCAGCAAGAAAGAGGTTGATAAAC
TGGTGGAGGAGGGTAAGCTGTATATGTTTCAGATTTATAACAAAGACTTTAGCGACAAAAGCCACGG
TACTCCTAATCTGCATACGATGTACTTTAAACTGCTGTTTGATGAGAATAACCACGGCCAAATCCGT
CTCTCCGGTGGAGCAGAACTTTTTATGCGGCGTGCGAGCCTAAAAAAGGAAGAACTGGTGGTGCATC
CCGCCAACAGCCCGATTGCTAACAAAAATCCAGATAATCCTAAGAAGACCACCACACTGTCGTACGA
TGTCTATAAGGATAAACGTTTCTCGGAAGACCAGTATGAATTGCATATACCGATAGCAATTAATAAA
TGCCCAAAAAACATTTTCAAAATCAACACTGAAGTTCGTGTGCTGCTGAAACATGATGATAATCCGT
ATGTGATCGGAATTGACCGTGGGGAGAGAAATCTGCTGTATATTGTAGTCGTTGATGGCAAGGGCAA
CATCGTTGAGCAGTATAGCCTGAATGAAATAATTAATAATTTTAACGGTATACGTATTAAAACCGAC
TATCATAGCCTGCTGGATAAAAAGGAGAAAGAGCGTTTTGAGGCACGCCAAAATTGGACGAGCATCG
AAAACATCAAGGAACTGAAGGCAGGATATATCAGCCAAGTAGTCCATAAAATCTGTGAACTGGTGGA
GAAGTACGACGCTGTCATTGCCCTGGAAGACCTCAATAGCGGCTTTAAAAACAGCCGGGTGAAGGTG
GAGAAACAGGTATACCAAAAGTTTGAAAAGATGCTCATTGATAAGCTGAACTATATGGTTGATAAAA
AGAGCAACCCGTGCGCCACTGGCGGTGCACTGAAAGGGTACCAAATTACCAATAAATTTGAAAGCTT
TAAAAGCATGAGCACGCAGAATGGGTTTATTTTTTATATACCAGCATGGCTGACGAGCAAGATTGAC
CCCAGCACTGGTTTTGTCAATCTGCTGAAAACCAAATACACAAGCATTGCGGATAGCAAAAAATTTA
TTTCGAGCTTCGACCGTATTATGTATGTTCCGGAGGAAGATCTGTTTGAATTTGCCCTGGATTATAA
AAACTTCAGCCGCACCGATGCAGATTATATCAAAAAATGGAAGCTGTACAGTTATGGTAATCGTATA
CGTATCTTCCGTAATCCGAAGAAAAACAATGTGTTCGATTGGGAAGAGGTCTGTCTGACCAGCGCGT
ATAAAGAACTGTTCAACAAGTACGGAATAAATTATCAGCAAGGTGACATTCGCGCACTGCTGTGTGA
ACAGTCAGATAAAGCATTTTATAGCAGCTTTATGGCGCTGATGAGCCTGATGCTCCAGATGCGCAAC
AGCATAACCGGTCGCACAGATGTTGACTTTCTGATCAGCCCTGTGAAGAATAGCGACGGCATCTTCT
ACGATTCCAGGAACTATGAAGCACAGGAAAACGCTATTCTGCCTAAAAATGCCGATGCCAACGGCGC
CTATAATATTGCACGGAAGGTTCTGTGGGCGATTGGACAGTTCAAGAAAGCGGAAGATGAGAAGCTG
GATAAGGTAAAAATTGCTATTAGCAATAAGGAATGGCTGGAGTACGCACAGACATCGGTTAAACACG
CGGCCGCTTCCCTGCAGGTAATTAAATAA
LbCpf1 Native Protein Sequence
SEQ ID NO: 4
MLKNVGIDRLDVEKGRKNMSKLEKFINCYSLSKTLRFKAIPVGKTQENIDNKRLLVEDEKRAEDYKG
VKKLLDRYYLSFINDVLHSIKLKNLNNYISLFRKKIRTEKENKELENLEINLRKEIAKAFKGNEGYK
SLFKKDIIETILPEFLDDKDEIALVNSFNGFITAFTGFFDNRENMFSEEAKSTSIAFRCINENLTRY
ISNMDIFEKVDAIFDKHEVQEIKEKILNSDYDVEDFFEGEFFNFVLIQEGIDVYNAIIGGFVTESGE
KIKGLNEYINLYNQKTKQKLPKFKPLYKQVLSDRESLSFYGEGYISDEEVLEVERNTLNKNSEIFSS
IKKLEKLFKNFDEYSSAGIFVKNGPAISTISKDIFGEWNVIRDKWNAEYDDIHLKKKAVVIEKYEDD
RRKSFKKIGSFSLEQLQEYADADLSVVEKLKEIIIQKVDEIYKVYGSSEKLFDADFVLEKSLKKNDA
VVAIMKDLLDSVKSFENYIKAFFGEGKETNRDESFYGDFVLAYDILLKVDHIYDAIRNYVTQKPYSK
DKFKLYFQNPQFMGGWDKDKETDYRATILRYGSKYYLAIMDKKYAKCLQKIDKDDVNGNYEKINYKL
LPGPNKMLPKVFFSKKWMAYYNPSEDIQKIYKNGTFKKGDMFNLNDCHKLIDFFKDSISRYPKWSNA
YDFNFSETEKYKDIAGFYREVEEQGYKVSFESASKKEVDKLVEEGKLYMFQIYNKDFSDKSHGTPNL
HIMYFKLLFDENNHGQIRLSGGAELFMRRASLKKEELVVHPANSPIANKNPDNPKKITTLSYDVYKD
KRFSEDQYELHIPIAINKCPKNIFKINTEVRVLLKHDDNPYVIGIDRGERNLLYIVVVDGKGNIVEQ
YSLNEIINNFNGIRIKTDYHSLLDKKEKERFEARQNWTSIENIKELKAGYISQVVHKICELVEKYDA
VIALEDLNSGFKNSRVKVEKQVYQKFEKMLIDKLNYMVDKKSNPCATGGALKGYQIINKFESFKSMS
TQNGFIFYIPAWLISKIDPSTGFVNLLKIKYTSIADSKKFISSFDRIMYVPEEDLFEFALDYKNFSR
IDADYIKKWKLYSYGNRIRIFRNPKKNNVFDWEEVCLISAYKELFNKYGINYQQGDIRALLCEQSDK
AFYSSFMALMSLMLQMRNSITGRIDVDFLISPVKNSDGIFYDSRNYEAQENAILPKNADANGAYNIA
RKVLWAIGQFKKAEDEKLDKVKIAISNKEWLEYAQTSVKH
E. coli optimized Lb Cpf1 DNA
SEQ ID NO: 6
ATGCTGAAAAACGTGGGTATTGATCGTCTGGATGTTGAAAAAGGTCGCAAAAATATGAGCAAACTGG
AAAAGTTCACCAACTGTTATAGCCTGAGCAAAACCCTGCGTTTTAAAGCAATTCCGGTTGGTAAAAC
CCAAGAGAACATTGATAATAAACGCCTGCTGGTCGAAGATGAAAAACGCGCTGAAGATTATAAAGGC
GTGAAAAAACTGCTGGATCGCTATTATCTGAGCTTCATTAACGATGTGCTGCACAGCATTAAACTGA
AGAACCTGAACAACTATATCAGCCTGTTTCGTAAAAAAACCCGCACCGAAAAAGAAAACAAAGAGCT
GGAAAACCTGGAAATCAATCTGCGTAAAGAAATCGCCAAAGCGTTTAAAGGTAACGAGGGTTATAAA
AGCCTGTTCAAGAAAGACATCATCGAAACCATTCTGCCGGAATTTCTGGATGATAAAGATGAAATTG
CCCTGGTGAATAGCTTTAATGGCTTTACCACCGCATTTACCGGCTTTTTTGATAATCGCGAAAACAT
GTTCAGCGAAGAAGCAAAAAGCACCAGCATTGCATTTCGCTGCATTAATGAAAATCTGACCCGCTAC
ATTAGCAACATGGATATCTTTGAAAAAGTGGACGCGATCTTCGATAAACACGAAGTGCAAGAGATCA
AAGAGAAAATCCTGAACAGCGATTATGACGTCGAAGATTTTTTTGAAGGCGAGTTCTTTAACTTCGT
TCTGACCCAAGAAGGTATCGACGTTTATAACGCAATTATTGGTGGTTTTGTTACCGAAAGCGGTGAG
AAAATCAAAGGCCTGAATGAATATATCAACCTGTATAACCAGAAAACCAAACAGAAACTGCCGAAAT
TCAAACCGCTGTATAAACAGGTTCTGAGCGATCGTGAAAGCCTGAGCTTTTATGGTGAAGGTTATAC
CAGTGATGAAGAGGTTCTGGAAGTTTTTCGTAACACCCTGAATAAAAACAGCGAGATCTTTAGCAGC
ATCAAAAAGCTTGAGAAACTGTTCAAAAACTTTGATGAGTATAGCAGCGCAGGCATCTTTGTTAAAA
ATGGTCCGGCAATTAGCACCATCAGCAAAGATATTTTTGGCGAATGGAATGTGATCCGCGATAAATG
GAATGCCGAATATGATGATATCCACCTGAAAAAAAAGGCCGTGGTGACCGAGAAATATGAAGATGAT
CGTCGTAAAAGCTTCAAGAAAATTGGTAGCTTTAGCCTGGAACAGCTGCAAGAATATGCAGATGCAG
ATCTGAGCGTTGTGGAAAAACTGAAAGAAATCATCATTCAGAAGGTGGACGAGATCTATAAAGTTTA
TGGTAGCAGCGAAAAACTGTTCGATGCAGATTTTGTTCTGGAAAAAAGCCTGAAAAAGAATGATGCC
GTTGTGGCCATTATGAAAGATCTGCTGGATAGCGTTAAGAGCTTCGAGAATTACATCAAAGCCTTTT
TTGGTGAGGGCAAAGAAACCAATCGTGATGAAAGTTTCTATGGCGATTTTGTGCTGGCCTATGATAT
TCTGCTGAAAGTGGACCATATTTATGATGCCATTCGCAATTATGTTACCCAGAAACCGTATAGCAAA
GACAAGTTCAAACTGTACTTTCAGAACCCGCAGTTTATGGGTGGTTGGGATAAAGATAAAGAAACCG
ATTATCGTGCCACCATCCTGCGTTATGGTAGTAAATACTATCTGGCCATCATGGACAAAAAATACGC
AAAATGCCTGCAGAAAATCGACAAAGATGATGTGAATGGCAACTATGAAAAAATCAACTACAAACTG
CTGCCTGGTCCGAATAAAATGCTGCCGAAAGTGTTCTTTAGCAAGAAATGGATGGCCTATTATAACC
CGAGCGAGGATATTCAAAAGATCTACAAAAATGGCACCTTTAAAAAGGGCGACATGTTCAATCTGAA
CGATTGCCACAAACTGATCGATTTCTTCAAAGATTCAATTTCGCGTTATCCGAAATGGTCCAATGCC
TATGATTTTAACTTTAGCGAAACCGAAAAATACAAAGACATTGCCGGTTTTTATCGCGAAGTGGAAG
AACAGGGCTATAAAGTGAGCTTTGAAAGCGCAAGCAAAAAAGAGGTTGATAAGCTGGTTGAAGAGGG
CAAACTGTATATGTTCCAGATTTACAACAAAGATTTTAGCGACAAAAGCCATGGCACCCCGAATCTG
CATACCATGTACTTTAAACTGCTGTTCGACGAAAATAACCATGGTCAGATTCGTCTGAGCGGTGGTG
CCGAACTGTTTATGCGTCGTGCAAGTCTGAAAAAAGAAGAACTGGTTGTTCATCCGGCAAATAGCCC
GATTGCAAACAAAAATCCGGACAATCCGAAAAAAACCACGACACTGAGCTATGATGTGTATAAAGAC
AAACGTTTTAGCGAGGATCAGTATGAACTGCATATCCCGATTGCCATCAATAAATGCCCGAAAAACA
TCTTTAAGATCAACACCGAAGTTCGCGTGCTGCTGAAACATGATGATAATCCGTATGTGATTGGCAT
TGATCGTGGTGAACGTAACCTGCTGTATATTGTTGTTGTTGATGGTAAAGGCAACATCGTGGAACAG
TATAGTCTGAACGAAATTATCAACAACTTTAACGGCATCCGCATCAAAACCGACTATCATAGCCTGC
TGGACAAGAAAGAAAAAGAACGTTTTGAAGCACGTCAGAACTGGACCAGTATTGAAAACATCAAAGA
ACTGAAAGCCGGTTATATTAGCCAGGTGGTTCATAAAATCTGTGAGCTGGTAGAAAAATACGATGCA
GTTATTGCACTGGAAGATCTGAATAGCGGTTTCAAAAATAGCCGTGTGAAAGTCGAAAAACAGGTGT
ATCAGAAATTCGAGAAAATGCTGATCGACAAACTGAACTACATGGTCGACAAAAAAAGCAATCCGTG
TGCAACCGGTGGTGCACTGAAAGGTTATCAGATTACCAACAAATTTGAAAGCTTTAAAAGCATGAGC
ACCCAGAACGGCTTTATCTTCTATATTCCGGCATGGCTGACCAGCAAAATTGATCCGAGCACCGGTT
TTGTGAACCTGCTGAAAACAAAATATACCTCCATTGCCGACAGCAAGAAGTTTATTAGCAGCTTTGA
TCGCATTATGTATGTTCCGGAAGAGGACCTGTTTGAATTCGCACTGGATTACAAAAATTTCAGCCGT
ACCGATGCCGACTACATCAAAAAATGGAAACTGTACAGCTATGGTAACCGCATTCGCATTTTTCGCA
ACCCGAAGAAAAACAATGTGTTCGATTGGGAAGAAGTTTGTCTGACCAGCGCATATAAAGAACTTTT
CAACAAATACGGCATCAACTATCAGCAGGGTGATATTCGTGCACTGCTGTGTGAACAGAGCGATAAA
GCGTTTTATAGCAGTTTTATGGCACTGATGAGCCTGATGCTGCAGATGCGTAATAGCATTACCGGTC
GCACCGATGTGGATTTTCTGATTAGTCCGGTGAAAAATTCCGATGGCATCTTTTATGATAGCCGCAA
TTACGAAGCACAAGAAAATGCAATTCTGCCGAAAAACGCAGATGCAAATGGTGCATATAACATTGCA
CGTAAAGTTCTGTGGGCAATTGGCCAGTTTAAGAAAGCAGAAGATGAGAAGCTGGACAAAGTGAAAA
TTGCGATCAGCAATAAAGAGTGGCTGGAATACGCACAGACCAGCGTTAAACATTGA
E. coli optimized Lb Cpf1 AA
SEQ ID NO: 7
MLKNVGIDRLDVEKGRKNMSKLEKFTNCYSLSKTLRFKAIPVGKTQENIDNKRLLVEDEKRAEDYKG
VKKLLDRYYLSFINDVLHSIKLKNLNNYISLFRKKTRTEKENKELENLEINLRKEIAKAFKGNEGYK
SLFKKDIIETILPEFLDDKDEIALVNSFNGFTTAFTGFFDNRENMFSEEAKSTSIAFRCINENLTRY
ISNMDIFEKVDAIFDKHEVQEIKEKILNSDYDVEDFFEGEFFNFVLTQEGIDVYNAIIGGFVTESGE
KIKGLNEYINLYNQKTKQKLPKFKPLYKQVLSDRESLSFYGEGYTSDEEVLEVERNTLNKNSEIFSS
IKKLEKLFKNFDEYSSAGIFVKNGPAISTISKDIFGEWNVIRDKWNAEYDDIHLKKKAVVTEKYEDD
RRKSFKKIGSFSLEQLQEYADADLSVVEKLKEIIIQKVDEIYKVYGSSEKLFDADFVLEKSLKKNDA
VVAIMKDLLDSVKSFENYIKAFFGEGKETNRDESFYGDFVLAYDILLKVDHIYDAIRNYVTQKPYSK
DKFKLYFQNPQFMGGWDKDKETDYRATILRYGSKYYLAIMDKKYAKCLQKIDKDDVNGNYEKINYKL
LPGPNKMLPKVFFSKKWMAYYNPSEDIQKIYKNGTFKKGDMFNLNDCHKLIDFFKDSISRYPKWSNA
YDFNFSETEKYKDIAGFYREVEEQGYKVSFESASKKEVDKLVEEGKLYMFQTYNKDFSDKSHGTPNL
HTMYFKLLFDENNHGQIRLSGGAELFMRRASLKKEELVVHPANSPIANKNPDNPKKTTTLSYDVYKD
KRFSEDQYELHIPIAINKCPKNIFKINTEVRVLLKHDDNPYVIGIDRGERNLLYIVVVDGKGNIVEQ
YSLNEIINNFNGIRIKTDYHSLLDKKEKERFEARQNWTSIENIKELKAGYISQVVHKICELVEKYDA
VIALEDLNSGFKNSRVKVEKQVYQKFEKMLIDKLNYMVDKKSNPCATGGALKGYQITNKFESFKSMS
TQNGFIFYIPAWLTSKIDPSTGFVNLLKTKYTSIADSKKFISSFDRIMYVPEEDLFEFALDYKNFSR
TDADYIKKWKLYSYGNRIRIFRNPKKNNVFDWEEVCLTSAYKELFNKYGINYQQGDIRALLCEQSDK
AFYSSFMALMSLMLQMRNSITGRTDVDFLISPVKNSDGIFYDSRNYEAQENAILPKNADANGAYNIA
RKVLWAIGQFKKAEDEKLDKVKIAISNKEWLEYAQTSVKH
Hs optimized Lb Cpf1 DNA
SEQ ID NO: 9
ATGCTGAAGAACGTGGGCATCGACCGGCTGGACGTGGAAAAGGGCAGAAAGAACATGAGCAAGCTCG
AGAAGTTCACCAACTGCTACAGCCTGAGCAAGACCCTGCGGTTCAAGGCCATTCCTGTGGGCAAGAC
CCAAGAGAACATCGACAACAAGCGGCTGCTGGTGGAAGATGAGAAGAGAGCCGAGGACTACAAGGGC
GTGAAGAAGCTGCTGGACCGGTACTACCTGAGCTTCATCAACGACGTGCTGCACAGCATCAAGCTCA
AGAACCTGAACAACTACATCAGCCTGTTCCGGAAGAAAACCCGGACCGAGAAAGAGAACAAAGAGCT
GGAAAACCTCGAGATCAACCTGCGGAAAGAGATCGCCAAGGCCTTCAAGGGCAACGAGGGCTACAAG
AGCCTGTTCAAGAAGGACATCATCGAGACAATCCTGCCTGAGTTCCTGGACGACAAGGACGAGATCG
CCCTGGTCAACAGCTTCAACGGCTTCACAACCGCCTTCACCGGCTTTTTCGACAACCGCGAGAATAT
GTTCAGCGAGGAAGCCAAGAGCACCTCTATCGCCTTCCGGTGCATCAACGAGAATCTGACCCGGTAC
ATCAGCAACATGGATATCTTCGAGAAGGTGGACGCCATCTTCGACAAGCACGAGGTGCAAGAGATCA
AAGAAAAGATCCTGAACAGCGACTACGACGTCGAGGACTTCTTCGAGGGCGAGTTCTTCAACTTCGT
GCTGACACAAGAGGGCATCGATGTGTACAACGCCATCATCGGCGGCTTCGTGACAGAGAGCGGCGAG
AAGATCAAGGGCCTGAACGAGTACATCAACCTCTACAACCAGAAAACGAAGCAGAAGCTGCCCAAGT
TCAAGCCCCTGTACAAACAGGTGCTGAGCGACAGAGAGAGCCTGTCCTTTTACGGCGAGGGCTATAC
CAGCGACGAAGAGGTGCTGGAAGTGTTCAGAAACACCCTGAACAAGAACAGCGAGATCTTCAGCTCC
ATCAAGAAGCTCGAAAAGCTGTTTAAGAACTTCGACGAGTACAGCAGCGCCGGCATCTTCGTGAAGA
ATGGCCCTGCCATCAGCACCATCTCCAAGGACATCTTCGGCGAGTGGAACGTGATCCGGGACAAGTG
GAACGCCGAGTACGACGACATCCACCTGAAGAAAAAGGCCGTGGTCACCGAGAAGTACGAGGACGAC
AGAAGAAAGAGCTTCAAGAAGATCGGCAGCTTCAGCCTGGAACAGCTGCAAGAGTACGCCGACGCCG
ATCTGAGCGTGGTGGAAAAGCTGAAAGAGATTATCATCCAGAAGGTCGACGAGATCTACAAGGTGTA
CGGCAGCAGCGAGAAGCTGTTCGACGCCGACTTTGTGCTGGAAAAGAGCCTCAAAAAGAACGACGCC
GTGGTGGCCATCATGAAGGACCTGCTGGATAGCGTGAAGTCCTTCGAGAACTATATTAAGGCCTTCT
TTGGCGAGGGCAAAGAGACAAACCGGGACGAGAGCTTCTACGGCGATTTCGTGCTGGCCTACGACAT
CCTGCTGAAAGTGGACCACATCTACGACGCCATCCGGAACTACGTGACCCAGAAGCCTTACAGCAAG
GACAAGTTTAAGCTGTACTTCCAGAATCCGCAGTTCATGGGCGGCTGGGACAAAGACAAAGAAACCG
ACTACCGGGCCACCATCCTGAGATACGGCTCCAAGTACTATCTGGCCATTATGGACAAGAAATACGC
CAAGTGCCTGCAGAAGATCGATAAGGACGACGTGAACGGCAACTACGAGAAGATTAACTACAAGCTG
CTGCCCGGACCTAACAAGATGCTGCCTAAGGTGTTCTTTAGCAAGAAATGGATGGCCTACTACAACC
CCAGCGAGGATATCCAGAAAATCTACAAGAACGGCACCTTCAAGAAAGGCGACATGTTCAACCTGAA
CGACTGCCACAAGCTGATCGATTTCTTCAAGGACAGCATCAGCAGATACCCCAAGTGGTCCAACGCC
TACGACTTCAATTTCAGCGAGACAGAGAAGTATAAGGATATCGCCGGGTTCTACCGCGAGGTGGAAG
AACAGGGCTATAAGGTGTCCTTTGAGAGCGCCAGCAAGAAAGAGGTGGACAAGCTGGTCGAAGAGGG
CAAGCTGTACATGTTCCAGATCTATAACAAGGACTTCTCCGACAAGAGCCACGGCACCCCTAACCTG
CACACCATGTACTTTAAGCTGCTGTTCGATGAGAACAACCACGGCCAGATCAGACTGTCTGGCGGAG
CCGAGCTGTTTATGAGAAGGGCCAGCCTGAAAAAAGAGGAACTGGTCGTTCACCCCGCCAACTCTCC
AATCGCCAACAAGAACCCCGACAATCCCAAGAAAACCACCACACTGAGCTACGACGTGTACAAGGAT
AAGCGGTTCTCCGAGGACCAGTACGAGCTGCACATCCCTATCGCCATCAACAAGTGCCCCAAGAATA
TCTTCAAGATCAACACCGAAGTGCGGGTGCTGCTGAAGCACGACGACAACCCTTACGTGATCGGCAT
CGACAGAGGCGAGCGGAACCTGCTGTATATCGTGGTGGTGGACGGCAAGGGCAATATCGTGGAACAG
TACTCCCTGAATGAGATCATCAACAACTTCAATGGCATCCGGATCAAGACGGACTACCACAGCCTGC
TGGACAAAAAAGAGAAAGAACGCTTCGAGGCCCGGCAGAACTGGACCAGCATCGAGAACATCAAAGA
ACTGAAGGCCGGCTACATCTCCCAGGTGGTGCACAAGATCTGCGAGCTGGTTGAGAAGTATGACGCC
GTGATTGCCCTGGAAGATCTGAATAGCGGCTTTAAGAACAGCCGCGTGAAGGTCGAGAAACAGGTGT
ACCAGAAATTCGAGAAGATGCTGATCGACAAGCTGAACTACATGGTCGACAAGAAGTCTAACCCCTG
CGCCACAGGCGGAGCCCTGAAGGGATATCAGATCACCAACAAGTTCGAGTCCTTCAAGAGCATGAGC
ACCCAGAATGGCTTCATCTTCTACATCCCCGCCTGGCTGACCAGCAAGATCGATCCTAGCACCGGAT
TCGTGAACCTGCTCAAGACCAAGTACACCAGCATTGCCGACAGCAAGAAGTTCATCTCCAGCTTCGA
CCGGATTATGTACGTGCCCGAAGAGGACCTGTTCGAATTCGCCCTGGATTACAAGAACTTCAGCCGG
ACCGATGCCGACTATATCAAGAAGTGGAAGCTGTATAGCTACGGCAACCGCATCCGCATCTTCAGAA
ACCCGAAGAAAAACAACGTGTTCGACTGGGAAGAAGTGTGCCTGACCAGCGCCTACAAAGAACTCTT
CAACAAATACGGCATCAACTACCAGCAGGGCGACATCAGAGCCCTGCTGTGCGAGCAGAGCGACAAG
GCCTTTTACAGCTCCTTCATGGCCCTGATGTCCCTGATGCTGCAGATGCGGAATAGCATCACCGGCA
GGACCGACGTGGACTTCCTGATCAGCCCTGTGAAGAATTCCGACGGGATCTTCTACGACAGCAGAAA
CTACGAGGCTCAAGAGAACGCCATCCTGCCTAAGAACGCCGATGCCAACGGCGCCTATAATATCGCC
AGAAAGGTGCTGTGGGCCATCGGCCAGTTTAAGAAGGCCGAGGACGAGAAACTGGACAAAGTGAAGA
TCGCCATCTCTAACAAAGAGTGGCTGGAATACGCCCAGACCAGCGTGAAACAC
Hs optimized Lb Cpf1 AA
SEQ ID NO: 10
MLKNVGIDRLDVEKGRKNMSKLEKFTNCYSLSKTLRFKAIPVGKTQENIDNKRLLVEDEKRAEDYKG
VKKLLDRYYLSFINDVLHSIKLKNLNNYISLFRKKTRTEKENKELENLEINLRKEIAKAFKGNEGYK
SLFKKDIIETILPEFLDDKDEIALVNSFNGFTTAFTGFFDNRENMFSEEAKSTSIAFRCINENLTRY
ISNMDIFEKVDAIFDKHEVQEIKEKILNSDYDVEDFFEGEFFNFVLTQEGIDVYNAIIGGFVTESGE
KIKGLNEYINLYNQKTKQKLPKFKPLYKQVLSDRESLSFYGEGYTSDEEVLEVERNTLNKNSEIFSS
IKKLEKLFKNFDEYSSAGIFVKNGPAISTISKDIFGEWNVIRDKWNAEYDDIHLKKKAVVTEKYEDD
RRKSFKKIGSFSLEQLQEYADADLSVVEKLKEIIIQKVDEIYKVYGSSEKLFDADFVLEKSLKKNDA
VVAIMKDLLDSVKSFENYIKAFFGEGKETNRDESFYGDFVLAYDILLKVDHIYDAIRNYVTQKPYSK
DKFKLYFQNPQFMGGWDKDKETDYRATILRYGSKYYLAIMDKKYAKCLQKIDKDDVNGNYEKINYKL
LPGPNKMLPKVFFSKKWMAYYNPSEDIQKIYKNGTFKKGDMFNLNDCHKLIDFFKDSISRYPKWSNA
YDFNFSETEKYKDIAGFYREVEEQGYKVSFESASKKEVDKLVEEGKLYMFQTYNKDFSDKSHGTPNL
HTMYFKLLFDENNHGQIRLSGGAELFMRRASLKKEELVVHPANSPIANKNPDNPKKTTTLSYDVYKD
KRFSEDQYELHIPIAINKCPKNIFKINTEVRVLLKHDDNPYVIGIDRGERNLLYIVVVDGKGNIVEQ
YSLNEIINNFNGIRIKTDYHSLLDKKEKERFEARQNWTSIENIKELKAGYISQVVHKICELVEKYDA
VIALEDLNSGFKNSRVKVEKQVYQKFEKMLIDKLNYMVDKKSNPCATGGALKGYQITNKFESFKSMS
TQNGFIFYIPAWLTSKIDPSTGFVNLLKTKYTSIADSKKFISSFDRIMYVPEEDLFEFALDYKNFSR
TDADYIKKWKLYSYGNRIRIFRNPKKNNVFDWEEVCLTSAYKELFNKYGINYQQGDIRALLCEQSDK
AFYSSFMALMSLMLQMRNSITGRTDVDFLISPVKNSDGIFYDSRNYEAQENAILPKNADANGAYNIA
RKVLWAIGQFKKAEDEKLDKVKIAISNKEWLEYAQTSVKH
E. coli optimized Lb Cpf1 with flanking NLS's, V5 tag and 6x
His-DNA
SEQ ID NO: 13
ATGGGTAAACCGATTCCGAATCCGCTGCTGGGTCTGGATAGCACCGCACCGAAAAAAAAACGTAAAG
TTGGTATTCATGGTGTTCCGGCAGCACTGAAAAACGTGGGTATTGATCGTCTGGATGTTGAAAAAGG
TCGCAAAAATATGAGCAAACTGGAAAAGTTCACCAACTGTTATAGCCTGAGCAAAACCCTGCGTTTT
AAAGCAATTCCGGTTGGTAAAACCCAAGAGAACATTGATAATAAACGCCTGCTGGTCGAAGATGAAA
AACGCGCTGAAGATTATAAAGGCGTGAAAAAACTGCTGGATCGCTATTATCTGAGCTTCATTAACGA
TGTGCTGCACAGCATTAAACTGAAGAACCTGAACAACTATATCAGCCTGTTTCGTAAAAAAACCCGC
ACCGAAAAAGAAAACAAAGAGCTGGAAAACCTGGAAATCAATCTGCGTAAAGAAATCGCCAAAGCGT
TTAAAGGTAACGAGGGTTATAAAAGCCTGTTCAAGAAAGACATCATCGAAACCATTCTGCCGGAATT
TCTGGATGATAAAGATGAAATTGCCCTGGTGAATAGCTTTAATGGCTTTACCACCGCATTTACCGGC
TTTTTTGATAATCGCGAAAACATGTTCAGCGAAGAAGCAAAAAGCACCAGCATTGCATTTCGCTGCA
TTAATGAAAATCTGACCCGCTACATTAGCAACATGGATATCTTTGAAAAAGTGGACGCGATCTTCGA
TAAACACGAAGTGCAAGAGATCAAAGAGAAAATCCTGAACAGCGATTATGACGTCGAAGATTTTTTT
GAAGGCGAGTTCTTTAACTTCGTTCTGACCCAAGAAGGTATCGACGTTTATAACGCAATTATTGGTG
GTTTTGTTACCGAAAGCGGTGAGAAAATCAAAGGCCTGAATGAATATATCAACCTGTATAACCAGAA
AACCAAACAGAAACTGCCGAAATTCAAACCGCTGTATAAACAGGTTCTGAGCGATCGTGAAAGCCTG
AGCTTTTATGGTGAAGGTTATACCAGTGATGAAGAGGTTCTGGAAGTTTTTCGTAACACCCTGAATA
AAAACAGCGAGATCTTTAGCAGCATCAAAAAGCTTGAGAAACTGTTCAAAAACTTTGATGAGTATAG
CAGCGCAGGCATCTTTGTTAAAAATGGTCCGGCAATTAGCACCATCAGCAAAGATATTTTTGGCGAA
TGGAATGTGATCCGCGATAAATGGAATGCCGAATATGATGATATCCACCTGAAAAAAAAGGCCGTGG
TGACCGAGAAATATGAAGATGATCGTCGTAAAAGCTTCAAGAAAATTGGTAGCTTTAGCCTGGAACA
GCTGCAAGAATATGCAGATGCAGATCTGAGCGTTGTGGAAAAACTGAAAGAAATCATCATTCAGAAG
GTGGACGAGATCTATAAAGTTTATGGTAGCAGCGAAAAACTGTTCGATGCAGATTTTGTTCTGGAAA
AAAGCCTGAAAAAGAATGATGCCGTTGTGGCCATTATGAAAGATCTGCTGGATAGCGTTAAGAGCTT
CGAGAATTACATCAAAGCCTTTTTTGGTGAGGGCAAAGAAACCAATCGTGATGAAAGTTTCTATGGC
GATTTTGTGCTGGCCTATGATATTCTGCTGAAAGTGGACCATATTTATGATGCCATTCGCAATTATG
TTACCCAGAAACCGTATAGCAAAGACAAGTTCAAACTGTACTTTCAGAACCCGCAGTTTATGGGTGG
TTGGGATAAAGATAAAGAAACCGATTATCGTGCCACCATCCTGCGTTATGGTAGTAAATACTATCTG
GCCATCATGGACAAAAAATACGCAAAATGCCTGCAGAAAATCGACAAAGATGATGTGAATGGCAACT
ATGAAAAAATCAACTACAAACTGCTGCCTGGTCCGAATAAAATGCTGCCGAAAGTGTTCTTTAGCAA
GAAATGGATGGCCTATTATAACCCGAGCGAGGATATTCAAAAGATCTACAAAAATGGCACCTTTAAA
AAGGGCGACATGTTCAATCTGAACGATTGCCACAAACTGATCGATTTCTTCAAAGATTCAATTTCGC
GTTATCCGAAATGGTCCAATGCCTATGATTTTAACTTTAGCGAAACCGAAAAATACAAAGACATTGC
CGGTTTTTATCGCGAAGTGGAAGAACAGGGCTATAAAGTGAGCTTTGAAAGCGCAAGCAAAAAAGAG
GTTGATAAGCTGGTTGAAGAGGGCAAACTGTATATGTTCCAGATTTACAACAAAGATTTTAGCGACA
AAAGCCATGGCACCCCGAATCTGCATACCATGTACTTTAAACTGCTGTTCGACGAAAATAACCATGG
TCAGATTCGTCTGAGCGGTGGTGCCGAACTGTTTATGCGTCGTGCAAGTCTGAAAAAAGAAGAACTG
GTTGTTCATCCGGCAAATAGCCCGATTGCAAACAAAAATCCGGACAATCCGAAAAAAACCACGACAC
TGAGCTATGATGTGTATAAAGACAAACGTTTTAGCGAGGATCAGTATGAACTGCATATCCCGATTGC
CATCAATAAATGCCCGAAAAACATCTTTAAGATCAACACCGAAGTTCGCGTGCTGCTGAAACATGAT
GATAATCCGTATGTGATTGGCATTGATCGTGGTGAACGTAACCTGCTGTATATTGTTGTTGTTGATG
GTAAAGGCAACATCGTGGAACAGTATAGTCTGAACGAAATTATCAACAACTTTAACGGCATCCGCAT
CAAAACCGACTATCATAGCCTGCTGGACAAGAAAGAAAAAGAACGTTTTGAAGCACGTCAGAACTGG
ACCAGTATTGAAAACATCAAAGAACTGAAAGCCGGTTATATTAGCCAGGTGGTTCATAAAATCTGTG
AGCTGGTAGAAAAATACGATGCAGTTATTGCACTGGAAGATCTGAATAGCGGTTTCAAAAATAGCCG
TGTGAAAGTCGAAAAACAGGTGTATCAGAAATTCGAGAAAATGCTGATCGACAAACTGAACTACATG
GTCGACAAAAAAAGCAATCCGTGTGCAACCGGTGGTGCACTGAAAGGTTATCAGATTACCAACAAAT
TTGAAAGCTTTAAAAGCATGAGCACCCAGAACGGCTTTATCTTCTATATTCCGGCATGGCTGACCAG
CAAAATTGATCCGAGCACCGGTTTTGTGAACCTGCTGAAAACAAAATATACCTCCATTGCCGACAGC
AAGAAGTTTATTAGCAGCTTTGATCGCATTATGTATGTTCCGGAAGAGGACCTGTTTGAATTCGCAC
TGGATTACAAAAATTTCAGCCGTACCGATGCCGACTACATCAAAAAATGGAAACTGTACAGCTATGG
TAACCGCATTCGCATTTTTCGCAACCCGAAGAAAAACAATGTGTTCGATTGGGAAGAAGTTTGTCTG
ACCAGCGCATATAAAGAACTTTTCAACAAATACGGCATCAACTATCAGCAGGGTGATATTCGTGCAC
TGCTGTGTGAACAGAGCGATAAAGCGTTTTATAGCAGTTTTATGGCACTGATGAGCCTGATGCTGCA
GATGCGTAATAGCATTACCGGTCGCACCGATGTGGATTTTCTGATTAGTCCGGTGAAAAATTCCGAT
GGCATCTTTTATGATAGCCGCAATTACGAAGCACAAGAAAATGCAATTCTGCCGAAAAACGCAGATG
CAAATGGTGCATATAACATTGCACGTAAAGTTCTGTGGGCAATTGGCCAGTTTAAGAAAGCAGAAGA
TGAGAAGCTGGACAAAGTGAAAATTGCGATCAGCAATAAAGAGTGGCTGGAATACGCACAGACCAGC
GTTAAACATCCGAAAAAAAAACGCAAAGTGCTCGAGCACCACCACCACCACCACTGA
Amino acid sequence for LbCpf1 fusion, with 5′-and 3′-flanking
NLS's, 5′-V5 tag and 3′-6x His, used for gene editing in both
E. coli and human cells
SEQ ID NO: 14
MGKPIPNPLLGLDSTAPKKKRKVGIHGVPAALKNVGIDRLDVEKGRKNMSKLEKFTNCYSLSKTLRF
KAIPVGKTQENIDNKRLLVEDEKRAEDYKGVKKLLDRYYLSFINDVLHSIKLKNLNNYISLFRKKTR
TEKENKELENLEINLRKEIAKAFKGNEGYKSLFKKDIIETILPEFLDDKDEIALVNSFNGFTTAFTG
FFDNRENMFSEEAKSTSIAFRCINENLTRYISNMDIFEKVDAIFDKHEVQEIKEKILNSDYDVEDFF
EGEFFNFVLTQEGIDVYNAIIGGFVTESGEKIKGLNEYINLYNQKTKQKLPKFKPLYKQVLSDRESL
SFYGEGYTSDEEVLEVERNTLNKNSEIFSSIKKLEKLFKNFDEYSSAGIFVKNGPAISTISKDIFGE
WNVIRDKWNAEYDDIHLKKKAVVTEKYEDDRRKSFKKIGSFSLEQLQEYADADLSVVEKLKEIIIQK
VDEIYKVYGSSEKLFDADFVLEKSLKKNDAVVAIMKDLLDSVKSFENYIKAFFGEGKETNRDESFYG
DFVLAYDILLKVDHIYDAIRNYVTQKPYSKDKFKLYFQNPQFMGGWDKDKETDYRATILRYGSKYYL
AIMDKKYAKCLQKIDKDDVNGNYEKINYKLLPGPNKMLPKVFFSKKWMAYYNPSEDIQKIYKNGTFK
KGDMFNLNDCHKLIDFFKDSISRYPKWSNAYDFNFSETEKYKDIAGFYREVEEQGYKVSFESASKKE
VDKLVEEGKLYMFQTYNKDFSDKSHGTPNLHTMYFKLLFDENNHGQIRLSGGAELFMRRASLKKEEL
VVHPANSPIANKNPDNPKKTTTLSYDVYKDKRFSEDQYELHIPIAINKCPKNIFKINTEVRVLLKHD
DNPYVIGIDRGERNLLYIVVVDGKGNIVEQYSLNEIINNFNGIRIKTDYHSLLDKKEKERFEARQNW
TSIENIKELKAGYISQVVHKICELVEKYDAVIALEDLNSGFKNSRVKVEKQVYQKFEKMLIDKLNYM
VDKKSNPCATGGALKGYQITNKFESFKSMSTQNGFIFYIPAWLTSKIDPSTGFVNLLKTKYTSIADS
KKFISSFDRIMYVPEEDLFEFALDYKNFSRTDADYIKKWKLYSYGNRIRIFRNPKKNNVFDWEEVCL
TSAYKELFNKYGINYQQGDIRALLCEQSDKAFYSSFMALMSLMLQMRNSITGRTDVDFLISPVKNSD
GIFYDSRNYEAQENAILPKNADANGAYNIARKVLWAIGQFKKAEDEKLDKVKIAISNKEWLEYAQTS
VKHPKKKRKVLEHHHHHH
Hs optimized Lb Cpf1 with flanking NLS's, V5 tag and 6x His-DNA
SEQ ID NO: 17
ATGGGCAAGCCCATTCCTAATCCTCTGCTGGGCCTCGACAGCACAGCCCCTAAGAAAAAGCGGAAAG
TGGGCATCCATGGCGTGCCAGCCGCTCTGAAGAATGTGGGCATCGACAGACTGGACGTGGAAAAGGG
CAGAAAGAACATGAGCAAGCTCGAGAAGTTCACCAACTGCTACAGCCTGAGCAAGACCCTGCGGTTC
AAGGCCATTCCTGTGGGCAAGACCCAAGAGAACATCGACAACAAGCGGCTGCTGGTGGAAGATGAGA
AGAGAGCCGAGGACTACAAGGGCGTGAAGAAGCTGCTGGACCGGTACTACCTGAGCTTCATCAACGA
CGTGCTGCACAGCATCAAGCTGAAGAACCTGAACAACTACATCAGCCTGTTCCGGAAGAAAACCCGG
ACCGAGAAAGAGAACAAAGAGCTGGAAAACCTCGAGATCAACCTGCGGAAAGAGATCGCCAAGGCCT
TCAAGGGCAACGAGGGCTACAAGAGCCTGTTCAAGAAGGACATCATCGAGACAATCCTGCCTGAGTT
CCTGGACGACAAGGACGAGATCGCCCTGGTCAACAGCTTCAACGGCTTCACAACCGCCTTCACCGGC
TTTTTCGACAACCGCGAGAATATGTTCAGCGAGGAAGCCAAGAGCACCTCTATCGCCTTCCGGTGCA
TCAACGAGAATCTGACCCGGTACATCAGCAACATGGATATCTTCGAGAAGGTGGACGCCATCTTCGA
CAAGCACGAGGTGCAAGAGATCAAAGAAAAGATCCTGAACAGCGACTACGACGTCGAGGACTTCTTC
GAGGGCGAGTTCTTCAACTTCGTGCTGACACAAGAGGGCATCGATGTGTACAACGCCATCATCGGCG
GCTTCGTGACAGAGAGCGGCGAGAAGATCAAGGGCCTGAACGAGTACATCAACCTCTACAACCAGAA
AACGAAGCAGAAGCTGCCCAAGTTCAAGCCCCTGTACAAACAGGTGCTGAGCGACAGAGAGAGCCTG
TCCTTTTACGGCGAGGGCTATACCAGCGACGAAGAGGTGCTGGAAGTGTTCAGAAACACCCTGAACA
AGAACAGCGAGATCTTCAGCTCCATCAAGAAGCTCGAAAAGCTGTTTAAGAACTTCGACGAGTACAG
CAGCGCCGGCATCTTCGTGAAGAATGGCCCTGCCATCAGCACCATCTCCAAGGACATCTTCGGCGAG
TGGAACGTGATCCGGGACAAGTGGAACGCCGAGTACGACGACATCCACCTGAAGAAAAAGGCCGTGG
TCACCGAGAAGTACGAGGACGACAGAAGAAAGAGCTTCAAGAAGATCGGCAGCTTCAGCCTGGAACA
GCTGCAAGAGTACGCCGACGCCGATCTGAGCGTGGTGGAAAAGCTGAAAGAGATTATCATCCAGAAG
GTCGACGAGATCTACAAGGTGTACGGCAGCAGCGAGAAGCTGTTCGACGCCGACTTTGTGCTGGAAA
AGAGCCTCAAAAAGAACGACGCCGTGGTGGCCATCATGAAGGACCTGCTGGATAGCGTGAAGTCCTT
CGAGAACTATATTAAGGCCTTCTTTGGCGAGGGCAAAGAGACAAACCGGGACGAGAGCTTCTACGGC
GATTTCGTGCTGGCCTACGACATCCTGCTGAAAGTGGACCACATCTACGACGCCATCCGGAACTACG
TGACCCAGAAGCCTTACAGCAAGGACAAGTTTAAGCTGTACTTCCAGAATCCGCAGTTCATGGGCGG
CTGGGACAAAGACAAAGAAACCGACTACCGGGCCACCATCCTGAGATACGGCTCCAAGTACTATCTG
GCCATTATGGACAAGAAATACGCCAAGTGCCTGCAGAAGATCGATAAGGACGACGTGAACGGCAACT
ACGAGAAGATTAACTACAAGCTGCTGCCCGGACCTAACAAGATGCTGCCTAAGGTGTTCTTTAGCAA
GAAATGGATGGCCTACTACAACCCCAGCGAGGATATCCAGAAAATCTACAAGAACGGCACCTTCAAG
AAAGGCGACATGTTCAACCTGAACGACTGCCACAAGCTGATCGATTTCTTCAAGGACAGCATCAGCA
GATACCCCAAGTGGTCCAACGCCTACGACTTCAATTTCAGCGAGACAGAGAAGTATAAGGATATCGC
CGGGTTCTACCGCGAGGTGGAAGAACAGGGCTATAAGGTGTCCTTTGAGAGCGCCAGCAAGAAAGAG
GTGGACAAGCTGGTCGAAGAGGGCAAGCTGTACATGTTCCAGATCTATAACAAGGACTTCTCCGACA
AGAGCCACGGCACCCCTAACCTGCACACCATGTACTTTAAGCTGCTGTTCGATGAGAACAACCACGG
CCAGATCAGACTGTCTGGCGGAGCCGAGCTGTTTATGAGAAGGGCCAGCCTGAAAAAAGAGGAACTG
GTCGTTCACCCCGCCAACTCTCCAATCGCCAACAAGAACCCCGACAATCCCAAGAAAACCACCACAC
TGAGCTACGACGTGTACAAGGATAAGCGGTTCTCCGAGGACCAGTACGAGCTGCACATCCCTATCGC
CATCAACAAGTGCCCCAAGAATATCTTCAAGATCAACACCGAAGTGCGGGTGCTGCTGAAGCACGAC
GACAACCCTTACGTGATCGGCATCGATCGGGGCGAGAGAAACCTGCTGTATATCGTGGTGGTGGACG
GCAAGGGCAATATCGTGGAACAGTACTCCCTGAATGAGATCATCAACAACTTCAATGGCATCCGGAT
CAAGACGGACTACCACAGCCTGCTGGACAAAAAAGAGAAAGAACGCTTCGAGGCCAGGCAGAACTGG
ACCAGCATCGAGAACATCAAAGAACTGAAGGCCGGCTACATCTCCCAGGTGGTGCACAAGATCTGCG
AGCTGGTTGAGAAGTATGACGCCGTGATTGCCCTGGAAGATCTGAATAGCGGCTTTAAGAACAGCCG
CGTGAAGGTCGAGAAACAGGTGTACCAGAAATTCGAGAAGATGCTGATCGACAAGCTGAACTACATG
GTCGACAAGAAGTCTAACCCCTGCGCCACAGGCGGAGCCCTGAAGGGATATCAGATCACCAACAAGT
TCGAGTCCTTCAAGAGCATGAGCACCCAGAATGGCTTCATCTTCTACATCCCCGCCTGGCTGACCAG
CAAGATCGATCCTAGCACCGGATTCGTGAACCTGCTCAAGACCAAGTACACCAGCATTGCCGACAGC
AAGAAGTTCATCTCCAGCTTCGACCGGATTATGTACGTGCCCGAAGAGGACCTGTTCGAATTCGCCC
TGGATTACAAGAACTTCAGCCGGACCGATGCCGACTATATCAAGAAGTGGAAGCTGTATAGCTACGG
CAACCGCATCCGCATCTTCAGAAACCCGAAGAAAAACAACGTGTTCGACTGGGAAGAAGTGTGCCTG
ACCAGCGCCTACAAAGAACTCTTCAACAAATACGGCATCAACTACCAGCAGGGCGACATCAGAGCCC
TGCTGTGCGAGCAGAGCGACAAGGCCTTTTACAGCTCCTTCATGGCCCTGATGAGCCTGATGCTGCA
GATGCGGAATAGCATCACCGGCAGAACCGACGTGGACTTCCTGATCAGCCCCGTGAAAAACTCCGAC
GGCATCTTTTACGACAGCCGGAATTACGAGGCTCAAGAGAACGCCATCCTGCCTAAGAACGCCGATG
CCAACGGCGCCTATAATATCGCCAGAAAGGTGCTGTGGGCCATCGGCCAGTTTAAGAAGGCCGAGGA
CGAGAAACTGGACAAAGTGAAGATCGCCATCTCTAACAAAGAGTGGCTGGAATACGCCCAGACCAGC
GTGAAGCACCCCAAAAAGAAACGGAAAGTGCTGGAACACCACCACCATCACCAC
E. coli optimized Lb Cpf1 with OpTNLS and 6x His-AA
SEQ ID NO: 20
MGDPLKNVGIDRLDVEKGRKNMSKLEKFTNCYSLSKTLRFKAIPVGKTQENIDNKRLLVEDEKRAED
YKGVKKLLDRYYLSFINDVLHSIKLKNLNNYISLFRKKTRTEKENKELENLEINLRKEIAKAFKGNE
GYKSLFKKDIIETILPEFLDDKDEIALVNSFNGFTTAFTGFFDNRENMFSEEAKSTSIAFRCINENL
TRYISNMDIFEKVDAIFDKHEVQEIKEKILNSDYDVEDFFEGEFFNFVLTQEGIDVYNAIIGGFVTE
SGEKIKGLNEYINLYNQKTKQKLPKFKPLYKQVLSDRESLSFYGEGYTSDEEVLEVERNTLNKNSEI
FSSIKKLEKLFKNFDEYSSAGIFVKNGPAISTISKDIFGEWNVIRDKWNAEYDDIHLKKKAVVTEKY
EDDRRKSFKKIGSFSLEQLQEYADADLSVVEKLKEIIIQKVDEIYKVYGSSEKLFDADFVLEKSLKK
NDAVVAIMKDLLDSVKSFENYIKAFFGEGKETNRDESFYGDFVLAYDILLKVDHIYDAIRNYVTQKP
YSKDKFKLYFQNPQFMGGWDKDKETDYRATILRYGSKYYLAIMDKKYAKCLQKIDKDDVNGNYEKIN
YKLLPGPNKMLPKVFFSKKWMAYYNPSEDIQKIYKNGTFKKGDMFNLNDCHKLIDFFKDSISRYPKW
SNAYDFNFSETEKYKDIAGFYREVEEQGYKVSFESASKKEVDKLVEEGKLYMFQTYNKDFSDKSHGT
PNLHTMYFKLLFDENNHGQIRLSGGAELFMRRASLKKEELVVHPANSPIANKNPDNPKKTTTLSYDV
YKDKRFSEDQYELHIPIAINKCPKNIFKINTEVRVLLKHDDNPYVIGIDRGERNLLYIVVVDGKGNI
VEQYSLNEIINNFNGIRIKTDYHSLLDKKEKERFEARQNWTSIENIKELKAGYISQVVHKICELVEK
YDAVIALEDLNSGFKNSRVKVEKQVYQKFEKMLIDKLNYMVDKKSNPCATGGALKGYQITNKFESFK
SMSTQNGFIFYIPAWLTSKIDPSTGFVNLLKTKYTSIADSKKFISSFDRIMYVPEEDLFEFALDYKN
FSRTDADYIKKWKLYSYGNRIRIFRNPKKNNVFDWEEVCLTSAYKELFNKYGINYQQGDIRALLCEQ
SDKAFYSSFMALMSLMLQMRNSITGRTDVDFLISPVKNSDGIFYDSRNYEAQENAILPKNADANGAY
NIARKVLWAIGQFKKAEDEKLDKVKIAISNKEWLEYAQTSVKHGRSSDDEATADSQHAAPPKKKRKV
LEHHHHHH
E. coli optimized Lb Cpf1 with OpTNLS and 6x His-DNA
SEQ ID NO: 23
ATGGGGGATCCACTGAAAAACGTGGGTATTGATCGTCTGGATGTTGAAAAAGGTCGCAAAAATATGA
GCAAACTGGAAAAGTTCACCAACTGTTATAGCCTGAGCAAAACCCTGCGTTTTAAAGCAATTCCGGT
TGGTAAAACCCAAGAGAACATTGATAATAAACGCCTGCTGGTCGAAGATGAAAAACGCGCTGAAGAT
TATAAAGGCGTGAAAAAACTGCTGGATCGCTATTATCTGAGCTTCATTAACGATGTGCTGCACAGCA
TTAAACTGAAGAACCTGAACAACTATATCAGCCTGTTTCGTAAAAAAACCCGCACCGAAAAAGAAAA
CAAAGAGCTGGAAAACCTGGAAATCAATCTGCGTAAAGAAATCGCCAAAGCGTTTAAAGGTAACGAG
GGTTATAAAAGCCTGTTCAAGAAAGACATCATCGAAACCATTCTGCCGGAATTTCTGGATGATAAAG
ATGAAATTGCCCTGGTGAATAGCTTTAATGGCTTTACCACCGCATTTACCGGCTTTTTTGATAATCG
CGAAAACATGTTCAGCGAAGAAGCAAAAAGCACCAGCATTGCATTTCGCTGCATTAATGAAAATCTG
ACCCGCTACATTAGCAACATGGATATCTTTGAAAAAGTGGACGCGATCTTCGATAAACACGAAGTGC
AAGAGATCAAAGAGAAAATCCTGAACAGCGATTATGACGTCGAAGATTTTTTTGAAGGCGAGTTCTT
TAACTTCGTTCTGACCCAAGAAGGTATCGACGTTTATAACGCAATTATTGGTGGTTTTGTTACCGAA
AGCGGTGAGAAAATCAAAGGCCTGAATGAATATATCAACCTGTATAACCAGAAAACCAAACAGAAAC
TGCCGAAATTCAAACCGCTGTATAAACAGGTTCTGAGCGATCGTGAAAGCCTGAGCTTTTATGGTGA
AGGTTATACCAGTGATGAAGAGGTTCTGGAAGTTTTTCGTAACACCCTGAATAAAAACAGCGAGATC
TTTAGCAGCATCAAAAAGCTTGAGAAACTGTTCAAAAACTTTGATGAGTATAGCAGCGCAGGCATCT
TTGTTAAAAATGGTCCGGCAATTAGCACCATCAGCAAAGATATTTTTGGCGAATGGAATGTGATCCG
CGATAAATGGAATGCCGAATATGATGATATCCACCTGAAAAAAAAGGCCGTGGTGACCGAGAAATAT
GAAGATGATCGTCGTAAAAGCTTCAAGAAAATTGGTAGCTTTAGCCTGGAACAGCTGCAAGAATATG
CAGATGCAGATCTGAGCGTTGTGGAAAAACTGAAAGAAATCATCATTCAGAAGGTGGACGAGATCTA
TAAAGTTTATGGTAGCAGCGAAAAACTGTTCGATGCAGATTTTGTTCTGGAAAAAAGCCTGAAAAAG
AATGATGCCGTTGTGGCCATTATGAAAGATCTGCTGGATAGCGTTAAGAGCTTCGAGAATTACATCA
AAGCCTTTTTTGGTGAGGGCAAAGAAACCAATCGTGATGAAAGTTTCTATGGCGATTTTGTGCTGGC
CTATGATATTCTGCTGAAAGTGGACCATATTTATGATGCCATTCGCAATTATGTTACCCAGAAACCG
TATAGCAAAGACAAGTTCAAACTGTACTTTCAGAACCCGCAGTTTATGGGTGGTTGGGATAAAGATA
AAGAAACCGATTATCGTGCCACCATCCTGCGTTATGGTAGTAAATACTATCTGGCCATCATGGACAA
AAAATACGCAAAATGCCTGCAGAAAATCGACAAAGATGATGTGAATGGCAACTATGAAAAAATCAAC
TACAAACTGCTGCCTGGTCCGAATAAAATGCTGCCGAAAGTGTTCTTTAGCAAGAAATGGATGGCCT
ATTATAACCCGAGCGAGGATATTCAAAAGATCTACAAAAATGGCACCTTTAAAAAGGGCGACATGTT
CAATCTGAACGATTGCCACAAACTGATCGATTTCTTCAAAGATTCAATTTCGCGTTATCCGAAATGG
TCCAATGCCTATGATTTTAACTTTAGCGAAACCGAAAAATACAAAGACATTGCCGGTTTTTATCGCG
AAGTGGAAGAACAGGGCTATAAAGTGAGCTTTGAAAGCGCAAGCAAAAAAGAGGTTGATAAGCTGGT
TGAAGAGGGCAAACTGTATATGTTCCAGATTTACAACAAAGATTTTAGCGACAAAAGCCATGGCACC
CCGAATCTGCATACCATGTACTTTAAACTGCTGTTCGACGAAAATAACCATGGTCAGATTCGTCTGA
GCGGTGGTGCCGAACTGTTTATGCGTCGTGCAAGTCTGAAAAAAGAAGAACTGGTTGTTCATCCGGC
AAATAGCCCGATTGCAAACAAAAATCCGGACAATCCGAAAAAAACCACGACACTGAGCTATGATGTG
TATAAAGACAAACGTTTTAGCGAGGATCAGTATGAACTGCATATCCCGATTGCCATCAATAAATGCC
CGAAAAACATCTTTAAGATCAACACCGAAGTTCGCGTGCTGCTGAAACATGATGATAATCCGTATGT
GATTGGCATTGATCGTGGTGAACGTAACCTGCTGTATATTGTTGTTGTTGATGGTAAAGGCAACATC
GTGGAACAGTATAGTCTGAACGAAATTATCAACAACTTTAACGGCATCCGCATCAAAACCGACTATC
ATAGCCTGCTGGACAAGAAAGAAAAAGAACGTTTTGAAGCACGTCAGAACTGGACCAGTATTGAAAA
CATCAAAGAACTGAAAGCCGGTTATATTAGCCAGGTGGTTCATAAAATCTGTGAGCTGGTAGAAAAA
TACGATGCAGTTATTGCACTGGAAGATCTGAATAGCGGTTTCAAAAATAGCCGTGTGAAAGTCGAAA
AACAGGTGTATCAGAAATTCGAGAAAATGCTGATCGACAAACTGAACTACATGGTCGACAAAAAAAG
CAATCCGTGTGCAACCGGTGGTGCACTGAAAGGTTATCAGATTACCAACAAATTTGAAAGCTTTAAA
AGCATGAGCACCCAGAACGGCTTTATCTTCTATATTCCGGCATGGCTGACCAGCAAAATTGATCCGA
GCACCGGTTTTGTGAACCTGCTGAAAACAAAATATACCTCCATTGCCGACAGCAAGAAGTTTATTAG
CAGCTTTGATCGCATTATGTATGTTCCGGAAGAGGACCTGTTTGAATTCGCACTGGATTACAAAAAT
TTCAGCCGTACCGATGCCGACTACATCAAAAAATGGAAACTGTACAGCTATGGTAACCGCATTCGCA
TTTTTCGCAACCCGAAGAAAAACAATGTGTTCGATTGGGAAGAAGTTTGTCTGACCAGCGCATATAA
AGAACTTTTCAACAAATACGGCATCAACTATCAGCAGGGTGATATTCGTGCACTGCTGTGTGAACAG
AGCGATAAAGCGTTTTATAGCAGTTTTATGGCACTGATGAGCCTGATGCTGCAGATGCGTAATAGCA
TTACCGGTCGCACCGATGTGGATTTTCTGATTAGTCCGGTGAAAAATTCCGATGGCATCTTTTATGA
TAGCCGCAATTACGAAGCACAAGAAAATGCAATTCTGCCGAAAAACGCAGATGCAAATGGTGCATAT
AACATTGCACGTAAAGTTCTGTGGGCAATTGGCCAGTTTAAGAAAGCAGAAGATGAGAAGCTGGACA
AAGTGAAAATTGCGATCAGCAATAAAGAGTGGCTGGAATACGCACAGACCAGCGTTAAACATGGTCG
TAGCAGTGATGATGAAGCAACCGCAGATAGCCAGCATGCAGCACCGCCGAAAAAAAAACGCAAAGTG
CTCGAGCACCACCACCACCACCACTGA
Hs optimized Lb Cpf1 with OpT NLS and 6x His-DNA
SEQ ID NO: 396
ATGCTGAAGAACGTGGGCATCGACCGGCTGGACGTGGAAAAGGGCAGAAAGAACATGAGCAAGCTCG
AGAAGTTCACCAACTGCTACAGCCTGAGCAAGACCCTGCGGTTCAAGGCCATTCCTGTGGGCAAGAC
CCAAGAGAACATCGACAACAAGCGGCTGCTGGTGGAAGATGAGAAGAGAGCCGAGGACTACAAGGGC
GTGAAGAAGCTGCTGGACCGGTACTACCTGAGCTTCATCAACGACGTGCTGCACAGCATCAAGCTCA
AGAACCTGAACAACTACATCAGCCTGTTCCGGAAGAAAACCCGGACCGAGAAAGAGAACAAAGAGCT
GGAAAACCTCGAGATCAACCTGCGGAAAGAGATCGCCAAGGCCTTCAAGGGCAACGAGGGCTACAAG
AGCCTGTTCAAGAAGGACATCATCGAGACAATCCTGCCTGAGTTCCTGGACGACAAGGACGAGATCG
CCCTGGTCAACAGCTTCAACGGCTTCACAACCGCCTTCACCGGCTTTTTCGACAACCGCGAGAATAT
GTTCAGCGAGGAAGCCAAGAGCACCTCTATCGCCTTCCGGTGCATCAACGAGAATCTGACCCGGTAC
ATCAGCAACATGGATATCTTCGAGAAGGTGGACGCCATCTTCGACAAGCACGAGGTGCAAGAGATCA
AAGAAAAGATCCTGAACAGCGACTACGACGTCGAGGACTTCTTCGAGGGCGAGTTCTTCAACTTCGT
GCTGACACAAGAGGGCATCGATGTGTACAACGCCATCATCGGCGGCTTCGTGACAGAGAGCGGCGAG
AAGATCAAGGGCCTGAACGAGTACATCAACCTCTACAACCAGAAAACGAAGCAGAAGCTGCCCAAGT
TCAAGCCCCTGTACAAACAGGTGCTGAGCGACAGAGAGAGCCTGTCCTTTTACGGCGAGGGCTATAC
CAGCGACGAAGAGGTGCTGGAAGTGTTCAGAAACACCCTGAACAAGAACAGCGAGATCTTCAGCTCC
ATCAAGAAGCTCGAAAAGCTGTTTAAGAACTTCGACGAGTACAGCAGCGCCGGCATCTTCGTGAAGA
ATGGCCCTGCCATCAGCACCATCTCCAAGGACATCTTCGGCGAGTGGAACGTGATCCGGGACAAGTG
GAACGCCGAGTACGACGACATCCACCTGAAGAAAAAGGCCGTGGTCACCGAGAAGTACGAGGACGAC
AGAAGAAAGAGCTTCAAGAAGATCGGCAGCTTCAGCCTGGAACAGCTGCAAGAGTACGCCGACGCCG
ATCTGAGCGTGGTGGAAAAGCTGAAAGAGATTATCATCCAGAAGGTCGACGAGATCTACAAGGTGTA
CGGCAGCAGCGAGAAGCTGTTCGACGCCGACTTTGTGCTGGAAAAGAGCCTCAAAAAGAACGACGCC
GTGGTGGCCATCATGAAGGACCTGCTGGATAGCGTGAAGTCCTTCGAGAACTATATTAAGGCCTTCT
TTGGCGAGGGCAAAGAGACAAACCGGGACGAGAGCTTCTACGGCGATTTCGTGCTGGCCTACGACAT
CCTGCTGAAAGTGGACCACATCTACGACGCCATCCGGAACTACGTGACCCAGAAGCCTTACAGCAAG
GACAAGTTTAAGCTGTACTTCCAGAATCCGCAGTTCATGGGCGGCTGGGACAAAGACAAAGAAACCG
ACTACCGGGCCACCATCCTGAGATACGGCTCCAAGTACTATCTGGCCATTATGGACAAGAAATACGC
CAAGTGCCTGCAGAAGATCGATAAGGACGACGTGAACGGCAACTACGAGAAGATTAACTACAAGCTG
CTGCCCGGACCTAACAAGATGCTGCCTAAGGTGTTCTTTAGCAAGAAATGGATGGCCTACTACAACC
CCAGCGAGGATATCCAGAAAATCTACAAGAACGGCACCTTCAAGAAAGGCGACATGTTCAACCTGAA
CGACTGCCACAAGCTGATCGATTTCTTCAAGGACAGCATCAGCAGATACCCCAAGTGGTCCAACGCC
TACGACTTCAATTTCAGCGAGACAGAGAAGTATAAGGATATCGCCGGGTTCTACCGCGAGGTGGAAG
AACAGGGCTATAAGGTGTCCTTTGAGAGCGCCAGCAAGAAAGAGGTGGACAAGCTGGTCGAAGAGGG
CAAGCTGTACATGTTCCAGATCTATAACAAGGACTTCTCCGACAAGAGCCACGGCACCCCTAACCTG
CACACCATGTACTTTAAGCTGCTGTTCGATGAGAACAACCACGGCCAGATCAGACTGTCTGGCGGAG
CCGAGCTGTTTATGAGAAGGGCCAGCCTGAAAAAAGAGGAACTGGTCGTTCACCCCGCCAACTCTCC
AATCGCCAACAAGAACCCCGACAATCCCAAGAAAACCACCACACTGAGCTACGACGTGTACAAGGAT
AAGCGGTTCTCCGAGGACCAGTACGAGCTGCACATCCCTATCGCCATCAACAAGTGCCCCAAGAATA
TCTTCAAGATCAACACCGAAGTGCGGGTGCTGCTGAAGCACGACGACAACCCTTACGTGATCGGCAT
CGACAGAGGCGAGCGGAACCTGCTGTATATCGTGGTGGTGGACGGCAAGGGCAATATCGTGGAACAG
TACTCCCTGAATGAGATCATCAACAACTTCAATGGCATCCGGATCAAGACGGACTACCACAGCCTGC
TGGACAAAAAAGAGAAAGAACGCTTCGAGGCCCGGCAGAACTGGACCAGCATCGAGAACATCAAAGA
ACTGAAGGCCGGCTACATCTCCCAGGTGGTGCACAAGATCTGCGAGCTGGTTGAGAAGTATGACGCC
GTGATTGCCCTGGAAGATCTGAATAGCGGCTTTAAGAACAGCCGCGTGAAGGTCGAGAAACAGGTGT
ACCAGAAATTCGAGAAGATGCTGATCGACAAGCTGAACTACATGGTCGACAAGAAGTCTAACCCCTG
CGCCACAGGCGGAGCCCTGAAGGGATATCAGATCACCAACAAGTTCGAGTCCTTCAAGAGCATGAGC
ACCCAGAATGGCTTCATCTTCTACATCCCCGCCTGGCTGACCAGCAAGATCGATCCTAGCACCGGAT
TCGTGAACCTGCTCAAGACCAAGTACACCAGCATTGCCGACAGCAAGAAGTTCATCTCCAGCTTCGA
CCGGATTATGTACGTGCCCGAAGAGGACCTGTTCGAATTCGCCCTGGATTACAAGAACTTCAGCCGG
ACCGATGCCGACTATATCAAGAAGTGGAAGCTGTATAGCTACGGCAACCGCATCCGCATCTTCAGAA
ACCCGAAGAAAAACAACGTGTTCGACTGGGAAGAAGTGTGCCTGACCAGCGCCTACAAAGAACTCTT
CAACAAATACGGCATCAACTACCAGCAGGGCGACATCAGAGCCCTGCTGTGCGAGCAGAGCGACAAG
GCCTTTTACAGCTCCTTCATGGCCCTGATGTCCCTGATGCTGCAGATGCGGAATAGCATCACCGGCA
GGACCGACGTGGACTTCCTGATCAGCCCTGTGAAGAATTCCGACGGGATCTTCTACGACAGCAGAAA
CTACGAGGCTCAAGAGAACGCCATCCTGCCTAAGAACGCCGATGCCAACGGCGCCTATAATATCGCC
AGAAAGGTGCTGTGGGCCATCGGCCAGTTTAAGAAGGCCGAGGACGAGAAACTGGACAAAGTGAAGA
TCGCCATCTCTAACAAAGAGTGGCTGGAATACGCCCAGACCAGCGTGAAGCACGGCAGATCTAGTGA
CGATGAGGCCACCGCCGATAGCCAGCATGCAGCCCCTCCAAAGAAAAAGCGGAAAGTGCTGGAACAC
CACCACCATCACCAC
Hs optimized Lb Cpf1 with OpTNLS and 6x His-AA
SEQ ID NO: 24
MLKNVGIDRLDVEKGRKNMSKLEKFTNCYSLSKTLRFKAIPVGKTQENIDNKRLLVEDEKRAEDYKG
VKKLLDRYYLSFINDVLHSIKLKNLNNYISLFRKKTRTEKENKELENLEINLRKEIAKAFKGNEGYK
SLFKKDIIETILPEFLDDKDEIALVNSFNGFTTAFTGFFDNRENMFSEEAKSTSIAFRCINENLTRY
ISNMDIFEKVDAIFDKHEVQEIKEKILNSDYDVEDFFEGEFFNFVLTQEGIDVYNAIIGGFVTESGE
KIKGLNEYINLYNQKTKQKLPKFKPLYKQVLSDRESLSFYGEGYTSDEEVLEVERNTLNKNSEIFSS
IKKLEKLFKNFDEYSSAGIFVKNGPAISTISKDIFGEWNVIRDKWNAEYDDIHLKKKAVVTEKYEDD
RRKSFKKIGSFSLEQLQEYADADLSVVEKLKEIIIQKVDEIYKVYGSSEKLFDADFVLEKSLKKNDA
VVAIMKDLLDSVKSFENYIKAFFGEGKETNRDESFYGDFVLAYDILLKVDHIYDAIRNYVTQKPYSK
DKFKLYFQNPQFMGGWDKDKETDYRATILRYGSKYYLAIMDKKYAKCLQKIDKDDVNGNYEKINYKL
LPGPNKMLPKVFFSKKWMAYYNPSEDIQKIYKNGTFKKGDMFNLNDCHKLIDFFKDSISRYPKWSNA
YDFNFSETEKYKDIAGFYREVEEQGYKVSFESASKKEVDKLVEEGKLYMFQTYNKDFSDKSHGTPNL
HTMYFKLLFDENNHGQIRLSGGAELFMRRASLKKEELVVHPANSPIANKNPDNPKKTTTLSYDVYKD
KRFSEDQYELHIPIAINKCPKNIFKINTEVRVLLKHDDNPYVIGIDRGERNLLYIVVVDGKGNIVEQ
YSLNEIINNFNGIRIKTDYHSLLDKKEKERFEARQNWTSIENIKELKAGYISQVVHKICELVEKYDA
VIALEDLNSGFKNSRVKVEKQVYQKFEKMLIDKLNYMVDKKSNPCATGGALKGYQITNKFESFKSMS
TQNGFIFYIPAWLTSKIDPSTGFVNLLKTKYTSIADSKKFISSFDRIMYVPEEDLFEFALDYKNFSR
TDADYIKKWKLYSYGNRIRIFRNPKKNNVFDWEEVCLTSAYKELFNKYGINYQQGDIRALLCEQSDK
AFYSSFMALMSLMLQMRNSITGRTDVDFLISPVKNSDGIFYDSRNYEAQENAILPKNADANGAYNIA
RKVLWAIGQFKKAEDEKLDKVKIAISNKEWLEYAQTSVKHGRSSDDEATADSQHAAPPKKKRKVLEH
HHHHH

### Example 10

Use of Modified crRNAs with LbCpf1 Protein Delivered as an RNP Complex.

Twelve sites in the human HPRT1 gene, 38094-S(SEQ ID No. 358), 38104-S (SEQ ID No. 361), 38115-AS (SEQ ID No. 364), 38146-AS (SEQ ID No. 367), 38164-AS (SEQ ID No. 370), 38164-S(SEQ ID No. 372), 38186-S(SEQ ID No. 376), 38228-S(SEQ ID No. 379), 38330-AS (SEQ ID No. 382), 38343-S(SEQ ID No. 385), 38455-S(SEQ ID No. 388) and 38486-S(SEQ ID No. 391) (where A and AS represent the sense and antisense strand, respectively), were chosen to study the target editing activity of LbCpf1, as compared to that of AsCpf1 and SpyCas9. Studies were done comparing the ability to use chemically modified crRNAs with LbCpf1 protein to perform genome editing in HEK-293 cells using electroporation to deliver the ribonucleoprotein protein (RNP) complexes into cells.

Purified recombinant LbCpf1 protein was employed in this example, isolated from E. coli using standard techniques. The amino-acid sequence of the recombinant protein is shown in SEQ ID NO:14.

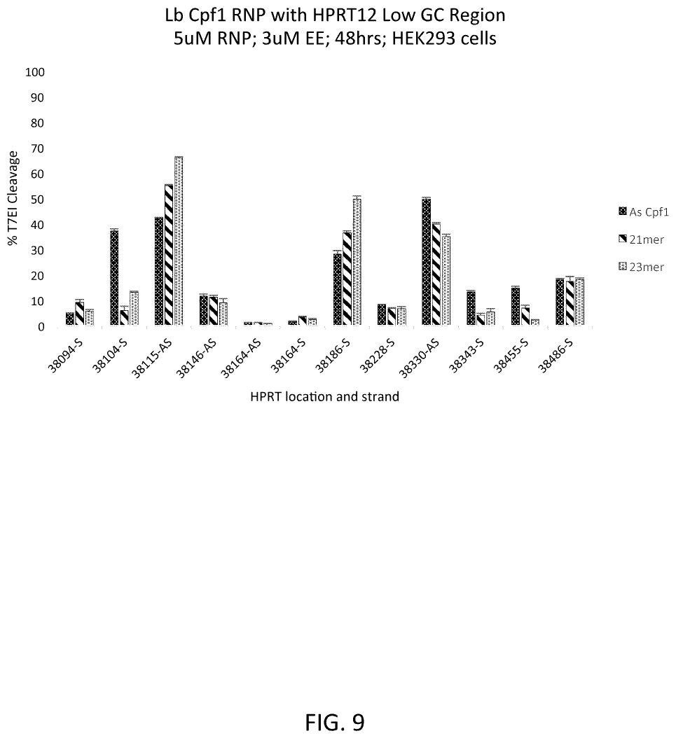

The LbCpf1 crRNAs, and AsCpf1 control crRNAs, were heated to 95° C. for 5 minutes then allowed to cool to room temperature. The crRNAs were mixed with LbCpf1, or AsCpf1, at a molar ratio of 1:1 RNA:protein in PBS (5 μM RNP complex in 10 μL volume, for a single transfection). The RNP complex was allowed to form at room temperature for 15 minutes. HEK293 cells were resuspended following trypsinization and washed in medium and washed a second time in PBS before use. Cells were resuspended in at a final concentration of 3.5×10⁵ cells in 20 μL of Nucleofection solution. 20 μL of cell suspension was placed in the V-bottom 96-well plate and 5 μL of the Cpf1 RNP complex was added to each well (5 M final concentration) and 3 μM of Cpf1 Electroporation Enhancer Solution was added to each well (Integrated DNA Technologies). 25 μL of the final mixture was transferred to each well of a 96 well Nucleocuvette electroporation module. Cells were electroporated using Amaxa 96 well shuttle protocol, program 96-DS-150. Following electroporation, 75 μL of medium was added to each well and 25 μL of the final cell mixture was transferred to 175 μL of pre-warmed medium in 96 well incubation plates (final volume 200 μL). Cells were incubated at 37° C. for 48 hours. Genomic DNA was isolated using QuickExtract solution (Epicentre). Genomic DNA was amplified with KAPA HiFi DNA Polymerase (Roche) and primers targeting the HPRT region of interest (HPRT-low forward primer: AAGAATGTTGTGATAAAAGGTGATGCT (SEQ ID No. 394); HPRT-low reverse primer: ACACATCCATGGGACTTCTGCCTC (SEQ ID No. 395)). PCR products were melted and re-annealed in NEB buffer 2 (New England Biolabs) to allow for heteroduplex formation followed by digestion with 2 units of T7 endonuclease 1 (T7EI; New England Biolabs) for 1 hour at 37° C. The digested products were visualized on a Fragment Analyzer (Advanced Analytical Technologies). Percent cleavage of targeted DNA was calculated as the average molar concentration of the cut products/(average molar concentration of the cut products+molar concentration of the uncut band)×100. The sequences are shown in Table 10, and the results are graphically represented in FIG. 9.

TABLE 10
Sequences of modified AsCpf1 and LbCpf1 crRNAs tested

| Seq Name | Sequence 5′-3′ | SEQ ID NO: |
|---|---|---|
| 38094-S-Control | C3-uaauuucuacucuuguagauauagucuuuccuugggugugu-C3 | 358 |
| 38094-S-21 | C3-uaauuucuacuaaguguagauauagucuuuccuugggugugu-C3 | 359 |
| 38094-S-23 | C3-uaauuucuacuaaguguagauauagucuuuccuuggguguguua-C3 | 360 |
| 38104-S-Cpf1 | C3-uaauuucuacucuuguagaucuuggguguguuaaaagugac-C3 | 361 |
| 38104-S-41-97 | C3-uaauuucuacuaaguguagaucuuggguguguuaaaagugac-C3 | 362 |
| 38104-S-23 | C3-uaauuucuacuaaguguagaucuuggguguguuaaaagugacca-C3 | 363 |
| 38115-AS-Cpf1 | C3-uaauuucuacucuuguagauacacacccaaggaaagacuau-C3 | 364 |
| 38115-AS-21 | C3-uaauuucuacuaaguguagauacacacccaaggaaagacuau-C3 | 365 |
| 38115-AS-23 | C3-uaauuucuacuaaguguagauacacacccaaggaaagacuauga-C3 | 366 |
| 38146-AS-Cpf1 | C3-uaauuucuacucuuguagauauccgugcugaguguaccaug-C3 | 367 |
| 38146-AS-21 | C3-uaauuucuacuaaguguagauauccgugcugaguguaccaug-C3 | 368 |
| 38146-AS-23 | C3-uaauuucuacuaaguguagauauccgugcugaguguaccaugca-C3 | 369 |
| 38164-AS-Cpf1 | C3-uaauuucuacucuuguagauuaaacacuguuucauuucauc-C3 | 370 |
| 38164-AS-21 | C3-uaauuucuacuaaguguagauuaaacacuguuucauuucauc-C3 | 371 |
| 38164-AS-23 | C3-uaauuucuacuaaguguagauuaaacacuguuucauuucauccg-C3 | 372 |
| 38164-S-Cpf1 | C3-uaauuucuacucuuguagaugaaacgucagucuucucuuuu-C3 | 373 |
| 38164-S-21 | C3-uaauuucuacuaaguguagaugaaacgucagucuucucuuuu-C3 | 374 |
| 38164-S-23 | C3-uaauuucuacuaaguguagaugaaacgucagucuucucuuuugu-C3 | 375 |
| 38186-S-Cpf1 | C3-uaauuucuacucuuguagauuaaugcccuguagucucucug-C3 | 376 |
| 38186-S-21 | C3-uaauuucuacuaaguguagauuaaugcccuguagucucucug-C3 | 377 |
| 38186-S-23 | C3-uaauuucuacuaaguguagauuaaugcccuguagucucucugua-C3 | 378 |
| 38228-S-Cpf1 | C3-uaauuucuacucuuguagauuaauuaacagcuugcugguga-C3 | 379 |
| 38228-S-21 | C3-uaauuucuacuaaguguagauuaauuaacagcuugcugguga-C3 | 380 |
| 38228-S-23 | C3-uaauuucuacuaaguguagauuaauuaacagcuugcuggugaaa-C3 | 381 |
| 38330-AS-Cpf1 | C3-uaauuucuacucuuguagaugguuaaagaugguuaaaugau-C3 | 382 |
| 38330-AS-21 | C3-uaauuucuacuaaguguagaugguuaaagaugguuaaaugau-C3 | 383 |
| 38330-AS-23 | C3-uaauuucuacuaaguguagaugguuaaagaugguuaaaugauug-C3 | 384 |
| 38343-S-Cpf1 | C3-uaauuucuacucuuguagauugugaaauggcuuauaauugc-C3 | 385 |
| 38343-S-21 | C3-uaauuucuacuaaguguagauugugaaauggcuuauaauugc-C3 | 386 |
| 38343-S-23 | C3-uaauuucuacuaaguguagauugugaaauggcuuauaauugcuu-C3 | 387 |
| 38455-S-Cpf1 | C3-uaauuucuacucuuguagauguuguuggauuugaaauucca-C3 | 388 |
| 38455-S-21 | C3-uaauuucuacuaaguguagauguuguuggauuugaaauucca-C3 | 389 |
| 38455-S-23 | C3-uaauuucuacuaaguguagauguuguuggauuugaaauuccaga-C3 | 390 |
| 38486-S-Cpf1 | C3-uaauuucuacucuuguagauuuguaggauaugcccuugacu-C3 | 391 |
| 38486-S-21 | C3-uaauuucuacuaaguguagauuuguaggauaugcccuugacu-C3 | 392 |
| 38486-S-23 | C3-uaauuucuacuaaguguagauuuguaggauaugcccuugacuau-C3 | 393 |

RNA bases are shown 5′-3′ orientation, RNA bases are shown in lower case. Locations are specified within the human HPRT1 gene with orientation relative to the sense coding strand indicated (S = sense, AS = antisense). C3 = C3 spacer (propanediol modifier). Cpf1 = Cpf1 crRNA control. 21 and 23 represent the length of the 3′ protospacer for each crRNA.

### Biological Material Deposit Information

The cell lines described herein (e.g., 1A1, 2A2 and 2B1) are deposited with the American Type Culture Collection (ATCC), located at 10801 University Blvd, Manassas, VA 20110, on ______ and assigned the following Accession Nos.: ______.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-24. (canceled)

25. An isolated nucleic acid, wherein the isolated nucleic acid encodes an Lb Cpf1 polypeptide codon optimized for expression in H. sapiens.

26. The isolated nucleic acid of claim 25, wherein the isolated nucleic acid comprises SEQ ID NO:17 or SEQ ID NO:396.

27. An isolated polypeptide comprising a wild-type Lp Cpf1 protein.

28. The isolated polypeptide of claim 27, wherein the isolated polypeptide comprises SEQ ID NO:14 or SEQ ID NO:24.

29. An isolated expression vector encoding SEQ ID NO:17 or SEQ ID NO:396.

30. A host cell comprising an isolated expression vector encoding SEQ ID NO:17 or SEQ ID NO:396, wherein the isolated expression vector encoding SEQ ID NO:17 or SEQ ID NO: 396 is operably linked to a suitable promoter to permit expression of a polypeptide comprising SEQ ID NO:14 or SEQ ID NO:24, respectively.

31. The host cell of claim 30, wherein host cell comprises a human cell.

32. (canceled)

33. (canceled)

34. The host cell line of claim 30, further comprising an isolated Lb Cpf1 crRNA capable of forming a ribonucleoprotein complex with the polypeptide selected from the group consisting of SEQ ID NO:4, SEQ ID NO: 14, SEQ ID NO:20 and SEQ ID NO:24 to form a wild-type CRISPR/Cpf1 endonuclease.

35. An isolated CRISPR/Cpf1 endonuclease system, comprising:
an Lb Cpf1 polypeptide, and
a suitable Cpf1 crRNA.

36. The isolated CRISPR/Cpf1 endonuclease system of claim 35, wherein the Lb Cpf1 polypeptide comprises SEQ ID NO: 14.

37. The isolated CRISPR/Cpf1 endonuclease system of claim 35, wherein the suitable Cpf1 crRNA is selected from a length-truncated Cpf1 crRNA or a chemically-modified Cpf1 crRNA, or a Cpf1 crRNA comprising both length truncations and chemical modifications.

38. An isolated CRISPR/Cpf1 endonuclease system, comprising:
a human cell line expressing a Lb Cpf1 polypeptide and a suitable Cpf1 crRNA.

39. The isolated CRISPR/Cpf1 endonuclease system of claim 38, wherein the Lb Cpf1 polypeptide comprises SEQ ID NO: 14 or SEQ ID NO:24.

40. The isolated CRISPR/Cpf1 endonuclease system of claim 38, wherein the suitable Cpf1 crRNA is selected from a length-truncated Cpf1 crRNA or a chemically-modified Cpf1 crRNA, or an Cpf1 crRNA comprising both length truncations and chemical modifications.

41. A method of performing gene editing, comprising:
contacting a candidate editing target site locus with an active CRISPR/Cpf1 endonuclease system having a wild-type Lb Cpf1 polypeptide and a suitable Cpf1 crRNA.

42. The method of claim 41, wherein the wild-type Lb Cpf1 polypeptide selected from the group consisting of SEQ ID NO:4, SEQ ID NO: 14, SEQ ID NO:20 and SEQ ID NO:24.

43. The method of claim 41, wherein the suitable Cpf1 crRNA is selected from a length-truncated Cpf1 crRNA, a chemically-modified Cpf1 crRNA, or an Cpf1 crRNA comprising both length truncations and chemical modifications.

44. The method of claim 41, wherein the suitable Cpf1 crRNA is a length-truncated Cpf1 crRNA comprising a 5′-universal loop domain of 19 to 20 nucleotides in length and a 3′-target specific protospacer domain of 19 to 21 nucleotides in length.

45. The method of claim 41, wherein the suitable Cpf1 crRNA comprises both a length truncation and a chemical modification.

46. The method of claim 45, wherein the chemical modification is selected from the group consisting of an end-group modification (e.g., C3 spacer), 2′OMe modification, 2′-fluoro modification and LNA modification.
